(19) United States
(12) Patent Application Publication
(10) Pub. No.: US 2024/0382629 A1
(43) Pub. Date: Nov. 21, 2024

(54) HETEROBIVALENT AND HOMOBIVALENT AGENTS TARGETING FIBROBLAST ACTIVATION PROTEIN ALPHA AND/OR PROSTATE-SPECIFIC MEMBRANE ANTIGEN

(71) Applicant: The Johns Hopkins University, Baltimore, MD (US)

(72) Inventors: Sangeeta Banerjee Ray, Ellicott City, MD (US); Srikanth Boinapally, Baltimore, MD (US); Martin Gilbert Pomper, Baltimore, MD (US); Andrew Horti, Baltimore, MD (US); Deepankar Das, Baltimore, MD (US); IL Minn, Baltimore, MD (US); Laurence Carroll, Baltimore, MD (US); Hyojin Cha, Baltimore, MD (US)

(21) Appl. No.: 18/553,092

(22) PCT Filed: Apr. 4, 2022
(86) PCT No.: PCT/US2022/023374

§ 371 Date: Sep. 28, 2023

Related U.S. Application Data
(60) Provisional application No. 63/170,035, filed on Apr. 2, 2021.

(51) Int. Cl. A61K 51/04; C07B 59/00

(52) U.S. Cl. CPC A61K 51/0455; A61K 51/0497; C07B 59/002; A61K 2121/00; A61K 2123/00; C07B 2200/05

(57) ABSTRACT

Imaging and radiotherapeutic agents targeting fibroblast-activation protein-α (FAP-α) and/or prostate-specific membrane antigen (PSMA) and their use in imaging and treating FAP-α and/or PSMA-related diseases and disorders are disclosed.

### STATEMENT OF GOVERNMENTAL INTEREST

This invention was made with government support under grants CA134675 and EB024495 awarded by the National Institutes of Health. The government has certain rights in the invention.

### BACKGROUND

Prostate-specific membrane antigen (PSMA) can act a target for a wide variety of cancers, including prostate cancer and clear cell renal carcinoma. In addition to being expressed in the tumor epithelium, PSMA also is expressed in the neovasculature of essentially all solid tumors. Fibroblast activation protein alpha (FAP-α), on the other hand, is expressed in cancer-associated fibroblasts, which are important promoters of the malignant phenotype and are likewise in nearly all cancers. Accordingly, PSMA and FAP-α can serve as markers for different aspects of cancer, i.e., epithelium and the tumor microenvironment. A platform targeting both PSMA and FAP-α simultaneously would not only be able to image, but if suitably functionalized with therapeutic agents, also would be able to treat cancers associated with PSMA and FAP-α in a superior fashion to any one agent alone.

### SUMMARY

In some aspects, the presently disclosed subject matter provides a compound of Formula (I):

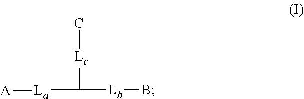

wherein:
A is a targeting moiety for fibroblast activation protein alpha (FAP-α);
B is a targeting moiety for prostate-specific membrane antigen (PSMA) or FAP-α, wherein if A and B are each a targeting moiety for FAP-α, they can be the same or different;
C is any optical or radiolabeled functional group suitable for optical imaging, photoacoustic imaging, positron emission tomography (PET) imaging, single-photon emission computed tomography (SPECT) imaging, or radiotherapy; and
Lₐ, L_{b}, and L_{c} are each a bi-functionalized linker capable of forming a chemical bond with each other and A, B, and C, respectively.

In other aspects, the presently disclosed subject matter provides a compound of formula (II):

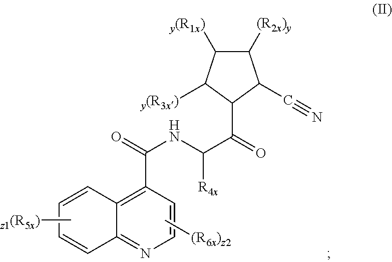

wherein each y is independently an integer selected from the group consisting of 0, 1, and 2;
R₁ₓ, R₂ₓ, and R_{3x′}, are each independently selected from the group consisting of H, OH, halogen, C₁₋₆alkyl, —O—C₁₋₆alkyl, and —S—C₁₋₆alkyl;
R₄ₓ is selected from the group consisting of H, straight-chain or branched C₁₋₆ alkyl, —(CH₂)_{q4}-aryl, and hydroxyl-substituted straight-chain or branched C₁₋₆ alkyl, wherein q4 is an integer selected from the group consisting of 0, 1, 2, 3, 4, 5, and 6;
R₅ₓ and R₆ₓ are each independently H, halogen, or —O—(CH₂)_{z3}—Xₐ, wherein z1 is an integer from 0 to 4, z2 is an integer from 0 to 2, z3 is an integer from 1 to 6, and Xₐ is halogen, provided that R₅ₓ and R₆ₓ cannot both be H at the same time; and stereoisomers and pharmaceutically acceptable salts thereof.

In other aspects, the presently disclosed subject matter provides a compound of formula (III):

In other embodiments, the presently disclosed subject matter provides a compound of formula (III):

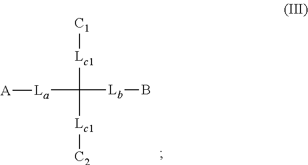

wherein:
A is a targeting moiety for fibroblast activation protein alpha (FAP-α);
B is absent or a targeting moiety for FAP-α, wherein A and B can be the same or different;
C₁ can be absent or present and when present is a chelating group;
C₂ is a prosthetic group;
Lₐ and L_{b} are each a bi-functionalized linker capable of forming a chemical bond with each other and A, B, C₁ and C₂;
L_{c1} and L_{c2} are each independently a bi-functionalized linker capable of forming a chemical bond with each other and A, B, Lₐ, and L_{b};
wherein if C₁ is absent, L_{c1} also is absent and wherein if B is absent, L_{b} also is absent;
and stereoisomers and pharmaceutically acceptable salts thereof.

In other aspects, the presently disclosed subject matter provides a compound of formula (IV):

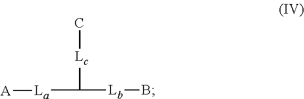

wherein:
A is a targeting moiety for fibroblast activation protein alpha (FAP-α);
B is a targeting moiety for prostate-specific membrane antigen (PSMA);
C is any optical or radiolabeled functional group suitable for optical imaging, photoacoustic imaging, positron emission tomography (PET) imaging, single-photon emission computed tomography (SPECT) imaging, or radiotherapy; and
Lₐ, L_{b}, and L_{c} are each a bi-functionalized linker capable of forming a chemical bond with each other and A, B, and C, respectively; and
stereoisomers and pharmaceutically acceptable salts thereof.

In other aspects, the presently disclosed subject matter provides a pharmaceutical composition comprising the compound of formula (I-IV). In some aspects, the pharmaceutical composition comprises one or more of pharmaceutically acceptable carriers, diluents, excipients, or adjuvants.

In other aspects, the presently disclosed subject matter provides a method for imaging a disease or disorder associated with fibroblast-activation protein-α (FAP-α) and/or prostate-specific membrane antigen (PSMA), the method comprising administering a compound of formula (I-IV), or a pharmaceutical composition thereof, wherein the compound of formula (I-IV) comprises an optical or radiolabeled functional group suitable for optical imaging, photoacoustic imaging, PET imaging, or SPECT imaging; and obtaining an image.

In other aspects, the presently disclosed subject matter provides a method for inhibiting fibroblast-activation protein-α (FAP-α) and/or prostate-specific membrane antigen (PSMA), the method comprising administering to a subject in need thereof an effective amount of a compound of formula (I-IV), or a pharmaceutical composition thereof.

In other aspects, the presently disclosed subject matter provides a method for treating a fibroblast-activation protein-α (FAP-α)- and/or a prostate-specific membrane antigen (PSMA)-related disease or disorder, the method comprising administering to a subject in need of treatment thereof an effective amount of a compound of formula (I-IV), or a pharmaceutical composition thereof, wherein the compound of formula (I-IV) comprises a radiolabeled functional group suitable for radiotherapy.

In some aspects, the (FAP-α)-related disease or disorder is selected from the group consisting of a proliferative disease; diseases characterized by tissue remodeling and/or chronic inflammation; disorders involving endocrinological dysfunction; and blood clotting disorders.

In certain aspects, the proliferative disease is selected from the group consisting of breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma.

In certain aspects, the prostate-specific membrane antigen (PSMA)-related disease or disorder is selected from the group consisting of prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature. In particular aspects, the prostate-specific membrane antigen (PSMA)-related disease or disorder comprises prostate cancer.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one figure executed in color. Copies of this patent or patent application publication with color figures will be provided by the Office upon request and payment of the necessary fee.

Having thus described the presently disclosed subject matter in general terms, reference will now be made to the accompanying Figures, which are not necessarily drawn to scale, and wherein:

FIG. 1 is the chemical structure of a representative compound of formula (I), e.g., [M]-SB-FAP-01, wherein M is ⁶⁸Ga, ²²⁵Ac, ¹⁷⁷Lu, and ⁶⁴Cu, having dual modalities for PSMA and FAP;

FIG. 2 is a scheme for the synthesis of FAP-Acetylene1;

FIG. 3 is a scheme for the synthesis of SB-FAP-01;

FIG. 4 is the HPLC report for SB-FAP-01;

FIG. 5 is the HRMS report for SB-FAP-01;

FIG. 6 shows HPLC chromatograms of ⁶⁸Ga-SB-FAP-01; radioactive peak at 31.1 min (bottom), UV peak at 29.9 min (λ=254 nm) is related to the unbound ligand SB-FAP-01 (top);

FIG. 7 demonstrates that compound SB-FAP-01, a heterobivalent ⁶⁸Ga-labeled imaging agent for positron emission tomography (PET), is able to image tumors derived from FAP+ HT-1080 human fibrosis cells engineered to overexpressed FAP, but not those which are not, i.e., wild type. Small animal PET/CT imaging was performed at 60 minutes after intravenous administration to this mirroring experimental model; and

FIG. 8 is the cell-binding data of SB-FAP-01 in PSMA+PIP cells (blue) and PSMA-flu cells (red) (uptake calculated as percent incubated dose (ID %) per 1 million cells).

FIG. 9A and FIG. 9B show structures of clinically relevant FAP-targeted scaffolds (FIG. 9A) and ⁶⁴Cu-FP-L1 and ⁶⁴Cu-FP-L2 (FIG. 9B);

FIG. 10A and FIG. 10B show (FIG. 10A) In cellulo binding specificity of ⁶⁴Cu-FP-L1 (mean±SD, n=3) in U87, SK-MEL-24 and isogenic PSMA+/PSMA− PC3 PIP/flu, PSMA+/PSMA− 786-O cells at 37□C. Data were obtained in two independent experiments with data presented from one. Receptor blocking studies were performed by co-incubation of either 10 μM FAPI-04 (for FAP blockade) or 10 μM ZJ43 (for PSMA blockade) to assess for binding specificity; (FIG. 10B) Cell surface FAP and PSMA expression by antibody-based flow cytometry illustrated by percentage of positive FAP and/or PSMA-expressing cells for the following lines: human U87 glioma (no staining for PSMA, high staining for FAP); human SK-MEL-24 melanoma (modest staining for PSMA, high staining for FAP); PC3 PIP (high staining for PSMA, no staining for FAP); PC3 flu (no staining for PSMA, no staining for FAP), PSMA+ 786-O (high staining for PSMA, low/no staining for FAP) and 786-O vector (no staining for PSMA, no staining for FAP). Data were obtained in three independent experiments with data presented from one;

FIG. 11A, FIG. 11B, FIG. 11C, FIG. 11D, FIG. 11E, and FIG. 11F show: FIG. 11A. Experiment 1: scheme; FIG. 11B, FIG. 11C. Quantitative PET/CT imaging and region-of-interest (ROI) analysis of ⁶⁴Cu-FP-L1 (7.4 MBq in 150 μL saline) in U87 tumor-bearing mice (n=3/timepoint). FIG. 11D. Biodistribution data are shown as percentage of injected dose per gram of tissue (% ID/g), mean±SD; FIG. 11E. In vivo specificity using either 10 nmol FAPI-04 (FAP blockade) or ZJ43 (PSMA blockade) per mouse, co-injection, 0.74 MBq in 150 μL saline (n=4); FIG. 11F. Head-to-head comparison H&E and IHC (at 10× magnification) of U87 tumor sections from the same cohort of mice displayed high FAP (brown staining) and no PSMA (no staining) expression, and in PSMA+PC3 PIP tumor with medium FAP and high PSMA expression. Significance was determined by the unpaired t test;

FIG. 12A, FIG. 12B, FIG. 12C, FIG. 12D, FIG. 12E, and FIG. 12F show FIG. 12A. Experiment 2: scheme; FIG. 12B. Quantitative PET imaging and region-of-interest (ROI) analysis of ⁶⁴Cu-FAP-L1 in SK-MEL-24 tumor-bearing mice (n=4/group). Tumor uptake is indicated with a black arrow. FIG. 12C. Receptor blockade: tumor (red), kidney (yellow) dotted area [coinjection of PSMA-targeted ZJ43 or co-injection of FAP-targeted FAPI-04 or autoblockade with FP-L1 (10 nmol per mouse) was performed at 30 min]. Absence of renal uptake upon administration of ZJ43 or non-radiolabeled FP-L1 indicates PSMA binding specificity; FIG. 12D. Quantitative depiction of PET/CT data; FIG. 12E. Mice were euthanized for biodistribution study at 2 h after imaging. Biodistribution data are shown as percentage of injected dose per gram of tissue (% ID/g), mean±SD. FIG. 12F. H&E for morphology (at 10× magnification) and brown staining (IHC) for FAP and PSMA expression from the same cohort of mice used in this experiment showing high FAP expression and no PSMA expression;

FIG. 13A, FIG. 13B, FIG. 13C, FIG. 13D, and FIG. 13E show whole-body PET imaging of a KPC mouse using ⁶⁴Cu-FP-L1 showing localization in pancreatic lesions. FIG. 13A. Experiment 3: scheme; FIG. 13B. Age-matched healthy littermate (left) and KPC mouse (right) after 2 h. Intense uptake in the abdominal area (red arrows), kidney (yellow dotted area) and salivary glands (red circle); FIG. 13C. (Left) Ex vivo near-infrared fluorescence imaging of selected tissues at 2 h after administration of IRDye800-FP-L1 showing intense uptake in pancreas (yellow dotted area), metastatic lesions (yellow arrows), and (right) a white light photograph of the tissues; FIG. 13D. Left to right: H&E (whole tissue and at 40× magnification) were acquired, demonstrating the presence of PDAC and PanIN lesions; FAP-positive IHC (10× magnification) of PDAC; and, FAP-negative healthy tissues. FIG. 13E. PSMA-specific staining of healthy pancreas and kidney and tumor tissue sections;

FIG. 14A, FIG. 14B, and FIG. 14C show: FIG. 14A. PET/CT imaging of ⁶⁴Cu-FP-L1 in PSMA+ 786-O (n=2) tumor-bearing mice shows intense uptake in the tumor (red) and kidney (yellow) dotted areas; FIG. 14B. Biodistribution data are shown as percentage of injected dose per gram of tissue (% ID/g), mean±SD (n=4); FIG. 14C. Left to right: H&E and IHC microscopic image of the tumor tissues from the same cohort of mice showing clear cell morphology (10× magnification) and positive (brown) staining for FAP and PSMA expression from the same cohort of mice, 10× magnification;

FIG. 15 is a schematic of a synthesis scheme for FP-L1;

FIG. 16 is a schematic of a synthesis scheme for FP-L2;

FIG. 17 shows dose dependent responses (sigmoid curves) of FP-L1 and FPL2 against designated substrates of PSMA (NAAG), FAP and PREP (Z-Gly-Pro-AMC) and DPPIV (H-Gly-Pro-AMC), respectively, in presence of either the corresponding recombinant enzymes for FAP, PREP and DPPIV and LNCaP cell lysate for PSMA;

FIG. 18 shows static PET imaging of U87 tumor bearing mice during 1-4 h post-injection after administration of ⁶⁴Cu-FP-L1. A blocking study also was performed using co-injection of 50 nmol of FAPI-04 showing significant reduction of tumor and non-specific tissue uptake. This study was done from the batch of mice with small tumor underwent biodistribution study at 2 h;

FIG. 19 shows sequential PET imaging and biodistribution study of ⁶⁴Cu-FPL2 at 24 h;

FIG. 20 shows the synthesis of IR800Dye-FP-L1; and

FIG. 21 shows the H&E staining of lung, liver and gall bladder of the KPC mouse.

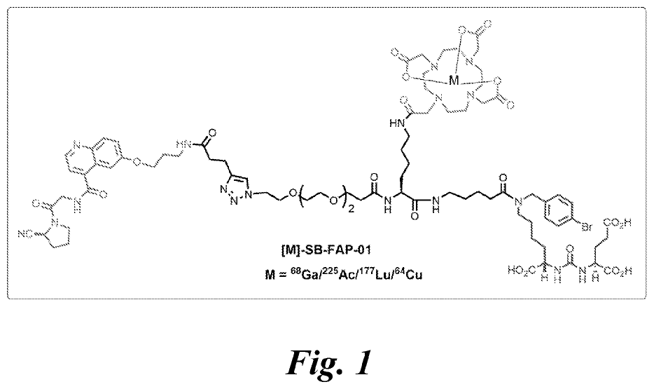

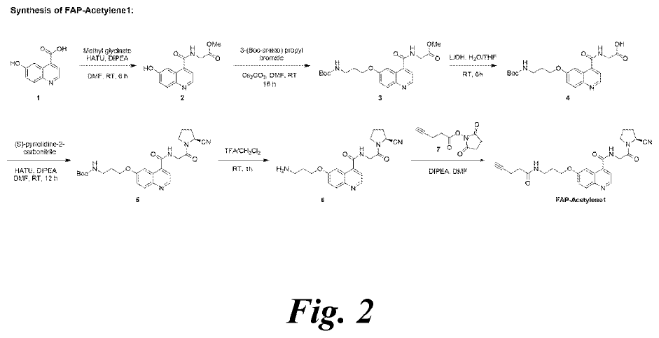

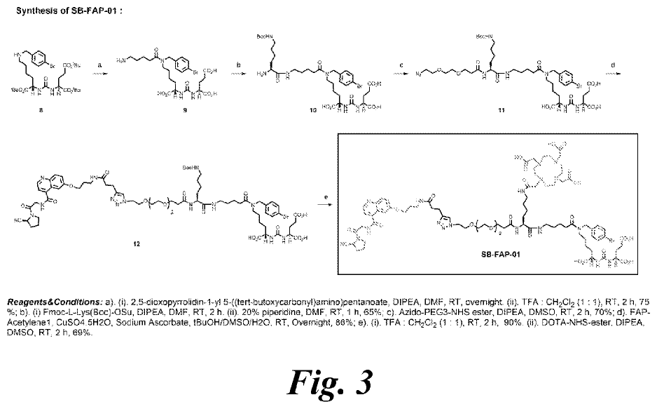

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Heterobivalent and Homobilvalent Agents Targeting Fibroblast Activation Protein Alpha and/or Prostate-Specific Membrane Antigen

Fibroblast-activation protein-α (FAP-α) is a type II integral membrane serine protease of the prolyl oligopeptidase family, which are distinguished by their ability to cleave the Pro-AA peptide bond (where AA represents any amino acid). FAP-α exists as a homodimer to carry out its enzymatic function. It has been shown to play a role in cancer by modifying bioactive signaling peptides through this enzymatic activity (Kelly, et al., 2005; Edosada, et al., 2006). FAP-α expression has been detected on the surface of fibroblasts in the stroma surrounding greater than 90% of the epithelial cancers, including, but not limited to, malignant breast, colorectal, skin, prostate, pancreatic cancers, and the like, and inflammation diseases, including, but not limited to, arthritis, fibrosis, and the like, with nearly no expression in healthy tissues. Inhibitors selectively targeting FAP-α has been reported (Lo, et al., 2009; Tsai, et al., 2010; Ryabtsova, et al., 2012; Poplawski, et al., 2013; Jansen, et al., 2013; Jansen, et al., 2014).

More particularly, FAP-α expression has been detected on the surface of fibroblasts in the stroma surrounding >90% of the epithelial cancers examined, including malignant breast, colorectal, skin, prostate, and pancreatic cancers. (Garin-Chesa, et al., 1990; Rettig, et al., 1993; Tuxhorn, et al., 2002; Scanlan, et al., 1994). It is a characteristic marker for carcinoma-associated-fibroblast (CAF), which plays a critical role in promoting angiogenesis, proliferation, invasion, and inhibition of tumor cell death. (Allinen, et al., 2004; Franco, et al., 2010). In healthy adult tissues, FAP-α expression is only limited to areas of tissue remodeling or wound healing. (Scanlan, et al., 1994; Yu, et al., 2010; Bae, et al., 2008; Kraman, et al., 2010). In addition, FAP-α-positive cells are observed during embryogenesis in areas of chronic inflammation, arthritis, and fibrosis, as well as in soft tissue and bone sarcomas. (Scanlan, et al., 1994; Yu, et al., 2010). These characteristics make FAP-α a potential imaging and radiotherapeutic target for cancer and inflammation diseases.

Because FAP-α is expressed in tumor stroma, anti-FAP antibodies have been investigated for radioimmunotargeting of malignancies, including murine F19, sibrotuzumab (a humanized version of the F19 antibody), ESC11, ESC14, and others. (Welt, et al., 1994; Scott, et al., 2003; Fischer, et al., 2012). Antibodies also demonstrated the feasibility of imaging inflammation, such as rheumatoid arthritis. (Laverman, et al., 2015). The use of antibodies as molecular imaging agents, however, suffers from pharmacokinetic limitations, including slow blood and non-target tissue clearance (normally 2-5 days or longer) and non-specific organ uptake. Low molecular weight (LMW) agents demonstrate faster pharmacokinetics and a higher specific signal within clinically convenient times after administration. They also can be synthesized in radiolabeled form more easily and may offer a shorter path to regulatory approval. (Coenen, et al., 2010; Coenen, et al., 2012; Reilly, et al., 2015). To date, however, no LMW ligand has been reported with ideal properties for nuclear imaging of FAP-α.

Likewise, the prostate-specific membrane antigen (PSMA) is a type II integral membrane protein expressed on the surface of prostate tumors, particularly in castrate-resistant, advanced, and metastatic disease (Huang, 2004; Schuelke, 2003). PSMA also is expressed in neovascular endothelium of most solid tumors, such as lung, colon, pancreatic, renal carcinoma, and skin melanoma, but not in normal vasculature (Liu, 1997; Chang, 1999), which makes it an excellent target for imaging and targeted therapy of these cancers.

The presently disclosed subject matter provides, in part, compound comprising a FAP-α selective targeting moiety and a PSMA selective targeting moiety that can be modified with an optical dye, a radiometal chelation complex, and other radiolabeled prosthetic groups, thus providing a platform for the imaging and radiotherapy targeting FAP-α and PSMA.

Radionuclide molecular imaging, including positron emission tomography (PET), is the most mature molecular imaging technique without tissue penetration limitations. Due to its advantages of high sensitivity and quantifiability, radionuclide molecular imaging plays an important role in clinical and preclinical research (Youn, et al., 2012; Chen, et al., 2014). Many radionuclides, primarily β- and alpha emitters, have been investigated for targeted radioimmunotherapy and include both radiohalogens and radiometals (see Table 1 for representative therapeutic radionuclides).

TABLE 1
Representative Therapeutic Radionuclides

| | |
|---|---|
| β-particle emitters | ⁹⁰Y, ¹³¹I, ¹⁷⁷Lu, ¹⁵³Sm, ¹⁸⁶Re, ¹⁸⁸Re, ⁶⁷Cu, ²¹²Pb |
| α-particle emitters | ²²⁵Ac, ²¹³Bi, ²¹²Bi, ²¹¹At, ²¹²Pb |
| Auger electron | ¹²⁵I, ¹²³I, ⁶⁷Ga, ¹¹¹In |
| emitters | |

Radioisotopes suitable for use with the presently disclosed subject matter also include, but are not limited to, ¹¹C, ¹⁸F, ⁵¹Cr, ⁶⁸Ga, ^{99m}Tc, ¹³⁰La, ¹⁴⁰La, ¹⁷⁵Yb, ¹⁵³Sm, ¹⁶⁶Ho, ⁸⁸Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ⁷²As, ⁷²Se, ⁹⁷Ru, ¹⁰⁹Pd, ¹⁰⁵Rh, ¹⁰¹mRh, ¹¹⁹Sb, ¹²⁸Ba, ¹²⁴I, ¹⁹⁷Hg, ¹⁵¹Eu, ¹⁵³Eu, ¹⁶⁹Eu, ²⁰¹Tl, ²⁰³Pb, ⁶⁴Cu, ¹⁹⁸Au, ²²⁵Ac, ²²⁷Th, and ¹⁹⁹Ag.

Fluorescent dyes suitable for use with the presently disclosed subject matter include, but are not limited to, xanthenes, acridines, oxazines, cyanines, styryl dyes, coumarines, porphines, fluorescent proteins, perylenes, boron-dipyrromethenes, and phtalocyanines.

The highly potent and specific binding moiety targeting FAP-α and PSMA enables its use in nuclear imaging and radiotherapy. The presently disclosed subject matter provides the first synthesis of nuclear imaging and radiotherapy agents based on this dual-targeting moiety to FAP-α and PSMA.

Accordingly, in some embodiments, the presently disclosed subject matter provides potent and selective low-molecular-weight (LMW) ligands of FAP-α, i.e., an FAP-α selective inhibitor and a PSMA selective inhibitor, conjugated with optical dyes or radiolabeling groups, including metal chelators and metal complexes, which enable in vivo optical imaging, nuclear imaging (optical, PET and SPECT), and radiotherapy targeting FAP-α and PSMA.

Importantly, the presently disclosed compounds can be modified, e.g., conjugated with, labeling groups without significantly losing their potency. The presently disclosed approach allows for the convenient labeling of the FAP-α ligand and PSMA ligand with optical dyes and PET or SPECT isotopes, including, but not limited to, ⁶⁸Ga, ⁶⁴Cu, ¹⁸F, ⁸⁶Y, ⁹⁰Y, ⁸⁹Zr, ¹¹¹In, ^{99m}Tc, ¹²⁵I, ¹²⁴I, for FAP-α and/or PSMA related imaging applications. Further, the presently disclosed approach allows for the radiolabeling of the FAP-α and PSMA targeting compound with radiotherapeutic isotopes, including but not limited to, ⁹⁰Y, ¹⁷⁷Lu, ¹²⁵I, ¹³¹I, ²¹¹At, ¹¹¹In, ¹⁵³Sm, ¹⁸⁶Re, ¹⁸⁸Re, ⁶⁷Cu, ²¹²Pb, ²²⁵Ac, ²¹³Bi, ²¹²Bi, ²¹²Pb, and ⁶⁷Ga, for FAP-α and/or PSMA related radiotherapy.

### A. Compounds

The presently disclosed subject matter provides a heterobivalent compound targeting the prostate-specific membrane antigen (PSMA) and fibroblast activation protein (FAP) that can serve as a platform for imaging and treating cancer. The imaging and therapeutic agents were developed independently targeting each of these cancer-associated proteins. Without wishing to be bound to any one particular theory, it is thought that it was necessary to combine agents targeting PSMA and FAP into one platform because they delineate different aspects of the tumor and its microenvironment.

The imaging aspect can involve a variety of modalities, including near-infrared optical imaging, positron emission tomography (PET), single photon emission computer tomography (SPECT) and magnetic resonance imaging (MRI), for example.

The therapeutic aspect can involve a variety of therapeutic nuclides, including, but not limited to, ¹⁷⁷Lu, ²¹¹At, ²²⁵Ac, among others. A chemical toxin also can be affixed to the platform.

In some embodiments, the presently disclosed subject matter provides a compound of Formula (I):

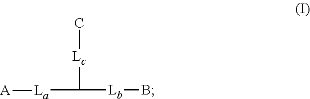

wherein:
A is a targeting moiety for fibroblast activation protein alpha (FAP-α);
B is a targeting moiety for prostate-specific membrane antigen (PSMA) or a targeting moiety for FAP-α, wherein if A and B are each a targeting moiety for FAP-α, they can be the same or different;
C is any optical or radiolabeled functional group suitable for optical imaging, photoacoustic imaging, positron emission tomography (PET) imaging, single-photon emission computed tomography (SPECT) imaging, or radiotherapy; and
Lₐ, L_{b}, and L_{c} are each a bi-functionalized linker capable of forming a chemical bond with each other and A, B, and C, respectively.

Suitable FAP-α specific targeting moieties are provided in International PCT Patent Application Publication No. WO2019/083990 A2 to Yang et al., for Imaging and Radiotherapeutic Agents Targeting Fibroblast-Activation Protein-α (FAP-α), published May 2, 2019, which is incorporated herein by reference in its entirety.

In certain embodiments, the targeting moiety for fibroblast activation protein alpha (FAP-α) has the following structure:

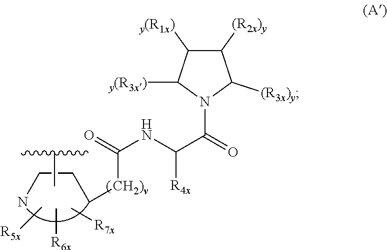

wherein each y is independently an integer selected from the group consisting of 0, 1, and 2;
R₁ₓ, R₂ₓ, and R_{3x′}, are each independently selected from the group consisting of H, OH, halogen, C₁₋₆alkyl, —O—C₁₋₆alkyl, and —S—C₁₋₆alkyl;
R₃ₓ is selected from the group consisting of H, —CN, —B(OH)₂, —C(O)alkyl, —C(O)aryl-, —C═C—C(O)aryl, —C—C—S(O)₂aryl, —CO₂H, —SO₃H, —SO₂NH₂, —PO₃H₂, and 5-tetrazolyl;
R₄ₓ is selected from the group consisting of H, straight-chain or branched C₁₋₆ alkyl, —(CH₂)_{q4}-aryl, and hydroxyl-substituted straight-chain or branched C₁₋₆ alkyl, wherein q4 is an integer selected from the group consisting of 0, 1, 2, 3, 4, 5, and 6;
R₅ₓ, R₆ₓ, and R₇ₓ are each independently selected from the group consisting of H, —OH, oxo, halogen, —C₁₋₆alkyl, —O—C₁₋₆alkyl, —S—C₁₋₆alkyl, —NR₈ₓR₉ₓ, —OR₁₂ₓ, -Het₂ and —Ar₂; each of C₁₋₆alkyl being optionally substituted with from 1 to 3 substituents selected from —OH and halogen; R₈ₓ, R₉ₓ, and R₁₂ₓ are each independently selected from the group consisting of H, —OH, halo, —C₁₋₆alkyl, —O—C₁₋₆alkyl, —S—C₁₋₆alkyl, and —Ar₃;
R₁₀ₓ, R₁₁ₓ, R₁₃ₓ and R₁₄ₓ are each independently selected from the group consisting of H, —OH, halogen, —C₁₋₆alkyl, —O—C₁₋₆alkyl, and —S—C₁₋₆alkyl; Ar₁, Ar₂ and Ar₃ are each independently a 5- or 6-membered aromatic monocycle optionally comprising 1 or 2 heteroatoms selected from O, N and S; each of Ar₁, Ar₂ and Ar₃ being optionally and independently substituted with from 1 to 3 substituents selected from —NR₁₀ₓR₁₁ₓ, —C₁₋₆alkyl, —O—C₁₋₆alkyl, and —S—C₁₋₆alkyl;
Het₂ is a 5- or 6-membered non-aromatic monocycle optionally comprising 1 or 2 heteroatoms selected from O, N and S; Het₂ being optionally substituted with from 1 to 3 substituents selected from —NR₁₃ₓR₁₄ₓ, —C₁₋₆alkyl, —O—C₁₋₆alkyl, and —S—C₁₋₆alkyl;
v is 0, 1, 2, or 3; and

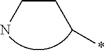 [SMILES: *CCCN]

represents a 5 to 10-membered N-containing aromatic or non-aromatic mono- or bicyclic heterocycle, said heterocycle optionally further comprising 1, 2 or 3 heteroatoms selected from O, N and S;
wherein [symbol] indicates a point of attachment of the FAP-α binding ligand to the linker, L₃, wherein the point of attachment can be through any of the carbon atoms of the 5 to 10-membered N-containing aromatic or non-aromatic mono- or bicyclic heterocycle thereof;
and stereoisomers and pharmaceutically acceptable salts thereof.

In some embodiments, the linker between the 5 to 10-membered N-containing aromatic or non-aromatic mono- or bicyclic heterocycle and the pyrrolidine ring is derived from an amino acid, e.g., glycine, alanine, phenyl alanine, valine, serine, and threonine:

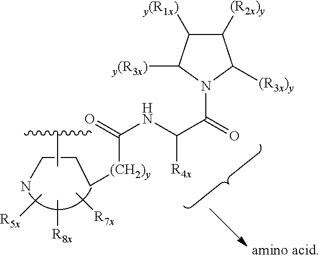

In particular embodiments, R₄ₓ is selected from the group consisting of H (glycine), —CH₃ (alanine), —CH₂-phenyl (phenyl alanine), —CH(CH₃)₂ (valine), —CH₂—OH (serine), and —CH(OH)CH₃ (threonine).

In certain embodiments,

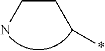 [SMILES: *CCCN]

is selected from the group consisting of:

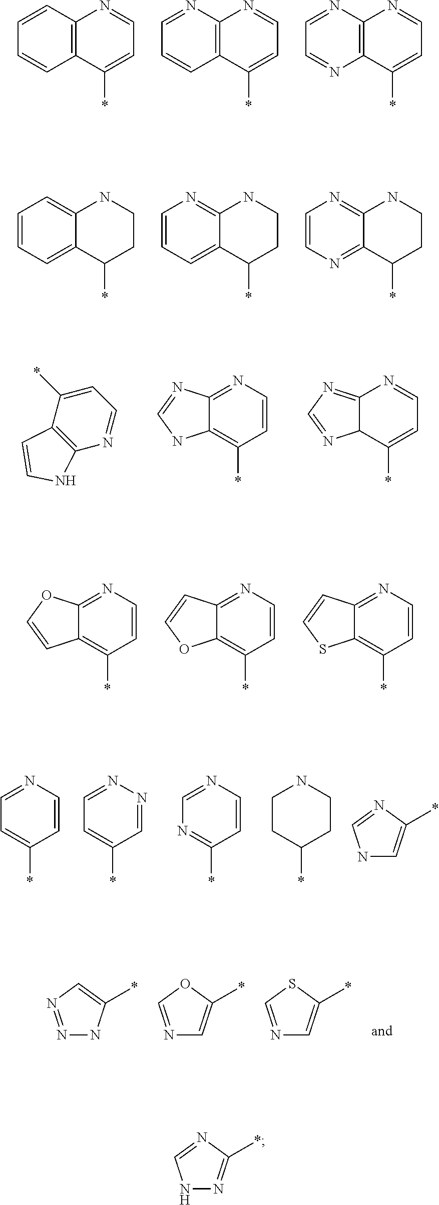

wherein * indicates the point of attachment of the 5 to 10-membered N-containing aromatic or non-aromatic mono- or bicyclic heterocycle to —(CH₂)ᵥ—.

In more certain embodiments, A is or A and B are an FAP-α targeting moiety having the structure of:

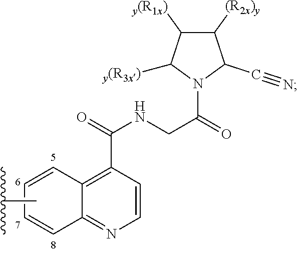

wherein [symbol] indicates a point of attachment of the FAP-α binding ligand to the linker, L₃, wherein the point of attachment can be through any of carbon atoms 5, 6, 7, or 8 of the quinolinyl ring thereof; and stereoisomers and pharmaceutically acceptable salts thereof.

In yet more certain embodiments, A is or A and B are each selected from the group consisting of:

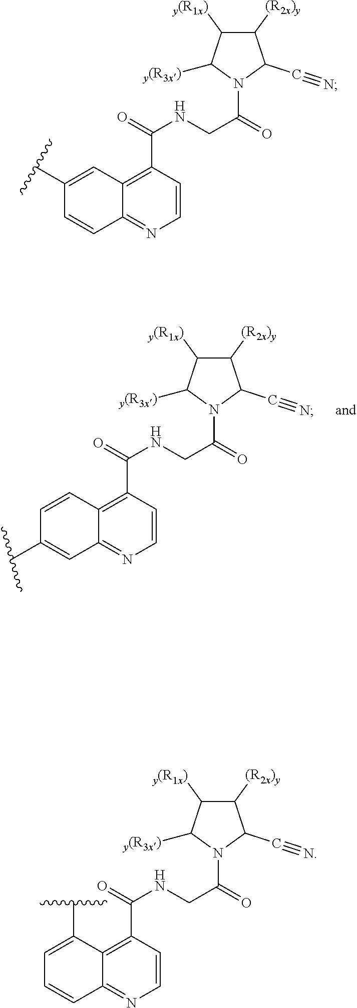

In even yet more certain embodiments, A is or A and B are each selected from the group consisting of:

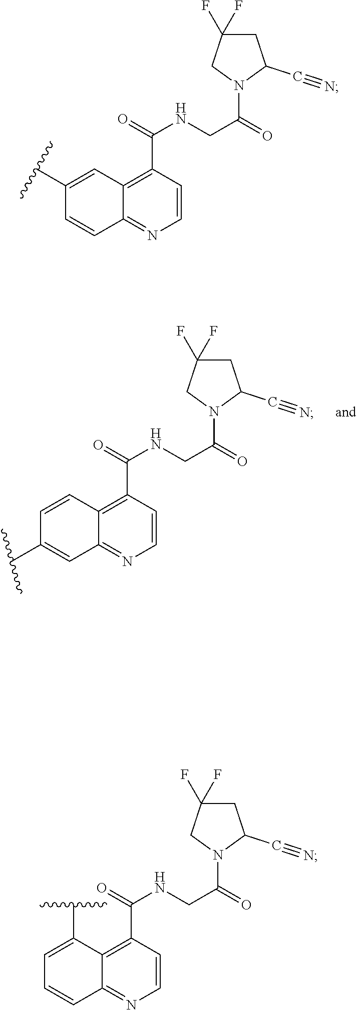 [SMILES: *c1ccc2nccc(C(=O)NCC(=O)N3CC(F)(F)CC3C#N)c2c1 ; *c1ccc2c(C(=O)NCC(=O)N3CC(F)(F)CC3C#N)ccnc2c1 ; *c1cccc2nccc(C(=O)NCC(=O)N3CC(F)(F)CC3C#N)c12]

and stereoisomers thereof.

In even yet more certain embodiments, A is or A and B are each selected from the group consisting of:

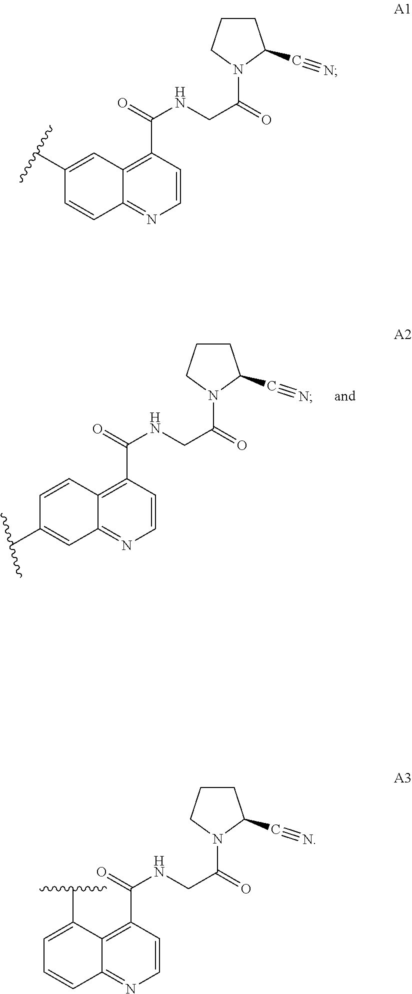 [SMILES: *c1ccc2nccc(C(=O)NCC(=O)N3CCC[C@H]3C#N)c2c1 ; *c1ccc2c(C(=O)NCC(=O)N3CCC[C@H]3C#N)ccnc2c1 ; *c1cccc2nccc(C(=O)NCC(=O)N3CCC[C@H]3C#N)c12]

Suitable FAP inhibitors are disclosed in International PCT Patent Application No. WO2019/154886 for FAP Inhibitor, to Haberkorn et al., published Aug. 15, 2019, which is incorporated herein by reference in its entirety. Representative FAP ligands, linkers, and reporting moieties include compounds of formula (I):

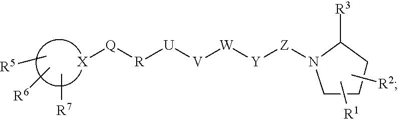

wherein:
Q, R, U, V, W, Y, Z are individually present or absent under the proviso that at least three of Q, R, U, V, W, Y, Z are present;
Q, R, U, V, W, Y, Z are independently selected form the group consisting of O, CH₂, NR⁴, C═O, C═S, C═NR⁴, HCR⁴ and R⁴CR⁴, with the proviso that two oxygen atoms are not directly adjacent to each other;
R¹ and R² are independently selected from the group consisting of —H, —OH, halo, C₁₋₆-alkyl, —O—C₁₋₆-alkyl, —S—C₁₋₆-alkyl;
R³ is selected from the group consisting of —H, —CN, —B(OH)₂, —C(O)-alkyl, —C(O)-aryl-, —C═C—C(O)-aryl, —C—C—S(O)₂-aryl, —CO₂H, —SO₃H, —SO₂H₂, —PO₃H₂, and 5-tetrazolyl;
R⁴ is selected from the group consisting of —H, —C₁₋₆-alkyl, —O—C₁₋₆-alkyl, —S—C₁₋₆-alkyl, alkenyl, heteroalkenyl, cycloalkenyl, cycloheteroalkenyl, alkynyl, aryl, and —C₁₋₆-aralkyl, each of said —C₁₋₆-alkyl being optionally substituted with from 1 to 3 substituents selected from —OH, oxo, halo and optionally connected to Q, R, U, V, W, Y or Z;
R⁵ is selected from the group consisting of —H, halo, and C₁₋₆-alkyl;
R⁶, and R⁷ are independently selected from the group consisting of —H,

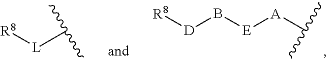

under the proviso that R⁶ and R⁷ are not at the same time H, wherein L is a linker, wherein D, A, E, and B are individually present or absent, preferably wherein at least A, E, and B are present, wherein when present:
D is a linker;
A is selected from the group consisting of NR⁴, O, S, and CH₂;
E is selected from the group consisting of C₁₋₆alkyl,

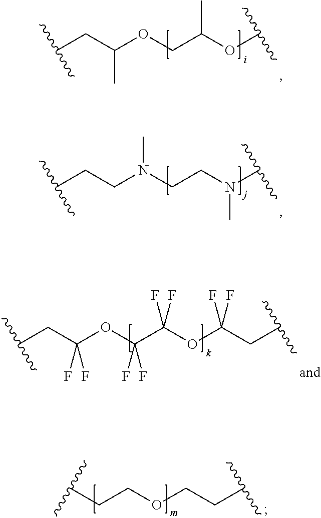

and
wherein i is 1, 2, or 3;
wherein j is 1, 2, or 3;
wherein k is 1, 2, or 3;
wherein m is 1, 2, or 3;
B is selected from the group consisting of S, NR⁴, NR⁴—O, NR⁴—C₁₋₆-alkyl, NR⁴—C₁₋₆-alkyl-NR⁴, and a 5- to 10-membered N-containing aromatic or non-aromatic mono- or bicyclic heterocycle, preferably further comprising 1 or 2 heteroatoms selected from O, N, and S, preferably further comprising 1 or 2 nitrogen atoms, preferably wherein NR⁴—C₁₋₆-alkyl-NR⁴ and the N-containing heterocycle is substituted with 1 to 3 substituents selected the group consisting of C₁₋₆-alkyl, aryl, C₁₋₆-aralkyl; and
R⁸ is selected from the group consisting of radioactive moiety, chelating agent, fluorescent dye, a contrast agent, and combinations thereof;

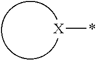

is a 1-naphthyl moiety or a 5 to 10-membered N-containing aromatic or non-aromatic mono- or bicyclic heterocycle, wherein there are 2 ring atoms between the N atom and X; said heterocycle optionally further comprising 1, 2 or 3 heteroatoms selected from ( ) N and S; and X is a C atom;
or a pharmaceutically acceptable tautomer, racemate, hydrate, solvate, or salt thereof.

In certain embodiments, A is an FAP-α targeting moiety or ligand having the structure of:

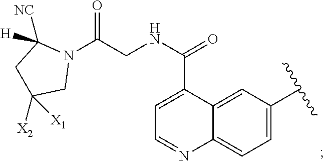

wherein X₁ and X₂ are each independently H or F.

In particular embodiments, the FAP inhibitor disclosed in WO2019/154886 is a compound, including the FAP ligand, linker, and reporting moiety, disclosed in one or more of Table 1, Table 2, Table 3, Table 4, and Table 5, or any compound disclosed on page 44, line 1, through page 75, line 6, which is incorporated herein by reference, including, but not limited to FAPI-1, FAPI-2, FAPI-3, FAPI-4, FAPI-5, FAPI-6, FAPI-7, FAPI-8, FAPI-9, FAPI-10, FAPI-11, FAPI-12, FAPI-13, FAPI-14, FAPI-15, FAPI-16, FAPI-17, FAPI-18, FAPI-19, FAPI-20, FAPI-21, FAPI-22, FAPI-23, FAPI-24, FAPI-25, FAPI-26, FAPI-27, FAPI-28, FAPI-29, FAPI-30, FAPI-31, FAPI-32, FAPI-33, FAPI-34, FAPI-35, FAPI-36, FAPI-37, FAPI-38, FAPI-39, FAPI-40, FAPI-41, FAPI-42, FAPI-43, FAPI-44, FAPI-45, FAPI-46, FAPI-47, FAPI-48, FAPI-49, FAPI-50, FAPI-51, FAPI-52, FAPI-53, FAPI-54, FAPI-55, FAPI-56, FAPI-57, FAPI-58, FAPI-60, FAPI-61, FAPI-62, FAPI-63, FAPI-64, FAPI-FAPI-65, FAPI-66, FAPI-67, FAPI-68, FAPI-69, FAPI-70, FAPI-71, FAPI-72, FAPI-73, FAPI-74, FAPI-75, FAPI-76, FAPI-77, FAPI-78, and FAPI-79, each of which incorporated herein by reference.

In some embodiments, the FAP-α ligand includes a substituted (4-Quinolinoyl)-glycyl-2-cyanopyrrolidine scaffold disclosed in Jansen et al., Selective Inhibitors of Fibroblast Activation Protein (FAP) with a (4-Quinolinoyl)-glycyl-2-cyanopyrrolidine Scaffold. ACS Med Chem Lett. 2013 Mar. 18; 4 (5): 491-6; Jansen et al., Extended structure-activity relationship and pharmacokinetic investigation of (4-quinolinoyl)glycyl-2-cyanopyrrolidine inhibitors of fibroblast activation protein (FAP). J Med Chem. 2014 Apr. 10; 57 (7): 3053-74, each of which is incorporated by reference in their entirety. Such FAP-α ligands include the following structure:

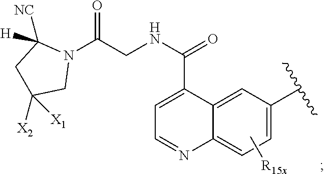

wherein:
   X₁ and X₂ are each independently H or F; and
   R₁₅ₓ is selected from the group consisting of H, C₁₋₆ alkyl, halogen, trihalomethoxyl, C₁₋₆ alkoxyl, and 4-methoxyphenyl.

Also included are FAP ligands disclosed in Roy et al., Design and validation of fibroblast activation protein alpha targeted imaging and therapeutic agents, Theranostics 2020, 10 (13), 5778-5789, which is incorporated herein by reference in its entirety, including, but not limited to:

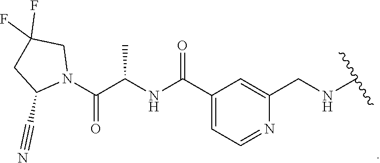 [SMILES: *NCc1cc(C(=O)N[C@@H](C)C(=O)N2CC(F)(F)C[C@H]2C#N)ccn1]

In certain embodiments, A is or A and B are each an FAP-α targeting moiety having the structure of:

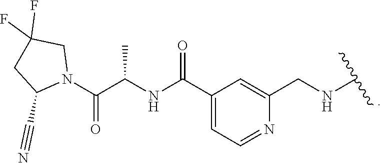 [SMILES: *NCc1cc(C(=O)N[C@@H](C)C(=O)N2CC(F)(F)C[C@H]2C#N)ccn1]

In other embodiments, B is a targeting moiety for FAP-α having the following structure:

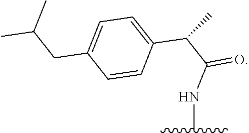 [SMILES: *NC(=O)[C@@H](C)c1ccc(CC(C)C)cc1]

In certain embodiments, B is a PSMA targeting moiety having the following structure:

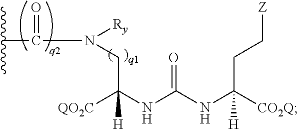

wherein:
   Z is tetrazole or CO₂Q;
   Q is H or a protecting group;
   q₁ is an integer selected from the group consisting of 1, 2, 3, 4, and 5;
   q₂ is an integer from selected from 0 or 1; and
   R_{y} is independently H or —(CH₂)_{q3}—R_{y1}, wherein q₃ is an integer selected from the group consisting of 0, 1, 2, 3, 4, 5, and 6 and R_{y1} is selected from the group consisting of substituted aryl, substituted pyridine, and unsubstituted isoquinoline;

In more certain embodiments, B is a PSMA targeting moiety having the following structure:

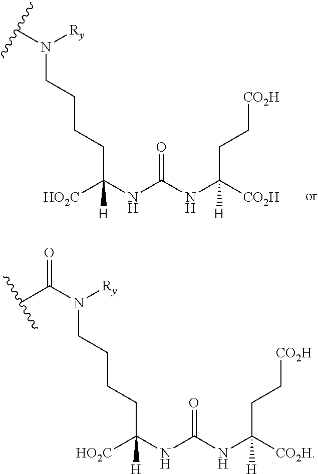

In certain embodiments, R_{y}1 is selected from the group consisting of:

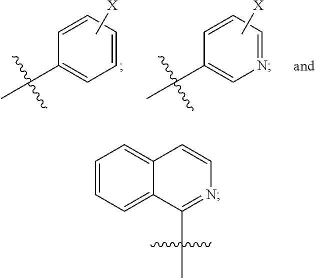

wherein X is independently selected from the group consisting of Br, ⁷⁵Br, ⁷⁶Br, ⁷⁷Br, ^{80m}Br, ⁸²Br, I, ¹²⁴I, ¹²³I, ¹²⁵I, ¹³¹I, At, and ²¹¹At.

Suitable linkers are disclosed in U.S. Patent Application Publication No. US2011/0064657 A1, for “Labeled Inhibitors of Prostate Specific Membrane Antigen (PSMA), Biological Evaluation, and Use as Imaging Agents,” published Mar. 17, 2011, to Pomper et al., and U.S. Patent Application Publication No. US2012/0009121 A1, for “PSMA-Targeting Compounds and Uses Thereof,” published Jan. 12, 2012, to Pomper et al, each of which is incorporated by reference in its entirety.

In certain embodiments, Lₐ, L_{b}, and L_{c} are each individually selected from the group consisting of (a), (b), (c), or (d):
(a)

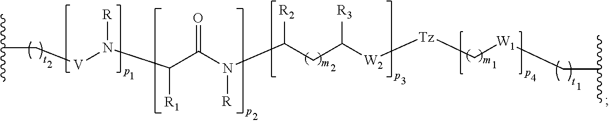

wherein:
p₁, p₂, p₃ and p₄ may be in any order;
t₁ and t₂ are each an integer selected from the group consisting of 0, 1, 2, 3, 4, 5, 6, 7, and 8;
p₁, p₃, and p₄ are each independently 0 or 1;
p₂ is an integer selected from the group consisting of 0, 1, 2, and 3, and when p₂ is 2 or 3, each R¹ is the same or different;
m₁ and m₂ are each an integer independently selected from the group consisting of 0, 1, 2, 3, 4, 5, 6, 7 and 8;
W¹ is selected from the group consisting of a bond, —S—, —C(═O)—, —C(═O)—NR—, and —NR—C(═O);
W² is selected from the group consisting of a bond, —S—, —CH₂—C(═O)—NR—, —C(═O), —NRC(═O)—, —NR′C(═O)NR—, —NRC(═S)NR′₂—, —NRC(═O)O—, —OC(═O)NR—, —OC(═O), —C(═O)NR—, —NR—C(═O)—, —C(═O)O—, —(O—CH₂CH₂)_{q}— and —(CH₂—CH₂—O)_{q}, wherein q is selected from the group consisting of 1, 2, 3, 4, 5, 6, 7, and 8;
each R or R′ is independently H, alkyl, substituted alkyl, cycloalkyl, substituted cycloalkyl, heterocycloalkyl, substituted heterocycloalkyl, aryl, substituted aryl, heteroaryl, substituted heteroaryl, and —OR₄, wherein R₄ is selected from the group consisting of H, alkyl, substituted alkyl, cycloalkyl, substituted cycloalkyl, heterocycloalkyl, and substituted heterocycloalkyl, wherein q is defined as immediately hereinabove;
Tz is a triazole group that can be present or absent and, if present, is selected from the group consisting of

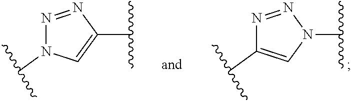 [SMILES: *c1cn(*)nn1 ; *c1cn(*)nn1]

each R₁ is independently H, C₁-C₆ alkyl, C₃-C₁₂ aryl, —(CH₂)_{q}—C₃-C₁₂ aryl, —C₄-C₁₆ alkylaryl, or —(CH₂)_{q}—C₄-C₁₆ alkylaryl; R₂ and R₃ are each independently H and —CO₂R⁵, wherein R₅ is selected from the group consisting of H, C₁-C₆ alkyl, C₃-C₁₂ aryl, and C₄-C₁₆ alkylaryl, wherein when one of R₂ or R₃ is CO₂R₅, then the other is H;
V is selected from the group consisting of —C(O)—, —C(S)—, —NRC(O)—, —NRC(S)—, and —OC(O)—;
(b)

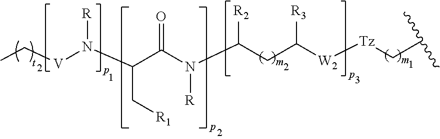

wherein p₁, p₂, p₃, m₁, m₂, Tz, W₂, R, R₁, R₂, R₃, and V are defined as hereinabove;
(c) -L₁-, -L₂-L₃-, or -L₁-L₂-L₃-, wherein:
L₁ is —NR—(CH₂)_{q}—[O—CH₂—CH₂—O]_{q}—(CH₂)_{q}—C(═O)—;
L₂ is —NR—(CH₂)_{q}—C(COOR₅)—NR—; and
L₃ is —(O═)C—(CH₂)_{q}—C(═O)—;
   wherein each q is independently an integer selected from the group consisting of 1, 2, 3, 4, 5, 6, 7, and 8; and R and R₅ are as defined hereinabove; and
(d) —(CR₆H)_{q}—(CH₂)_{q}—C(═O)—NR—(CH₂)_{q}—O— or —NR—(CH₂)_{q}—O—; wherein each q and R is defined hereinabove; and R⁶ is H or —COOR₅.

In more certain embodiments, one or more of Lₐ, L_{b}, and L_{c} is selected from the group consisting of:

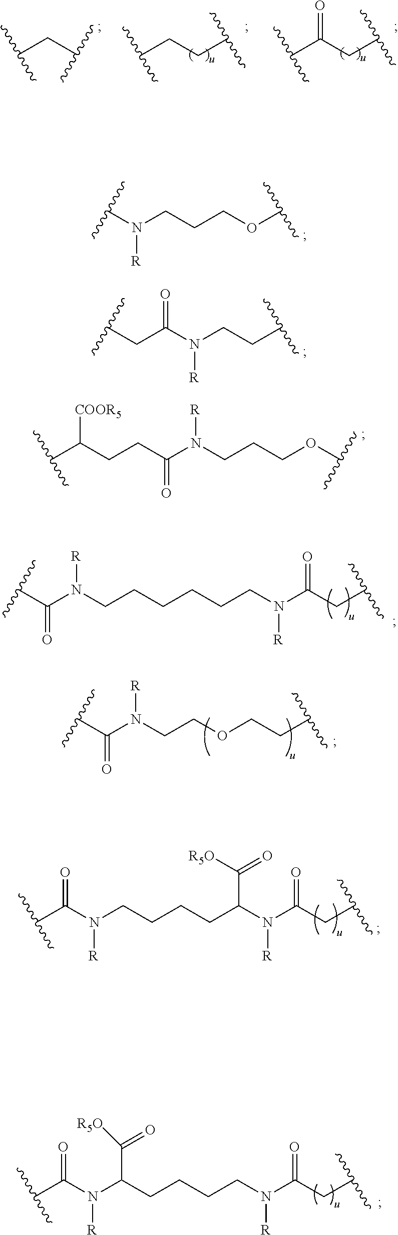

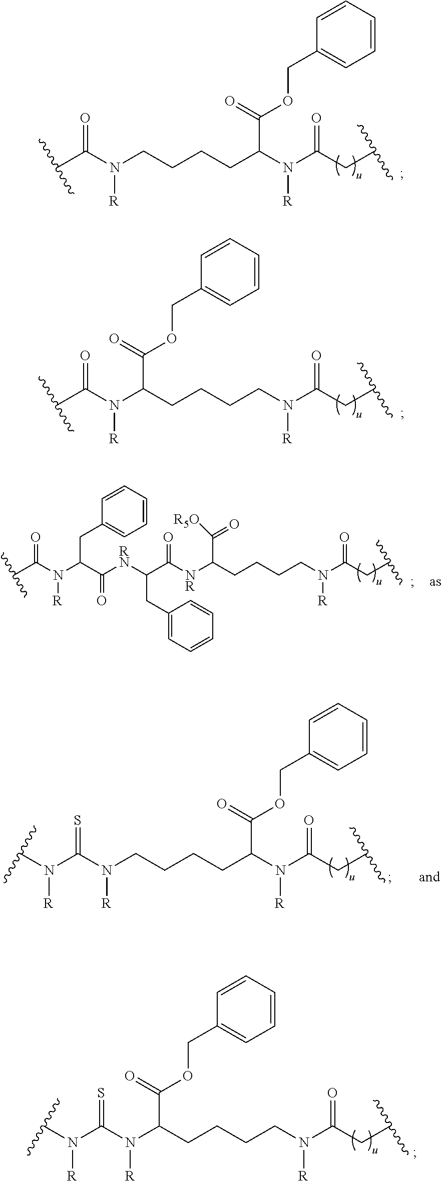

wherein u is an integer selected from 1, 2, 3, 4, 5, 6, 7, and 8; and R and R₅ are as defined hereinabove.

In certain embodiments, C is a radiolabeled prosthetic group comprising a radioisotope selected from the group consisting of ¹⁸F, ¹²⁴I, ¹²⁵I, ¹³¹I, and ²¹¹At.

In more certain embodiments, the radiolabeled prosthetic group is selected from the group consisting of:

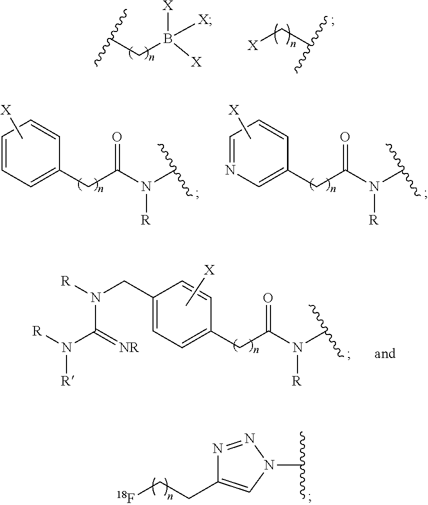

wherein each X is independently a radioisotope selected from the group consisting of ¹⁸F, ¹²⁴I, ¹²⁵I, ¹³¹I, and ²¹¹At; each R and R′ is defined hereinabove; and each n is independently an integer selected from the group consisting of 0, 1, 2, 3, 4, 5, 6, 7, 8, 9, 10, 11, 12, 13, 14, 15, 16, 17, 18, 19, and 20.

In yet more certain embodiments, the radiolabeled prosthetic group is selected from the group consisting of:

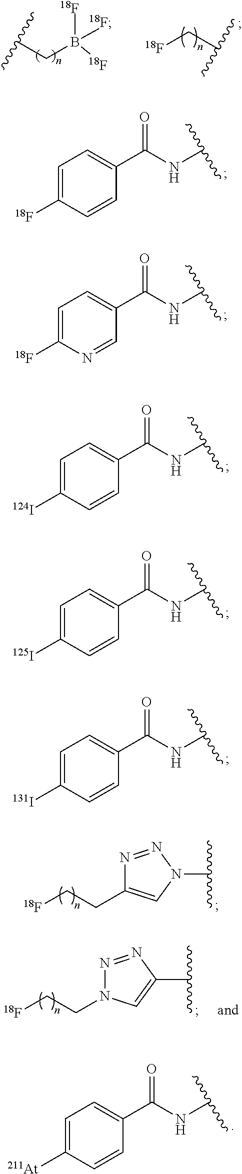

In certain embodiments, C comprises a chelating agent. In more certain embodiments, the chelating agent is selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), (NOTAGA) 1,4,7-triazonane-1,4-diyl)diacetic acid DFO (Desferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino)methyl) benzoic acid), and BaBaSar.

In yet more certain embodiments, the chelating agent is selected from the group consisting of:

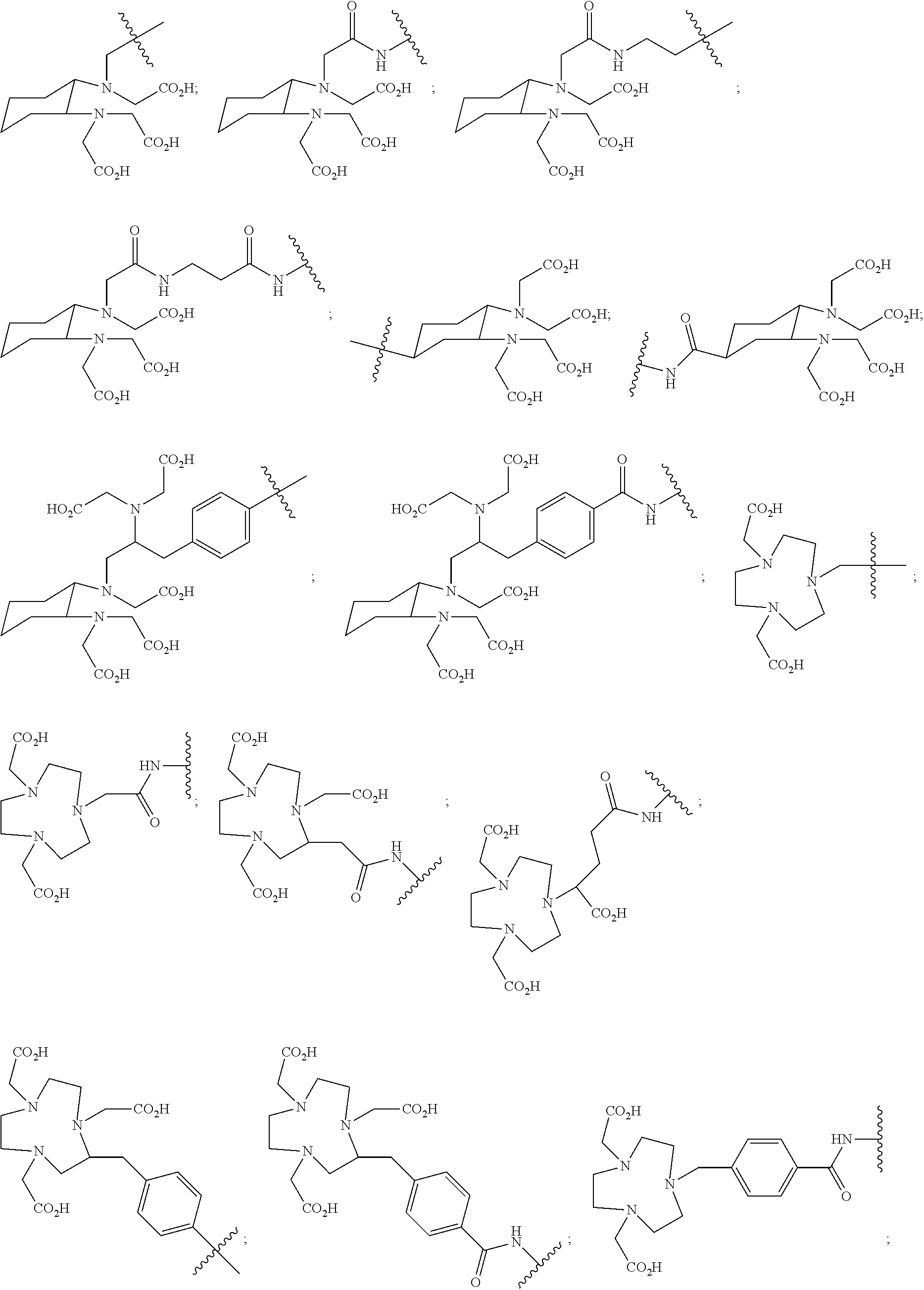

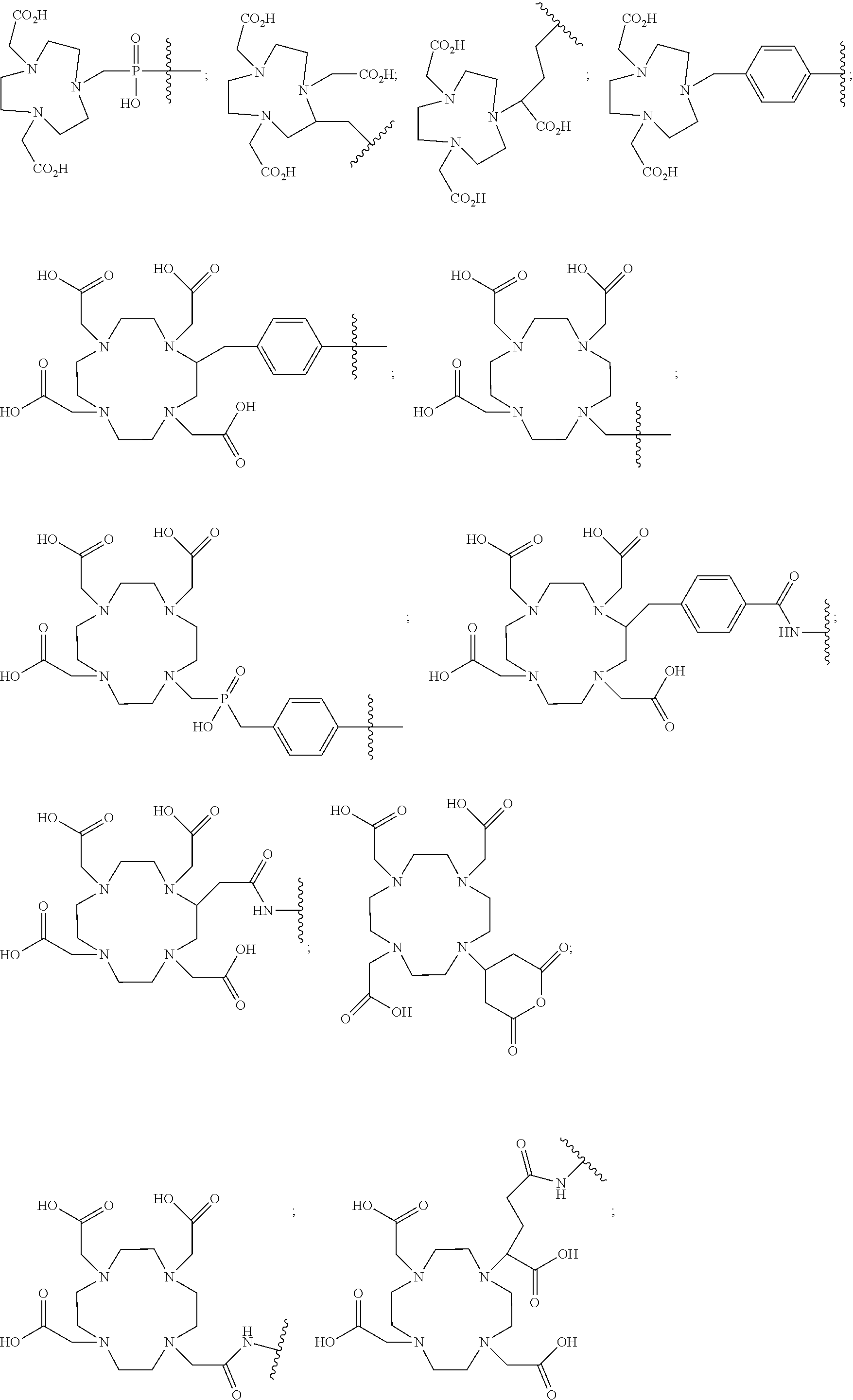

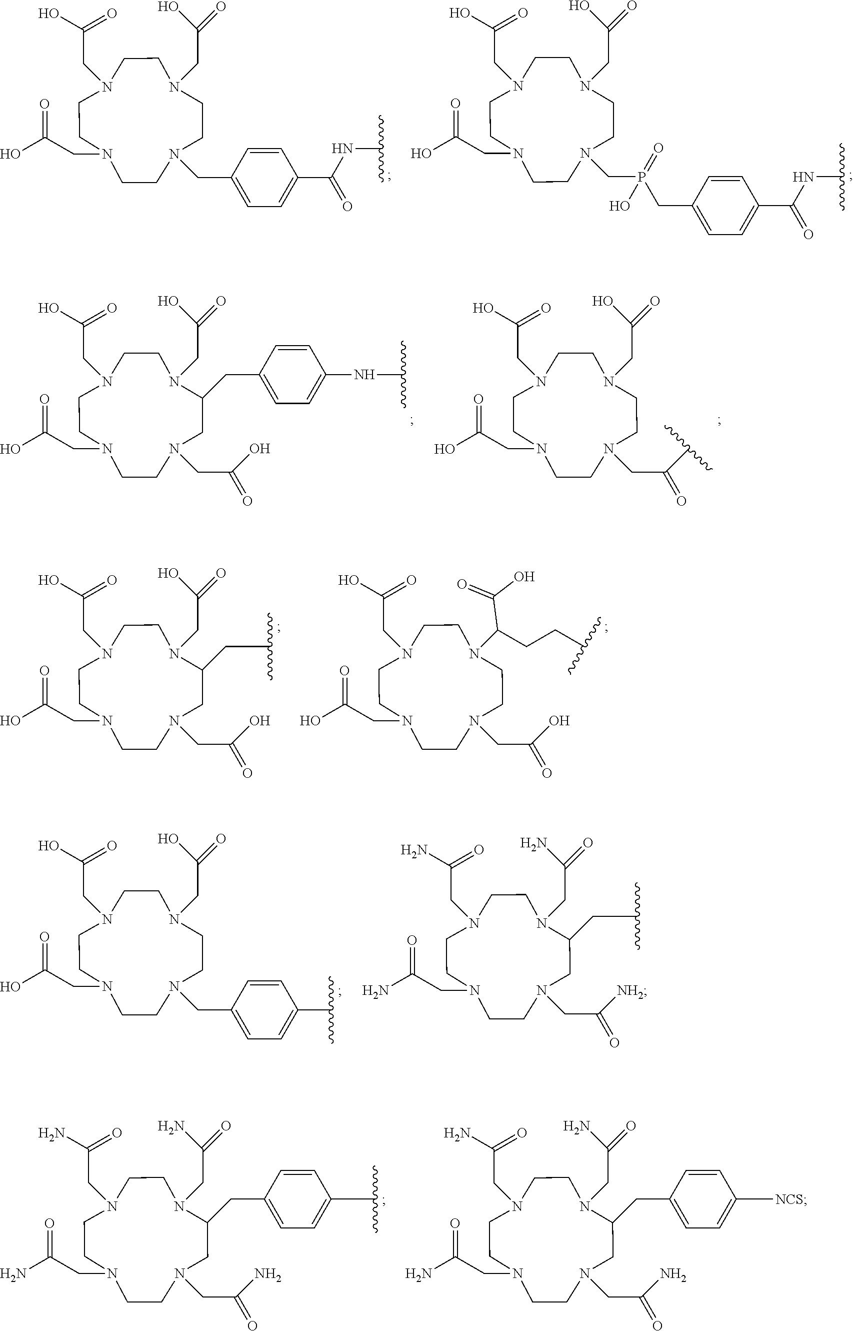

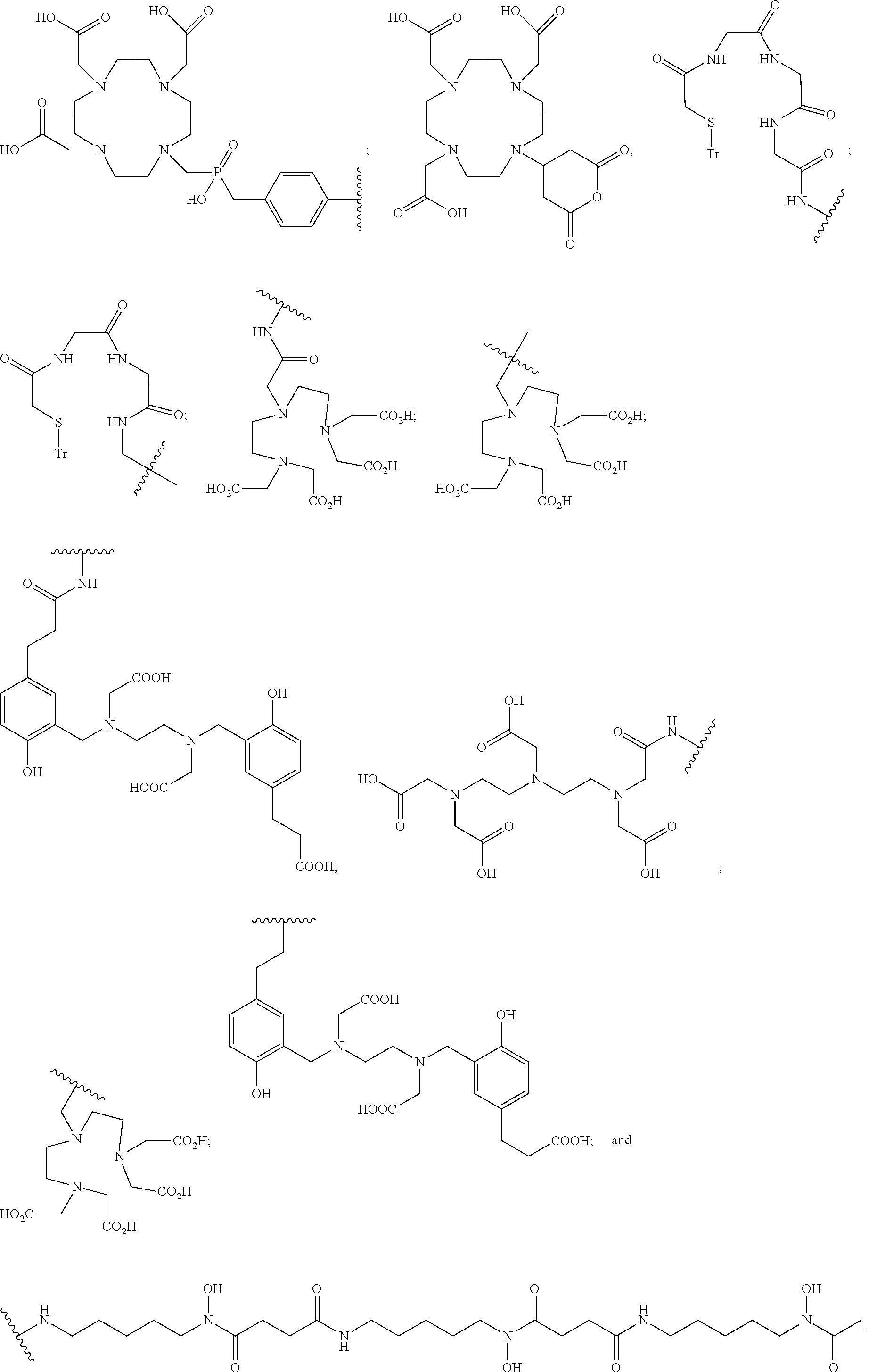

One of ordinary skilled in the arts would appreciate that commercially-available chelating agents can include activating agents, for example, at can react with a primary amine. Such agents include, but are not limited to, N-hydroxysuccinimide (NHS), N-hydroxysulfsuccinimide (sulfo-NHS), anhydride, maleimide, N-benzyl, 4-isothiocyanatobenzyl (p-NCS-Bz), NH₂-MPAA, propargyl, TA, N-(2-aminoethyl)ethanamide, NH₂-PEG4, and hydrophilic dPEG spacer bound to a tetrafluorophenyl (TFP) ester. These agents can be bound to or form part of the Bin hic is bound to the chelating agent.

In certain embodiments, the chelating agent further comprises a radiometal. In particular embodiments, the radiometal is selected from the group consisting of ⁶⁰Cu, ⁶²Cu, ⁶⁴Cu, ⁶⁷Cu, ²⁰³Pb, ²¹²Pb, ²²⁵Ac, ¹⁷⁷Lu, ^{99m}Tc, ⁶⁸Ga, ¹⁴⁹Tb, ⁸⁶Y, ⁹⁰Y, ¹¹¹In, ¹⁸⁶Re, ¹⁸⁸Re, ¹⁵³Sm, ⁸⁹Zr, ²¹³Bi, ²¹²Bi, ²¹²Pb, ⁶⁷Ga, ⁴⁷Sc, and ¹⁶⁶Ho.

In certain embodiments, C comprises an optical dye. In more certain embodiments, the optical dye comprises a fluorescent dye. In yet more certain embodiments, the fluorescent dye comprises a fluorescent dye that emits in the near infrared spectral region. In particular embodiments, the fluorescent dye is selected from the group consisting of a polymethine dye, a coumarin dye, a xanthene dye, and a boron-dipyrromethene (BODIPY) dye.

In certain embodiments, the polymethine dye is selected from the group consisting of a carbocyanine dye, an indocarbocyanine dye, an oxacarbocyanine dye, a thiacarbocyanine dye, and a merocyanine dye. In certain embodiments, the xanthene dye is selected from the group consisting of a fluorescein dye and a coumarin dye. In particular embodiments, the fluorescent dye is selected from the group consisting of:
BODIPY FL, BODIPY R6G, BODIPY TR, BODIPY TMR, BODIPY 493/503, BODIPY 530/550, BODIPY 558/568, BODIPY 564/570, BODIPY 576/589, BODIPY 581/591, BODIPY 630/650, and BODIPY 650/665;
Cy3, Cy3.5, Cy5, Cy5.5, Cy7, and Cy7.5;
VivoTag-645, VivoTag-680, VivoTag-S680, VivoTag-S750, VivoTag-800;
Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, Alexa Fluor 700, Alexa Fluor 750, and AlexaFluor790;
Dy677, Dy676, Dy682, Dy752, Dy780;
DyLight 350, DyLight 405, DyLight 488, DyLight 547, DyLight 550, DyLight 594, DyLight 633, DyLight 647, DyLight 650, DyLight 680, DyLight 755, and DyLight 800;
HiLyte Fluor 405, HiLyte Fluor 488, HiLyte Fluor 532, HiLyte Fluor 555, HiLyte™ Fluor 594, HiLyte Fluor 647, HiLyte Fluor 680, HiLyte Fluor 750;
IR800 (Dimethyl {4-[1,5,5-tris(4-dimethylaminophenyl)-2,4-pentadienylidene]-2,5-cyclohexadien-1-ylidene}ammonium perchlorate),
IRDye 650, IRDye 680RD, IRDye 680LT, IRDye 700, IRDye 700DX, IRDye 750, IRDye 800, IRDye 800CW, IRDye 800RS; and
ADS1065A, ADS1075A, ADS775MI, ADS775MP, ADS775PI, ADS775PP, ADS780HO, ADS780WS, ADS785WS, ADS790WS, ADS795WS, ADS798SM, ADS800AT, ADS815EI, ADS830AT, ADS830WS, ADS832WS, ADS845MC, and ADS920MC.

In more particular embodiments, the optical dye is selected from the group consisting of:

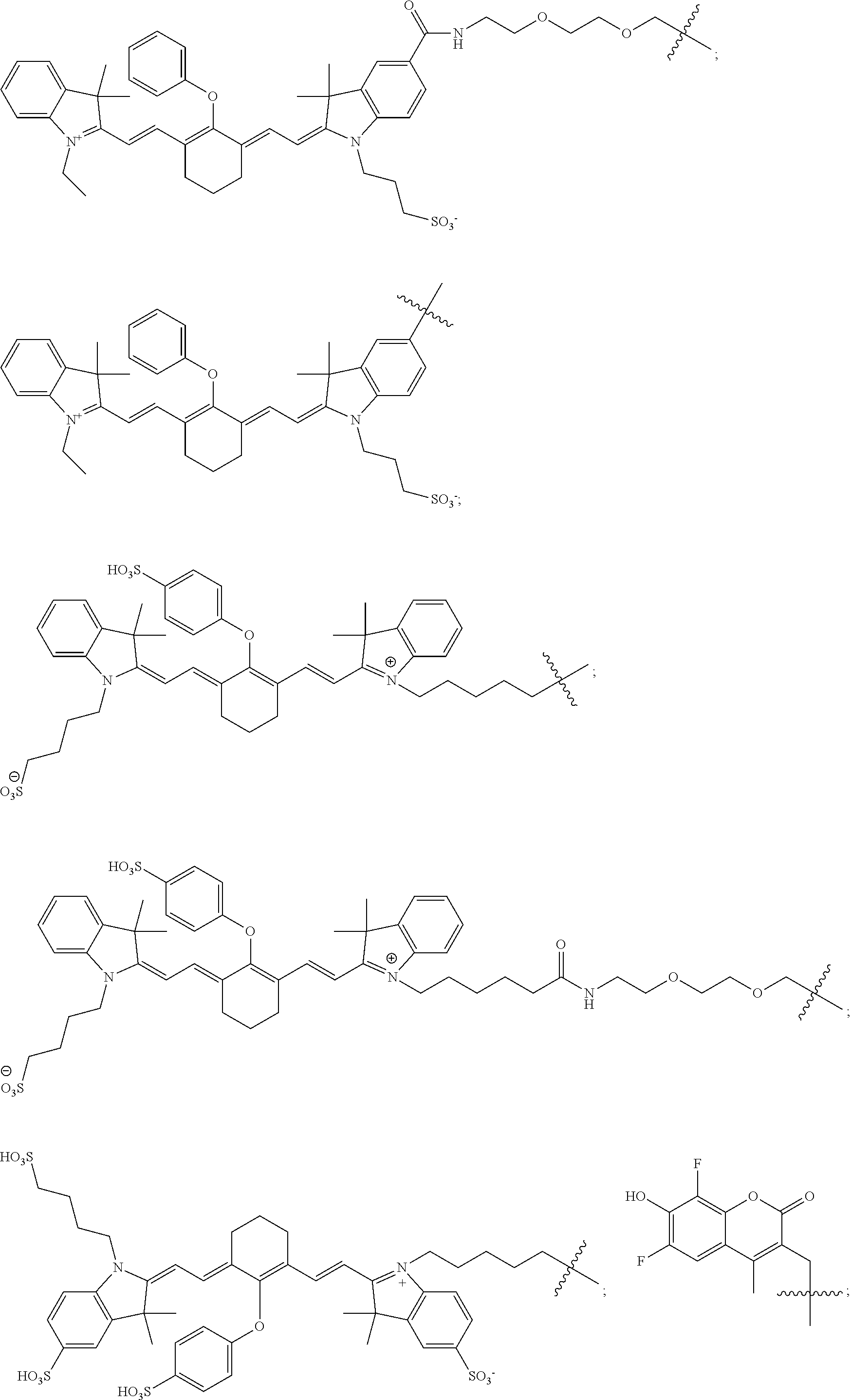

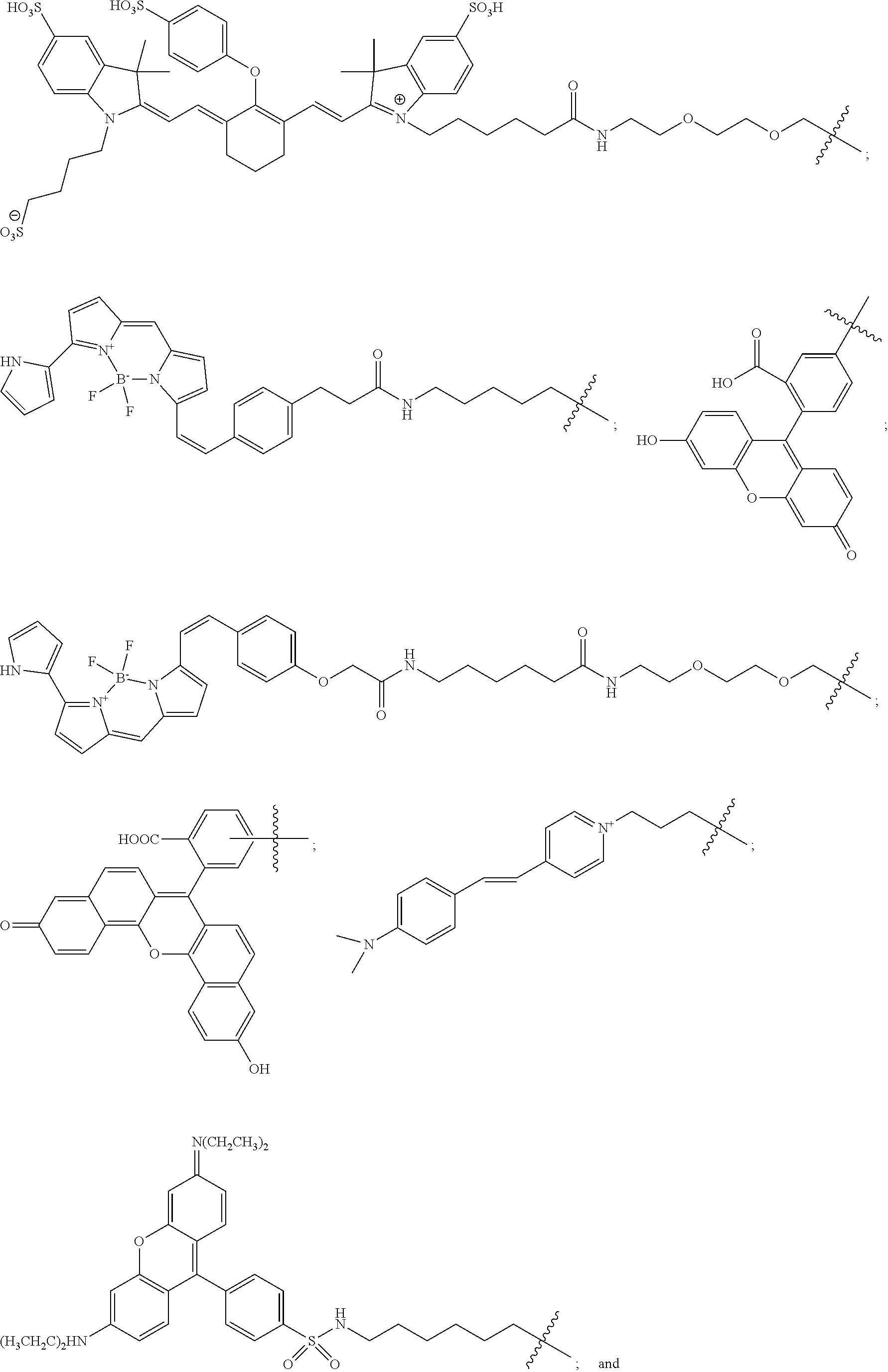

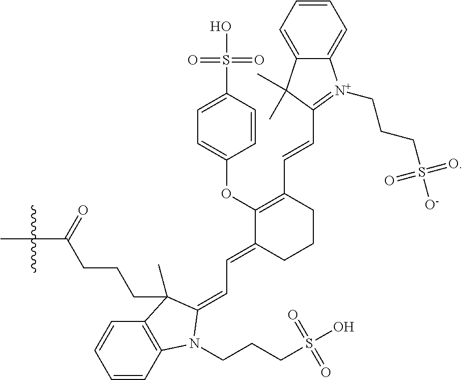 [SMILES: *C(=O)CCCC1(C)/C(=C\C=C2/CCCC(/C=C/C3=[N+](CCCS(=O)(=O)[O-])c4ccccc4C3(C)C)=C2Oc2ccc(S(=O)(=O)O)cc2)N(CCCS(=O)(=O)O)c2ccccc21]

Suitable fluorescent agents and linkers are disclosed in International PCT Patent Application No. WO2018232280 for PSMA Targeted Fluorescent Agents for Image Guided Surgery, to Pomper et al., published Dec. 20, 2018, which is incorporated herein by reference in its entirety.

In some embodiments, the presently disclosed compounds can be made by attaching near IR, closed chain, sulfo-cyanine dyes to prostate specific membrane antigen ligands via a linkage. For example, the prostate specific membrane antigen ligands used in the presently disclosed compounds can be synthesized as described in international PCT patent application publication no. WO 2010/108125, to Pomper et al., published Sep. 23, 2010, which is incorporated herein in its entirety.

Compounds can assembled by reactions between different components, to form linkages such as ureas (—NRC(O)NR—), thioureas (—NRC(S)NR—), amides (—C(O)NR— or —NRC(O)—), or esters (—C(O)O— or —OC(O)—). Urea linkages can be readily prepared by reaction between an amine and an isocyanate, or between an amine and an activated carbonamide (—NRC(O)—). Thioureas can be readily prepared from reaction of an amine with an isothiocyanate. Amides (—C(O)NR— or —NRC(O)—) can be readily prepared by reactions between amines and activated carboxylic acids or esters, such as an acyl halide or N-hydroxysuccinimide ester. Carboxylic acids may also be activated in situ, for example, with a coupling reagent, such as a carbodiimide, or carbonyldiimidazole (CDI). Esters may be formed by reaction between alcohols and 20 activated carboxylic acids. Triazoles are readily prepared by reaction between an azide and an alkyne, optionally in the presence of a copper (Cu) catalyst.

Prostate specific membrane antigen ligands can also be prepared by sequentially adding components to a preformed urea, such as the lysine-urea-glutamate compounds described in Banerjee et al. (J. Med. Chem. vol. 51, pp. 4504-4517, 2008). Other urea-based compounds may also be used as building blocks. Exemplary syntheses of the near IR, closed chain, sulfo-cyanine dyes used in the presently disclosed compositions are described in U.S. Pat. Nos. 6,887,854 and 6,159,657 and are incorporated herein in their entirety. Additionally, some IR, closed chain, sulfo-cyanine dyes of the presently disclosed subject matter are commercially available, including DyLight™ 800 (ThermoFisher).

In some embodiments, the presently disclosed compounds comprising a fluorescent dye and be used for photoacoustic imaging of tumors. See, for example, Zhang et al., Prostate-specific membrane antigen-targeted photoacoustic imaging of prostate cancer in vivo, J. of Biophotonics, 2018; 11: e201800021. Accordingly, in certain embodiments, C comprises a dye suitable for use with photoacoustic imaging.

In certain embodiments, C comprises a photosensitizer. Photodynamic therapy (PDT) is a minimally invasive cancer treatment and has been used in clinic to improve cancer patients' quality of life and survival time. The lack of specific delivery of the photosensitizers, however, is a significant limitation of PDT. Non-targeted, conventional photodynamic therapy cannot deliver the photosensitizers specifically to the tumor and the photosensitizers often circulate in the body long after treatment and cause sensitivity to light for several months.

Urea-based photosensitizers, which target prostate-specific membrane antigen (PSMA) for imaging and targeted therapy of PSMA-expressing tumors and cancers are disclosed in International PCT Patent Application No. WO2015057692 for Prostate-Specific Membrane Antigen-Targeted Photosensitizers for Photodynamic Therapy, to Pomper et al., published Apr. 23, 2015, and U.S. Pat. No. 10,232,058 for Prostate-Specific Membrane Antigen-Targeted Photosensitizers for Photodynamic Therapy, to Pomper et al., issued Mar. 19, 2019, each of which is incorporated by reference in their entirety.

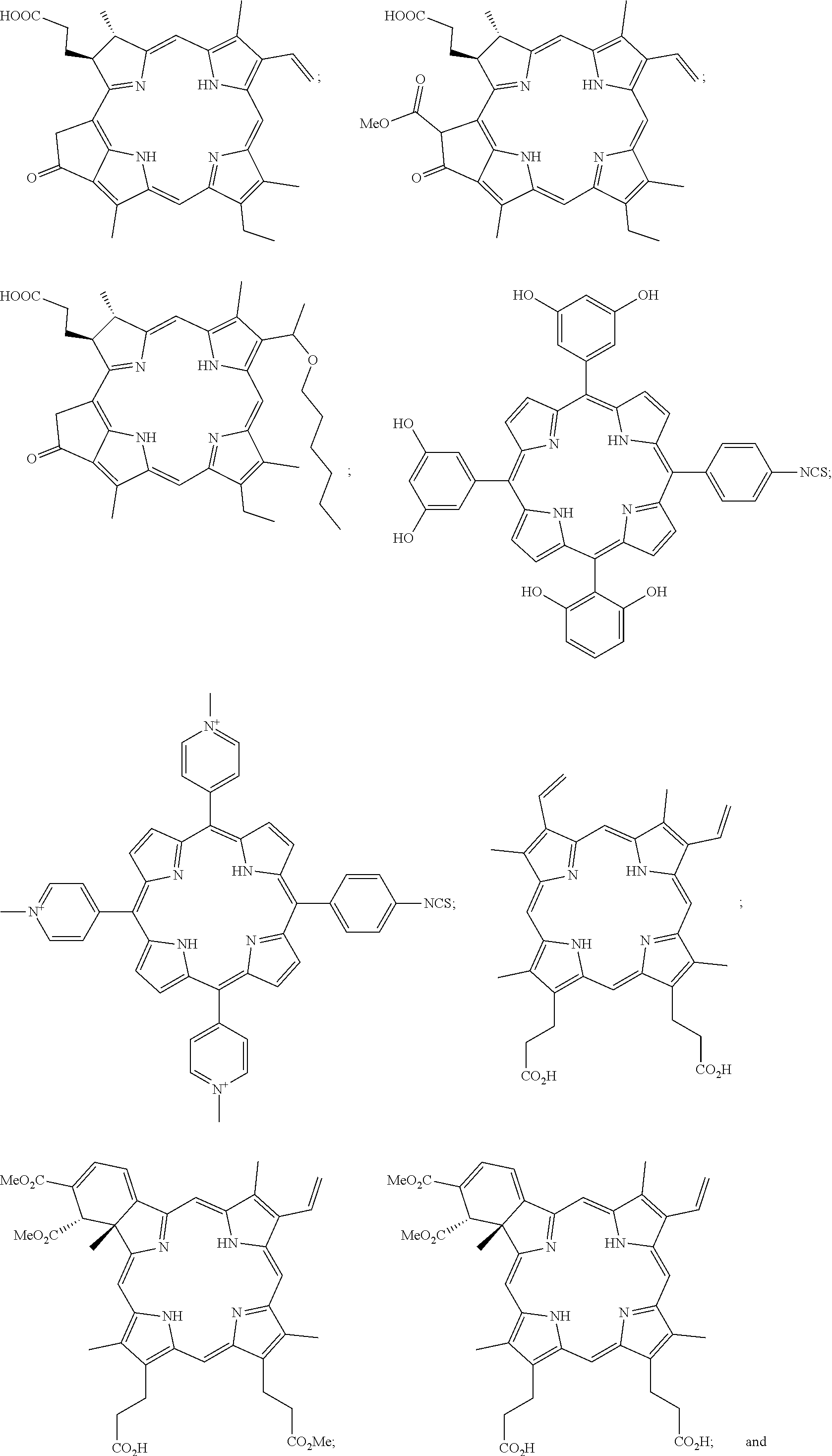

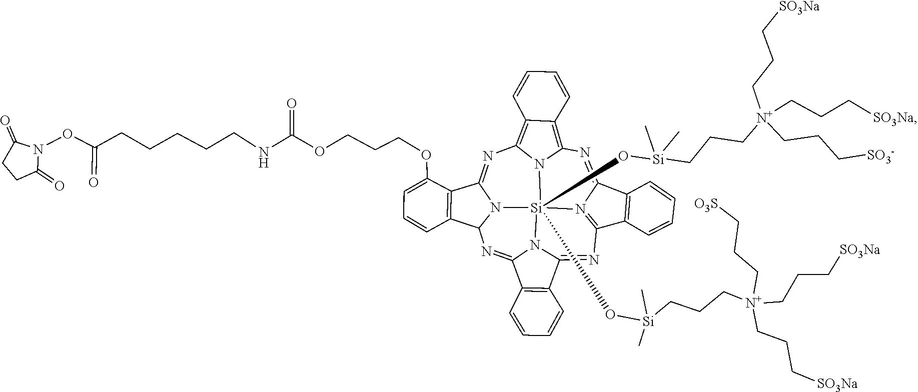

In certain embodiments, the compound of formula (I) has the following structure:

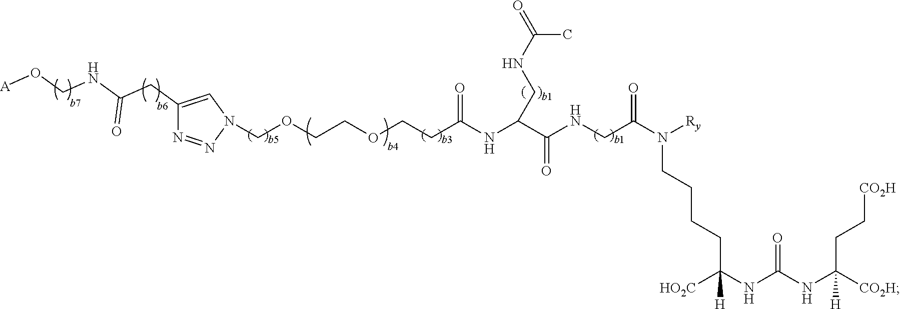

wherein b1, b2, b3, b4, b5, b6, and b7 are each independently an integer selected from the group consisting of 0, 1, 2, 3, 4, 5, 6, 7, and 8.

In particular embodiments, the compound of formula (I) has the following structure:

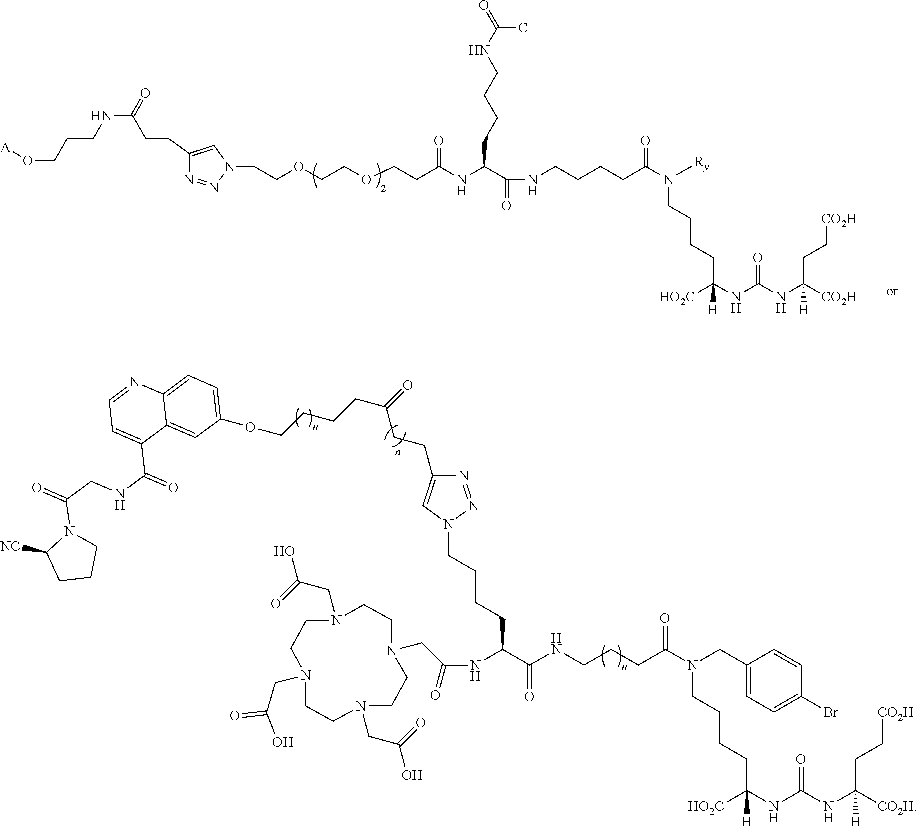

In more particular embodiments, the compound of formula (I) is:

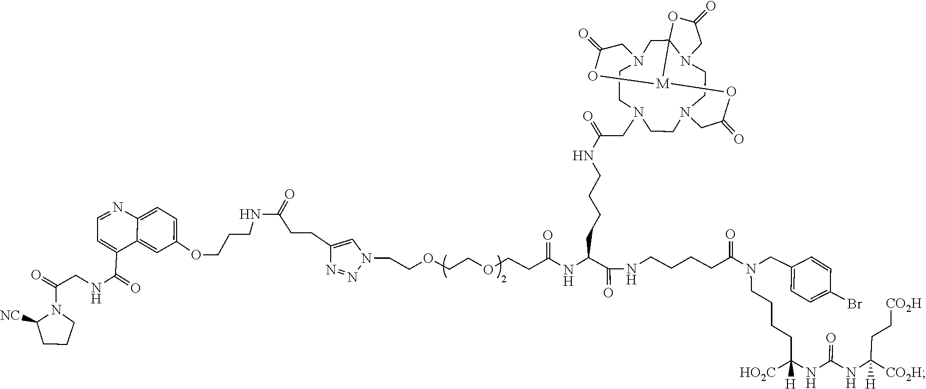

wherein M is selected from the group consisting of ⁶⁸Ga, ²²⁵Ac, ¹⁷⁷Lu, and ⁶⁴Cu.

In some embodiments, A and B are each an FAP-α targeting moiety having the structure of

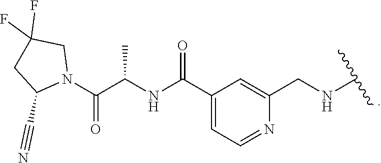 [SMILES: *NCc1cc(C(=O)N[C@@H](C)C(=O)N2CC(F)(F)C[C@H]2C#N)ccn1]

In particular embodiments, the compound is selected from the group consisting of:

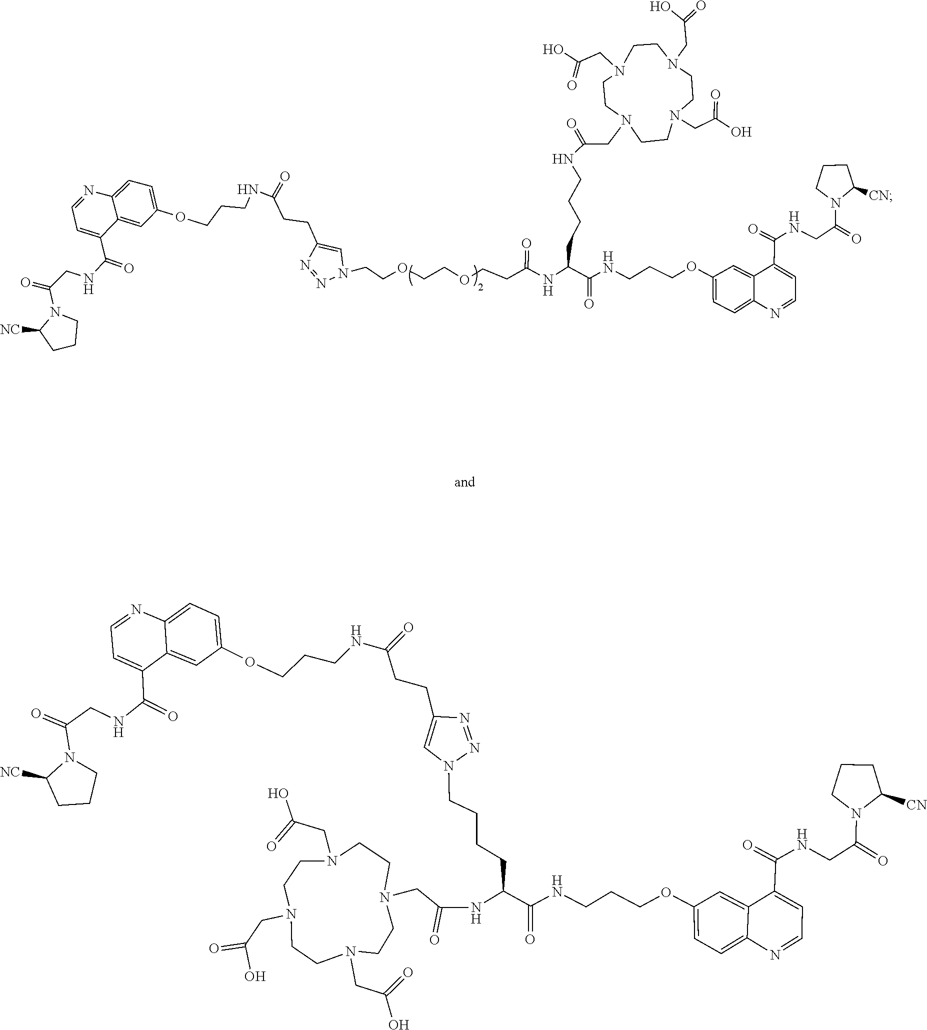

In certain embodiments, the compound is:

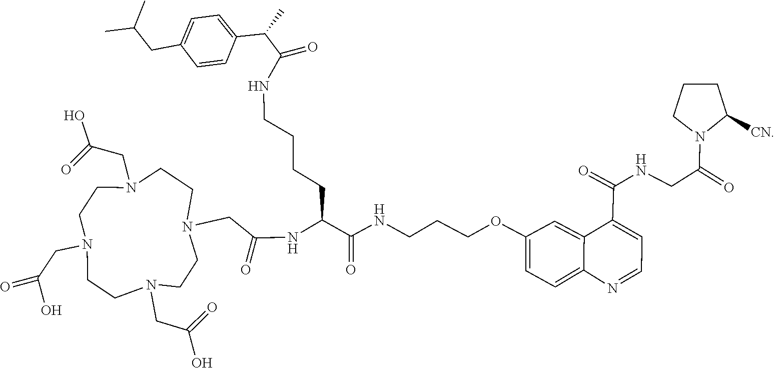 [SMILES: CC(C)Cc1ccc([C@H](C)C(=O)NCCCC[C@H](NC(=O)CN2CCN(CC(=O)O)CCN(CC(=O)O)CCN(CC(=O)O)CC2)C(=O)NCCCOc2ccc3nccc(C(=O)NCC(=O)N4CCC[C@H]4C#N)c3c2)cc1]

In other embodiments, the presently disclosed subject matter provides a pharmaceutical composition comprising the compound of formula (I). In some embodiments, the pharmaceutical composition comprises one or more of pharmaceutically acceptable carriers, diluents, excipients, or adjuvants.

In some embodiments, the presently disclosed subject matter provides a compound of formula (II):

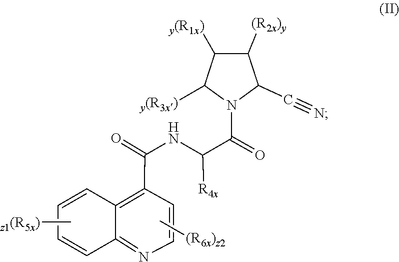

wherein each y is independently an integer selected from the group consisting of 0, 1, and 2;
R₁ₓ, R₂ₓ, and R_{3x′}, are each independently selected from the group consisting of H, OH, halogen, C₁₋₆alkyl, —O—C₁₋₆alkyl, and —S—C₁₋₆alkyl;
R₄ₓ is selected from the group consisting of H, straight-chain or branched C₁₋₆ alkyl, —(CH₂)_{q4}-aryl, and hydroxyl-substituted straight-chain or branched C₁₋₆ alkyl, wherein q4 is an integer selected from the group consisting of 0, 1, 2, 3, 4, 5, and 6;
R₅ₓ and R₆ₓ are each independently H, halogen, or —O—(CH₂)_{z3}—Xₐ, wherein z1 is an integer from 0 to 4, z2 is an integer from 0 to 2, z3 is an integer from 1 to 6, and Xₐ is halogen, provided that R₅ₓ and R₆ₓ cannot both be H at the same time;
and stereoisomers and pharmaceutically acceptable salts thereof.

In some embodiments, the compound of formula (II) is selected from:

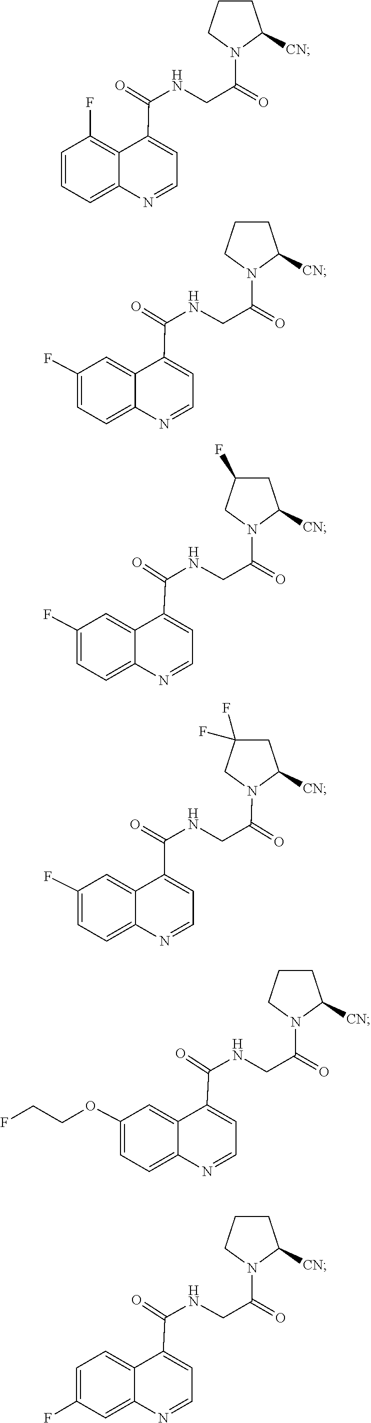 [SMILES: N#C[C@@H]1CCCN1C(=O)CNC(=O)c1ccnc2cccc(F)c12 ; N#C[C@@H]1CCCN1C(=O)CNC(=O)c1ccnc2ccc(F)cc12 ; N#C[C@@H]1C[C@H](F)CN1C(=O)CNC(=O)c1ccnc2ccc(F)cc12 ; N#C[C@@H]1CC(F)(F)CN1C(=O)CNC(=O)c1ccnc2ccc(F)cc12 ; N#C[C@@H]1CCCN1C(=O)CNC(=O)c1ccnc2ccc(OCCF)cc12 ; N#C[C@@H]1CCCN1C(=O)CNC(=O)c1ccnc2cc(F)ccc12]

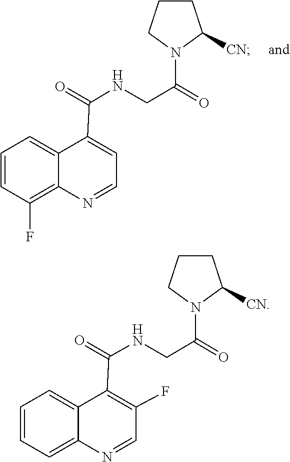 [SMILES: N#C[C@@H]1CCCN1C(=O)CNC(=O)c1ccnc2c(F)cccc12 ; N#C[C@@H]1CCCN1C(=O)CNC(=O)c1c(F)cnc2ccccc12]

In other embodiments, the presently disclosed subject matter provides a compound of formula (III):

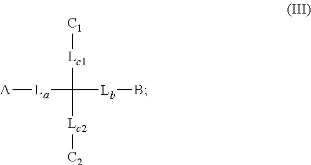

wherein:
A is a targeting moiety for fibroblast activation protein alpha (FAP-α);
B is absent or a targeting moiety for FAP-α, wherein A and B can be the same or different;
C₁ can be absent or present and when present is a chelating group;
C₂ is a prosthetic group;
Lₐ and L_{b} are each a bi-functionalized linker capable of forming a chemical bond with each other and A, B, C₁ and C₂;
L_{c1} and L_{c2} are each independently a bi-functionalized linker capable of forming a chemical bond with each other and A, B, Lₐ, and L_{b};
wherein if C₁ is absent, L_{c1} also is absent and wherein if B is absent, L_{b} also is absent;
and stereoisomers and pharmaceutically acceptable salts thereof.

In some embodiments, A is or, if B is present, A and B are each an FAP-α targeting moiety having the structure of:

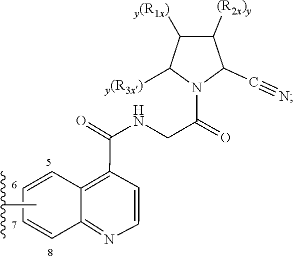

wherein [symbol] indicates a point of attachment of the FAP-α binding ligand to the linker Lₐ and/or L_{b} wherein the point of attachment can be through any of carbon atoms 5, 6, 7, or 8 of the quinolinyl ring thereof; and stereoisomers and pharmaceutically acceptable salts thereof.

In some embodiments, C₂ is a prosthetic group comprising a radioisotope selected from the group consisting of ¹⁸F, ¹²⁴I, ¹²⁵I, ¹³¹I, and ²¹¹At.

In some embodiments, the prosthetic group is selected from the group consisting of:

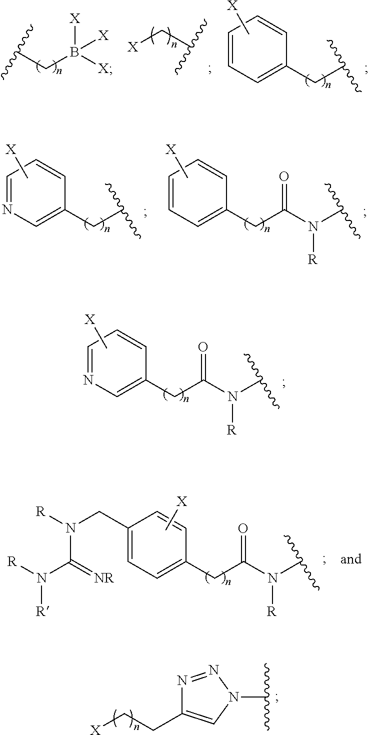

wherein each X is independently selected from a straightchain or branched C₁-C₈ alkyl, —SO₂, —C(═O)—, —C(═O)OR₂₀, wherein R₂₀ is H or C₁-C₄ alkyl, and a radioisotope selected from the group consisting of ¹⁸F, ¹²⁴I, ¹²⁵I, ¹³¹I, and ²¹¹At; each R and R′ is defined hereinabove; and each n is independently an integer selected from the group consisting of 0, 1, 2, 3, 4, 5, 6, 7, 8, 9, 10, 11, 12, 13, 14, 15, 16, 17, 18, 19, and 20, wherein each carbon of the

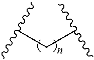

alkylene chain can be substituted with C₁-C₄ alkyl.

In some embodiments, the prosthetic group is selected from the group consisting of:

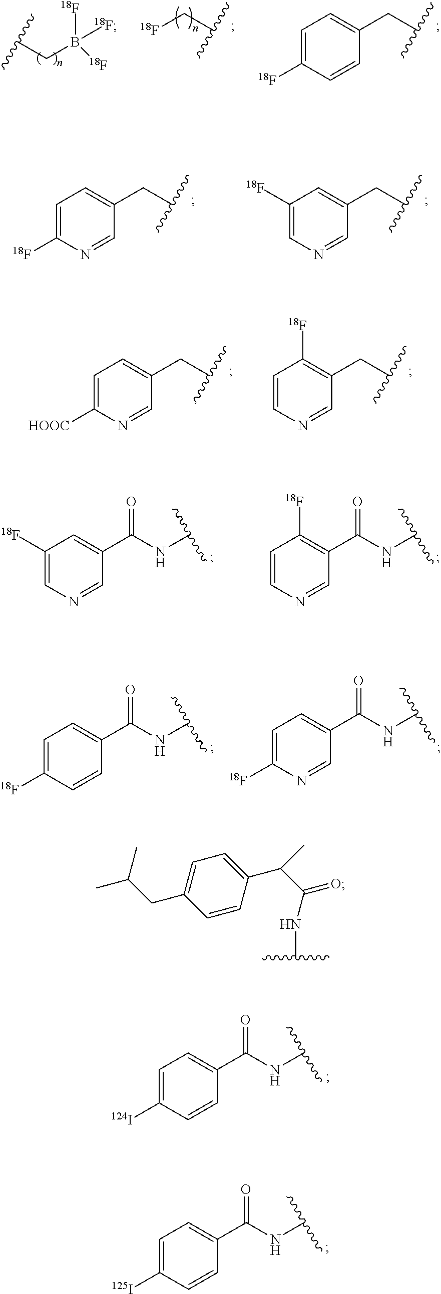

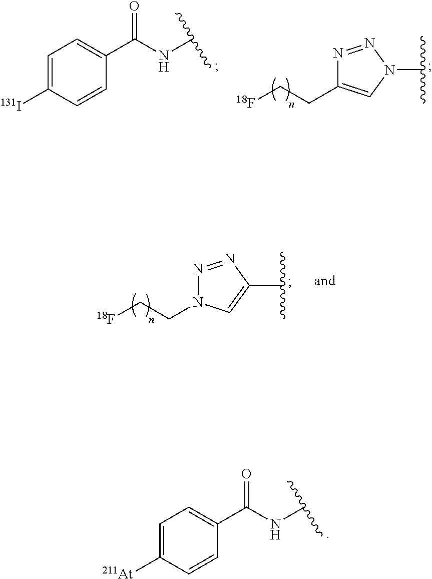

In some embodiments, C₁ comprises a chelating agent selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), (NOTAGA) 1,4,7-triazonane-1,4-diyl)diacetic acid DFO (Desferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino) methyl) benzoic acid), and BaBaSar.

In some embodiments, C₁ is a chelating agent is selected from the group consisting of:

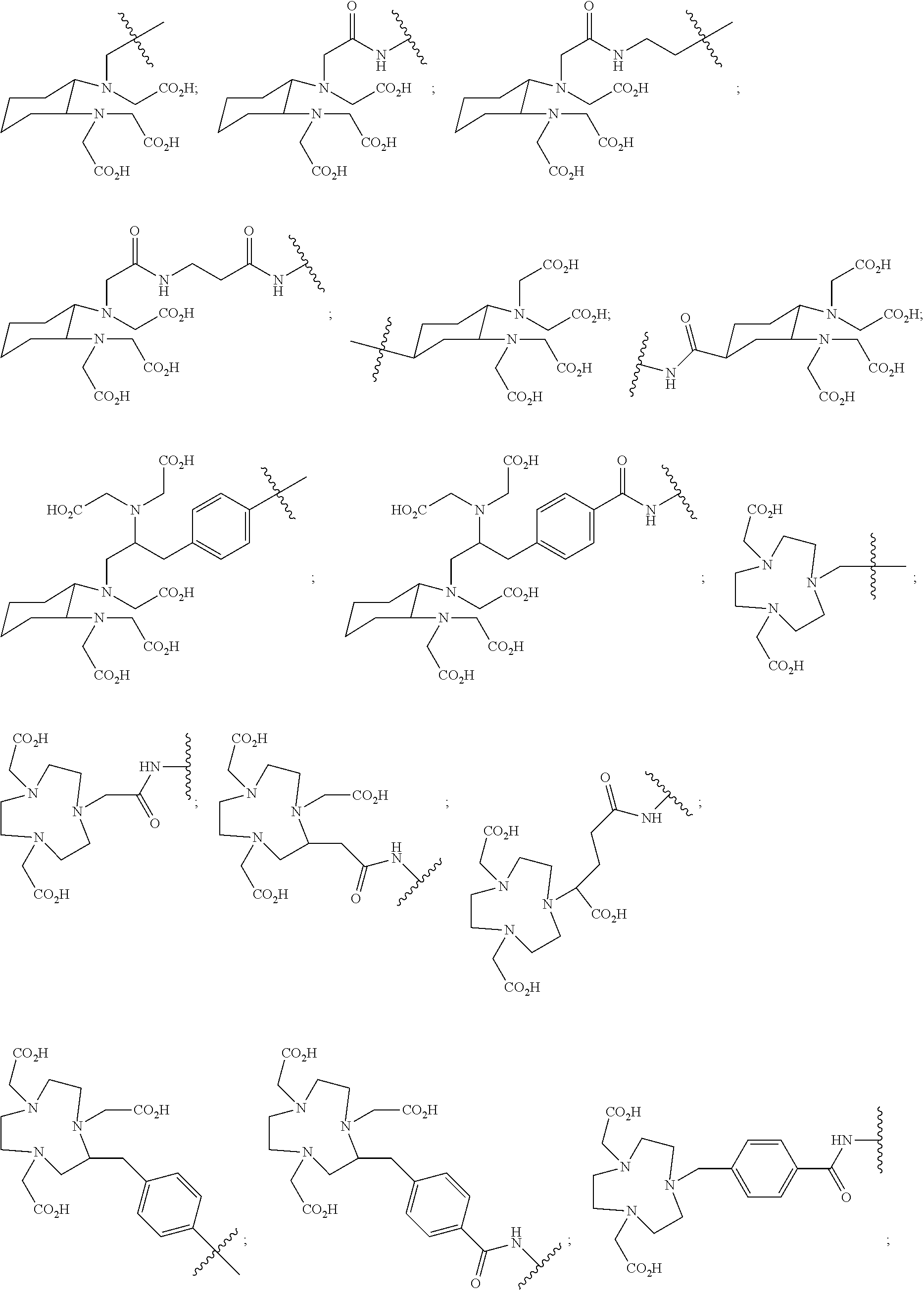

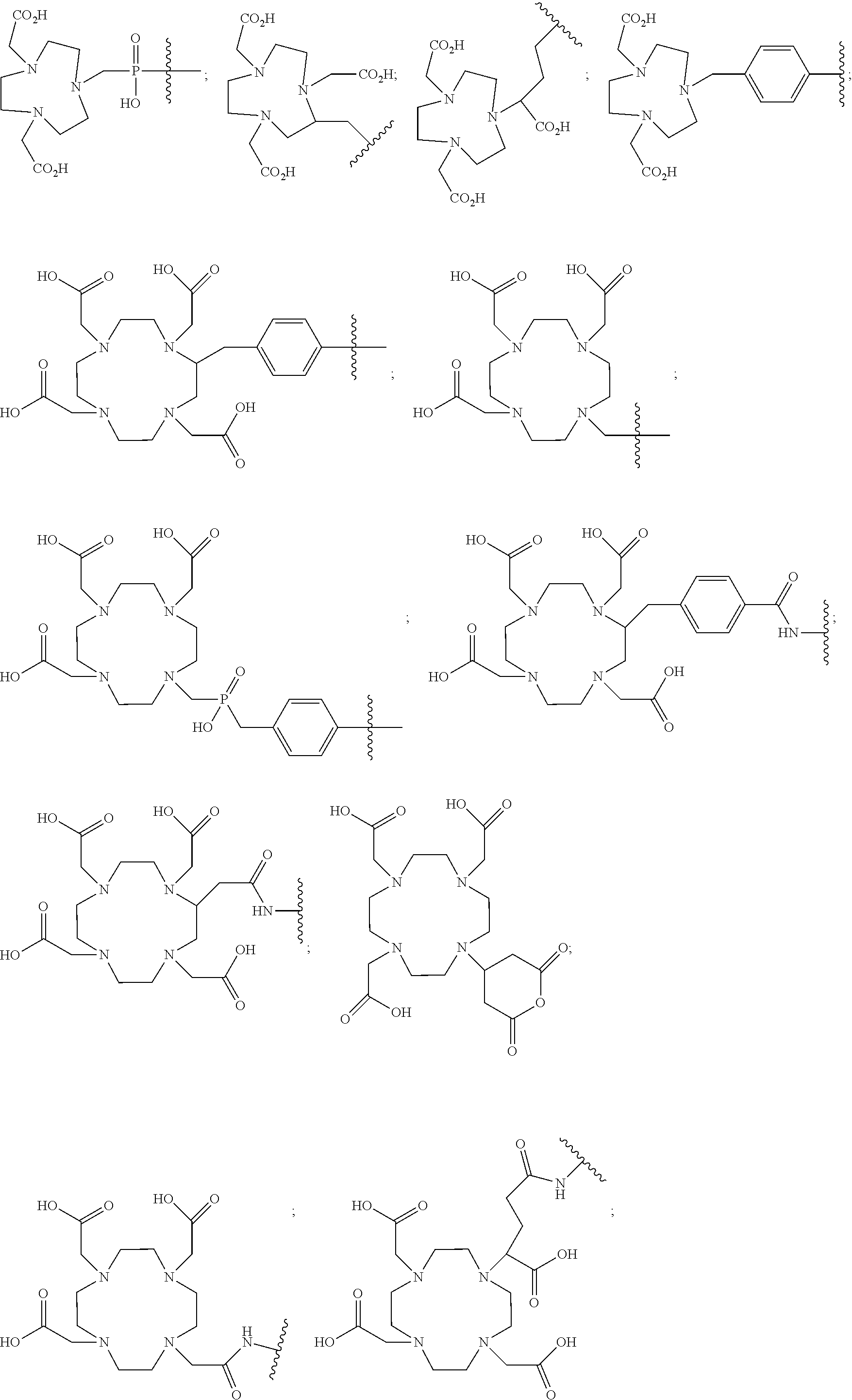

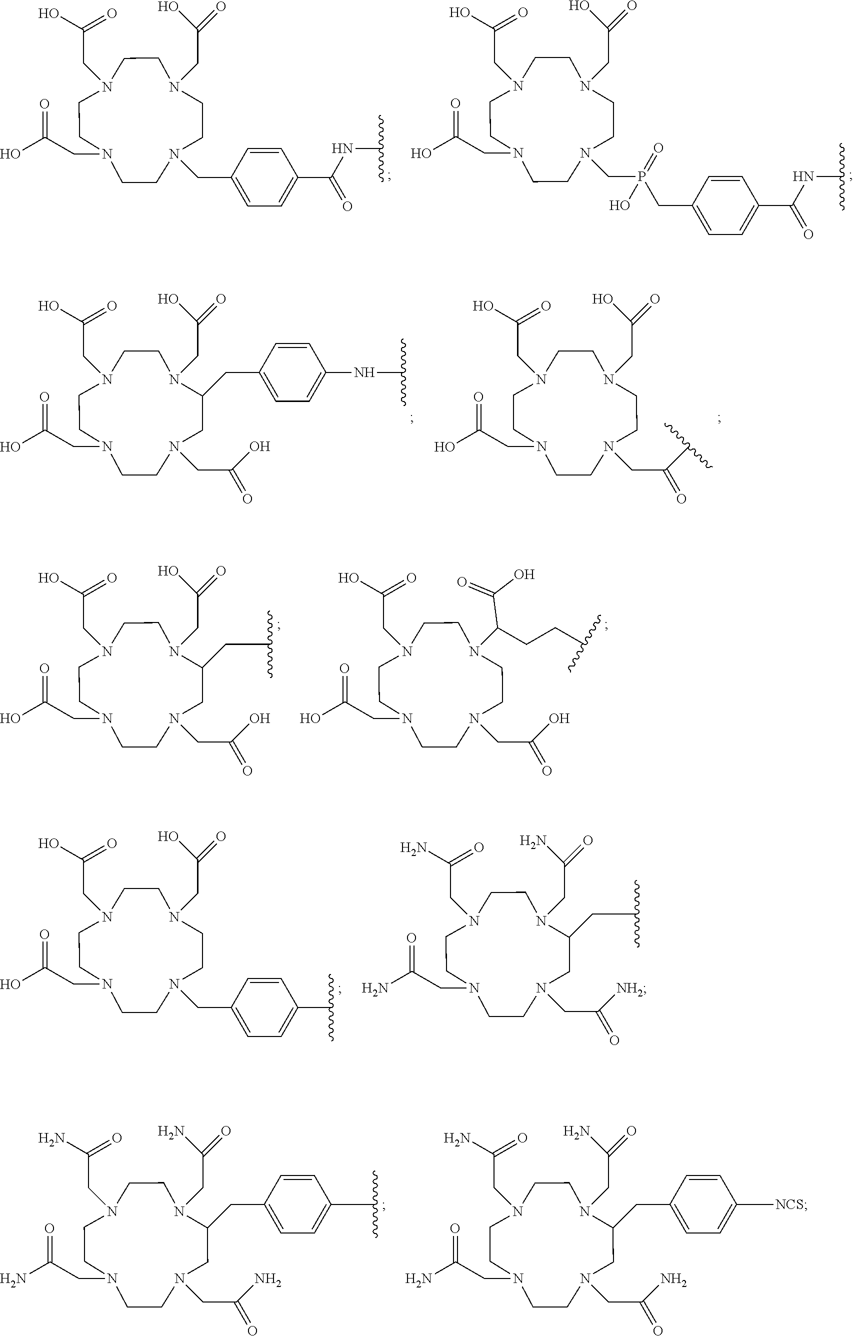

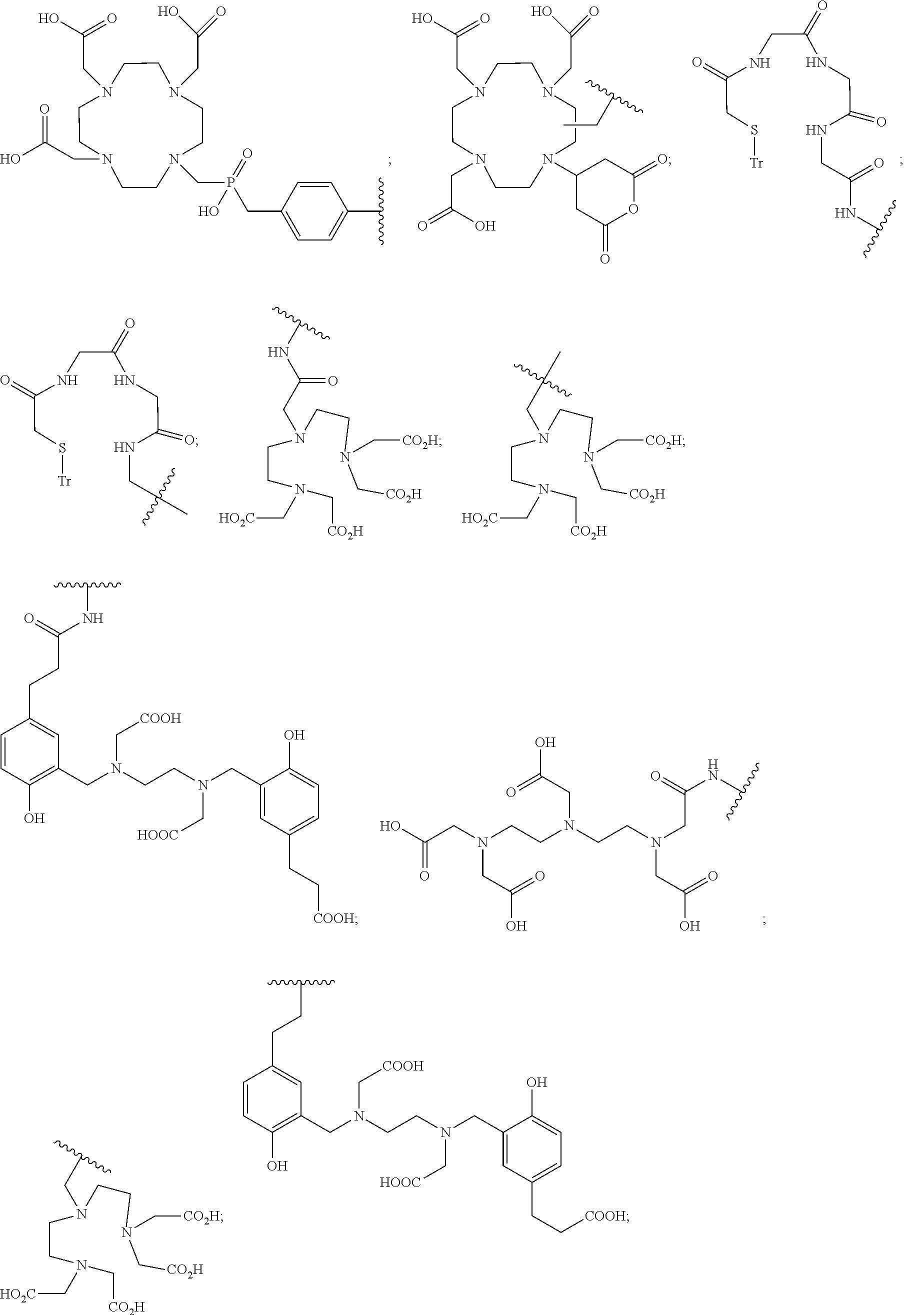

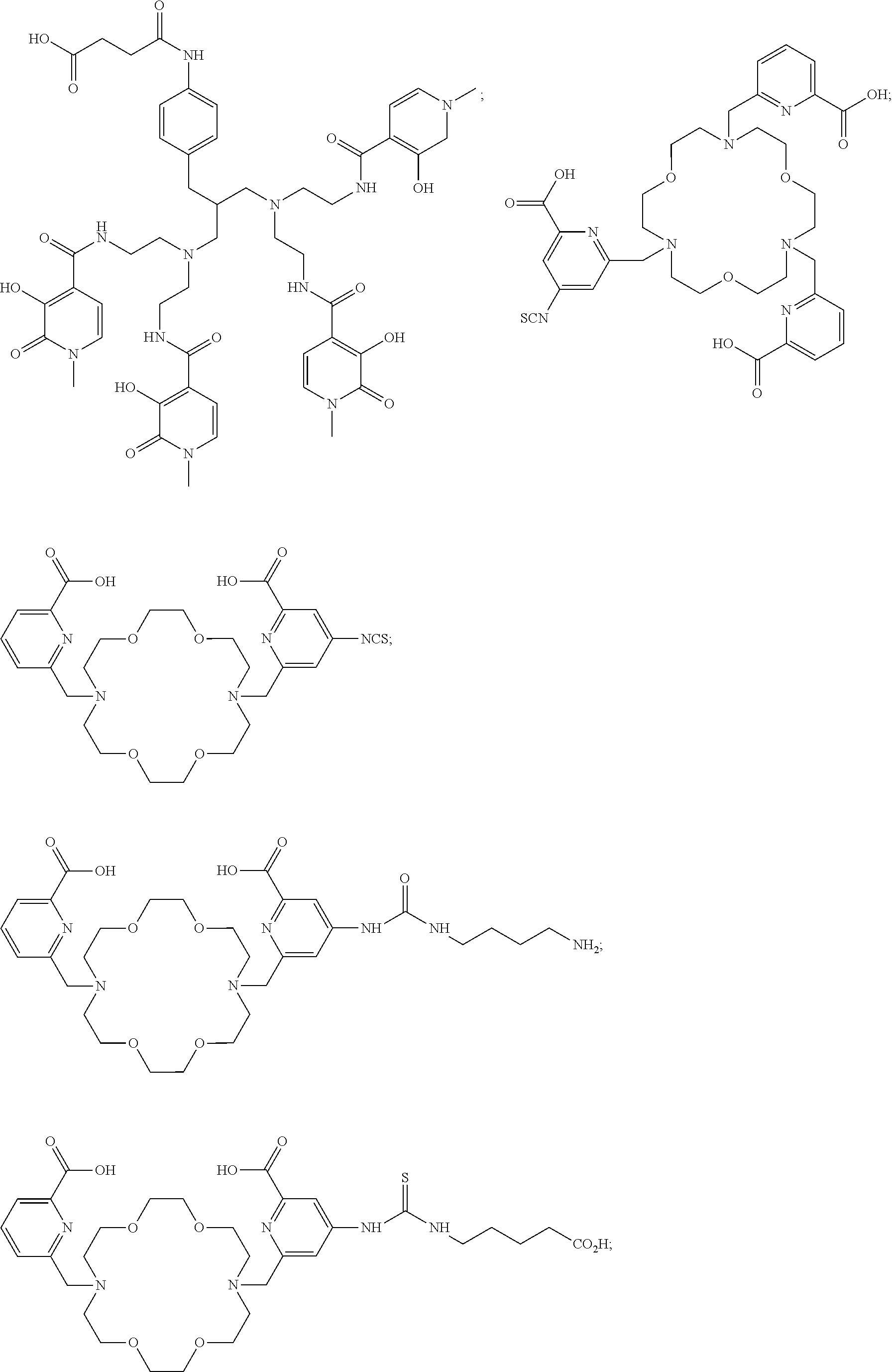

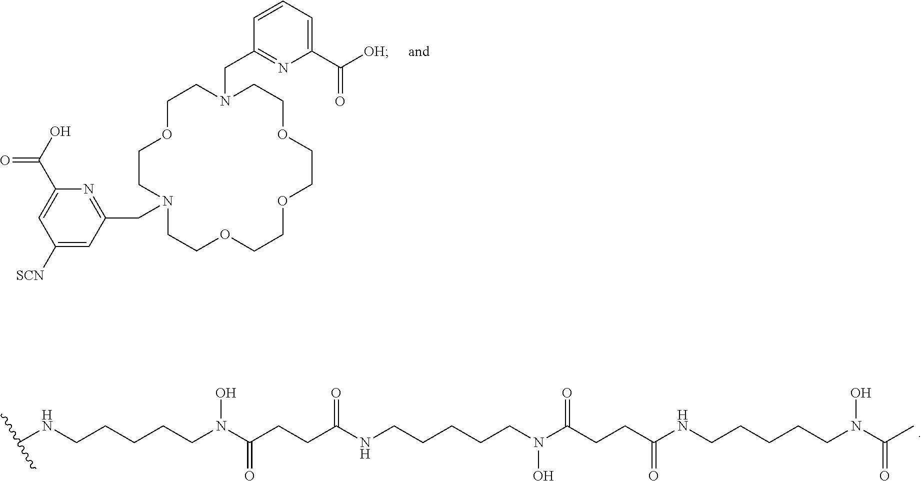

In some embodiments, the chelating agent further comprises a radiometal.

In some embodiments, the radiometal is selected from the group consisting of ⁶⁰Cu, ⁶²Cu, ⁶⁴Cu, ⁶⁷Cu, ²⁰³Pb, ²¹²Pb, ²²⁵Ac, ¹⁷⁷Lu, ^{99m}Tc, ⁶⁸Ga, ¹⁴⁹Tb, ⁸⁶Y, ⁹⁰Y, ¹¹¹In, ¹¹⁵In, ¹⁸⁶Re, ¹⁸⁸Re, ¹⁵³Sm, ⁸⁹Zr, ²¹³Bi, ²¹²Bi, ²¹²Pb, ⁶⁷Ga, ⁴⁷Sc, ¹⁶⁶Ho, ⁴³Sc, ²²³Ra, ^{226/227}Th, Al-¹⁸F, and Sc-¹⁸F.

In some embodiments, Lₐ, Lₐ, L_{c1}, and L_{c2} are each individually selected from the group consisting of (a), (b), (c), or (d):
(a)

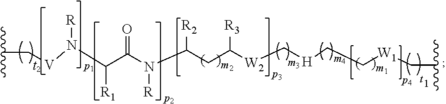

wherein:
p₁, p₂, p₃ and p₄ may be in any order;
t₁ and t₂ are each an integer selected from the group consisting of 0, 1, 2, 3, 4, 5, 6, 7, and 8;
p₁, p₃, and p₄ are each independently 0 or 1;
p₂ is an integer selected from the group consisting of 0, 1, 2, and 3, and when p₂ is 2 or 3, each R₁ is the same or different;
m₁, m₂, m₃, and m₄ are each an integer independently selected from the group consisting of 0, 1, 2, 3, 4, 5, 6, 7 and 8;
W₁ is selected from the group consisting of a bond, —S—, —C(═O)—, —C(═O)—NR—, and —NR—C(═O)—;
W₂ is selected from the group consisting of a bond, —S—, —CH₂—C(═O)—NR—, C(═O)—, —NRC(═O)—, —NR′C(═O)NR—, —NRC(═S)NR′₂—, —NRC(═O)O—, —OC(═O)NR—, —OC(═O)—, —C(═O)NR—, —NR—C(═O)—, —C(═O) O, (O—CH₂—CH₂)_{q}— and —(CH₂—CH₂—O)_{q}, wherein q is selected from the group consisting of 0, 1, 2, 3, 4, 5, 6, 7, and 8;
each R or R′ is independently H, alkyl, substituted alkyl, cycloalkyl, substituted cycloalkyl, heterocycloalkyl, substituted heterocycloalkyl, aryl, substituted aryl, heteroaryl, substituted heteroaryl, and —OR₄, wherein R₄ is selected from the group consisting of H, alkyl, substituted alkyl, cycloalkyl, substituted cycloalkyl, heterocycloalkyl, and substituted heterocycloalkyl, wherein q is defined as immediately hereinabove;
H can be present or absent and when present is selected from a nitrogen-containing heteroalkylene chain, a cyclic or bicylic heteroalkyl radical, and a triazole radical having the structure

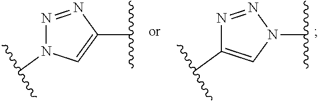 [SMILES: *c1cn(*)nn1 ; *c1cn(*)nn1]

each R₁ is independently H, C₁-C₆ alkyl, C₃-C₁₂ aryl, —(CH₂)_{q}—C₃-C₁₂ aryl, —C₄-C₁₆ alkylaryl, or —(CH₂)_{q}—C₄-C₁₆ alkylaryl; R² and R³ are each independently H, —(CH₂)_{q}—C₃-C₁₂ aryl, and —CO₂R₅, wherein R₅ is selected from the group consisting of H, C₁-C₆ alkyl, C₃-C₁₂ aryl, and C₄-C₁₆ alkylaryl, wherein R₂ and R₃ can be the same or different, wherein q is selected from the group consisting of 0, 1, 2, 3, 4, 5, 6, 7, and 8;
V is selected from the group consisting of —C(O)—, —C(S)—, —NRC(O), —NRC(S)—, and —OC(O)—;
(b)

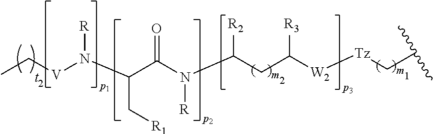

wherein p₁, p₂, p₃, m₁, m₂, Tz, W₂, R, R₁, R₂, R₃, and V are defined as hereinabove;
(c) -L₁-, -L₂-L₃-, or -L₁-L₂-L₃-, wherein:
L₁ is —NR—(CH₂)_{q}—[O—CH₂—CH₂—O]_{q}—(CH₂)_{q}—C(═O)—;
L₂ is —NR—(CH₂)_{q}—C(COOR₅)—NR—; and
L₃ is —(O═)C—(CH₂)_{q}—C(═O)—;
   wherein each q is independently an integer selected from the group consisting of 1, 2, 3, 4, 5, 6, 7, and 8; and R and R⁵ are as defined hereinabove; and

(d) —(CR₆H)_{q}—(CH₂)_{q}—C(═O)—NR—(CH₂)_{q}—O— or —NR—(CH₂)_{q}—O—; wherein each q and R is defined hereinabove; and R₆ is H or —COOR₅.

In some embodiments, H is selected from:

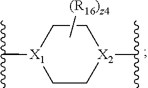

wherein X₁ and X₂ are each independently —CH— or N; each R₁₆ is independently H or —C(═O)—OR₁₇, wherein R₁₇ is C₁-C₄ alkyl;
—N(R₁₈)—(CH₂)_{z5}—N(R₁₉)—; wherein R₁₈ and R₁₉ are each independently H or C₁-C₄ alkyl and Z5 is an integer selected from 1, 2, 3, 4, 5, 6, 7, and 8;

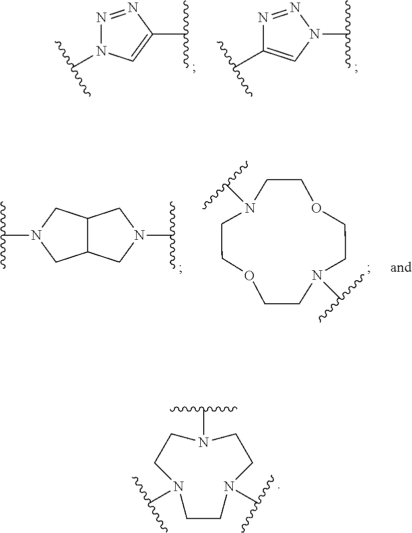 [SMILES: *c1cn(*)nn1 ; *c1cn(*)nn1 ; *N1CC2CN(*)CC2C1 ; *N1CCOCCN(*)CCOCC1 ; *N1CCN(*)CCN(*)CC1]

In some embodiments, one or more of Lₐ, L_{b}, L_{c1}, and L_{c2} include one or more units selected from:

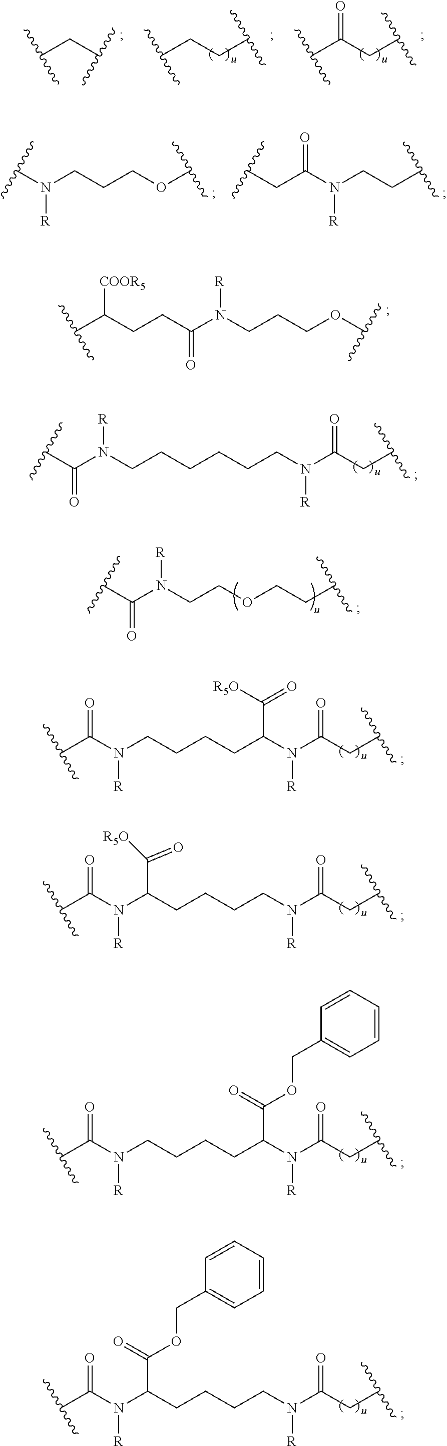

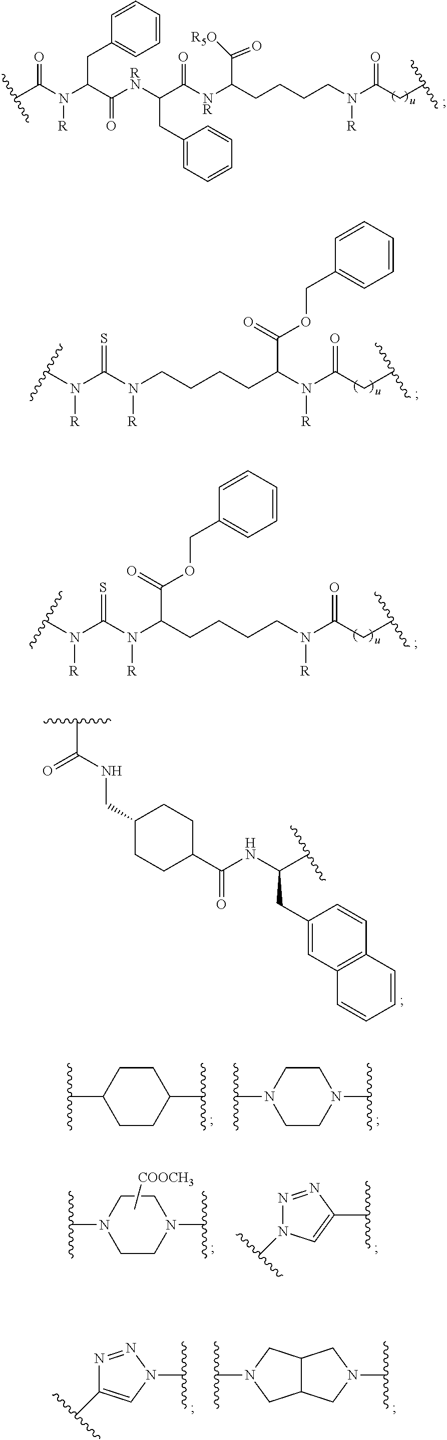

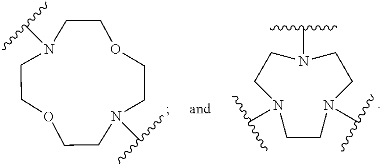

wherein u is an integer selected from 1, 2, 3, 4, 5, 6, 7, and 8; and R and R₅ are as defined hereinabove.

In some embodiments, the compound of formula (III) is selected from:

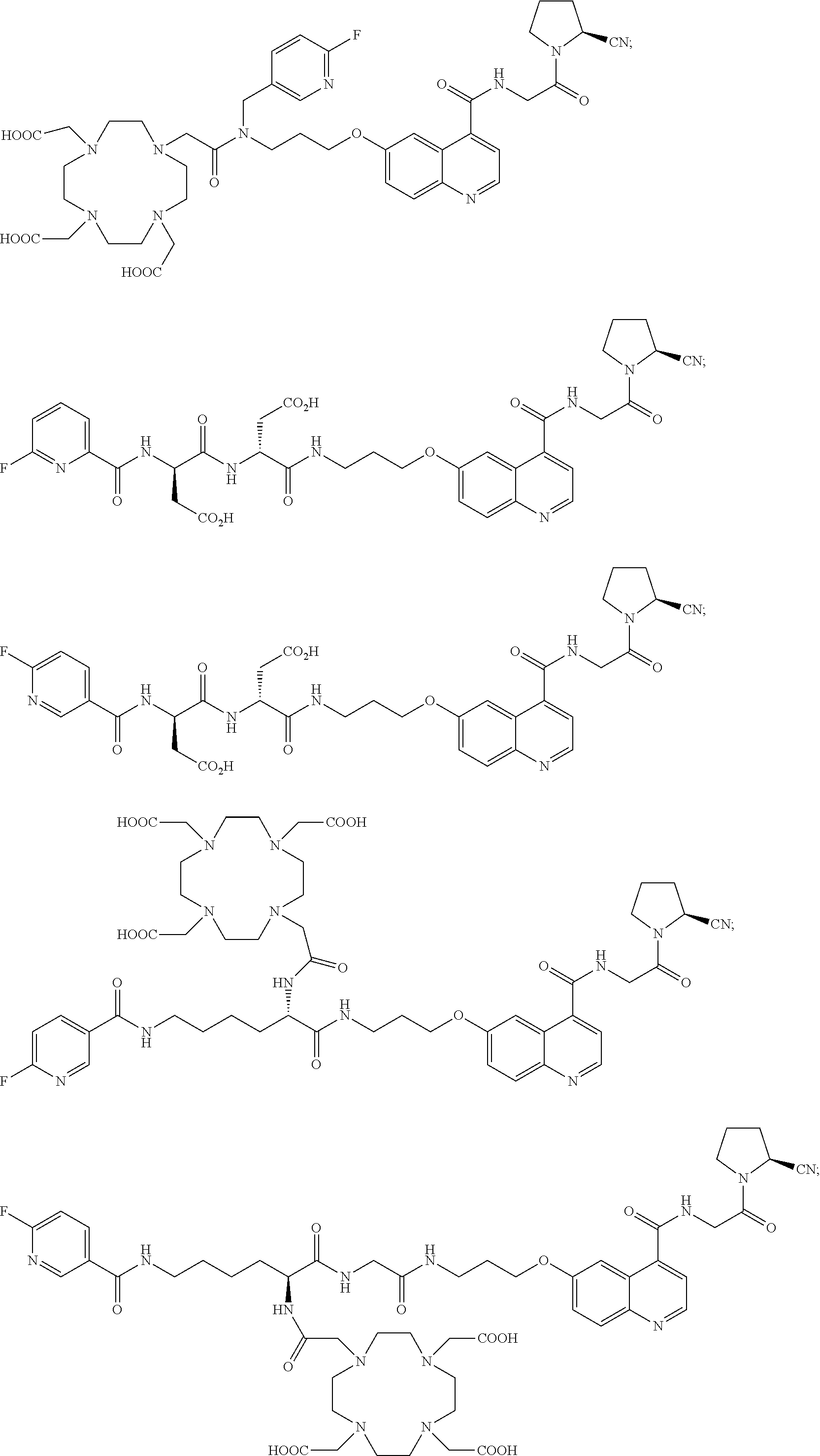

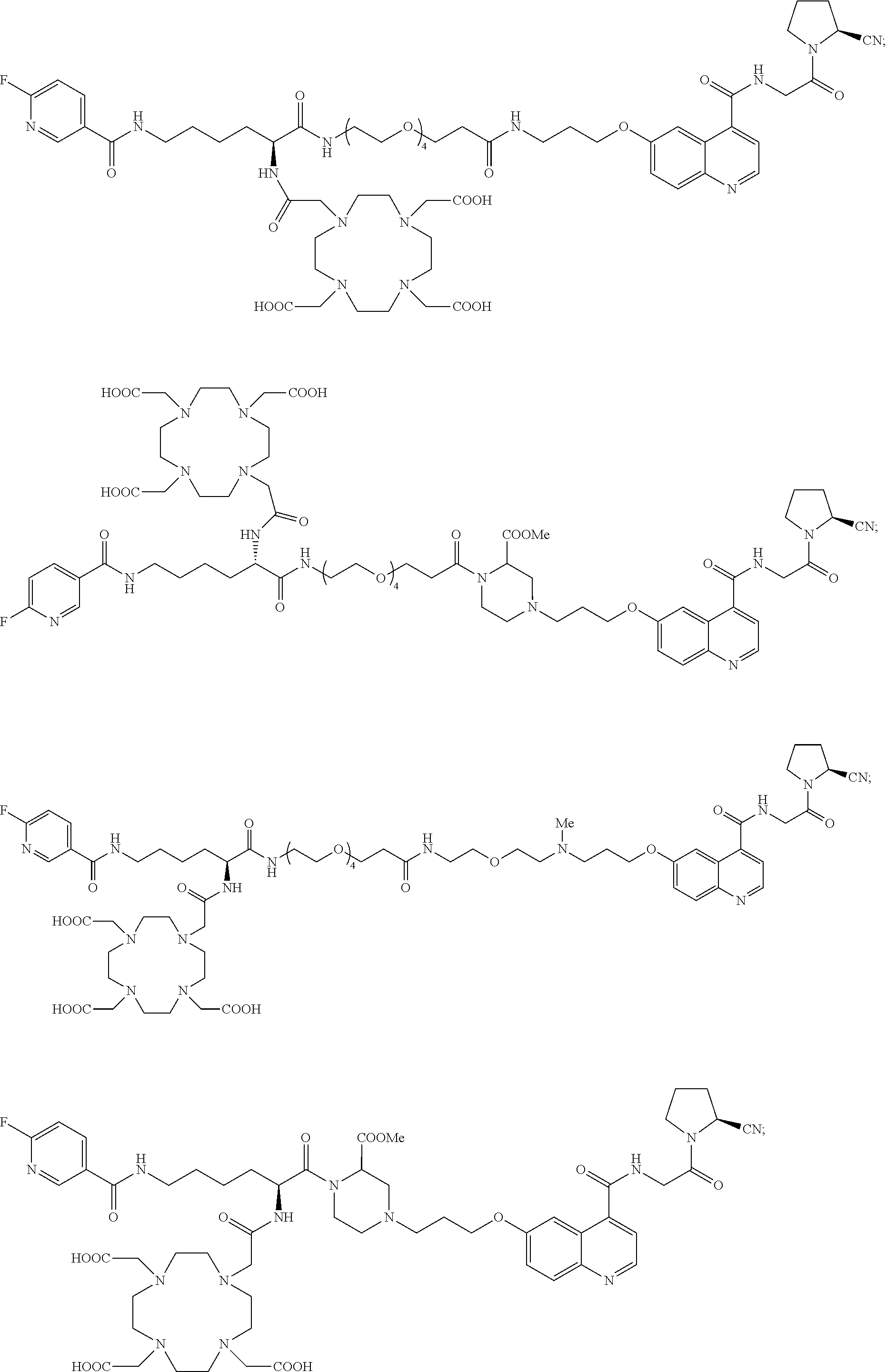

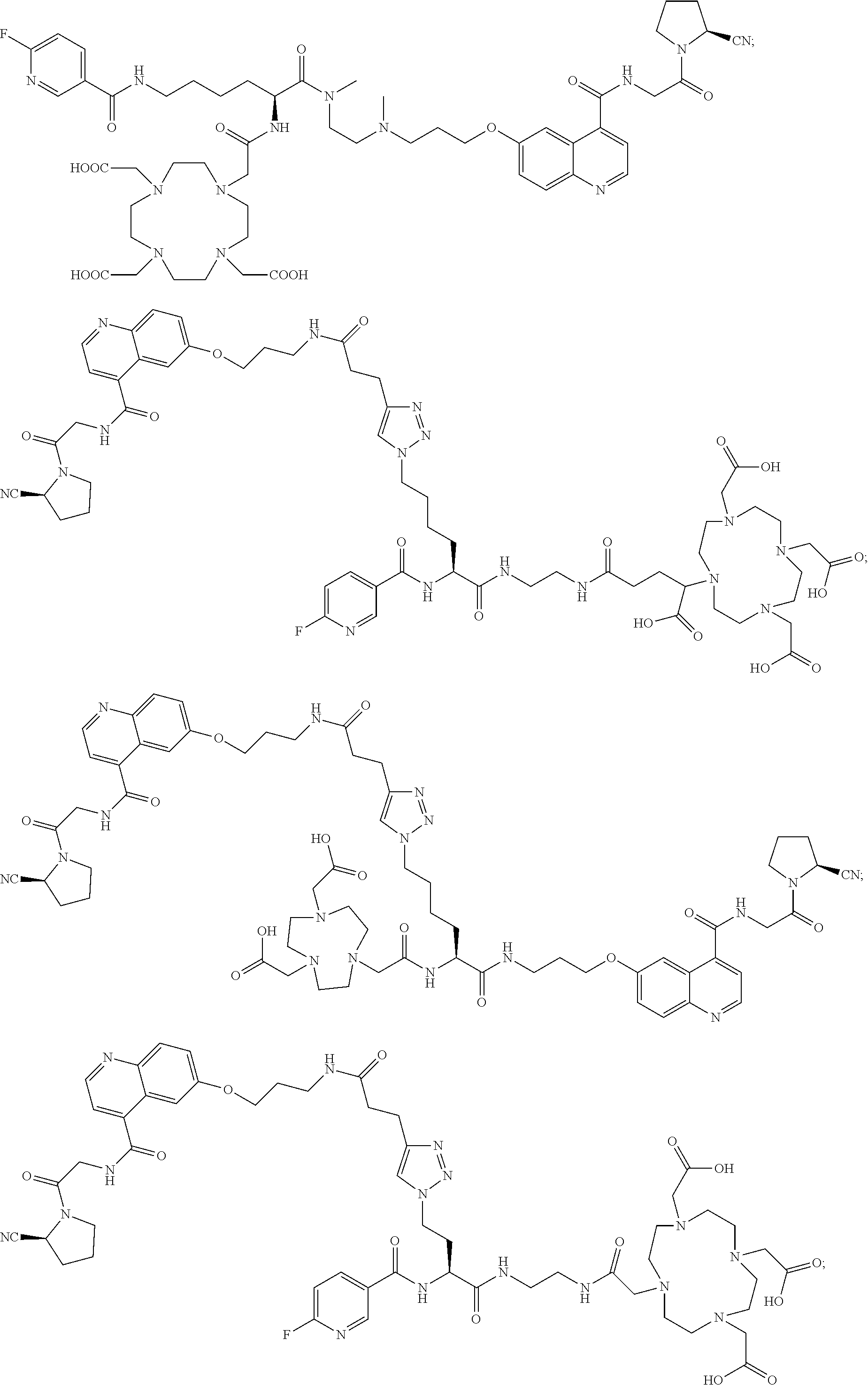

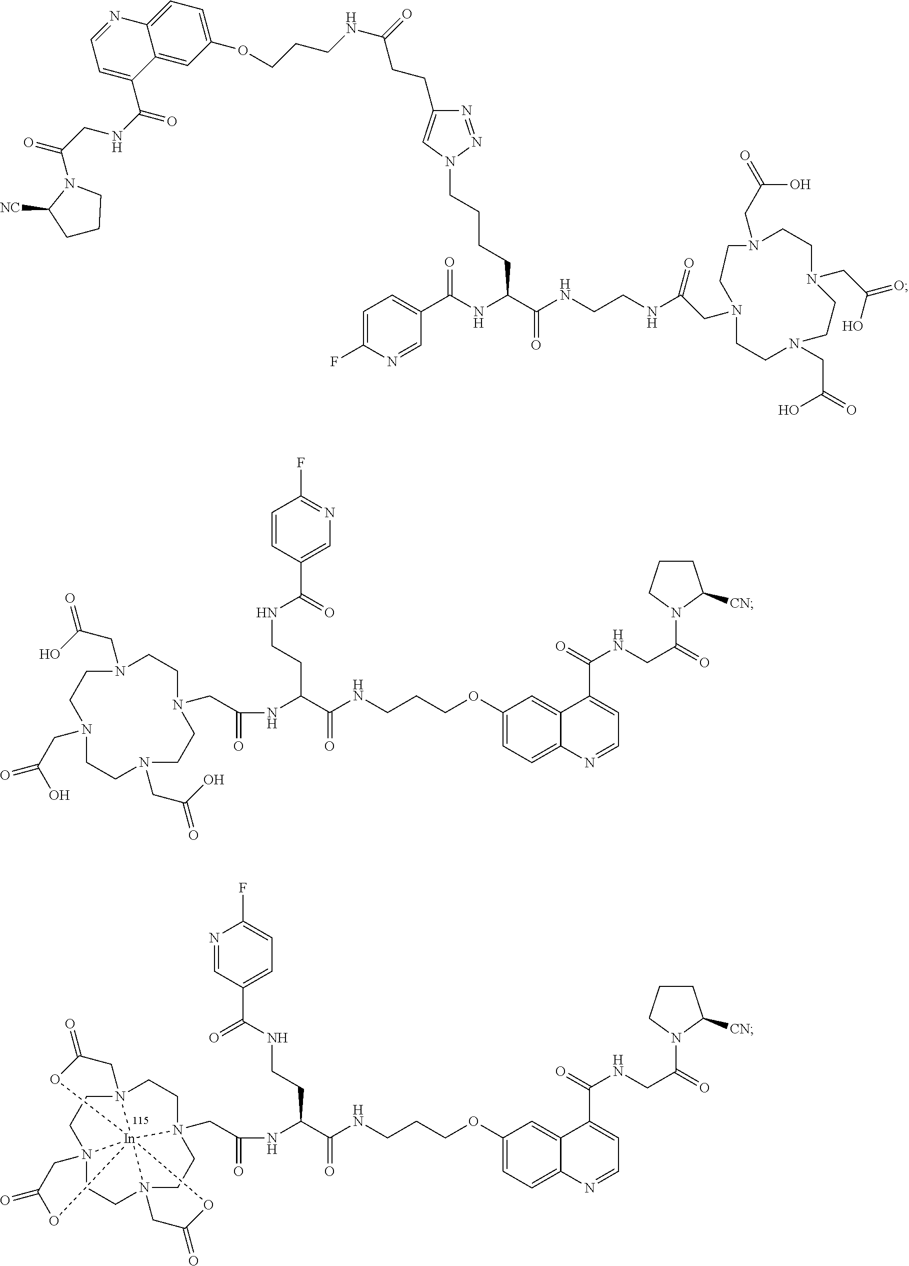

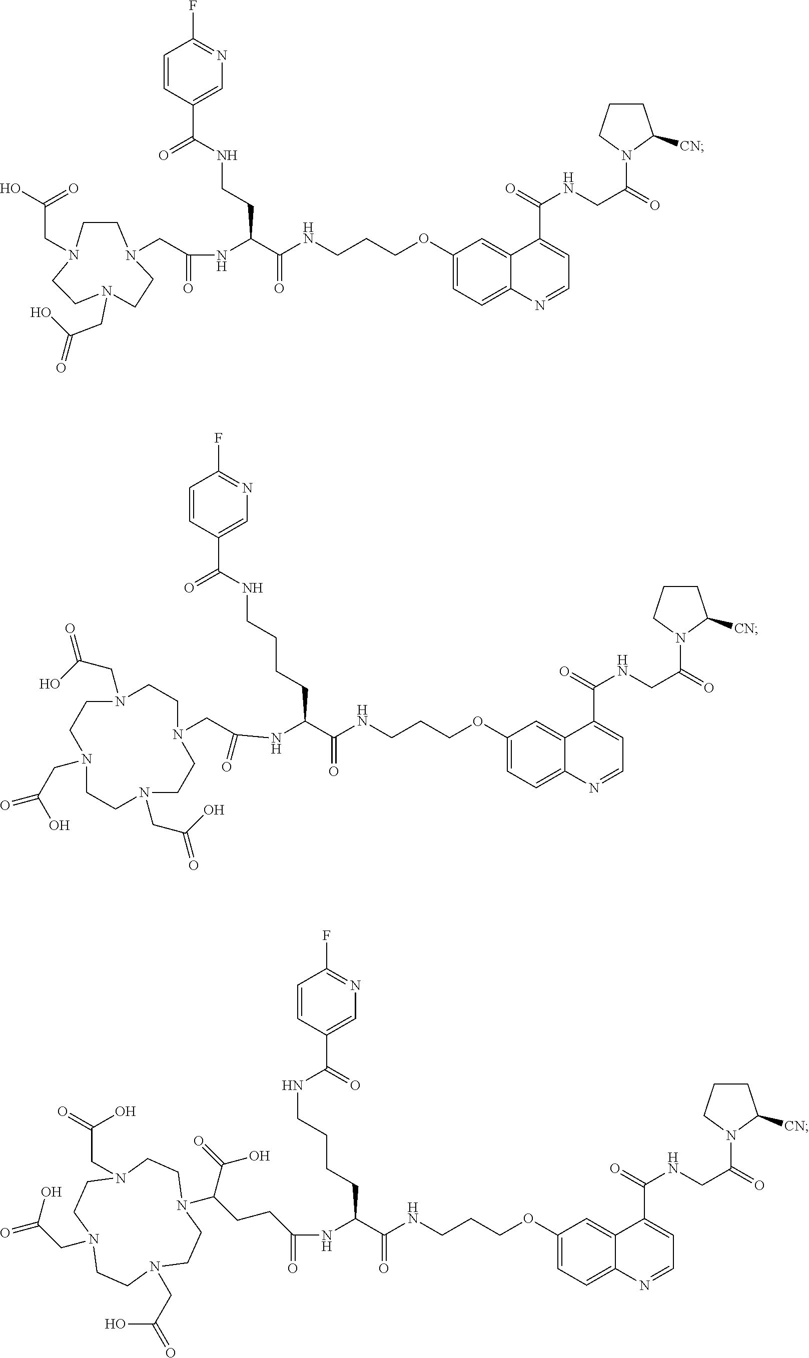 [SMILES: N#C[C@@H]1CCCN1C(=O)CNC(=O)c1ccnc2ccc(OCCCNC(=O)[C@H](CCNC(=O)c3ccc(F)nc3)NC(=O)CN3CCN(CC(=O)O)CCN(CC(=O)O)CC3)cc12 ; N#C[C@@H]1CCCN1C(=O)CNC(=O)c1ccnc2ccc(OCCCNC(=O)[C@H](CCCCNC(=O)c3ccc(F)nc3)NC(=O)CN3CCN(CC(=O)O)CCN(CC(=O)O)CCN(CC(=O)O)CC3)cc12 ; N#C[C@@H]1CCCN1C(=O)CNC(=O)c1ccnc2ccc(OCCCNC(=O)[C@H](CCCCNC(=O)c3ccc(F)nc3)NC(=O)CCC(C(=O)O)N3CCN(CC(=O)O)CCN(CC(=O)O)CCN(CC(=O)O)CC3)cc12]

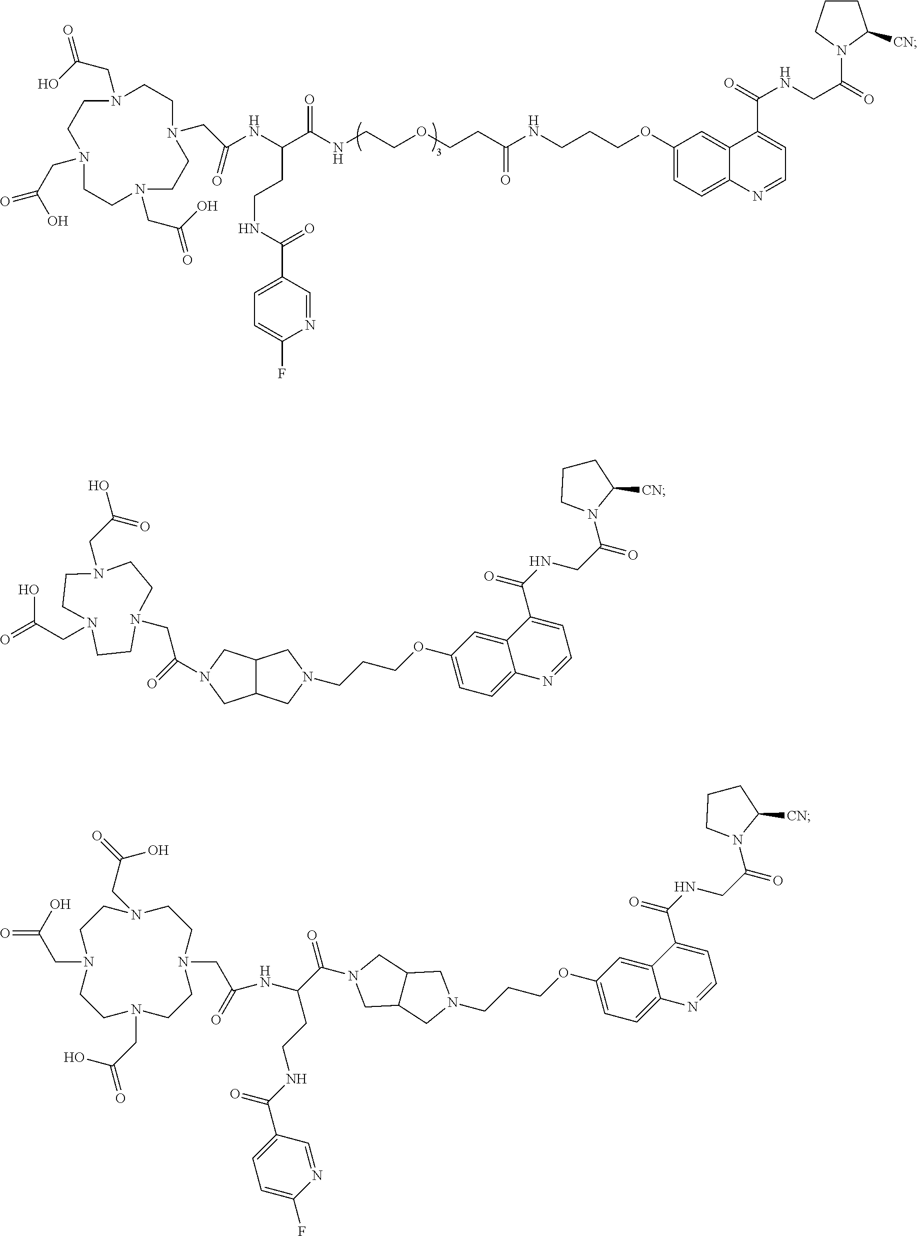

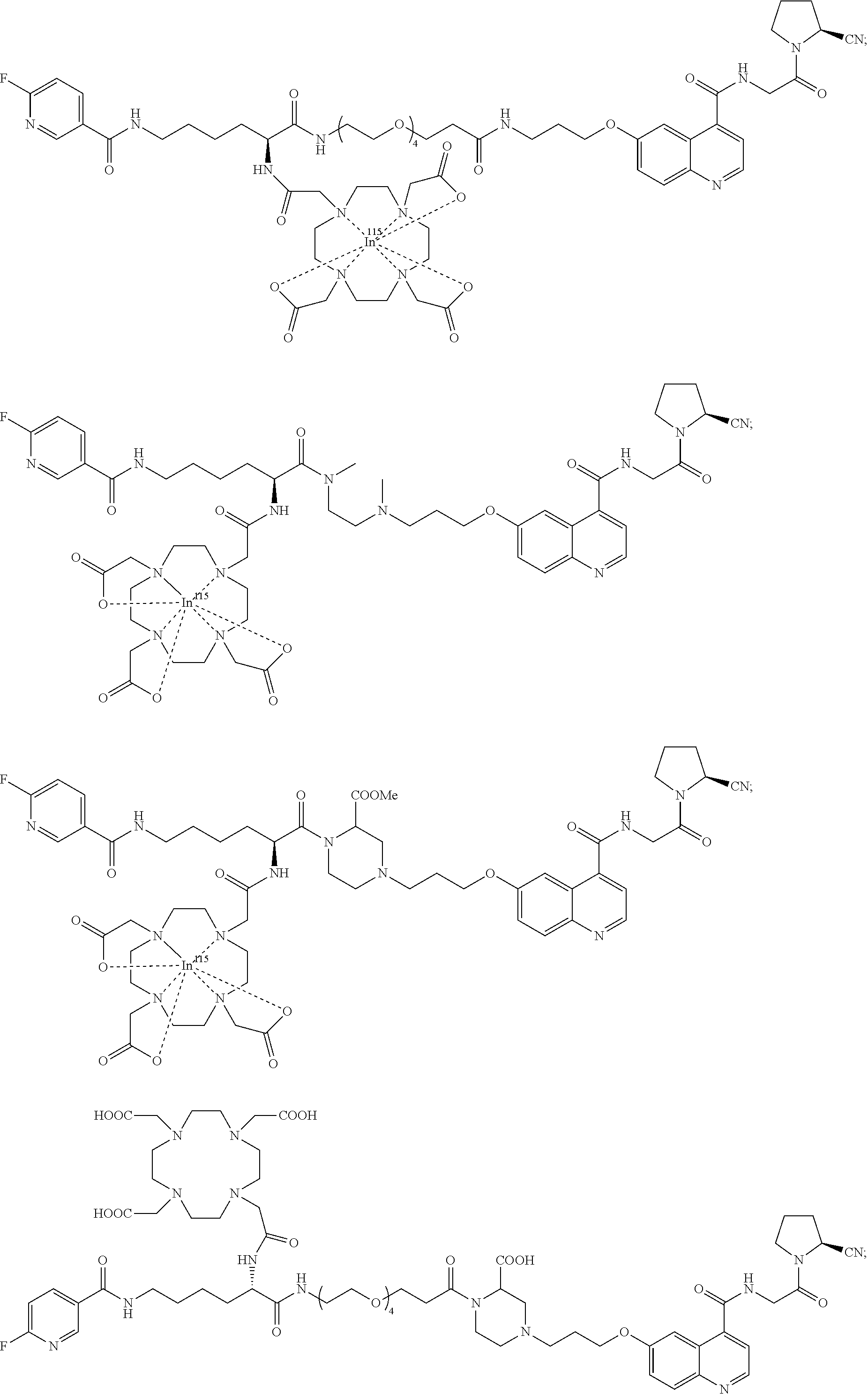

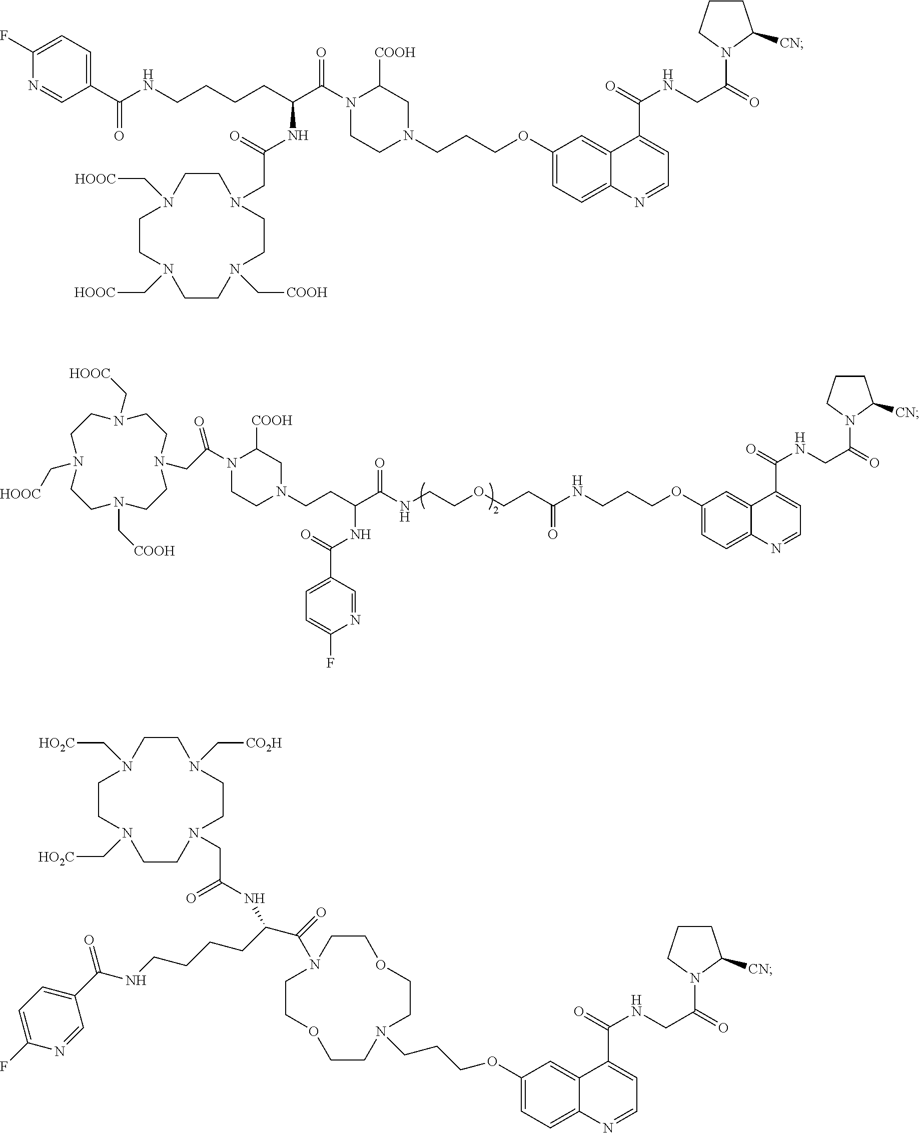

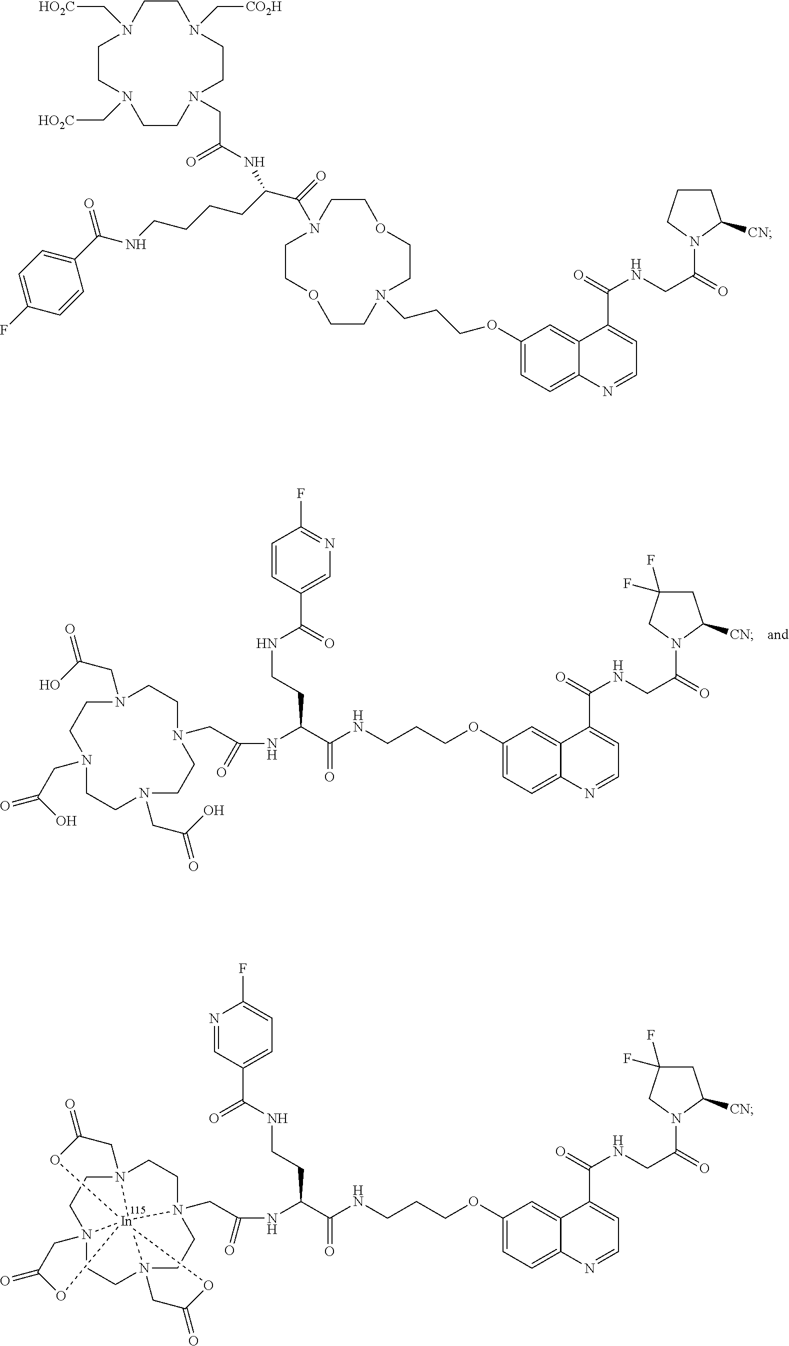

In some embodiments, the prosthetic group C₂ is covalently bound to the chelating group C₁.

In some embodiments, the compound is selected from:

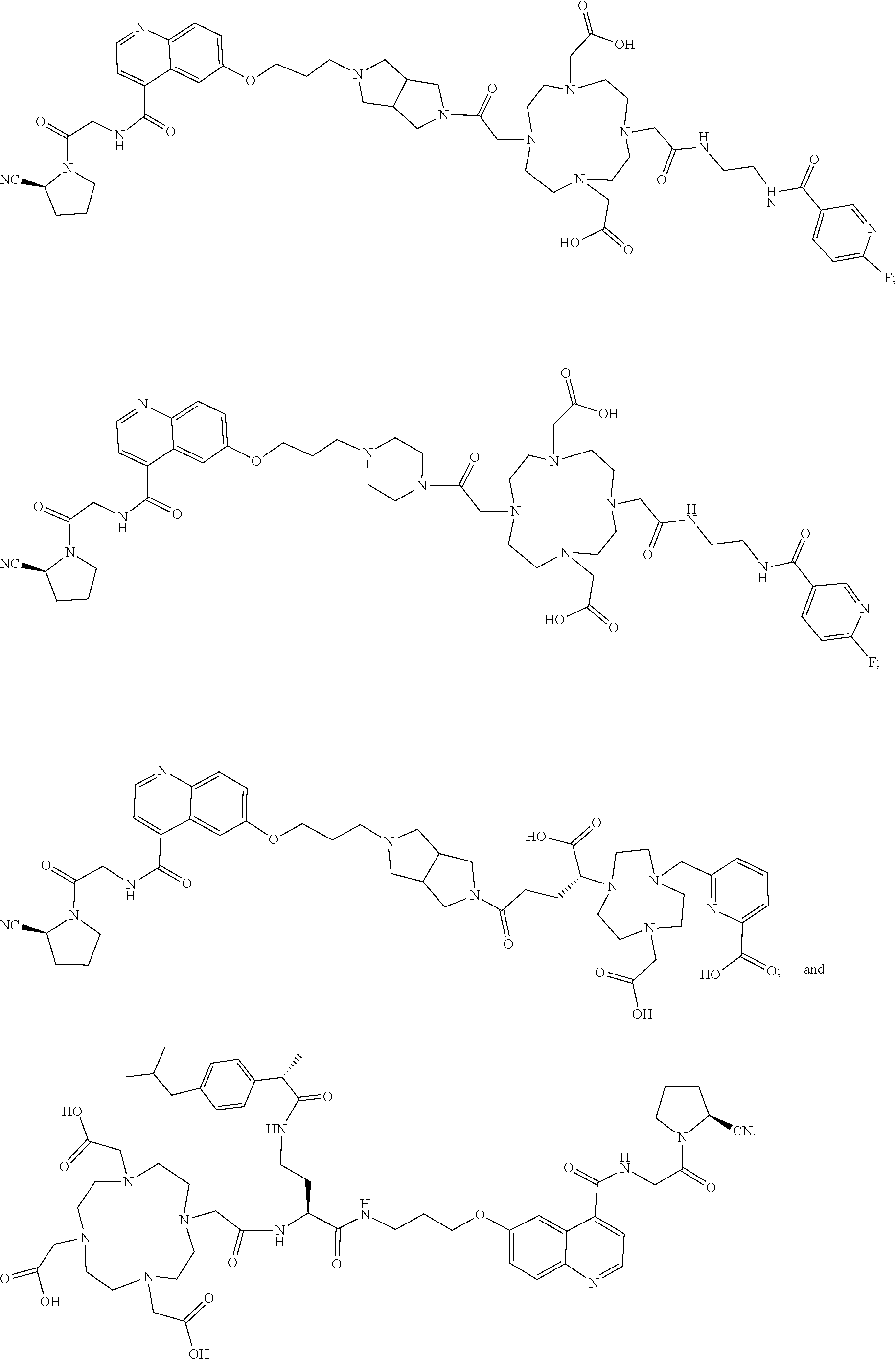

In some embodiments, the presently disclosed subject matter provides a compound of formula (IV):

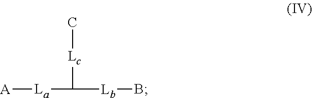

wherein:
A is a targeting moiety for fibroblast activation protein alpha (FAP-α);
B is a targeting moiety for prostate-specific membrane antigen (PSMA);
C is any optical or radiolabeled functional group suitable for optical imaging, photoacoustic imaging, positron emission tomography (PET) imaging, single-photon emission computed tomography (SPECT) imaging, or radiotherapy; and
Lₐ, L_{b}, and L_{c} are each a bi-functionalized linker capable of forming a chemical bond with each other and A, B, and C, respectively; and stereoisomers and pharmaceutically acceptable salts thereof.

In some embodiments, A is a FAP-α targeting moiety having the structure of:

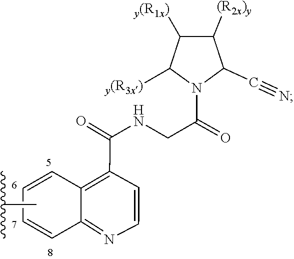

wherein [symbol] indicates a point of attachment of the FAP-α binding ligand to the linker Lₐ and/or L_{b} wherein the point of attachment can be through any of carbon atoms 5, 6, 7, or 8 of the quinolinyl ring thereof; and stereoisomers and pharmaceutically acceptable salts thereof.

In some embodiments, B is a targeting moiety for PSMA having the following structure:

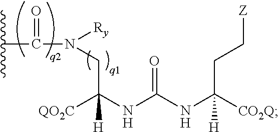

wherein:
   Z is tetrazole or CO₂Q;
   Q is H or a protecting group;
   q₁ is an integer selected from the group consisting of 1, 2, 3, 4, and 5;
   q₂ is an integer from selected from 0 or 1; and
   R_{y} is independently H or —(CH₂)_{q3}—R_{y1}, wherein q₃ is an integer selected from the group consisting of 0, 1, 2, 3, 4, 5, and 6 and R_{y1} is selected from the group consisting of substituted aryl, substituted pyridine, and unsubstituted isoquinoline;

In some embodiments, B is a PSMA targeting moiety having the following structure:

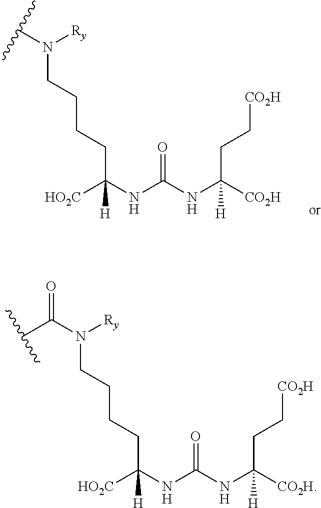

In some embodiments, R_{y1} is selected from the group consisting of:

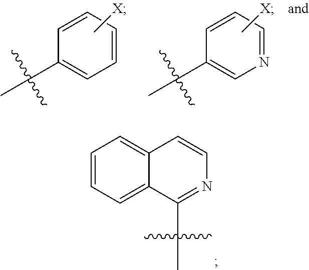

wherein X is independently selected from the group consisting of Br, ⁷⁵Br, ⁷⁶Br, ⁷⁷Br, ^{80m}Br, ⁸²Br, I, ¹²⁴I, ¹²³I, ¹²⁵I, ¹³¹I, At, and ²¹¹At.

In some embodiments, C is a radiolabeled prosthetic group comprising a radioisotope selected from the group consisting of ¹⁸F, ¹²⁴I, ¹²⁵I, ¹³¹I, and ²¹¹At.

In some embodiments, the radiolabeled prosthetic group is selected from the group consisting of:

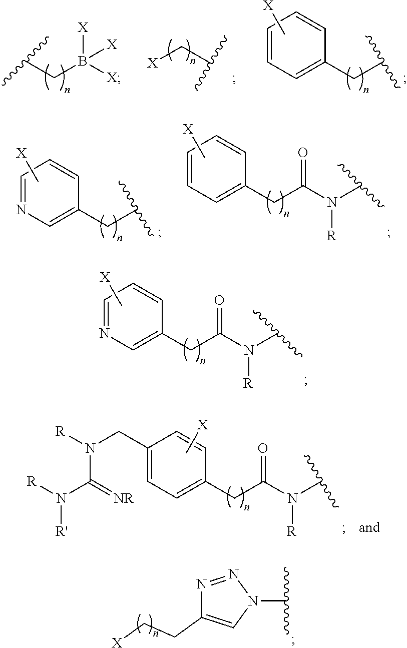

wherein each X is independently a radioisotope selected from the group consisting of ¹⁸F, ¹²⁴I, ¹²⁵I, ¹³¹I, and ²¹¹At; each R and R′ is defined hereinabove; and each n is independently an integer selected from the group consisting of 0, 1, 2, 3, 4, 5, 6, 7, 8, 9, 10, 11, 12, 13, 14, 15, 16, 17, 18, 19, and 20.

In some embodiments, the radiolabeled prosthetic group is selected from the group consisting of:

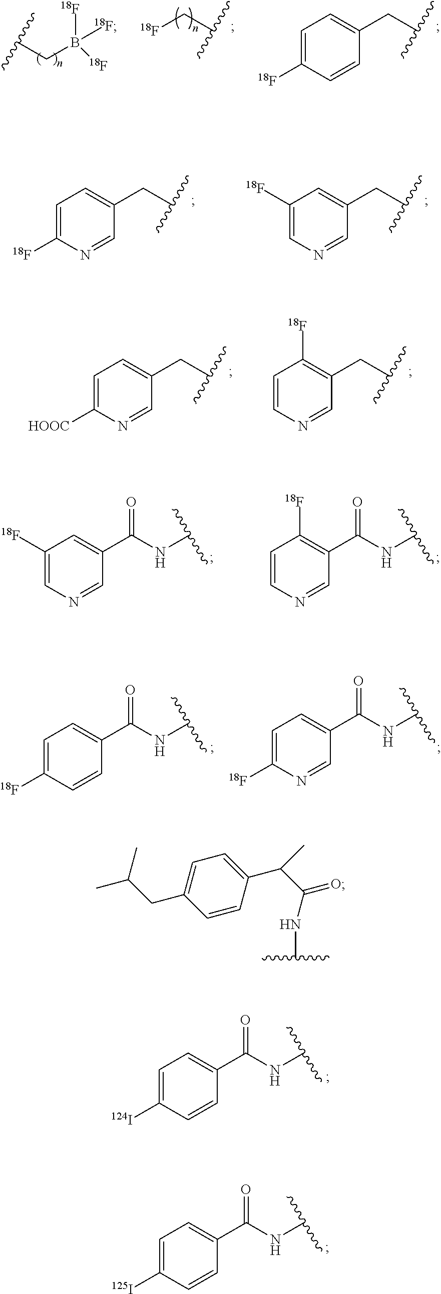

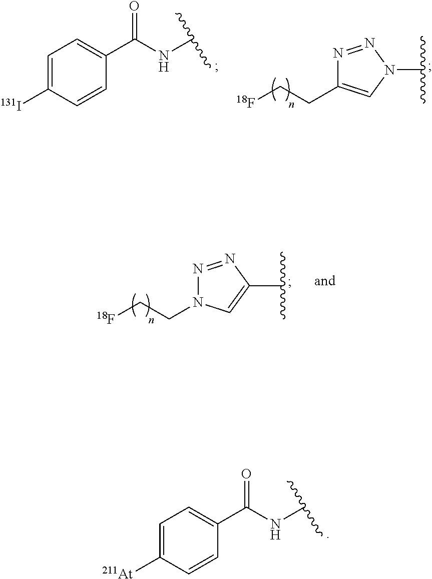

In some embodiments, C comprises a chelating agent selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), (NOTAGA) 1,4,7-triazonane-1,4-diyl)diacetic acid DFO (Desferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino) methyl) benzoic acid), and BaBaSar.

In some embodiments, C is a chelating agent is selected from the group consisting of:

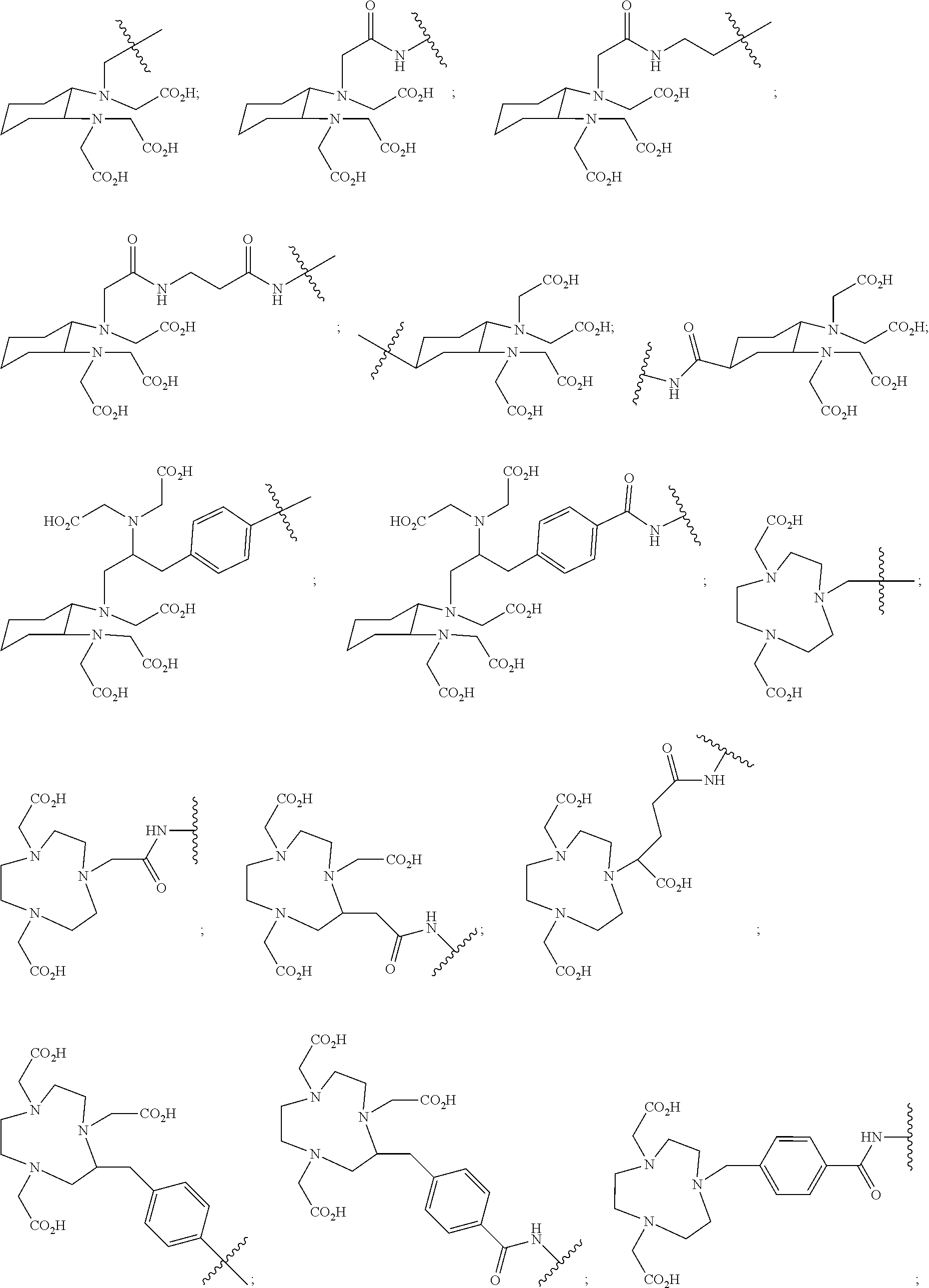

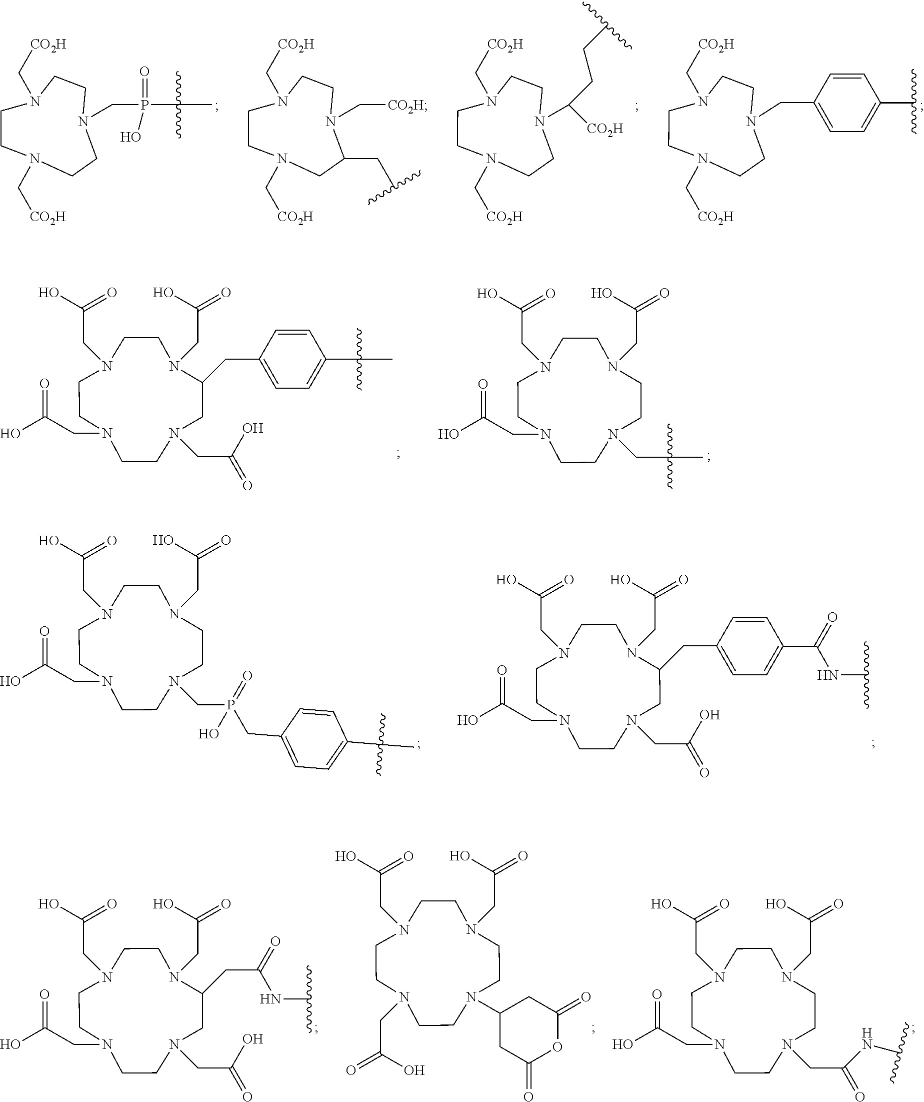

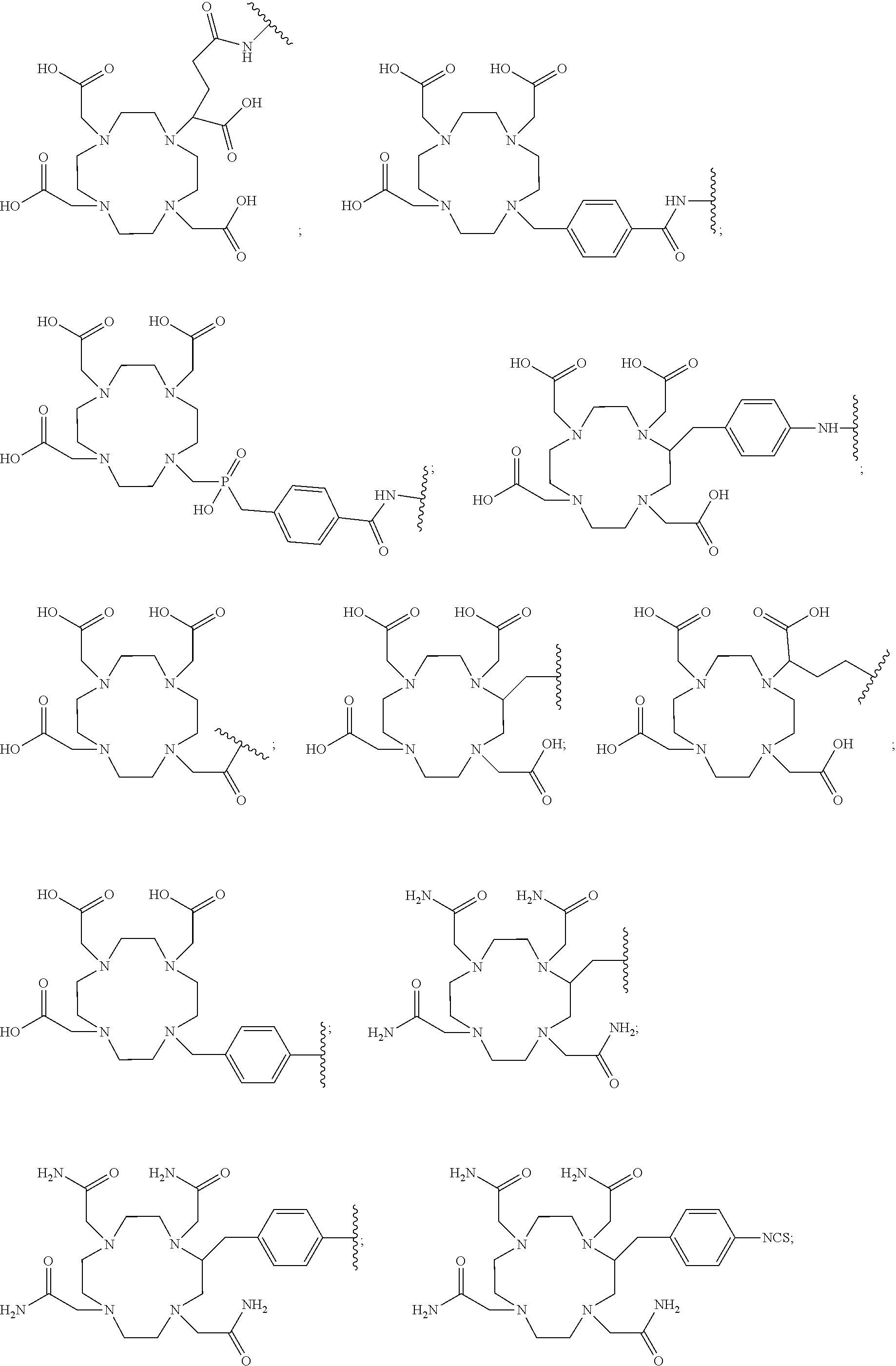

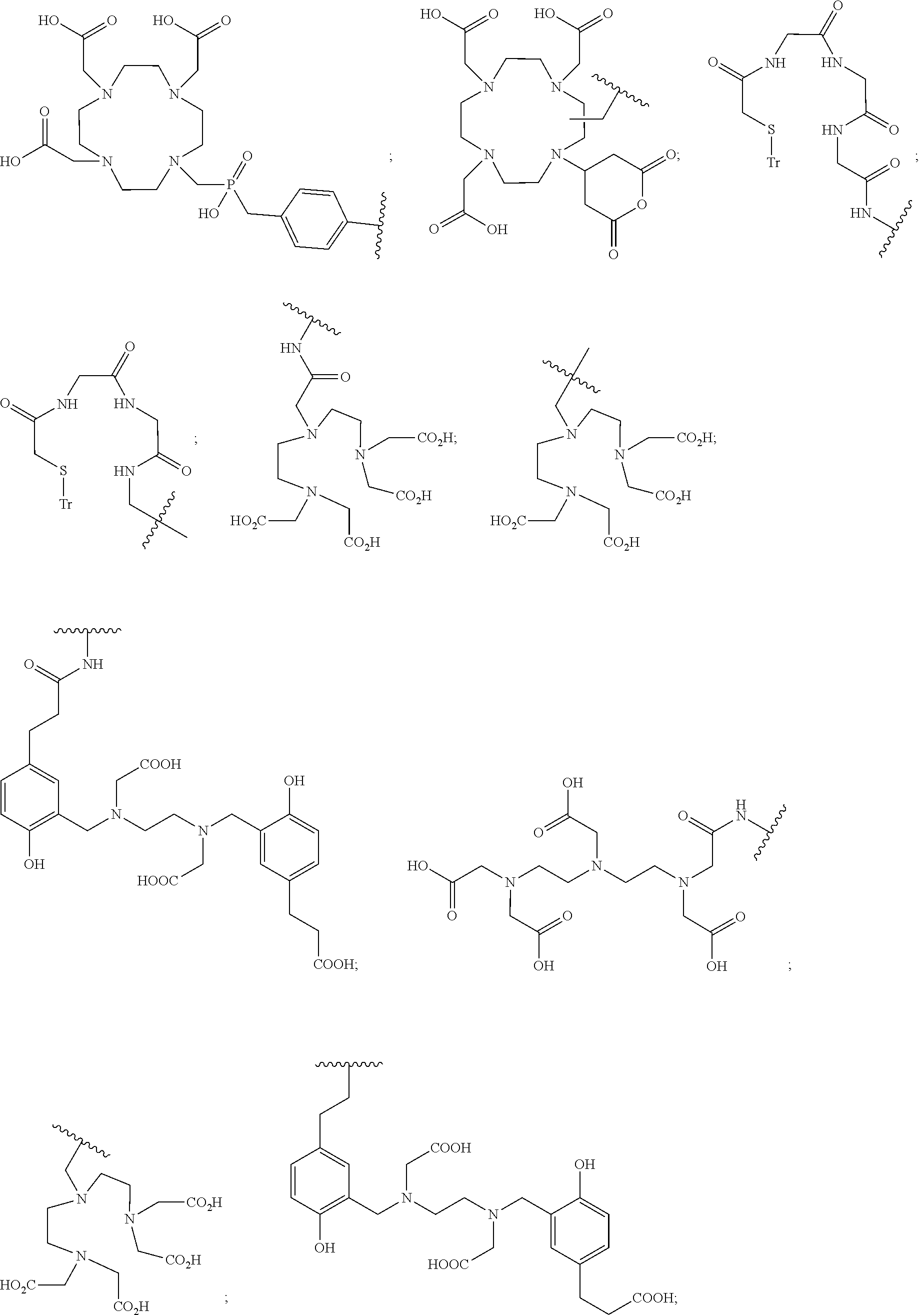

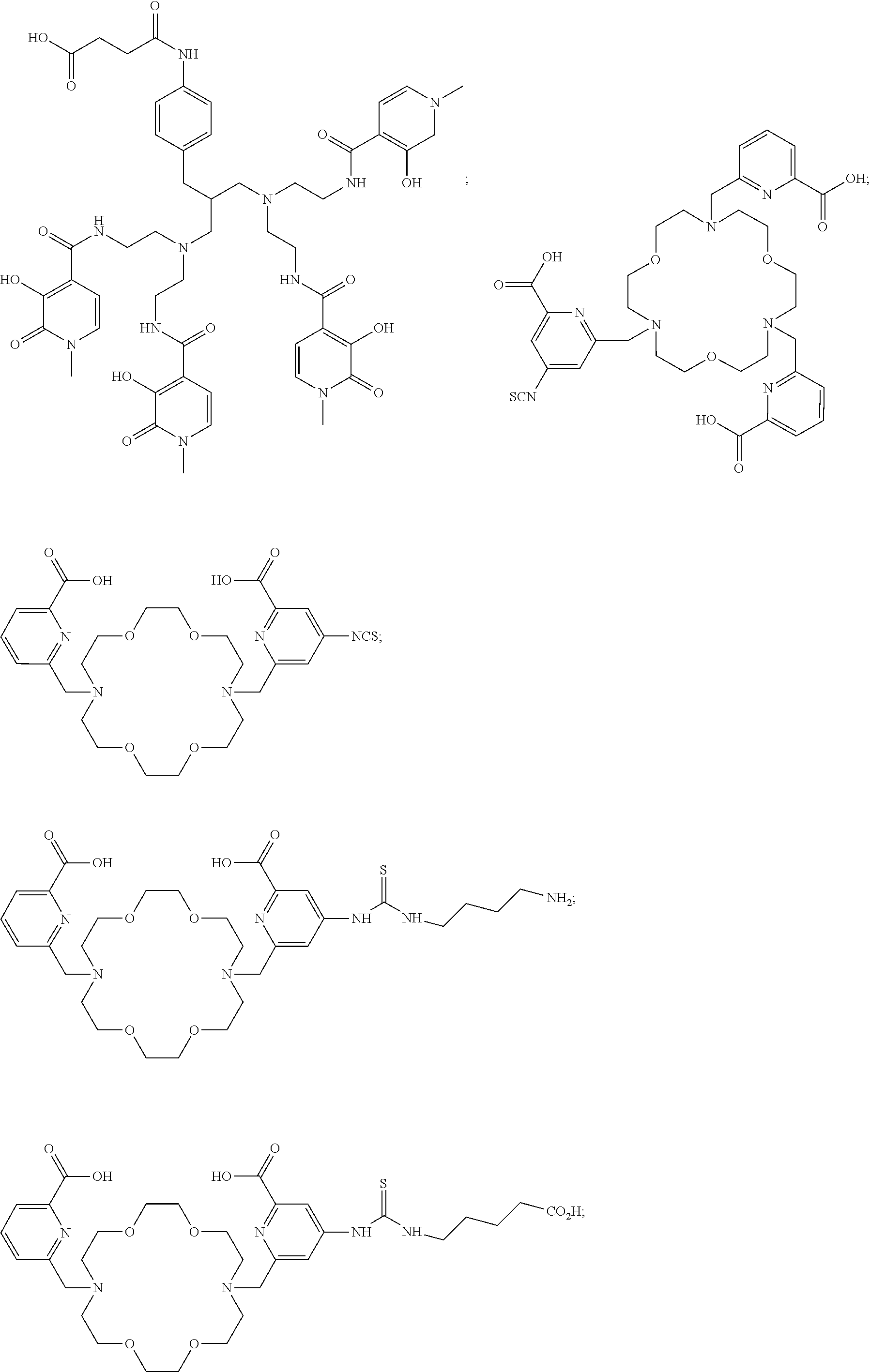

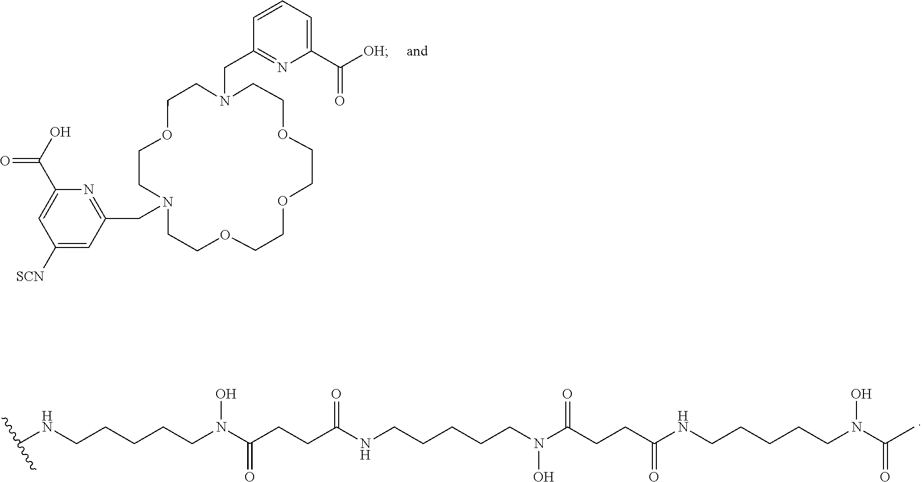

In some embodiments, the chelating agent further comprises a radiometal.

In some embodiments, the radiometal is selected from the group consisting of ⁶Cu, ⁶²Cu, ⁶⁴Cu, ⁶⁷Cu, ²⁰³Pb, ²¹²Pb, ²²⁵Ac, ¹⁷⁷Lu, ^{99m}Tc, ⁶⁸Ga, ¹⁴⁹Tb, ⁸⁶Y, ⁹⁰Y, ¹¹¹In, ¹¹⁵In, ¹⁸⁶Re, ¹⁸⁸Re, ¹⁵³Sm, ⁸⁹Zr, ²¹³Bi, ²¹²Bi, ²¹²Pb, ⁶⁷Ga, ⁴⁷Sc, ¹⁶⁶Ho, ⁴³Sc, ²²³Ra, ^{226/227}Th, Al-¹⁸F, and Sc-¹⁸F.

In some embodiments, C comprises an optical dye.

In some embodiments, the optical dye comprises a fluorescent dye.

In some embodiments, the fluorescent dye comprises a fluorescent dye that emits in the near infrared spectral region.

In some embodiments, the fluorescent dye is selected from the group consisting of a polymethine dye, a coumarin dye, a xanthene dye, and a boron-dipyrromethene (BODIPY) dye.

In some embodiments, the polymethine dye is selected from the group consisting of a carbocyanine dye, an indocarbocyanine dye, an oxacarbocyanine dye, a thiacarbocyanine dye, and a merocyanine dye.

In some embodiments, the xanthene dye is selected from the group consisting of a fluorescein dye and a coumarin dye.

In some embodiments, the fluorescent dye is selected from the group consisting of:
BODIPY FL, BODIPY R6G, BODIPY TR, BODIPY TMR, BODIPY 493/503, BODIPY 530/550, BODIPY 558/568, BODIPY 564/570, BODIPY 576/589, BODIPY 581/591, BODIPY 630/650, and BODIPY 650/665;
Cy3, Cy3.5, Cy5, Cy5.5, Cy7, and Cy7.5;
VivoTag-645, VivoTag-680, VivoTag-S680, VivoTag-S750, VivoTag-800;
Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, Alexa Fluor 700, Alexa Fluor 750, and AlexaFluor790;
Dy677, Dy676, Dy682, Dy752, Dy780; DyLight 350, DyLight 405, DyLight 488, DyLight 547, DyLight 550, DyLight 594, DyLight 633, DyLight 647, DyLight 650, DyLight 680, DyLight 755, and DyLight 800;
HiLyte Fluor 405, HiLyte Fluor 488, HiLyte Fluor 532, HiLyte Fluor 555, HiLyte™ Fluor 594, HiLyte Fluor 647, HiLyte Fluor 680, HiLyte Fluor 750;
IR800 (Dimethyl {4-[1,5,5-tris(4-dimethylaminophenyl)-2,4-pentadienylidene]-2,5-cyclohexadien-1-ylidene}ammonium perchlorate),
IRDye 650, IRDye 680RD, IRDye 680LT, IRDye 700, IRDye 700DX, IRDye 750, IRDye 800, IRDye 800CW, IRDye 800RS; and
ADS1065A, ADS1075A, ADS775MI, ADS775MP, ADS775PI, ADS775PP, ADS780HO, ADS780WS, ADS785WS, ADS790WS, ADS795WS, ADS798SM, ADS800AT, ADS815EI, ADS830AT, ADS830WS, ADS832WS, ADS845MC, and ADS920MC.

In some embodiments, the optical dye is selected from the group consisting of:

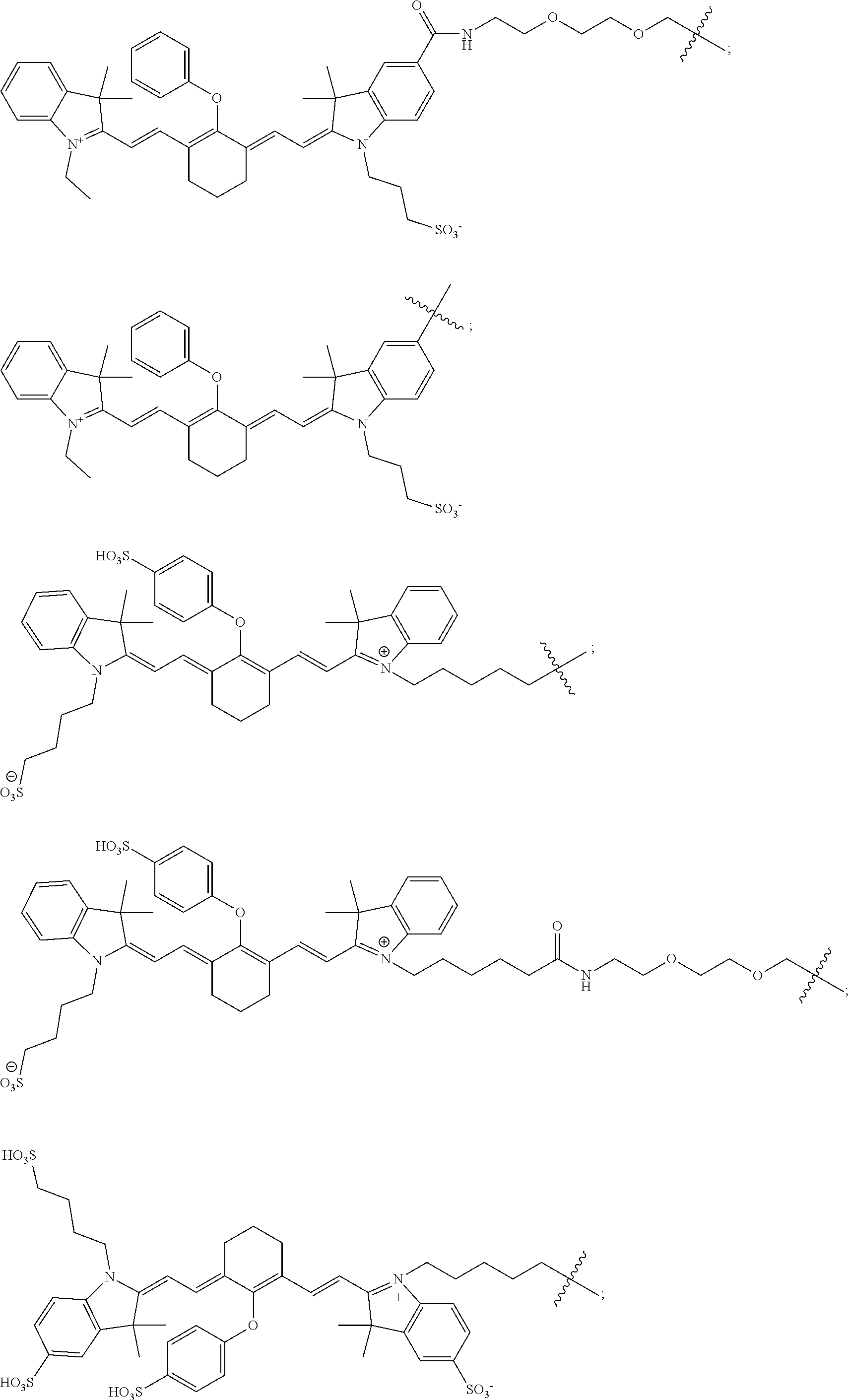

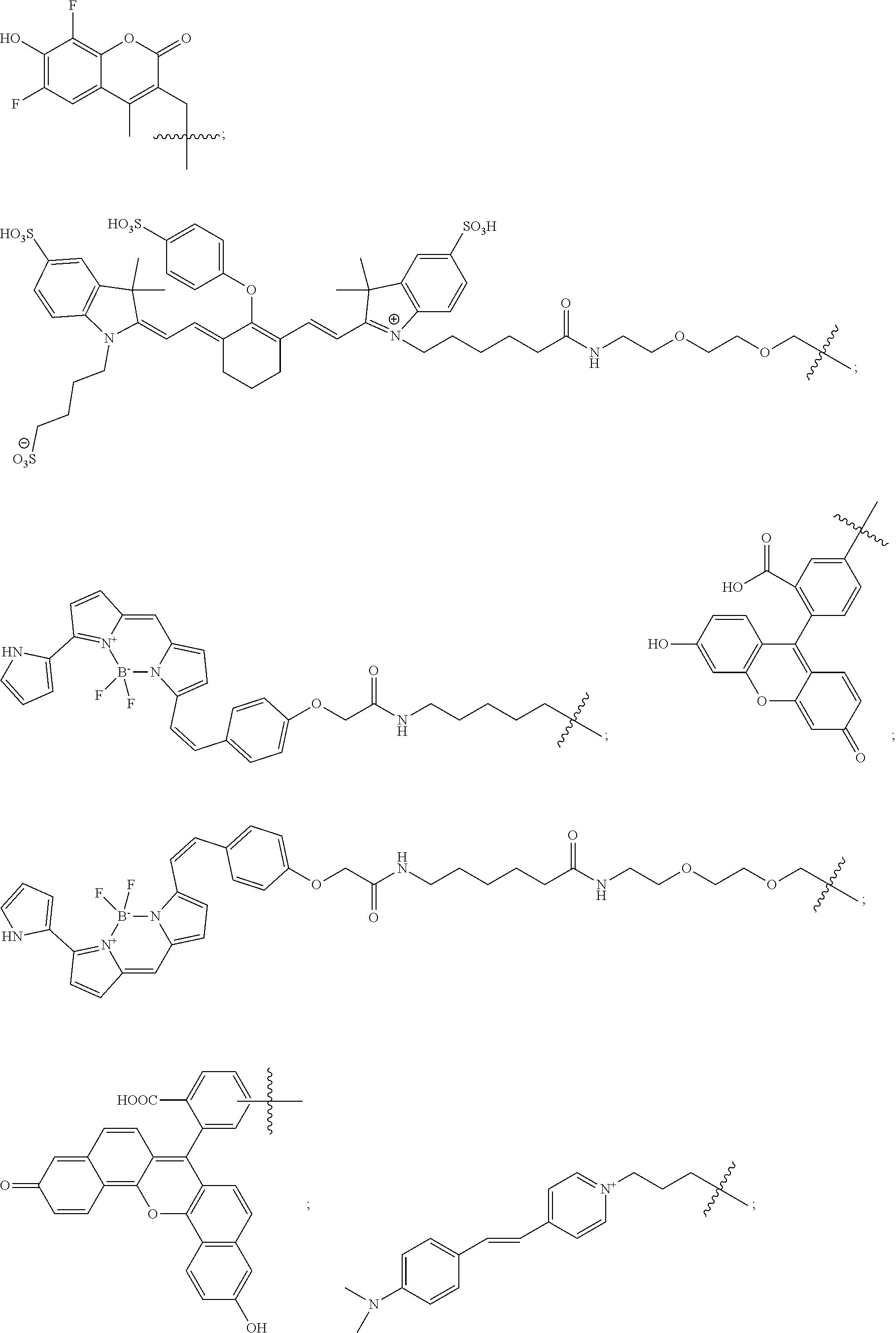

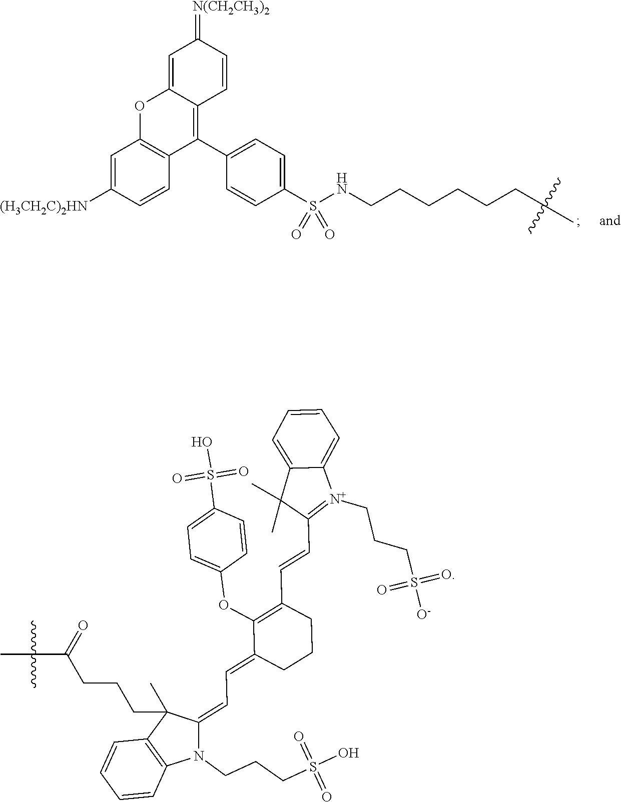

In some embodiments, Lₐ, L_{b}, and L_{c} are each individually selected from the group consisting of (a), (b), (c), or (d):
(a)

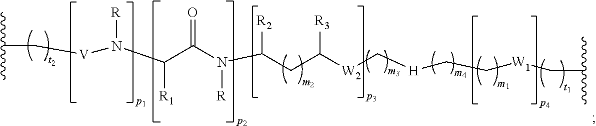

wherein:
p₁, p₂, p₃ and p₄ may be in any order;
t₁ and t₂ are each an integer selected from the group consisting of 0, 1, 2, 3, 4, 5, 6, 7, and 8;
p₁, p₃, and p₄ are each independently 0 or 1;
p₂ is an integer selected from the group consisting of 0, 1, 2, and 3, and when p₂ is 2 or 3, each R₁ is the same or different;
m₁, m₂, m₃, and m₄ are each an integer independently selected from the group consisting of 0, 1, 2, 3, 4, 5, 6, 7 and 8;
W₁ is selected from the group consisting of a bond, —S—, —C(═O), C(═O)—NR—, and —NR—C(═O);
W₂ is selected from the group consisting of a bond, —S—, —CH₂—C(═O)—NR—, —C(═O), —NRC(═O)—, —NR′C(═O)NR—, —NRC(═S)NR′₂—, —NRC(═O)O—, —OC(═O)NR—, —OC(═O), —C(═O)NR—, —NR—C(═O)—, —C(═O)O—, —(O—CH₂—CH₂)_{q}— and —(CH₂—CH₂—O)_{q}, wherein q is selected from the group consisting of 0, 1, 2, 3, 4, 5, 6, 7, and 8;
each R or R′ is independently H, alkyl, substituted alkyl, cycloalkyl, substituted cycloalkyl, heterocycloalkyl, substituted heterocycloalkyl, aryl, substituted aryl, heteroaryl, substituted heteroaryl, and —OR₄, wherein R₄ is selected from the group consisting of H, alkyl, substituted alkyl, cycloalkyl, substituted cycloalkyl, heterocycloalkyl, and substituted heterocycloalkyl, wherein q is defined as immediately hereinabove;
H can be present or absent and when present is selected from a nitrogen-containing heteroalkylene chain, a cyclic or bicylic heteroalkyl radical, and a triazole radical having the structure

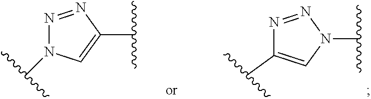 [SMILES: *c1cn(*)nn1 ; *c1cn(*)nn1]

each R₁ is independently H, C₁-C₆ alkyl, C₃-C₁₂ aryl, —(CH₂)_{q}—C₃-C₁₂ aryl, —C₄-C₁₆ alkylaryl, or —(CH₂)_{q}—C₄-C₁₆ alkylaryl; R₂ and R₃ are each independently H, —(CH₂)_{q}—C₃-C₁₂ aryl, and —CO₂R⁵, wherein R₅ is selected from the group consisting of H, C₁-C₆ alkyl, C₃-C₁₂ aryl, and C₄-C₁₆ alkylaryl, wherein R₂ and R₃ can be the same or different, wherein q is selected from the group consisting of 0, 1, 2, 3, 4, 5, 6, 7, and 8;
V is selected from the group consisting of C(O)—, —C(S)—, NRC(O)—, —NRC(S)—, and —OC(O);
(b)

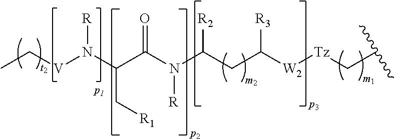

wherein p₁, p₂, p₃, m₁, m₂, Tz, W₂, R, R₁, R₂, R₃, and V are defined as hereinabove;
(c) -L₁-, -L₂-L₃-, or -L₁-L₂-L₃-, wherein:
L₁ is —NR—(CH₂)_{q}—[O—CH₂—CH₂—O]_{q}—(CH₂)_{q}—C(═O)—;
L₂ is —NR—(CH₂)_{q}—C(COOR₅)—NR—; and
L₃ is —(O═)C—(CH₂)_{q}—C(═O)—;
   wherein each q is independently an integer selected from the group consisting of 1, 2, 3, 4, 5, 6, 7, and 8; and R and R⁵ are as defined hereinabove; and
(d) —(CR₆H)_{q}—(CH₂)_{q}—C(═O)—NR—(CH₂)_{q}—O— or —NR—(CH₂)_{q}—O—; wherein each q and R is defined hereinabove; and R₆ is H or —COOR₅.

In some embodiments, H is selected from:

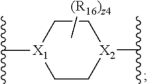

wherein X₁ and X₂ are each independently —CH— or N; each R₁₆ is independently H or —C(═O)—OR₁₇, wherein R₁₇ is C₁-C₄ alkyl;
—N(R₁₈)—(CH₂)_{z5}—N(R₁₉)—; wherein R₁₈ and R₁₉ are each independently H or C₁-C₄ alkyl and Z5 is an integer selected from 1, 2, 3, 4, 5, 6, 7, and 8;

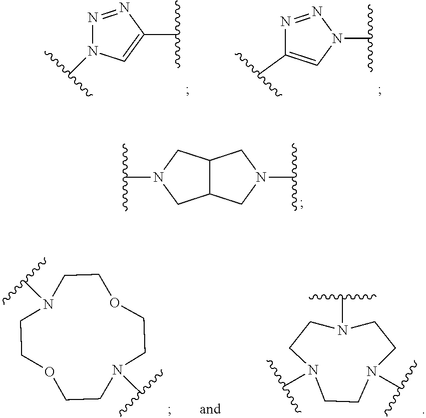 [SMILES: *c1cn(*)nn1 ; *c1cn(*)nn1 ; *N1CC2CN(*)CC2C1 ; *N1CCOCCN(*)CCOCC1 ; *N1CCN(*)CCN(*)CC1]

In some embodiments, one or more of Lₐ, L_{b}, and L_{c} include one or more units selected from:

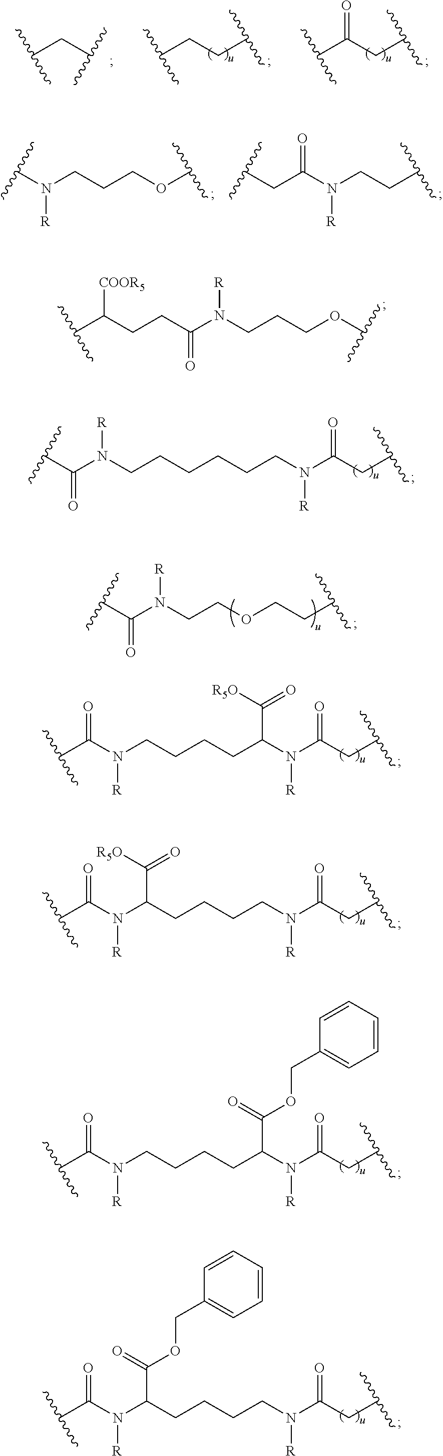

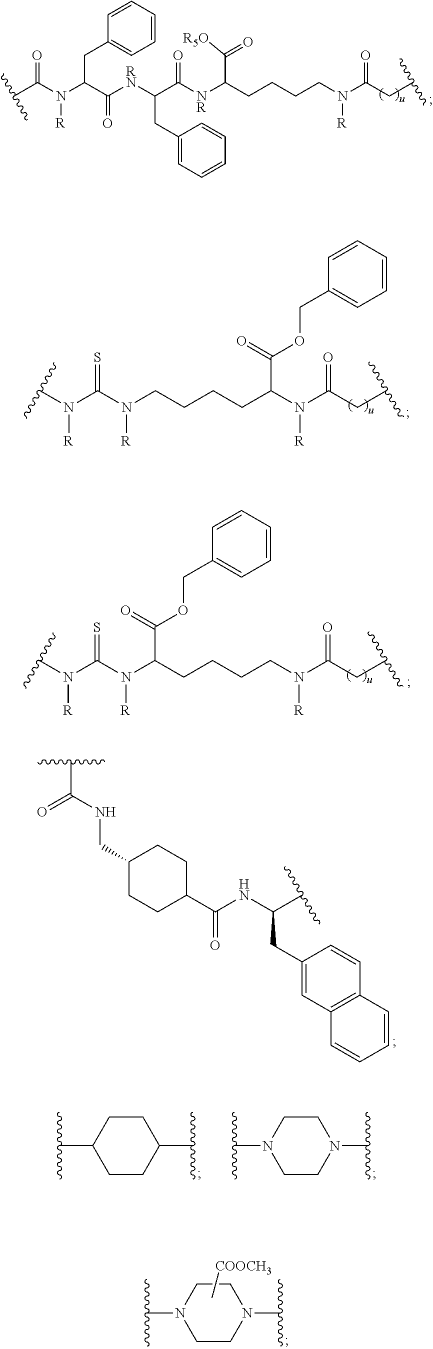

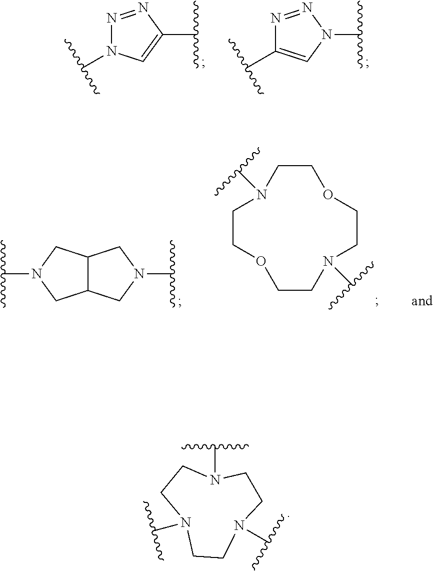

wherein u is an integer selected from 1, 2, 3, 4, 5, 6, 7, and 8; and R and R₅ are as defined hereinabove.

In some embodiments, the compound of formula (IV) is selected from the group consisting of:

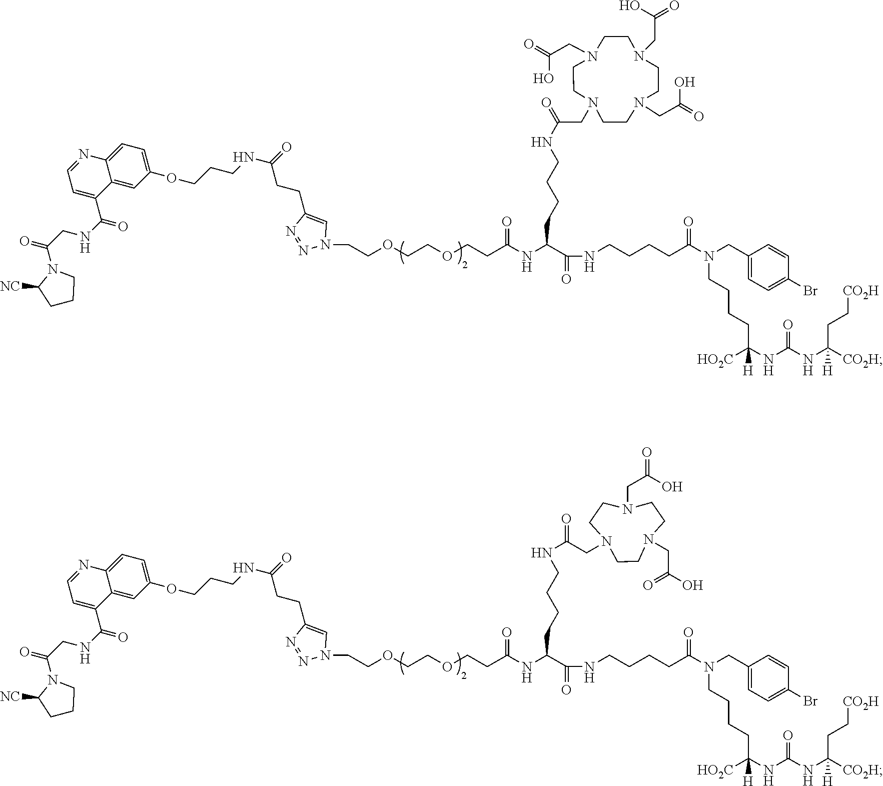

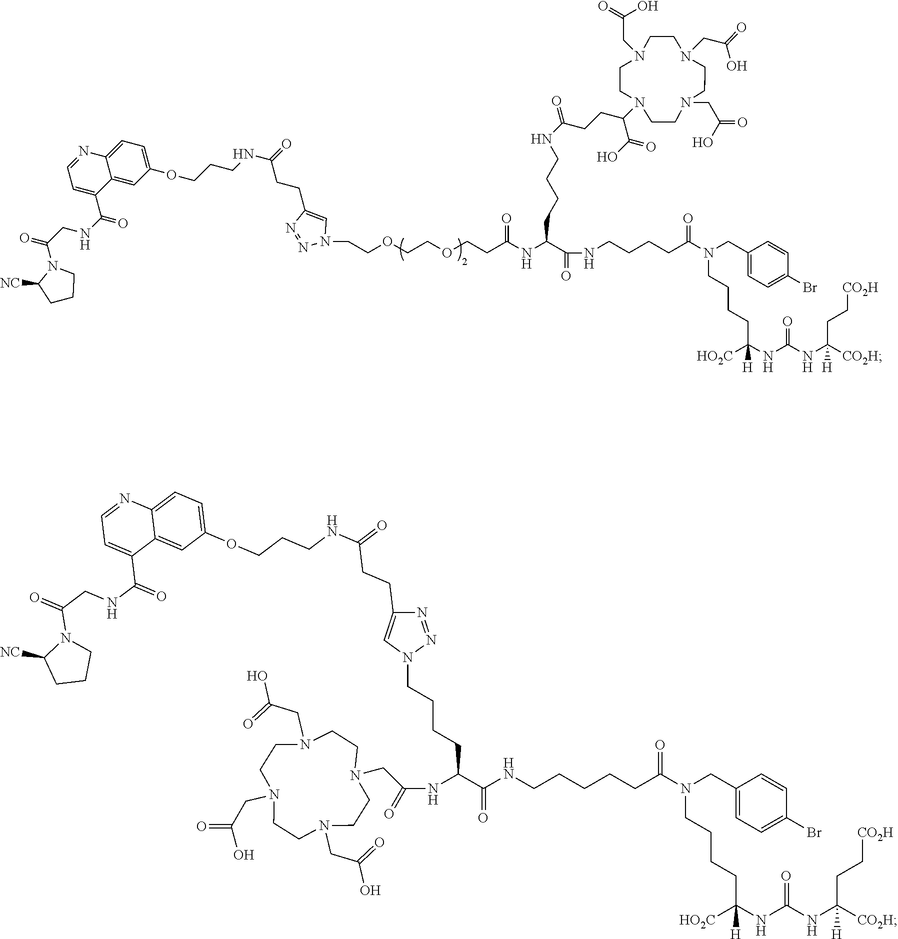

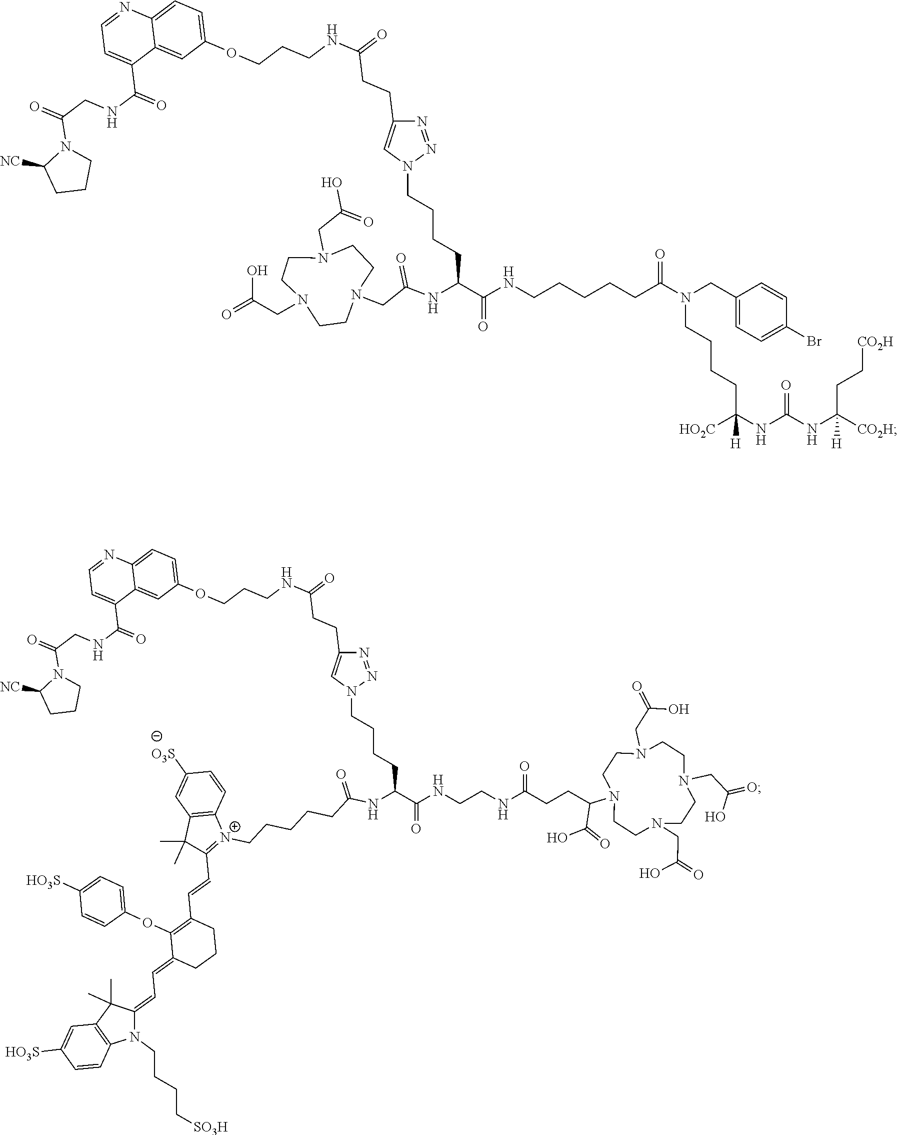

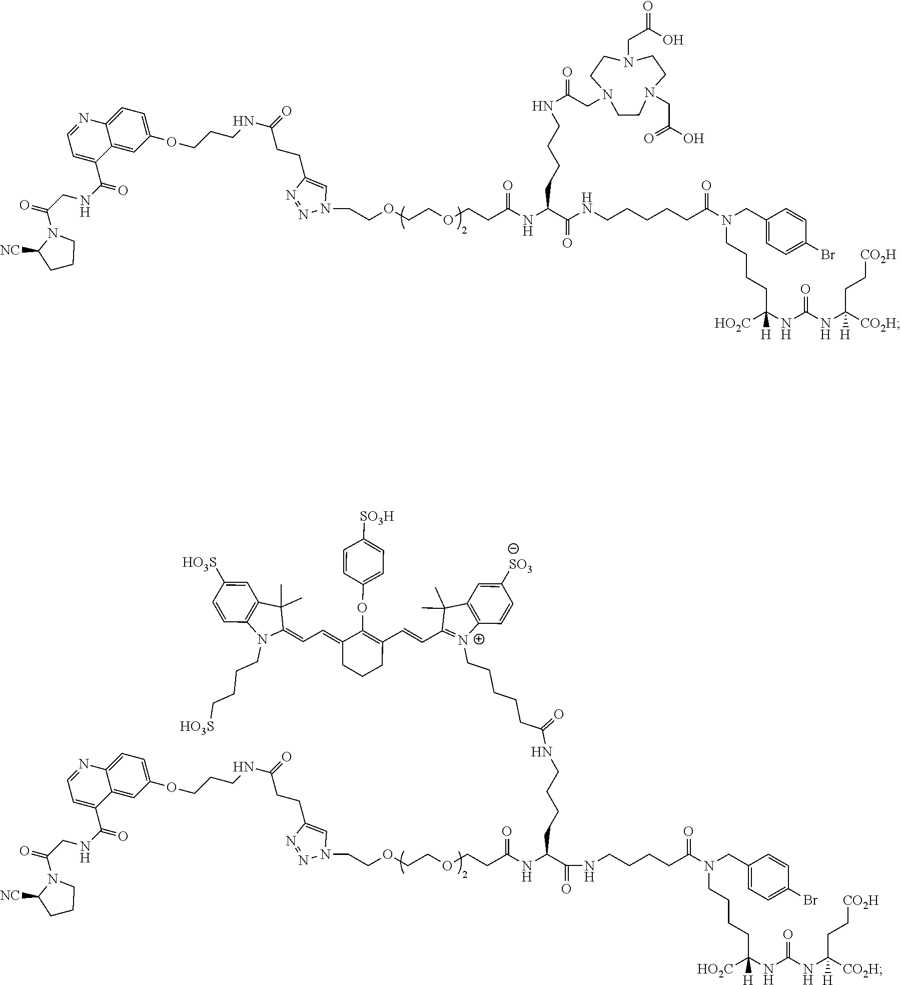

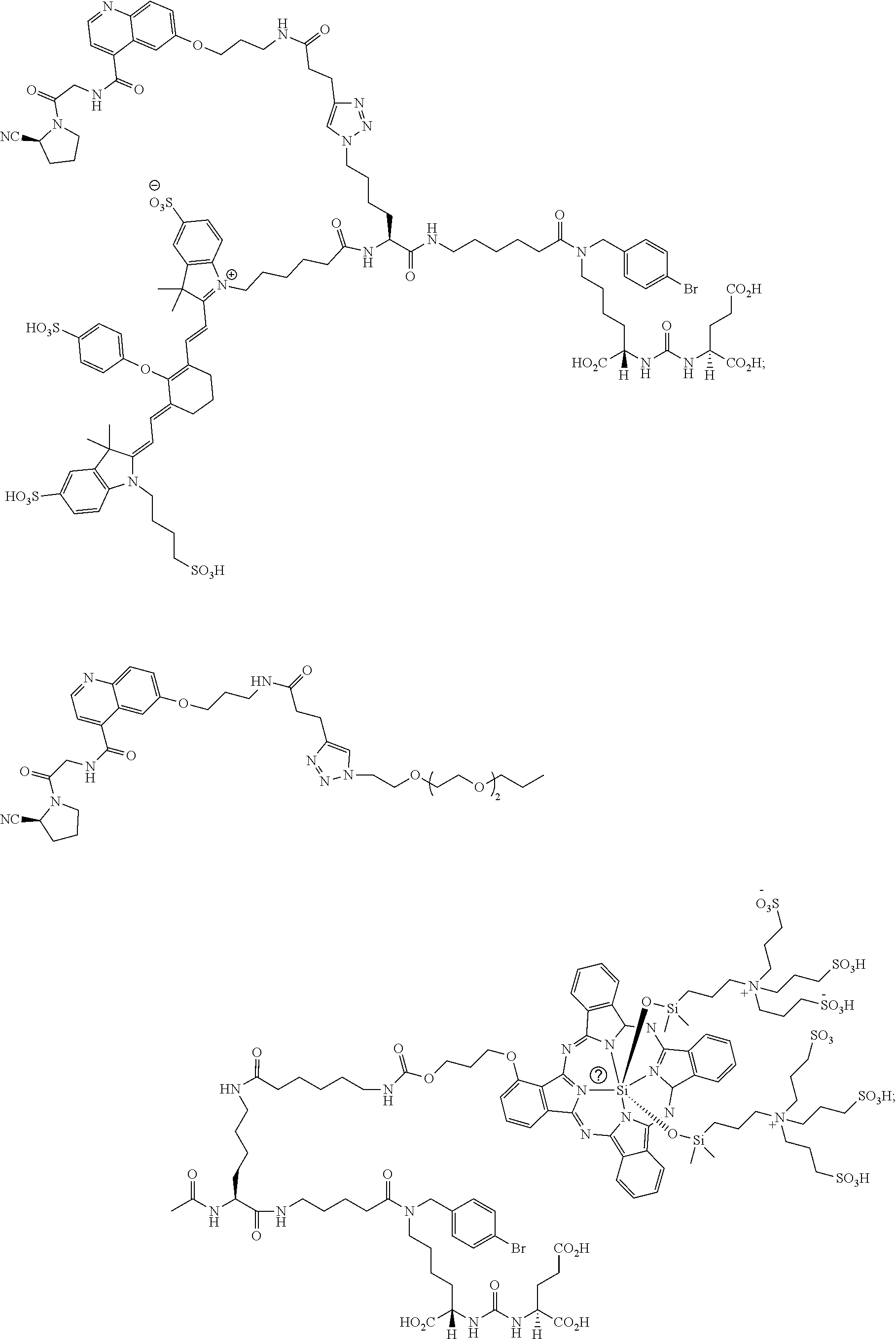

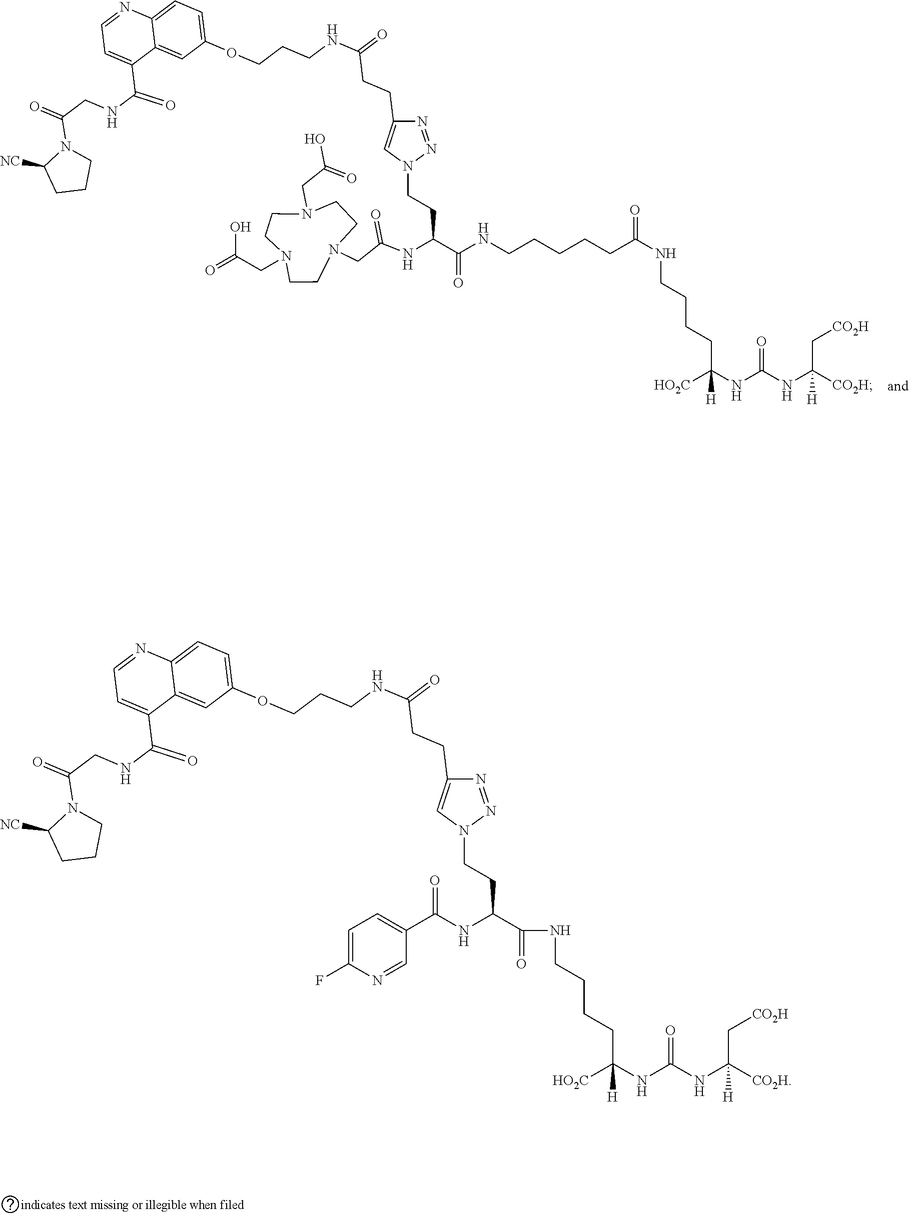 [SMILES: N#C[C@@H]1CCCN1C(=O)CNC(=O)c1ccnc2ccc(OCCCNC(=O)CCc3cn(CC[C@H](NC(=O)CN4CCN(CC(=O)O)CCN(CC(=O)O)CC4)C(=O)NCCCCCC(=O)NCCCC[C@H](NC(=O)N[C@@H](CC(=O)O)C(=O)O)C(=O)O)nn3)cc12 ; N#C[C@@H]1CCCN1C(=O)CNC(=O)c1ccnc2ccc(OCCCNC(=O)CCc3cn(CC[C@H](NC(=O)c4ccc(F)nc4)C(=O)NCCCC[C@H](NC(=O)N[C@@H](CC(=O)O)C(=O)O)C(=O)O)nn3)cc12]

### B. Methods of Use

### B.1. Methods of Imaging

In other embodiments, the presently disclosed subject matter provides a method for imaging a disease or disorder associated with fibroblast-activation protein-α (FAP-α) and/or prostate-specific membrane antigen (PSMA), the method comprising administering a compound of formula (I-IV), or a pharmaceutical composition thereof, wherein the compound of formula (I-IV) comprises an optical or radiolabeled functional group suitable for optical imaging, photoacoustic imaging, PET imaging, or SPECT imaging; and obtaining an image.

Accordingly, in some embodiments, the presently disclosed subject matter provides a method for imaging one or more cells, organs, or tissues, the method comprising exposing cells or administering to a subject an effective amount of a compound of formula (I-IV) with an optical or radioisotopic label suitable for imaging. In some embodiments, the one or more organs or tissues include prostate tissue, kidney tissue, brain tissue, vascular tissue, or tumor tissue.

The imaging methods of the invention are suitable for imaging any physiological process or feature in which FAP-α and/or PSMA is involved, for example, identifying areas of tissues or targets which exhibit or express high concentrations of FAP-α and/or PSMA.

Physiological processes in which FAP-α is involved include, but are not limited to: (a) proliferation diseases (including but not limited to cancer); (b) tissue remodeling and/or chronic inflammation (including but not limited to fibrotic disease, wound healing, keloid formation, osteoarthritis, rheumatoid arthritis, and related disorders involving cartilage degradation); and (c) endocrinological disorders (including but not limited to disorders of glucose metabolism).

In certain embodiments, the radiolabeled compound is stable in vivo.

In certain embodiments, the radiolabeled compound is detected by positron emission tomography (PET) or single photon emission computed tomography (SPECT).

In certain embodiments, the optical reporting moiety is detected by fluorescence, such as fluorescence microscopy.

In certain embodiments, the presently disclosed compounds are excreted from tissues of the body quickly to prevent prolonged exposure to the radiation of the radiolabeled compound administered to the subject. Typically, the presently disclosed compounds are eliminated from the body in less than about 24 hours. More typically, the presently disclosed compounds are eliminated from the body in less than about 16 hours, 12 hours, 8 hours, 6 hours, 4 hours, 2 hours, 90 minutes, or 60 minutes. Exemplary compounds are eliminated in between about 60 minutes and about 120 minutes. In certain embodiments, the presently disclosed compounds are stable in vivo such that substantially all, e.g., more than about 50%, 60%, 70%, 80%, or 90% of the injected compound is not metabolized by the body prior to excretion.

Additionally, for in vitro applications, such as in vitro diagnostic and research applications, body fluids and cell samples of the above subjects will be suitable for use, such as mammalian, particularly primate such as human, blood, urine or tissue samples, or blood urine or tissue samples of the animals mentioned for veterinary applications.

Other embodiments provide kits comprising a compound of formula (I-IV). In certain embodiments, the kit provides packaged pharmaceutical compositions comprising a pharmaceutically acceptable carrier and a compound of formula (I-IV). In certain embodiments the packaged pharmaceutical composition will comprise the reaction precursors necessary to generate the compound of formula (I-IV) upon combination with a radiolabeled precursor. Other packaged pharmaceutical compositions further comprise indicia comprising at least one of: instructions for preparing compounds of formula (I-IV) from supplied precursors, instructions for using the composition to image cells or tissues expressing FAP-α or PSMA.

In certain embodiments, a kit containing from about 1 to about 30 mCi of the radionuclide-labeled imaging agent described above, in combination with a pharmaceutically acceptable carrier, is provided. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like. The kit may provide a compound of formula (I-IV) in solution or in lyophilized form, and these components of the kit may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form. Determination and optimization of such stabilizers and stabilization methods are well within the level of skill in the art.

In certain embodiments, a kit provides a non-radiolabeled precursor to be combined with a radiolabeled reagent on-site.

Imaging agents may be used in accordance with the presently disclosed methods by one of skill in the art. Images can be generated by virtue of differences in the spatial distribution of the imaging agents which accumulate at a site when contacted with FAP-α and/or PSMA. The spatial distribution may be measured using any means suitable for the particular label, for example, a gamma camera, a PET apparatus, a SPECT apparatus, and the like. The extent of accumulation of the imaging agent may be quantified using known methods for quantifying radioactive emissions or fluorescence. A particularly useful imaging approach employs more than one imaging agent to perform simultaneous studies.

In general, a detectably effective amount of the imaging agent of the invention is administered to a subject. A “detectably effective amount” of the imaging agent is defined as an amount sufficient to yield an acceptable image using equipment which is available for clinical use. A detectably effective amount of the imaging agent may be administered in more than one injection. The detectably effective amount of the imaging agent of the invention can vary according to factors such as the degree of susceptibility of the individual, the age, sex, and weight of the individual, idiosyncratic responses of the individual, and the dosimetry. Detectably effective amounts of the imaging agent also can vary according to instrument and film-related factors. Optimization of such factors is well within the level of skill in the art. The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

D. Methods of Treating a FAP-α and/or PSMA Related Disease or Disorder using the Compounds of Formula (I-IV), or Pharmaceutical Compositions Thereof

In some embodiments, the presently disclosed subject matter provides a method for inhibiting fibroblast-activation protein-α (FAP-α) and/or prostate-specific membrane antigen (PSMA), the method comprising administering to a subject in need thereof an effective amount of a compound of formula (I-IV), or a pharmaceutical composition thereof.

In other embodiments, the presently disclosed subject matter provides a method for treating a fibroblast-activation protein-α (FAP-α)- and/or a prostate-specific membrane antigen (PSMA)-related disease or disorder, the method comprising administering to a subject in need of treatment thereof an effective amount of a compound of formula (I-IV), or a pharmaceutical composition thereof, wherein the compound of formula (I-IV) comprises a radiolabeled functional group suitable for radiotherapy.

In some embodiments, the presently disclosed compounds of formula (I-IV) can be used to treat a subject afflicted with one or more FAP-α related diseases or disorders including, but not limited to: (a) proliferation (including but not limited to cancer); (b) tissue remodeling and/or chronic inflammation (including but not limited to fibrotic disease, wound healing, keloid formation, osteoarthritis, rheumatoid arthritis and related disorders involving cartilage degradation); and (c) endocrinological disorders (including but not limited to disorders of glucose metabolism).

Accordingly, in some embodiments, the one or more FAP-α related disease or disorder is selected from the group consisting of a proliferative disease, including, but not limited to, breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma; diseases characterized by tissue remodeling and/or chronic inflammation; disorders involving endocrinological dysfunction; and blood clotting disorders.

In certain embodiments, the prostate-specific membrane antigen (PSMA)-related disease or disorder is selected from the group consisting of prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature. In particular embodiments, the prostate-specific membrane antigen (PSMA)-related disease or disorder comprises prostate cancer.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( S I )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In other embodiments, the method can be practiced in vitro or ex vivo by introducing, and preferably mixing, the compound and cell(s) or tumor(s) in a controlled environment, such as a culture dish or tube. The method can be practiced in vivo, in which case contacting means exposing the target in a subject to at least one compound of the presently disclosed subject matter, such as administering the compound to a subject via any suitable route. According to the presently disclosed subject matter, contacting may comprise introducing, exposing, and the like, the compound at a site distant to the cells to be contacted, and allowing the bodily functions of the subject, or natural (e.g., diffusion) or man-induced (e.g., swirling) movements of fluids to result in contact of the compound and the target.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

As used herein, the term “treating” can include reversing, alleviating, inhibiting the progression of, preventing, or reducing the likelihood of the disease, or condition to which such term applies, or one or more symptoms or manifestations of such disease or condition.

“Preventing” refers to causing a disease, condition, or symptom or manifestation of such, or worsening of the severity of such, not to occur. Accordingly, the presently disclosed compounds can be administered prophylactically to prevent or reduce the incidence or recurrence of the disease, or condition.

### C. Pharmaceutical Compositions and Administration

The present disclosure provides a pharmaceutical composition including one compound of formula (I-IV) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions, and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### D. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I-IV) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a (n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur(S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂CH₂); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy) propyl, and the like). However, the term “haloaryl,” as used herein is meant to cover only aryls substituted with one or more halogens.

Where a heteroalkyl, heterocycloalkyl, or heteroaryl includes a specific number of members (e.g., “3 to 7 membered”), the term “member” refers to a carbon or heteroatom.

Further, a structure represented generally by the formula:

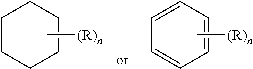

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

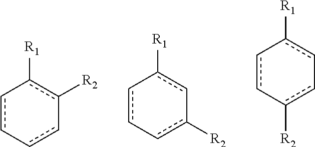

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl) acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described.

“Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium (O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

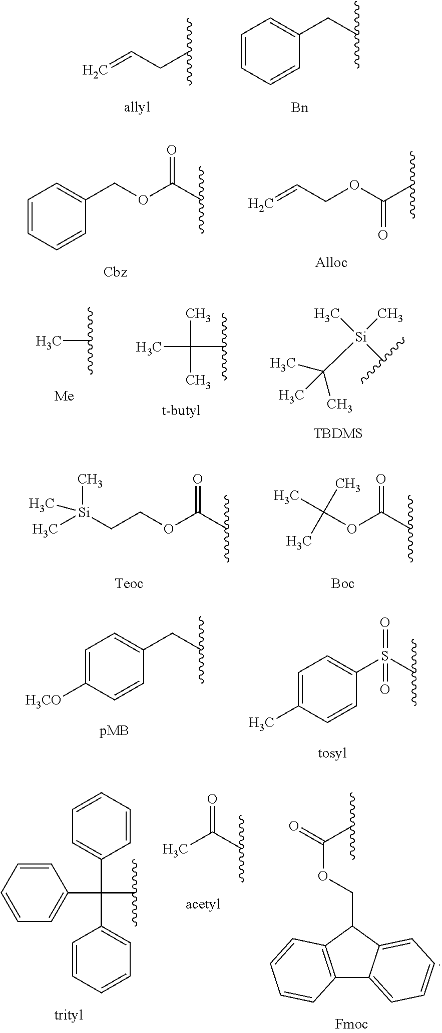

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Heterovalent FAP and PSMA Ligands

### 1.1 Experimental Procedures and Spectral Characterization

Methyl (6-hydroxyquinoline-4-carbonyl)glycinate (2): 6-Hydroxyquinoline-4-carboxylic acid (1) (200 mg, 1.05 mmol, 1.0 eq.), methyl glycinate HCl salt (200 mg, 1.58 mmol, 1.5 eq.) and HATU (603 mg, 1.58 mmol, 1.5 eq.) were dissolved in 5 mL anhydrous DMF. To the solution, DIPEA (0.46 mL, 2.64 mmol, 2.5 eq.) was added. The reaction was stirred at room temperature for 6 h. After the solvent was removed under vacuum, the mixture was loaded onto a 25 g C18 cartridge and the product was purified with a MeCN/water/TFA gradient (0/100/0.1 to 90/10/0.1). After lyophilization, the product 2 was obtained as a yellow powder with a yield of 76% (209 mg). ¹H-NMR (400 MHZ, CD₃OD): δ 8.69 (s, 1H), 7.94 (d, J=7.92 Hz, 1H), 7.57-7.51 (m, 3H), 7.42-7.37 (m, 1H), 4.21 (s, 2H), 3.81 (s, 3H). ¹³C-NMR (100 MHZ, CD₃OD): δ 172.4, 160.9, 145.1, 143.7, 129.7, 129.4, 128.3, 121.8, 119.6, 112.4, 109.1, 56.8, 44.8. ESI-MS: m/z calculated for C₁₃H₁₃N₂O₄ [M+H]⁺ 261.3; found 261.0.

Methyl (6-(3-((tert-butoxycarbonyl)amino)propoxy)quinoline-4-carbonyl)glycinate (3): Methyl (6-hydroxyquinoline-4-carbonyl)glycinate (2) (100 mg, 0.38 mmol, 1.0 eq.), 3-(Boc-amino) propyl bromide (230 mg, 0.96 mmol, 2.5 eq.) were dissolved in 4 mL anhydrous DMF. Cs₂CO₃ (376 mg, 1.15 mmol, 3.0 eq.) was added to the solution and the reaction was stirred at room temperature overnight. After filtration, the solvent was removed under vacuum and the remaining mixture was loaded onto a 25 g C18 cartridge. The product was purified with a MeCN/water/TFA gradient (0/100/0.1 to 90/10/0.1). After lyophilization, the product 3 was obtained as a yellow powder with a yield of 54% (86 mg). ¹H-NMR (400 MHZ, CDCl₃): δ 8.68-8.37 (m, 2H), 8.02 (d, J=9.1 Hz, 1H), 7.80 (s, 1H), 7.72-7.64 (m, 1H), 7.40 (d, J=9.1 Hz, 1H), 4.94 (br s, 1H), 4.41-4.31 (m, 2H), 4.27-4.18 (m, 2H), 3.85 (s, 3H), 3.44-3.30 (m, 2H), 2.13-2.00 (m, 2H), 1.43 (s, 9H). ¹³C NMR (100 MHz, CDCl₃): δ 170.1, 167.2, 158.4, 144.7, 142.3, 128.4, 126.1, 124.7, 119.1, 103.7, 79.5, 60.4, 52.5, 41.4, 37.7, 29.3, 28.4. ESI-MS: m/z calculated for C₂₁H₂₈N₃O₆ [M+H]⁺ 418.5; found 418.1.

tert-Butyl(S)-(3-((4-((2-(2-cyanopyrrolidin-1-yl)-2-oxoethyl)carbamoyl)quinolin-6-yl)oxy)propyl)carbamate (5): Compound 3 (145 mg, 0.347 mmol, 1.0 eq.) and LiOH (58.4 mg, 1.39 mmol, 4.0 eq.) was stirred in 5 mL of H₂O/THF (1/1) for 6 hours. After most of the THF was removed under vacuum, the mixture was loaded onto a 25 g C18 cartridge and eluted with a MeCN/water/TFA gradient (0/100/0.1 to 90/10/0.1) to remove the salts. After removal of solvent and drying, the product 4 was obtained in quantitative yield. To a stirred solution of 4 (144 mg, 0.357 mmol, 1.0 eq.) in anhydrous DMF (4 mL) was added(S)-pyrrolidine-2-carbonitrile (56.8 mg, 0.428 mmol, 1.2 eq.), HATU (163 mg, 0.428 mmol, 1.2 eq.) and DIPEA (125 μL, 0.714 mmol, 2.0 eq.). After 6 hours, the solvent was removed under vacuum and the crude mixture was loaded onto a 25 g C18 cartridge (Silicycle, Canada). The product was purified with a MeCN/water/TFA gradient (0/100/0.1 to 90/10/0.1). After lyophilization, the product 5 was obtained with a yield of 80% (135 mg). ¹H NMR (400 MHZ, CDCl₃): δ 8.73 (s, 1H), 7.95 (d, J=10.2 Hz, 1H), 7.68 (br s, 1H), 7.63-7.56 (m, 1H), 7.56-7.48 (m, 1H), 7.38-7.29 (m, 1H), 5.27 (br s, 1H), 4.84-4.72 (m, 1H), 4.46-4.35 (m, 1H), 4.33-4.20 (m, 1H), 4.17-4.09 (m, 2H), 3.78-3.64 (m, 1H), 3.59-3.46 (m, 1H), 3.36 (s, 2H), 2.38-2.17 (m, 4H), 1.42 (s, 9H), 1.35-1.27 (m, 2H). ¹³C NMR (100 MHZ, CDCl₃): δ 167.6, 167.5, 157.9, 156.2, 146.3, 130.2, 125.7, 123.7, 119.3, 118.0, 103.3, 79.0, 65.9, 46.8, 45.7, 42.2, 37.6, 29.8, 29.3, 28.4, 25.1. ESI-MS: m/z calculated for C₂₅H₃₂N₅O₅ [M+H]⁺ 482.6; found 482.2.

(S)—N-(2-(2-cyanopyrrolidin-1-yl)-2-oxoethyl)-6-(3-(pent-4-ynamido)propoxy)quinoline-4-carboxamide (FAP-Acetylene1): Compound 5 (100 mg, 0.207 mmol.) was treated with a 1 mL solution of TFA/methylene chloride (1/1) for 2 h. The solvent was removed under vacuum, and the crude mixture was loaded onto a 12 g C18 cartridge (Silicycle, Canada). The product was purified with a MeCN/water/TFA gradient (0/100/0.1 to 90/10/0.1). After lyophilization, product 6 was obtained with a quantitative yield. To the stirred solution of 6 (10 mg, 0.026 mmol, 1.0 eq.) in DMF (1 mL) was added 2,5-dioxopyrrolidin-1-yl pent-4-ynoate (7) (5.12 mg, 0.026 mmol, 1.0 eq.) and DIPEA (13.5 μL, 0.078 mmol, 3.0 eq.). After 2 hours, the solvent was removed under vacuum and the crude mixture was loaded onto a 12 g C18 cartridge (Silicycle, Canada). The product was purified with a MeCN/water/TFA gradient (0/100/0.1 to 90/10/0.1). After lyophilization, 8.6 mg of FAP-Acetylene1 was obtained with a yield of 71%. ESI-MS: m/z calculated for C₂₅H₂₈N₅O₄ [M+H]⁺ 462.2; found 462.1.

(((S)-5-(5-Amino-N-(4-bromobenzyl)pentanamido)-1-carboxypentyl)carbamoyl)-L-glutamic acid (9): To a stirred solution of 8 (214 mg, 0.326 mmol, 1.0 eq.) and 2,5-dioxopyrrolidin-1-yl 5-((tert-butoxycarbonyl)amino) pentanoate (102.5 mg, 0.326 mmol, 1.0 eq.) in DMF (2 mL) was added DIPEA (170 μL, 0.978 mmol, 3.0 eq.) at room temperature. The reaction mixture was stirred for 24 h and concentrated to get crude product. To the above crude was added 2 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 9 (143 mg, 75%) as a white solid. H¹-NMR (500 MHz, D₂O): δ 7.55 (d, J=8.5 Hz, 1H), 7.51 (d, J=8.5 Hz, 1H), 7.12 (dd, J=2.5, 8.0 Hz, 2H), 4.59 (s, 1H), 4.51 (s, 1H), 4.29-4.22 (m, 1H), 4.19-4.11 (m, 1H), 3.39-3.29 (m, 2H), 3.05-2.88 (m, 2H), 2.59-2.38 (m, 4H), 2.22-2.10 (m, 1H), 2.02-1.90 (m, 1H), 1.82-1.46 (m, 8H), 1.39-1.24 (m, 2H).

(10S,23S,27S)-10-Amino-18-(4-bromobenzyl)-2,2-dimethyl-4,11,17,25-tetraoxo-3-oxa-5,12,18,24,26-pentaazanonacosane-23,27,29-tricarboxylic acid (10): To a stirred solution of 9 (139 mg, 0.236 mmol, 1.0 eq.) and Fmoc-L-Lys (Boc)-OSu (133.9 mg, 0.236 mmol, 1.0 eq.) in DMF (2 mL) was added DIPEA (250 □L, 1.42 mmol, 6.0 eq.) at room temperature. The reaction mixture was stirred for 2 h and concentrated to get crude product. To the above crude was added 2 mL of 20% piperidine in DMF (3 mL) at room temperature and mixture was stirred for 1 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 10 (125 mg, 65%) as a white solid. H¹-NMR (500 MHz, DMSO-d₆): δ 8.43-8.32 (m, 1H), 8.08 (s, 2H), 7.93 (d, J=7.5 Hz, 1H), 7.74 (d, J=7.5 Hz, 1H), 7.55 (d, J=8.0 Hz, 1H), 7.41 (d, J=8.0 Hz, 1H), 6.74 (t, J=5.0 Hz, 1H), 6.40-6.26 (m, 2H), 4.52 (s, 1H), 4.44 (s, 1H), 4.14-3.98 (m, 2H), 3.23-2.97 (m, 5H), 2.93-2.82 (m, 2H), 2.37 (t, J=7.0 Hz, 1H), 2.31-2.15 (m, 3H), 1.96-1.43 (m, 14H), 1.35 (s, 9H), 1.29-1.15 (m, 4H); [M+H]⁺ calcd for C₃₅H₅₆BrN₆O₁₁, 815.3192; found, 815.3184.

(11S,24S,28S)-1-Azido-19-(4-bromobenzyl)-11-(4-((tert-butoxycarbonyl)amino)butyl)-9,12,18,26-tetraoxo-3,6-dioxa-10,13,19,25,27-pentaazatriacontane-24,28,30-tricarboxylic acid (11): To a stirred solution of 10 (47.3 mg, 0.058 mmol, 1.0 eq.) and Azido-PEG3-NHS ester (20 mg, 0.058 mmol, 1.0 eq.) in DMSO (100 μL) was added DIPEA (80 μL, 0.464 mmol, 8.0 eq.) at room temperature. The reaction mixture was stirred for 2 h and concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 11 (42 mg, 70%) as a white solid. ESI-MS: m/z calculated for C₄₄H₇₁BrN₉O₁₅ [M+H]⁺ 1044.4; found 1044.3.

(14S,27S,31S)-22-(4-Bromobenzyl)-14-(4-((tert-butoxycarbonyl)amino)butyl)-1-(4-(3-((3-((4-((2-((S)-2-cyanopyrrolidin-1-yl)-2-oxoethyl)carbamoyl)quinolin-6-yl)oxy)propyl)amino)-3-oxopropyl)-1H-1,2,3-triazol-1-yl)-12,15,21,29-tetraoxo-3,6,9-trioxa-13,16,22,28,30-pentaazatritriacontane-27,31,33-tricarboxylic acid (12): To a stirred solution of FAP-Acetylene1 (6 mg, 0.0129 mmol, 1.0 eq.) in t-BuOH:H2O (1:2, 300 μL) was added CuSO₄·5H₂O (0.486 mg, 0.0019 mmol, 0.15 eq.) and sodium ascorbate (1.15 mg, 0.0058 mmol, 0.45 eq.). To the above reaction mixture was added solution of product 11 (13.5 mg, 0.0129 mmol, 1.0 eq.) in DMSO (100 L) and stirred for overnight at room temperature. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 12 (16.7 mg, 86%) as a white solid. ESI-MS: m/z calculated for C₆₉H₉₈BrN₁₄O₁₉ [M+H]⁺ 1505.6; found 1505.4.

(14S,27S,31S)-22-(4-bromobenzyl)-1-(4-(3-((3-((4-((2-((S)-2-cyanopyrrolidin-1-yl)-2-oxoethyl)carbamoyl)quinolin-6-yl)oxy)propyl)amino)-3-oxopropyl)-1H-1,2,3-triazol-1-yl)-12,15,21,29-tetraoxo-14-(4-(2-(4,7,10-tris(carboxymethyl)-1,4,7,10-tetraazacyclododecan-1-yl)acetamido)butyl)-3,6,9-trioxa-13,16,22,28,30-pentaazatritriacontane-27,31,33-tricarboxylic acid (SB-FAP-01): To the compound 12 (9 mg, 0.0059 mmol) was added 1 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded free amine in quantitative yield. To a stirred solution of amine (3.8 mg, 0.0027 mmol, 1.0 eq.) and DOTA-NHS-ester (3.08 mg, 0.004 mmol, 1.5 eq.) in DMSO (50 μL) was added DIPEA (4.7 AL, 0.027 mmol, 10.0 eq.) at room temperature. The reaction mixture was stirred for 2 h and crude was purified by preparative RP-HPLC chromatography using 0.1% TFA in H2O and 0.1% TFA in acetonitrile as eluents followed by lyophilization afforded compound SB-FAP-01 (3.3 mg, 69%) as a white solid. [RP-HPLC purification was achieved using Agilent System, λ 254 nm, 250 mm×10 mm Phenomenex Luna C18 column, solvent gradient: 90% H2O (0.1% TFA) and 10% ACN (0.1% TFA), reaching 90% of ACN in 25 min at a flow rate of 5 mL/min, product eluted at 13.8 min]. HRMS (ESI) m/z: [M+H]⁺ calcd for C₈₀H₁₁₄BrN₁₈O₂₄, 1789.7456; found, 1789.7442.

### 1.2 Binding Affinity (Ki) of SB-FAP-01

Binding affinity (Ki) of SB-FAP-01 is provided in Table 2.

TABLE 2
Binding Affinity (Ki) for SB-FAP-01

| | FAP Binding | PSMA Binding |
|---|---|---|
| | affinity | affinity |
| Ki (nM) | 0.36 | 16.63 |
| Ki (95% CI, nM) | 0.25-0.51 | 7.30-37.90 |

### Example 2

### Homobivalent FAP Ligands

In some embodiments, homobivalent FAP ligands can be prepared via the following scheme:

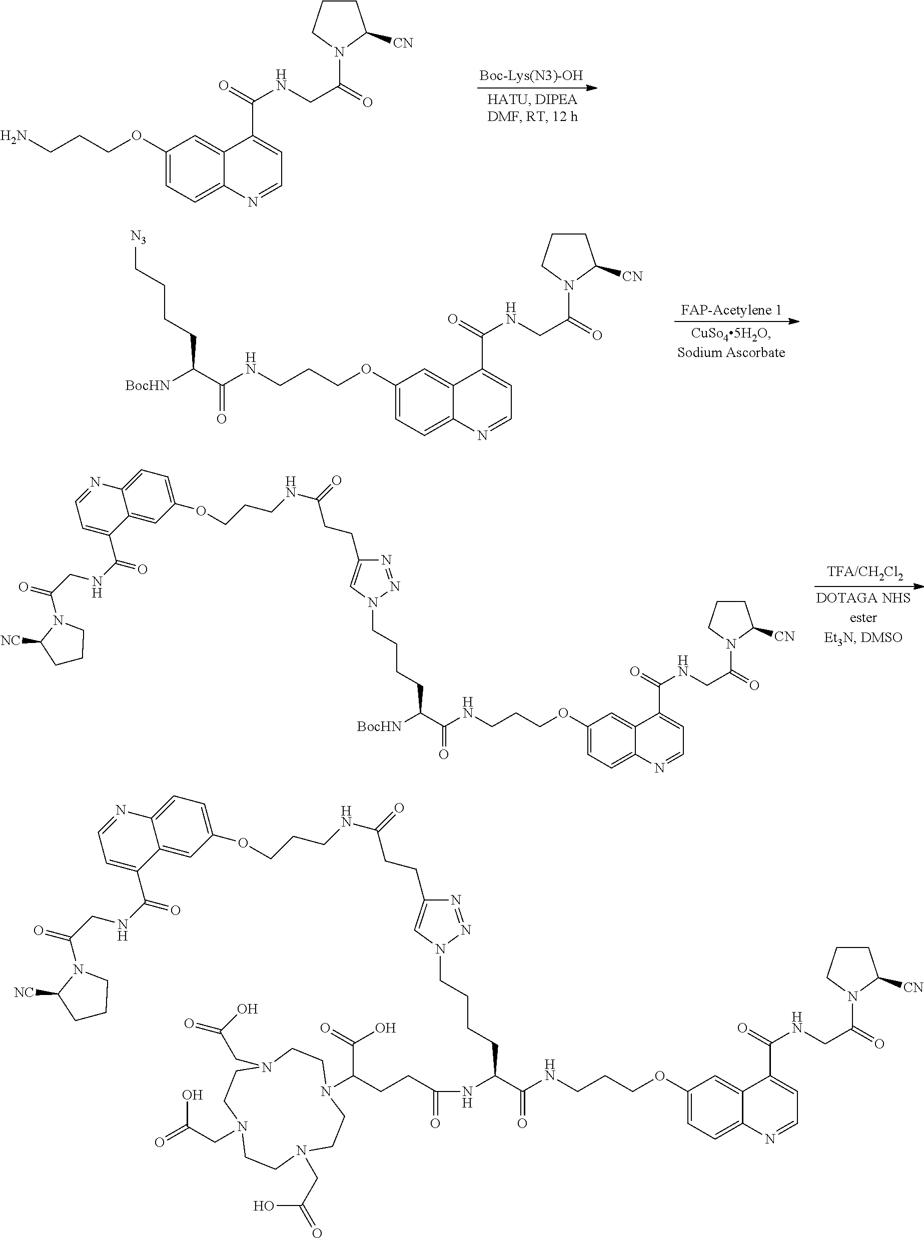 [SMILES: N#C[C@@H]1CCCN1C(=O)CNC(=O)c1ccnc2ccc(OCCCN)cc12 ; CC(C)(C)OC(=O)N[C@@H](CCCCN=[N+]=[N-])C(=O)NCCCOc1ccc2nccc(C(=O)NCC(=O)N3CCC[C@H]3C#N)c2c1 ; CC(C)(C)OC(=O)N[C@@H](CCCCn1cc(CCC(=O)NCCCOc2ccc3nccc(C(=O)NCC(=O)N4CCC[C@H]4C#N)c3c2)nn1)C(=O)NCCCOc1ccc2nccc(C(=O)NCC(=O)N3CCC[C@H]3C#N)c2c1 ; N#C[C@@H]1CCCN1C(=O)CNC(=O)c1ccnc2ccc(OCCCNC(=O)CCc3cn(CCCC[C@H](NC(=O)CCC(C(=O)O)N4CCN(CC(=O)O)CCN(CC(=O)O)CCN(CC(=O)O)CC4)C(=O)NCCCOc4ccc5nccc(C(=O)NCC(=O)N6CCC[C@H]6C#N)c5c4)nn3)cc12]

In some embodiments, the homobivalent FAP compound is selected from the group consisting of:

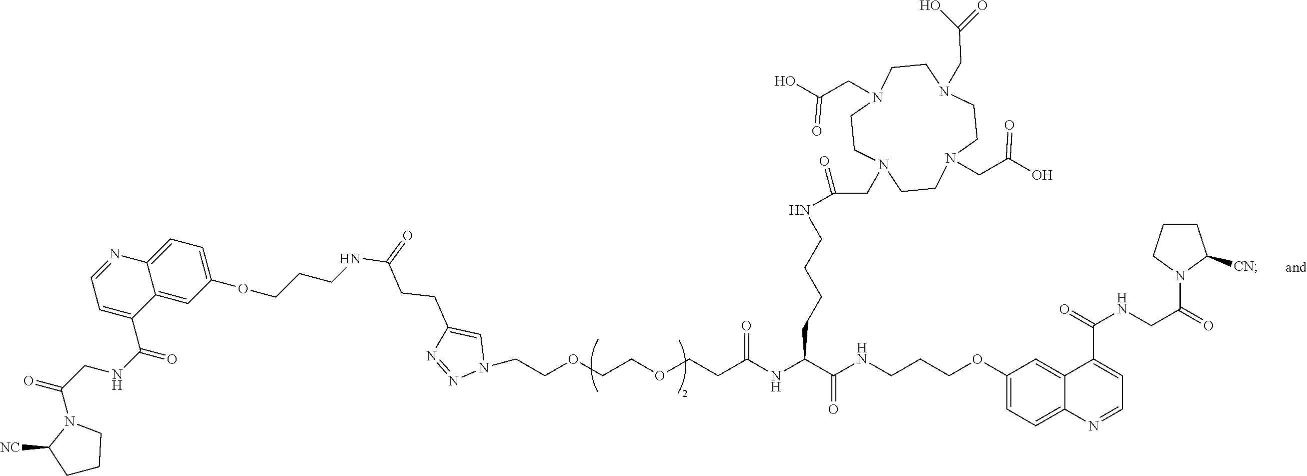

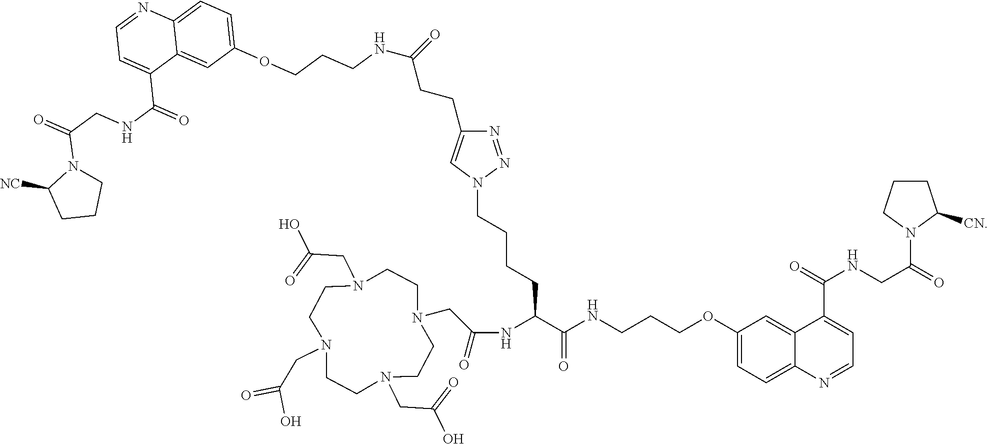 [SMILES: N#C[C@@H]1CCCN1C(=O)CNC(=O)c1ccnc2ccc(OCCCNC(=O)CCc3cn(CCCC[C@H](NC(=O)CN4CCN(CC(=O)O)CCN(CC(=O)O)CCN(CC(=O)O)CC4)C(=O)NCCCOc4ccc5nccc(C(=O)NCC(=O)N6CCC[C@H]6C#N)c5c4)nn3)cc12]

### Example 3

### Heterobivalent FAP Ligands

In other embodiments, the heterobivalent FAP ligand comprises a FAP-albumin heterobivalent compound having the following structure:

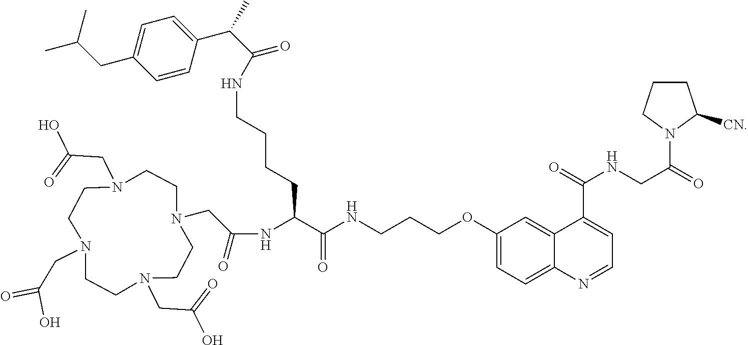 [SMILES: CC(C)Cc1ccc([C@H](C)C(=O)NCCCC[C@H](NC(=O)CN2CCN(CC(=O)O)CCN(CC(=O)O)CCN(CC(=O)O)CC2)C(=O)NCCCOc2ccc3nccc(C(=O)NCC(=O)N4CCC[C@H]4C#N)c3c2)cc1]

### Example 4

### Heterobivalent Theranostics Targeting FAP and PSMA

### 4.1 Overview

In some embodiments, the presently disclosed subject matter provides a theranostic radiopharmaceutical that engages two key cell surface proteases, fibroblast activation protein alpha (FAP) and prostate-specific membrane antigen (PSMA), each frequently overexpressed within the tumor microenvironment (TME). The latter also is expressed in most prostate tumor epithelium. To engage a broader spectrum of cancers for imaging and therapy, small-molecule FAP and PSMA-targeting moieties were conjugated using an optimized linker to provide ⁶⁴Cu-labeled compounds.

Representative compounds FP-L1 and FP-L2 were synthesized using two linker constructs attaching the FAP and PSMA-binding pharmacophores. In vitro inhibition constants (Ki) for FAP and PSMA were determined. Cell uptake assays and flow cytometry were conducted in human glioma (U87), melanoma (SK-MEL-24), and prostate cancer (PSMA+ PC3 PIP and PSMA− PC3 flu) and clear cell renal cell carcinoma lines (PSMA+/PSMA− 786-O). Quantitative positron emission tomography/computed tomography (PET/CT) and tissue biodistribution studies were performed using U87, SK-MEL-24, PSMA+ PC3 PIP, and PSMA+ 786-O experimental xenograft models and the KPC genetically engineered mouse model of pancreatic cancer.

⁶⁴Cu-FP-L1 and -L2 were produced in high radiochemical yield and molar activity. Ki values were in the nanomolar range for both FAP and PSMA. PET imaging and biodistribution studies revealed high and specific targeting of ⁶⁴Cu-FP-L1 and ⁶⁴Cu-FP-L2 for FAP and PSMA. ⁶⁴Cu-FP-L1 displayed more favorable pharmacokinetics than ⁶⁴Cu-FP-L2. ⁶⁴Cu-FP-L1 demonstrated similar tumor uptake to ⁶⁴Cu-FAPI-04 in the U87 model at 2 h. ⁶⁴Cu-FP-L1 showed high tumor uptake and retention at >10% injected dose per gram of tissue from 1 to 24 h post-injection in PSMA+PC3 PIP tumor.

In sum, ⁶⁴Cu-FP-L1 demonstrated high and specific tumor targeting of FAP and PSMA. This compound should enable imaging of lesions expressing FAP, PSMA, or both on the tumor cell surface or within the TME. FP-L1 can readily be converted into a theranostic agent for the management of heterogeneous tumors.

### 4.2 Background

Theranostic radiopharmaceuticals are used to treat patients with metastatic cancer with high efficacy and low toxicity. Herrmann et al., 2020; Siva et al., 2020; Imlimthan et al., 2021; Uijen et al., 2021. During the past decade, the development of radiopharmaceuticals has focused on targeting cell surface receptors that are selective for specific biological targets, one target at a time. That “one-molecule, one receptor” strategy has provided considerable achievements. One successful low-molecular-weight radiotheranostic agent, ⁶⁸Ga-/¹⁷⁷Lu-DOTATATE, has received regulatory approval to treat somatostatin receptor-positive gastroenteropancreatic neuroendocrine tumors. Additionally, several promising agents, including prostate-specific membrane antigen (PSMA)-based theranostic radiopharmaceuticals, are in the pipeline. Herrmann et al., 2020.

Due to heterogeneity and the ability of many cancers to develop resistance rapidly, such highly selective agents, however, might provide only temporary relief. Targeting appropriate cells in the tumor microenvironment (TME), differentially expressed on the tumor cells, vasculature, and tumor stroma, may complement direct tumor targeting to enhance efficacy, particularly if done concurrently. Bejarano et al., 2021. Without wishing to be bound to any one particular theory, it was thought that a heterobivalent agent targeting fibroblast activation protein alpha (FAP) and PSMA, as both are abundantly expressed within the TME, and the latter on prostate tumor cells, may enhance cancer detection and therapy. FAP is expressed on cancer-associated fibroblasts (CAFs), Garin-Chesa et al., 1990, while PSMA is expressed on most prostate cancers and in most solid tumor neovasculature. Chang et al., 1999; Wernicke et al., 2017; Spatz et al., 2018. Both FAP and PSMA are known to be present during disease progression in many aggressive cancers and demonstrate increased expression in aggressive and metastatic disease. Cohen et al., 2008; Busek et al., 2016; López et al., 2016; Solano-Iturri et al., 2020a; Solano-Iturri et al., 2020b; Hofman et al., 2021.

PSMA-based radiotheranostics have proved beneficial compared to the standard-of-care in metastatic castration-resistant prostate cancer (mCRPC). Hofman et al., 2021; Hofman et al., 2020. Patients with mCRPC have lesions with heterogeneous and, in some cases, no expression of PSMA. Paschalis et al., 2019. Lesions that are PSMA-negative, e.g., neuroendocrine prostate cancer (NEPC), may represent particularly aggressive, often metabolically active, disease. That fact has been used to select patients for PSMA-directed therapy, by avoiding its use in patients with high uptake of 18F-fluordeoxyglucose (FDG) in their tumors. Paschalis et al., 2019.

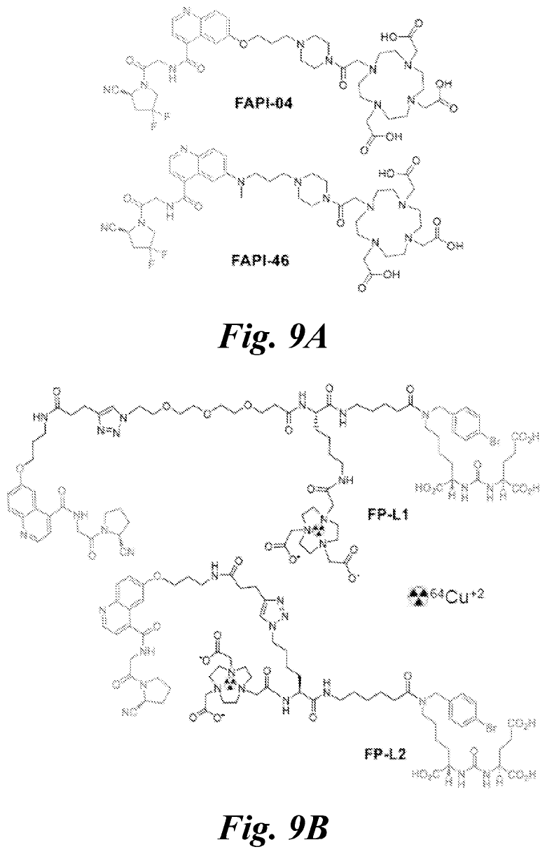

Recent immunohistochemistry (IHC) studies further begin to reveal that FAP expression is a characteristic of mCRPC regardless of genetic subtype, treatment regimen, or location of metastasis. Hintz et al., 2020; Kesch et al., 2021. Recent studies also have shown that FAP-based PET imaging is more sensitive for detecting PSMA-negative metastatic lesions than FDG PET/CT. Isik et al., 2021; Kessel et al., 2021. FAP-based PET imaging has emerged as a new diagnostic tool in a variety of malignancies. Kratochwil et al., 2019; Mona et al., 2021. FAP is an integral membrane protease overexpressed on CAFs in >90% of human epithelial tumors. Kalluri, 2016. It also is an independent negative prognostic factor for several malignancies, Fitzgerald and Weiner, 2020, and exists on the cell surface and in a soluble, circulating form in the blood in mice and humans. Keane et al., 2014. CAFs have an important role in producing cytokines, chemokines, metabolites, enzymes, and extracellular matrix molecules that fuel the growth of cancer cells. Kalluri, 2016. Like PSMA, FAP allows selective targeting of a variety of tumors employing high-affinity inhibitors, Brennen et al., 2012, including the promising clinical agents ⁶⁸Ga-FAPI-04 and ⁶⁸Ga-FAPI-46 (FIG. 9). Loktev et al., 2019. Like FAP, PSMA also is a protease known as glutamate carboxypeptidase II (GCP II) and increases endothelial cell invasion and angiogenesis in most aggressive solid tumors. Conway et al., 2013.

Copper-64 [half-life (t_{1/2})=12.7 hours; β+: 17.4%, Eβ+ (mean)=278 keV] has a unique decay profile and is mainly used for PET imaging, while ⁶⁷Cu [t_{1/2}=61.9 h, β−=100%, Eβ− (mean)=141 keV] is a promising radioisotope for therapy. Copper-64 is an attractive alternative to short-lived radioisotopes for PET imaging of antibodies, peptides, and small molecules. Ling et al., 2019. The recent regulatory approval of ⁶⁴Cu-DOTATATE (LUTATHERA®) has triggered the development of ⁶⁴Cu-labeled compounds for several molecularly targeted radiotheranostic applications. Ling et al., 2019.

Without wishing to be bound to any one particular theory, it was thought that that heterobivalent compounds using two clinically tested, high-affinity FAP and PSMA-based targeting moieties would bind and enable imaging and therapy of a variety of cancers and cancer subtypes within a given malignancy, such as PSMA+ mCRPC and PSMANEPC. Such compounds also could enhance the retention of PSMA-based radiotheranostics in solid malignancies with PSMA+ neovasculature and FAP+ tumor cells or CAFs, for example, glioblastoma. Uijen et al., 2021; Busek et al., 2016; Röhrich et al., 2019. In the first step to test this hypothesis, an optimized heterobivalent compound with two distinctly binding pharmacophores targeting FAP and PSMA was developed.

Previously optimized small-molecule FAP targeting moiety, Jansen et al., 2014; Slania et al., 2021, and PSMA targeting platform were used to generate high-affinity heterobivalent compounds. Shallal et al., 2014. The compounds were labeled with ⁶⁴Cu and were evaluated in preclinical imaging studies in relevant human xenografts and in the KPC genetically engineered mouse model (GEMM) of pancreatic ductal adenocarcinoma (PDAC).

### 4.3 Materials and Methods

### 4.3.1 Reagents, Cell Lines, and Animal Models

Six cell lines for in vitro and in vivo evaluation were used: U87 (glioblastoma), SK-MEL-24 (melanoma), PSMA+ PC3 PIP, and PSMAPC3 flu (prostate carcinoma), PSMA+ 786-O, PSMA-786-O (renal cell carcinoma). Six- to 8-wk old male, nonobese diabetic/shi-scid/IL-2rγ (null) (NSG) mice (Johns Hopkins Animal Resources Core) were implanted subcutaneously with the indicated cell lines.

### 4.3.2 In Vitro Assays

### 4.3.2.1 Competitive Inhibition Assays

PSMA binding affinities of the compounds were determined using a competitive inhibition assay as previously reported. Banerjee et al., 2011. FAP, prolyl endopeptidase (PREP), and dipeptidyl dipeptidase 4 (DPPIV) inhibition assays: Recombinant enzymes (FAP, PREP, DPPIV) were purchased from R&D Systems (Minneapolis, MN). FAPI-04 was used as a positive control. Z-Gly-Pro-AMC was used as a substrate for FAP and PREP. H-Gly-Pro-AMC was used as a substrate for DPPIV. The recombinant enzyme (0.4 μg/mL) was incubated with varying amounts of the test articles in the presence of the designated substrate (80 μM) for 10 min at room temperature. Fluorescence intensity was measured with 380 nm excitation and 460 nm emission using the Cytation 5 Cell Imaging Multi-Mode Reader (BioTek, Winooski, VT). IC50 and Ki values were obtained using a sigmoidal dose-response function. Cheng and Prusoff, 1973.

### 4.3.2.2 Cell Uptake, Flow Cytometry, and IHC

Cell uptake assays, flow cytometry, and IHC were performed as described previously. Banerjee et al., 2019; Nimmagadda et al., 2018. PET imaging and biodistribution Sequential PET imaging and biodistribution studies were conducted to quantify and validate PET imaging data. Briefly, tumor-bearing mice were administrated approximately 7.4 MBq of radiotracer in 150 μL saline via tail-vein injection. They were randomized into the indicated groups of 3-4 mice before radiotracer injection.

In Experiment 1, bilateral xenografts of U87 (right flank) and PSMA+ PC3 PIP (left flank) tumors were established in male NSG mice (n=3), which underwent imaging followed by biodistribution over 24 h. To demonstrate FAP or PSMA binding specificity, blocking studies were performed by co-injection of FAPI-04 (for FAP), Loktev et al., 2019, or ZJ43 (for PSMA), Olszewski et al., 2004, using a separate cohort of U87 and PSMA+ PC3 PIP bilateral xenografts (n=3-4). A biodistribution study was further conducted with U87 tumors at 2 h post-injection (n=3) to compare the tissue distribution properties of ⁶⁴Cu-FP-L1 and ⁶⁴Cu-FAPI-04.

In Experiment 2, SK-MEL-24 tumor-bearing mice (n=3-4) were investigated.

In Experiment 3, PET studies were done using a GEMM, [6-mo old, male, LSL-KrasG12D; LSL-Trp53R173H; Pdx1-Cre (KPC) triple mutant], He et al., 2020, at 1 and 2 h post-injection. PET studies were concurrently done using an age-matched littermate (female). At 48 h, mice were injected with a near-infrared fluorescent (NIRF) compound, IRDye800-FP-L1, containing the same construct as FP-L1 for FAP and PSMA targeting, with the 1,4,7-triazacyclononane-1,4,7-triacetic acid (NOTA) chelating agent replaced with IRDye800CW (LICOR, Lincoln, NE). Mice were then sacrificed at 2 h, and an ex vivo optical imaging study was performed. A veterinary pathologist (Dr. Brayton) performed the histopathological evaluations.

Experiment 4 consisted of preliminary imaging (7.4 MBq) (n=2) and biodistribution studies (0.74 MBq) at 2 h in male NSG healthy mice (n=3) and tumor-bearing human clear cell renal cell carcinoma (ccRCC) PSMA+786-O tumors (n=4).

### 4.3.3 Statistical Analysis

Statistical significance was calculated using an unpaired two-tailed t-test using GraphPad Prism 9.0 software. Data were expressed as the mean±standard deviation (±SD). Statistical significance is defined at P≤0.05.

### 4.4 Results

### 4.4.1 Chemical and Radiochemical Syntheses

Structures of compounds FP-L1 and FP-L2 are shown in FIG. 9. Click chemistry was used to conjugate the FAP and PSMA targeting moieties with the selected linker. Radiolabeling employed a rapid microwave-assisted method to generate ⁶⁴Cu-FP-L1 in nearly quantitative yield. High-performance liquid chromatography (HPLC) was used to remove unreacted ligand to ensure high specific radioactivity (>19 MBq/nmol). ⁶⁴Cu-FP-L1 was stable for at least 4 h in PBS and bovine serum albumin at 37° C.

### 4.4.2 In Vitro Characterization

### 4.4.2.1 Inhibitory Capacities

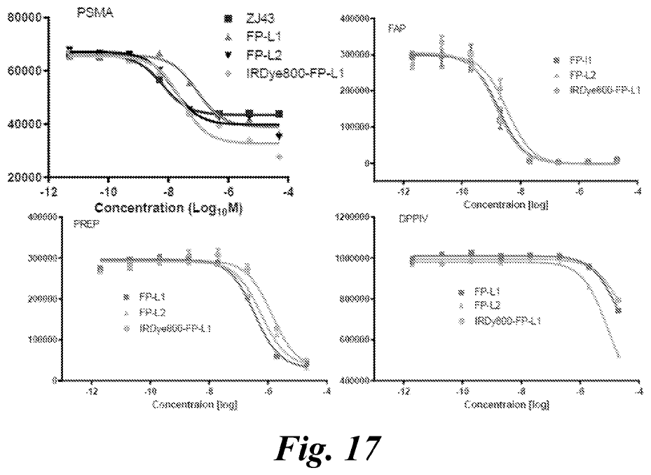

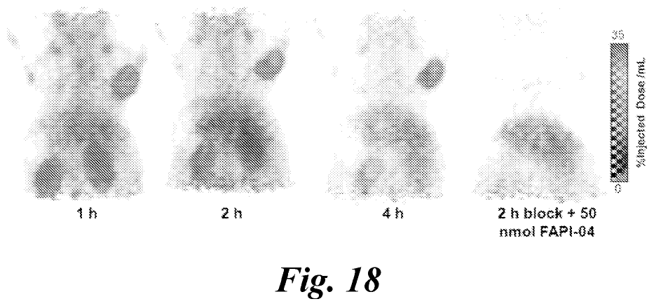

FP-L1 and FP-L2 displayed high binding affinity for FAP (Table 2, FIG. 17), comparable to that of FAPI-04, studied in the same assay as the reference compound. The PSMA binding affinity of FP-L1 bearing a polyethylene glycol (PEG) linker was 2-fold lower than that of FP-L2, which bears a pentamethylene linker. These values are lower than ZJ43, Olszewski et al., 2004, a known PSMA binding compound used as a standard for the assay.

TABLE 2
Inhibition constant (Kᵢ) of the agents
for FAP, DPPIV, PREP and PSMA.

| | | DPPIV | PREP | FAP | PSMA |
|---|---|---|---|---|---|
| Compound | MW | Ki | Ki | Ki | Ki |
| name | (g/mol) | (μM) | (μM) | (nM) | (nM) |
| FP-L1 | 1690.71 | 1.76 | 115.7 | 0.31 | 18.10 |
| FP-L2 | 1502.49 | 3.04 | 0.21 | 0.13 | 7.92 |
| IRDye800-FP-L1 | 2391.56 | 0.99 | 0.25 | 0.75 | 5.33 |
| ZJ-43 (standard for PSMA) | 304.3 | ND | ND | ND | 1.28 |
| FAPI-04 (standard for FAP) | 872.93 | 0.8 | 8.8 | 0.8 | ND |

### 4.4.2.2 Cell Uptake

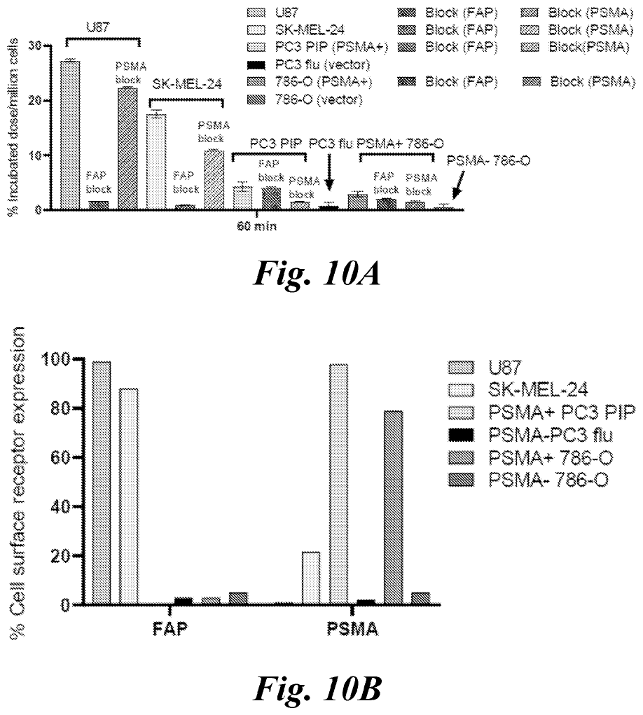

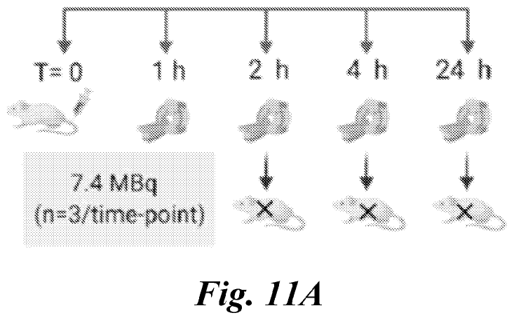

⁶⁴Cu-FP-L1 showed the highest uptake in FAP+U87 and SK-MEL-24 cells compared to PSMA+ PIP and PSMA-PC3 flu and 786 PSMA+/PSMA− cells (FIG. 10A). Blocking studies revealed a significant (P<0.001) lowering of uptake upon co-incubation with FAPI-04 (FAP blockade) or ZJ43 (PSMA blockade), indicating binding specificity of the compounds. Cell uptake was proportional to the surface expression of the target proteins of the studied cell lines as revealed by the flow cytometry (FIG. 10B).

### 4.4.3 In Vivo Characterization

### 4.4.3.1 Biodistribution and PET Imaging (Experiment 1)

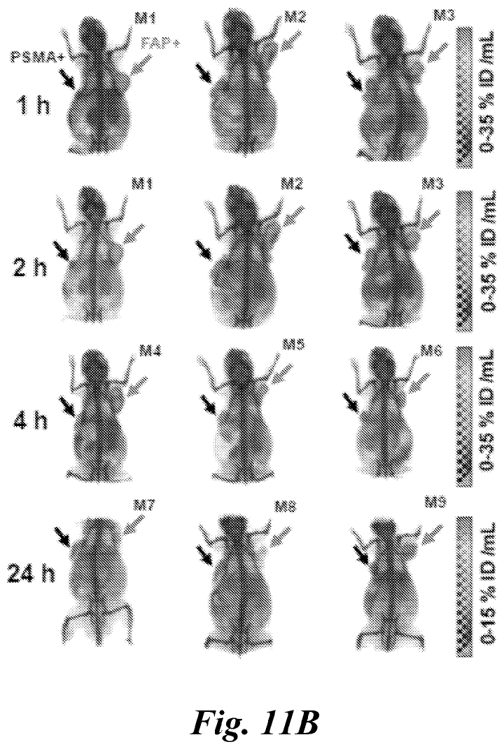

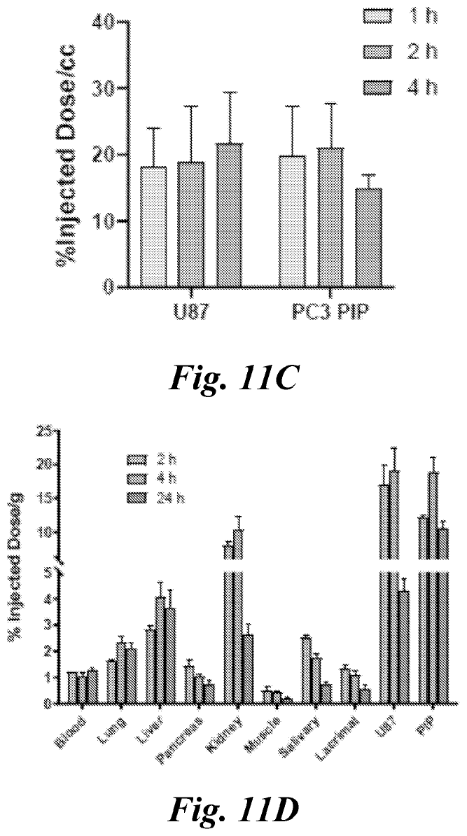

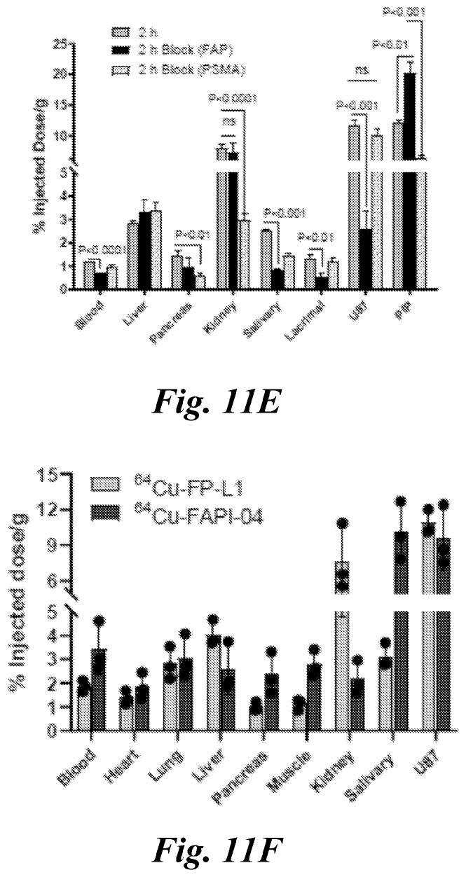

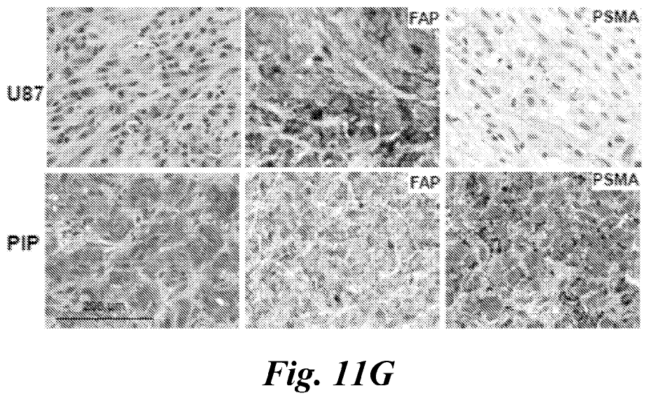

⁶⁴Cu-FP-L1 was evaluated in a mouse xenograft model bearing both PSMA+ human prostate cancer PC3 PIP and FAP+ U87 tumor in left and right upper flanks, respectively (FIG. 11A, FIG. 11B). Biodistribution data (FIG. 11C) revealed that tumor uptake was high (16.96±5.01% ID/g at 2 h, 19.05±5.89% ID/g at 4 h, and 4.31±0.75% ID/g at 24 h) in the FAP+ U87 tumor. Also, uptake in PSMA+ PC3 PIP tumor remained high, 12.06±0.78% ID/g at 2 h, 18.89±3.95% ID/g at 4 h and 10.51±1.82% ID/g at 24 h. Compared to a previously reported FAP-based compound, 111 In-QCP02, Slania et al., 2021, tumor uptake and retention of ⁶⁴Cu-FP-L1 was higher after 2 h post-injection, while the non-specific healthy tissue clearance was similar. Blocking studies to determine binding specificity (FIG. 11E) using FAPI-04 (500 nmol/kg) showed a significant decrease in uptake in U87 tumor (2.47±0.82% ID/g) and increased uptake in PSMA+ PC3 PIP tumors (18.56±2.02% ID/g) at 2 h. A significant decrease in activity was observed in healthy tissues, specifically in blood, salivary and lacrimal glands. Relatively high bone uptake (>1% ID/g) could be related to the initial high blood and marrow uptake of ⁶⁴Cu-FP-L1. In contrast, PSMA blocking using ZJ43 (500 nmol/kg) caused significantly lower uptake in PSMA+ PIP tumor (7.48±0.5% ID/g) indicating PSMA specificity of the agent. Specificity was further supported by >2-fold lower kidney uptake (2.97±0.23% ID/g) compared to unblocked agent (8.03±0.89% ID/g), a known endogenous PSMA-expressing site. Silver et al., 1997.

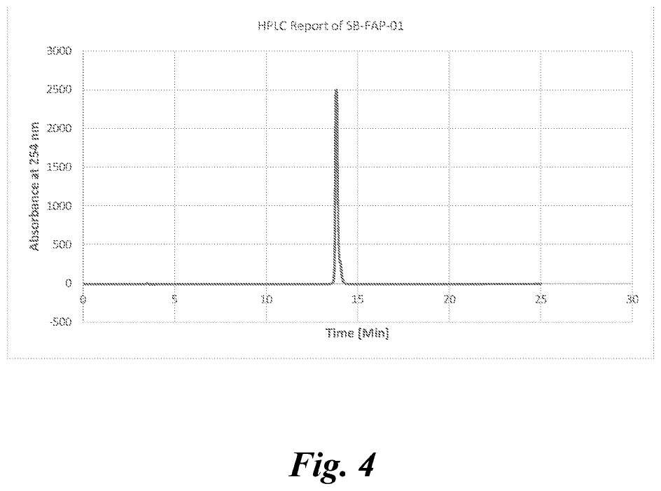

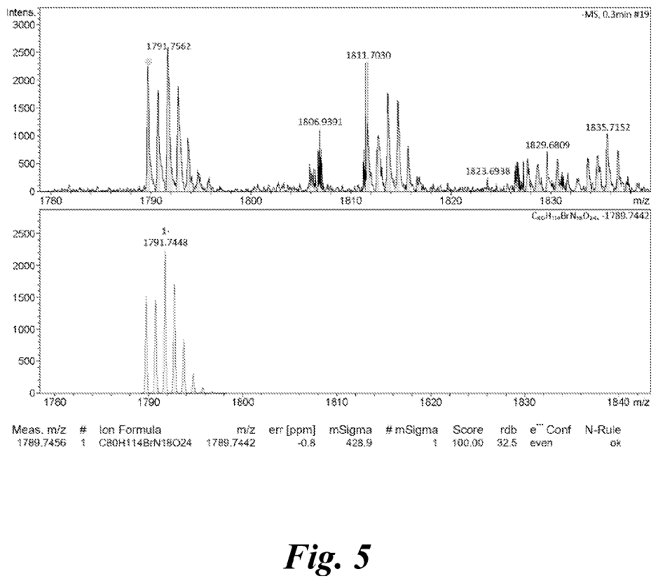

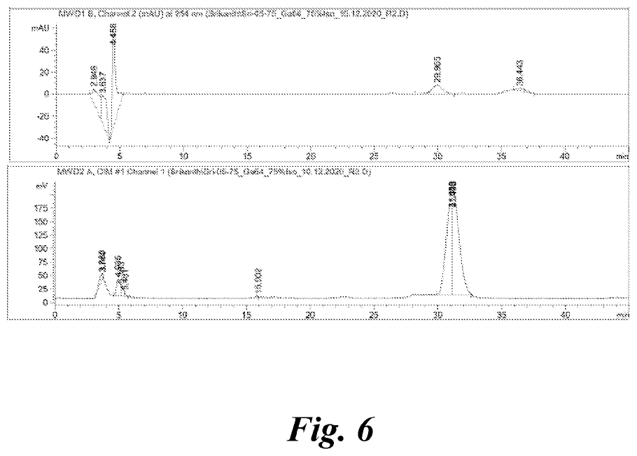

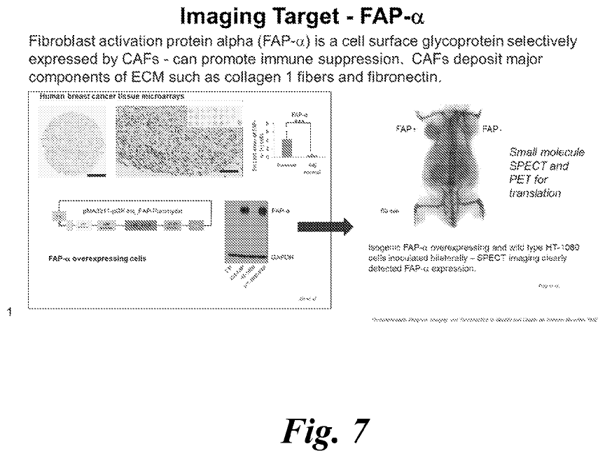

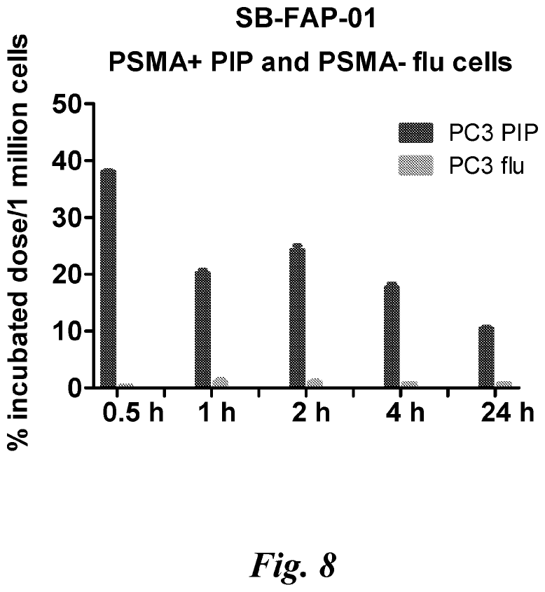

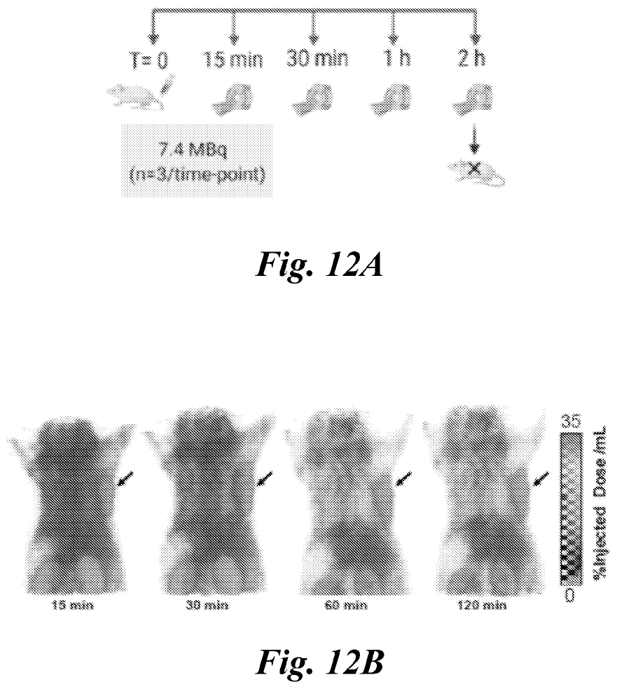

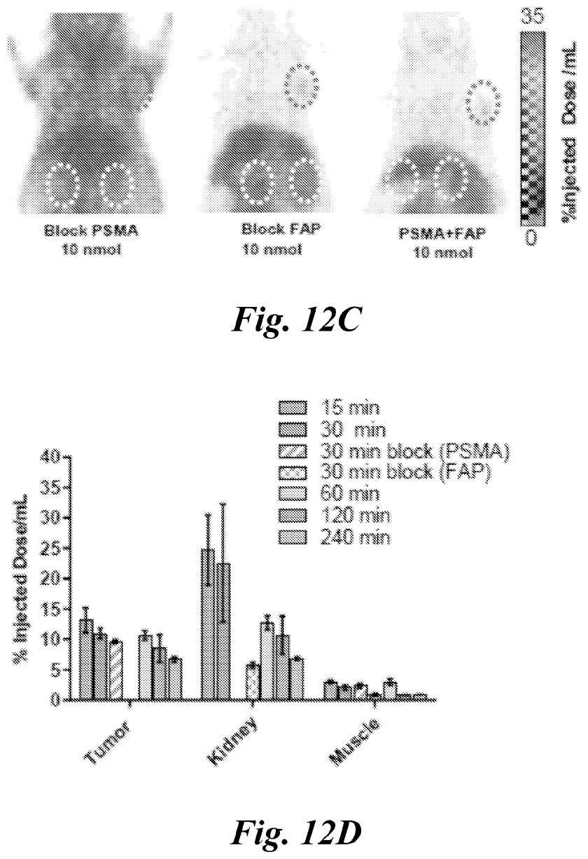

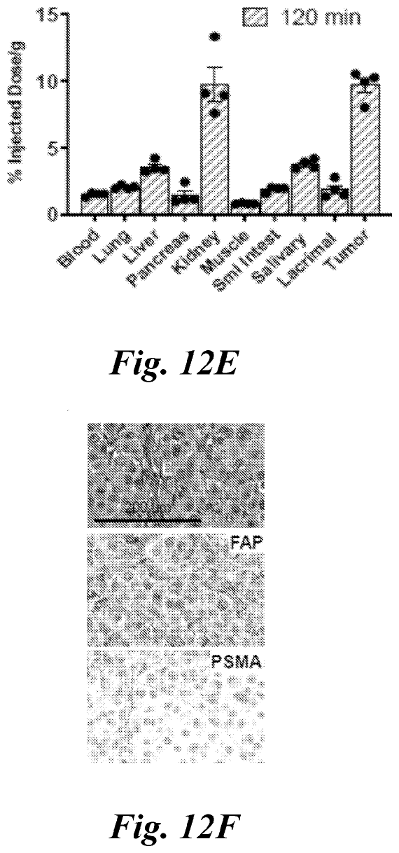

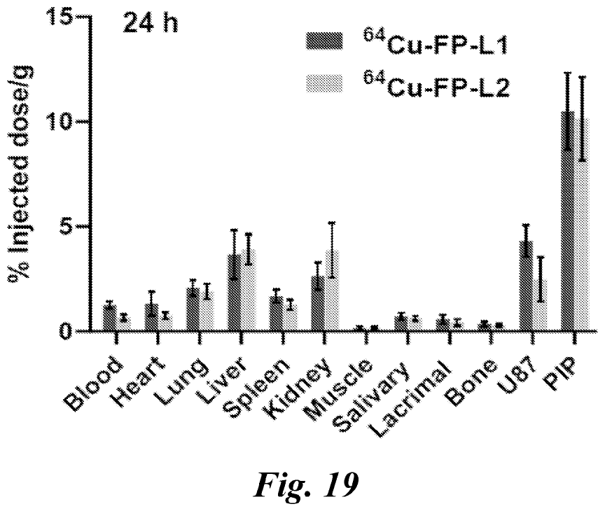

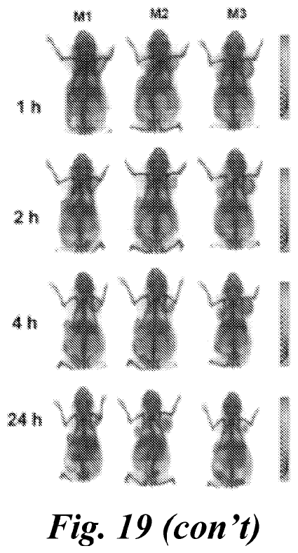

This model (FAP+ U87 and PSMA+ PC3 PIP) was evaluated using ⁶⁴Cu-FP-L2, the agent, however, displayed significantly higher kidney uptake than ⁶⁴Cu-FP-L1 (FIG. 19) and relatively lower tumor uptake and was not studied further. A biodistribution study using ⁶⁴Cu-FP-L1 also was performed using small tumors (approximately 51.9±16.5 mm³) at 2 h post-injection and was compared with ⁶⁴Cu-FAPI-04. Tumor uptake was comparable, 10.84±1.02% ID/g for ⁶⁴Cu-FP-L1 and 9.53±2.55% ID/g for ⁶⁴Cu-FAPI-04 (FIG. 11F). Non-specific uptake in kidney was high for ⁶⁴Cu-FP-L1 while high salivary gland uptake was observed for ⁶⁴Cu-FAPI-04. IHC studies validated high FAP expression and no/low PSMA expression in U87 tumor and relatively lower FAP and high PSMA expression in PSMA+ PC3 PIP tumors (FIG. 11G). Imaging and biodistribution in the SK-MEL-24 human melanoma model (Experiment 2). ⁶⁴Cu-FPL1 was evaluated in FAP+ SK-MEL-24 tumor xenografts (n=4, FIGS. 4A-4E). Intense tumor accumulation was found at 15 min post-injection, while activity cleared over the next 2 h. At 2 h, the tumor uptake was 9.66±1.14% ID/g. Among healthy tissues, kidney (9.71±2.48% ID/g), liver (3.54±0.46% ID/g), salivary glands (3.77±0.35% ID/g) and bone (2.91±0.59% ID/g) displayed relatively high uptake. No detectable tumor accumulation was found at 24 h post-injection. Uptake of the other healthy tissues was in the range of that of blood (1.51±0.15 ID/g). Receptor blocking studies were performed using FAPI-04 and ZJ43, and dual blockade using FP-L1 (autoblockade) (FIG. 12C). Blocking studies were monitored by PET and showed significantly lower tumor uptake for the FAP blockade and dual FAP and PSMA blockade compared to PSMA blockade. PSMA blockade was revealed by the lowering of signal intensity in the kidneys, a known PSMA-expressing tissue. Although, SK-MEL-24 expresses medium-low PSMA, it was previously shown that PSMA levels in these cells were approximately 10-fold lower compared to PSMA+ PC3 PIP tumor, Nimmagadda et al., 2018, however, the studied cohort of tumors displayed low PSMA expression as revealed in IHC studies (FIG. 12F).

### 4.4.3.2 Imaging in a GEMM of Pancreatic Cancer (Experiment 3)

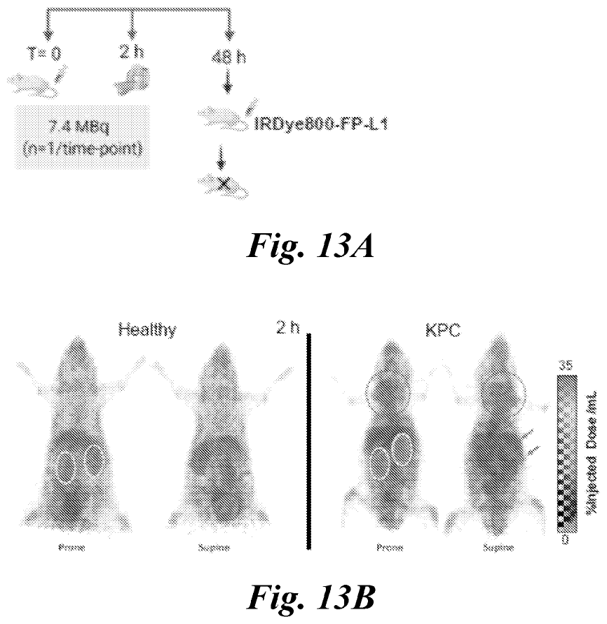

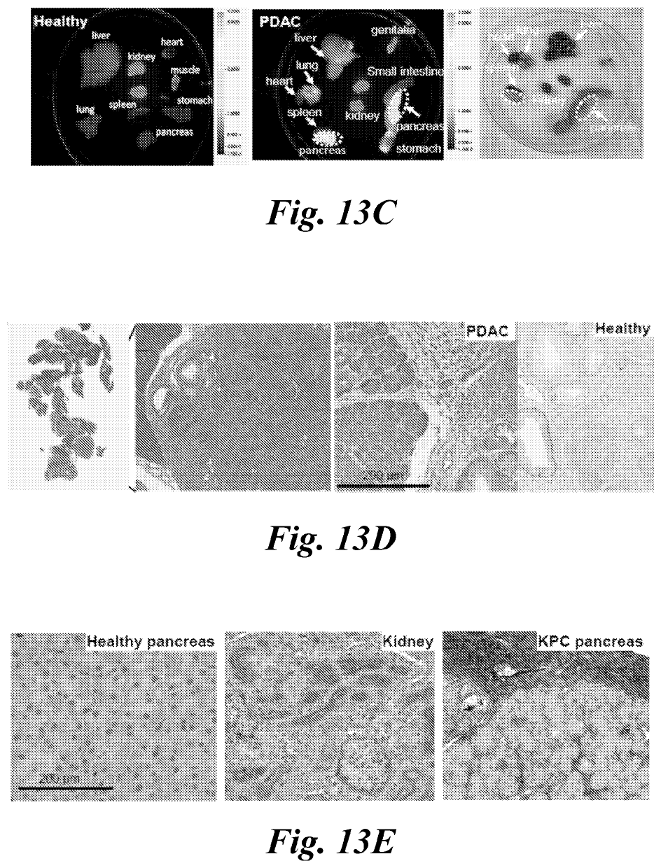

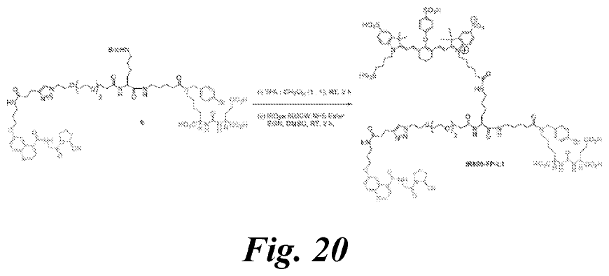

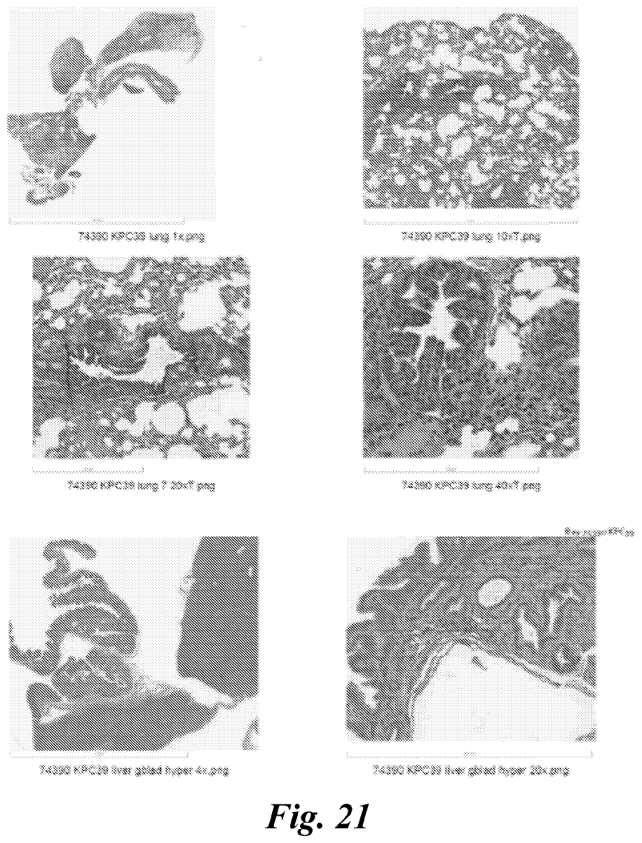

Imaging of ⁶⁴Cu-FP-L1 was performed in immunocompetent KPC mice (FIG. 13). Pancreatic ductal carcinoma (PDAC) progression in KPC mice recapitulates human PDAC. He et al., 2020. PET demonstrated higher uptake in the abdominal area in the KPC mice than the healthy mouse at 2 h post-injection (FIG. 13B). For confirmation of radiotracer distribution, mice were injected with the corresponding NIRF imaging agent, IRDye800-FP-L1 (5 nmol) (FIG. 20), bearing the same heterobivalent construct as FP-L1, followed by NIRF imaging of dissected organs at 2 h. As shown in FIG. 13C, IRDye800-FP-L1 accumulated specifically within the pancreas of the KPC mouse, demonstrating a clear margin between it and the healthy spleen. In contrast, the pancreas of the control mouse displayed minimal uptake. Both mice showed high uptake in kidneys, due to renal clearance. Histopathologic evaluation further confirmed the presence of PDAC lesions. PDAC lesions were <2 mm and associated with high FAP expression as confirmed by IHC (FIG. 13D). IHC data also revealed moderate PSMA expression within the TME (FIG. 13D). Lung metastases (<1 mm) also were observed, suggesting the high sensitivity of radiotracer (histology, FIG. 21). ⁶⁴Cu-FP-L1 and IRDye800-FP-L1 can delineate pancreatic tumor and metastases in the KPC model of PDAC through PET and NIRF imaging, respectively.

4.4.3.3 Imaging in a PSMA+ 786-O of ccRCC (Experiment 4)

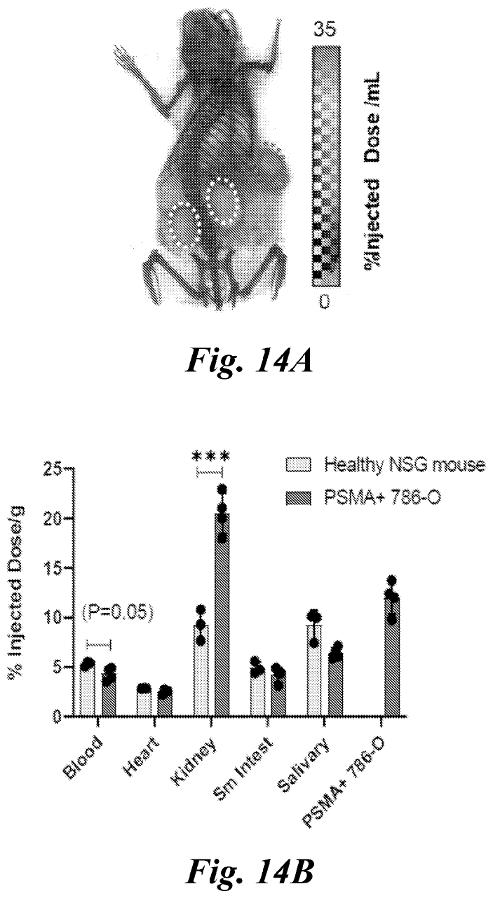

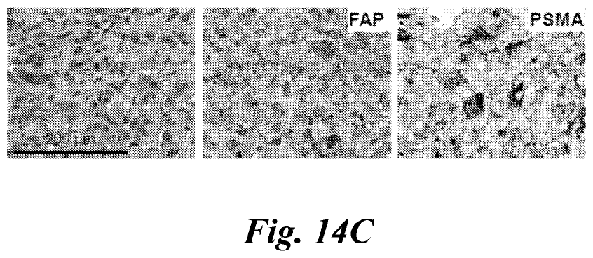

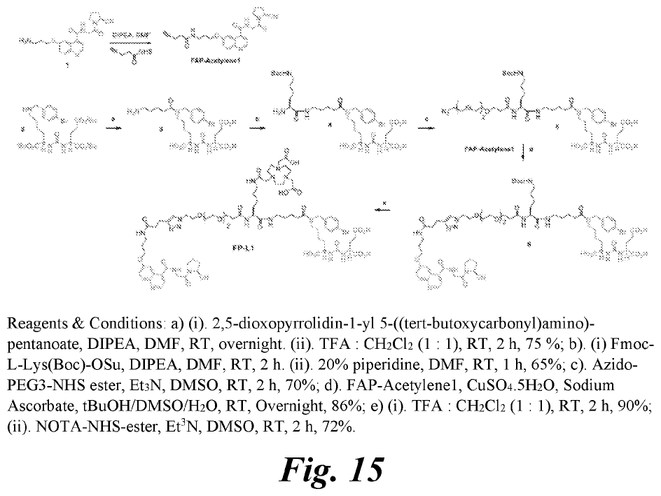

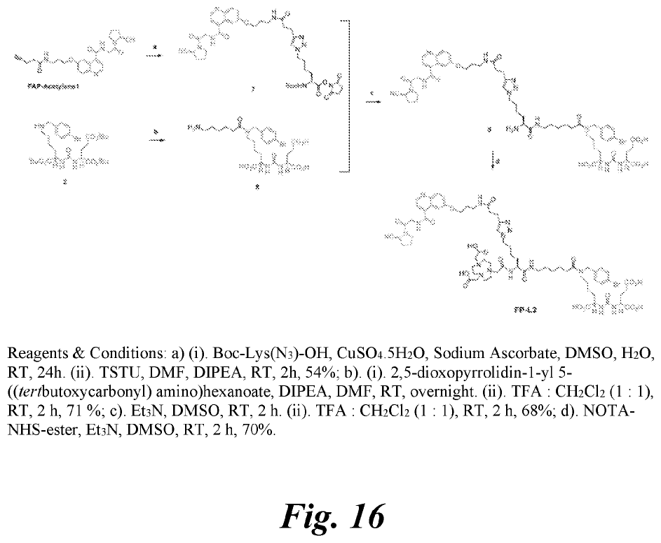

FAP expression is associated with tumor aggressiveness and poor survival in ccRCC. López et al., 2016. Lower levels of soluble FAP in the plasma of patients with ccRCC compared to healthy controls predicts tumor progression. Solano-Iturri et al., 2020. To test these clinical events, a PSMA+ 786-O tumor model that retains the ccRCC phenotype was recently developed. This model displayed lower FAP expression compared to U87 and SK-MEL-24, and lower PSMA expression than PSMA+ PC3 PIP tumors as revealed by flow cytometry (FIG. 10B). A head-to-head biodistribution performed in PSMA+ 786-O xenograft-bearing NSG mice and healthy NSG mice using ⁶⁴Cu-FP-L1 demonstrated high renal uptake compared to healthy mice (FIG. 14B). High tumor uptake also was noted in PET imaging and biodistribution studies (11.97±1.63% ID/g) at 2 h. IHC revealed high PSMA and FAP expression within the tumor sections as (FIG. 14C).

### 4.5 Discussion

One goal of the presently disclosed subject matter was to develop a dual-targeting radioligand capable of detecting a wider range of cancers than possible with current agents. Because of the medical importance of FAP and PSMA as cancer biomarkers, and their expression in many cancers, these two cell surface proteins were selected to be targeted with the same compound. Also of interest was having one versatile agent that might enable detection and treatment of a wide range of prostate cancers, from castration sensitive, through mCRPC including NEPC, the latter of which does not express PSMA, but has proved detectable by ⁶⁸Ga-FAPI-04. Kesch et al., 2021. One agent would likely have more uniform dosimetry and pharmacokinetics compared to administration of a cocktail including radioactive FAP and PSMA targeting compounds, and, by extension, might be easier to translate clinically. Note that it is not necessarily expected or intended for these heterobivalent compounds to bind both FAP and PSMA simultaneously. FAP and PSMA are on different cells and tissues within the TME or, in the case of PSMA and prostate cancer, within the tumor epithelium.

Reprogramming the TME by targeting specific, contributory cells, e.g., CAFs, macrophages or T cell subtypes, is a new and promising approach to treating cancer and overcoming resistance. A dual-targeting approach has the advantage of managing two biologically disparate targets and could lead to synergy. In certain cancers it may be beneficial to engage both the associated CAFs, as well as the neovasculature with a therapeutic radiopharmaceutical. One such example is clear cell renal cell carcinoma (ccRCC), where FAP expression has been correlated with more aggressive and metastatic disease, as the cancer cells undergo epithelial to mesenchymal transition. Errarte et al., 2016. It has been shown that ccRCC can be imaged to good advantage with a PSMA-targeted PET agent, Meyer et al., 2019, by virtue of the chimeric neovasculature of ccRCC, Delgado-Bellido et al., 2017; Zhou et al., 2016, in which the tumor vessels are comprised of both endothelial and cancer cells. Antivascular agents are used to treat ccRCC, more recently in combination with immune checkpoint inhibitors, Rini et al., 2019, suggesting that targeting the neovasculature—with a radiotheranostic—may enable tumor growth control. Other such cancers, which express FAP and PSMA highly, Slania et al., 2021; Nimmagadda et al., 2018; Puré and Blomberg, 2018, and for which a dual-targeting approach may prove helpful, include melanoma, breast, glioma, lung, ovary, upper aerodigestive cancers, and pancreas, the last as demonstrated in the KPC mouse (FIG. 13). The KPC mouse was selected because pancreatic cancer is known to express both FAP and PSMA, Stock et al., 2017; 2017; Pereira et al., 2019; Poels et al., 2021; Krishnaraju et al., 2021, and is notoriously difficult to image. A dual-targeting approach also provides a default mechanism if one of the targets is pharmacokinetically inaccessible in a particular lesion or is expressed at different stages of disease from the other. Examples of that strategy include heterobivalent, bispecific antibodies that have received regulatory approval for treating certain cancers, Sheridan, 2015; heterobivalent immunoligands also have demonstrated enhanced tumor uptake and retention for PET. Luo et al., 2015. The presently disclosed subject matter also adopted a heterobivalent approach, however, focusing on small-molecule targeting moieties. It was previously shown that such a strategy was possible by targeting PSMA and integrin αvβ3 to provide synergy in targeting tumor neovasculature. Shallal et al., 2014. Others have similarly attempted dual targeting of PSMA and other cell surface proteins, most notably gastrin-releasing peptide receptor (GRPR). Bandari et al., 2021; Eder et al., 2014.

The presently disclosed subject matter, in some embodiments, provides two orthogonal targets, one on CAFs (FAP) and the other on neovasculature (PSMA), to provide synergy, but in addition, to enable target engagement by a theranostic agent in the instance that one target is absent from the lesion. To test the ability of ⁶⁴Cu-FP-L1 to engage FAP and PSMA in the same administration tumor cell lines were sought in which both targets are known to be expressed at least moderately, such as SK-MEL-24. Nimmagadda et al., 2018.

Synergistic uptake, however, was not observed, namely, from the engagement of both FAP and PSMA, of ⁶⁴Cu-FP-L1 in that cell line (FIG. 10) or with in vivo targeting (FIG. 12) and was consistent with the IHC data showing low PSMA from the studied cohort of tumors. Notably, in vivo tumor targeting of ⁶⁴Cu-FP-L1 in PC3 PIP tumor was significantly higher than reported in PSMA-based heterobivalent compounds. Bandari et al., 2021. Biodistribution data of ⁶⁴Cu-FAPI-04 from the presently disclosed method demonstrated similar in vivo properties as the reported data. Watabe et al., 2020. It also should be noted that higher tumor uptake (% ID/g) was associated with tumor volumes >200 mm³ relative to tumors with volumes <100 mm³ in the U87 model. Without wishing to be bound to any one particular theory, it is thought that large tumors likely provided additional stromal or desmoplastic tissue uptake within the TME.

These studies suggest that ⁶⁴Cu-FP-L1 can detect both FAP+ and PSMA+ tumors in vivo with minimal non-specific tissue accumulation. In addition to detecting FAP and PSMA in the TME in a variety of solid tumors, ⁶⁴Cu-FP-L1 or suitable analogs may enable imaging of the spectrum of prostate cancer subtypes including those that no longer express PSMA. Isik et al., 2021. Although more lesions will be detected, a limitation of this approach is that it will not be known if the lesion is detected by virtue of FAP or by PSMA expression (or both), which would require tissue sampling for definitive characterization. This limitation is thought to be offset by the increased sensitivity. An agent similar to ⁶⁴Cu-FP-L1 could be used to obtain a fuller picture of lesions present in a patient being staged for prostate cancer, or to follow a patient undergoing PSMA-specific radiopharmaceutical therapy to understand why they may be failing to respond, for example, through localization of appearing neuroendocrine-differentiated lesions. The corresponding radiotherapeutic, 67Cu-FP-L1, might enable treatment of both PSMA+ and NEPC cancer concurrently.

### 4.6 Summary

The presently disclosed data show that ⁶⁴Cu-FP-L1 can target both PSMA and FAP expression in the same in vivo experiment. By targeting two prevalent targets, one (FAP) touted as a pan-cancer marker, ⁶⁴Cu-FP-L1 has the potential to detect more than one type of cell on the tumor cell surface or in the TME, and the corresponding therapeutic may address the issue of resistance due to tumor heterogeneity.

### Example 5

### Imaging Agents and Radiotherapeutics Targeting Fibroblast-Activation Protein-α (FAP-α)

### 5.1 Overview

Fibroblast-activation protein-α (FAP-α) expression has been detected on the surface of fibroblasts in the stroma surrounding >90% of the epithelial cancers (malignant breast, colorectal, skin, prostate, pancreatic cancers, and the like) and inflammation diseases (arthritis, fibrosis, and the like) with nearly no expression in healthy tissues. Imaging and radiotherapeutic agent specifically targeting FAP-α is of clinical importance. Since FAP shares a close similarity in the sequence and catalytic region with dipeptidyl peptidase IV (DPPIV) and propyl endopeptidase (PREP), most inhibitors of FAP also bind to DPPIV and PREP. Here, potent and selective low-molecular-weight (LMW) inhibitors of FAP-α with a targeting moiety feasible for modification with radiolabeling groups and optical groups were developed. These compounds enable in vivo nuclear imaging (such as PET and SPECT), optical imaging and radiotherapy targeting FAP-α. The presently disclosed targeting moiety can be adapted for other optical dyes and radioisotopes for imaging and therapeutic applications targeting FAP-α.

In part, the presently disclosed subject matter demonstrates that: (1) radiolabeled isotopes with sufficient half-lives conjugated to a low molecular weight (LMW) FAP-α selective inhibitor allows for successful in vivo and ex vivo imaging; (2) the presently disclosed compounds can be modified with labeling groups without significantly losing potency, allowing for labeling with optical dyes, PET and SPECT isotopes, including but not limited to ¹⁸F, ⁶⁸Ga, ⁶⁴Cu, ⁸⁶Y, ⁹⁰Y, ⁸⁹Zr, ¹¹¹In, ^{99m}Tc, ¹²⁵I, ¹²⁴I, ¹¹C, ⁷⁶BR for FAP-α related imaging applications; (3) the presently disclosed compounds additionally allow for radiolabeling with radiotherapeutic isotopes, including but not limited to ⁹⁰Y, ¹⁷⁷Lu, ¹²⁵I, ¹³¹I, ²¹¹At, ¹¹¹In, ¹⁵³Sm, ¹⁸⁶Re, ¹⁸⁸Re, ⁶⁷Cu, ²¹²Pb, ²²⁵Ac, ²¹³Bi, ²¹²Bi, ²¹²Pb, ⁶⁷Ga, ⁷⁷Br, ¹⁶¹Tb for FAP-α related radio-therapy; and (4) the presently disclosed compounds exhibited specific binding to FAP positive cell lines over closely related peptidases DPPIV and PREP.

### 5.2 Background

### 5.2.1 Fibroblast-Activation Protein-α (FAP-α)

FAP-α expression has been linked to the tumor microenvironment and has been detected on the surface of stromal fibroblasts surrounding greater than 90% of epithelial-derived cancers and their metastases. Garin-Chesa et al., 1990; Rettig et al., 1993; Tuxhorn et al., 2002; Scanlan et al., 1994. FAP-α plays a critical role in promoting angiogenesis, proliferation, invasion, and inhibition of tumor cell death. Allinen et al., 2004; Franco et al., 2010. In healthy adult tissues, FAP-α expression is only limited to areas of tissue remodeling or wound healing. Scanlan et al., 1994; Yu et al., 2010; Bae et al., 2008; Kraman et al., 2010.

FAP expression is extremely difficult to detect in non-diseased adult organs, but is greatly upregulated in sites of tissue remodeling, which include lung or liver fibrosis, arthritis and tumors. Scanlan et al., 1994; Yu et al., 2010. These characteristics make FAP-α a good imaging and radiotherapeutic target for cancer and inflammation diseases.

5.2.2 Significance for FAP-α Imaging with Low-Molecular-Weight (LMW) Agents.

FAP-α is expressed in tumor stroma, anti-FAP antibodies have been studied for radioimmunotargeting of malignancies, including murine F19, sibrotuzumab (a humanized version of the F19 antibody), ESC11, ESC14, among others. Welt et al., 1994; Scott et al., 2003; Fischer et al, 2012. Antibodies also demonstrated the feasibility to imaging inflammation, such as rheumatoid arthritis. Laverman et al., 2015. Antibodies as molecular imaging agents, however, suffer from pharmacokinetic limitations, including slow blood and non-target tissue clearance (normally 2-5 days or longer) and non-specific organ uptake. LMW agents demonstrate faster pharmacokinetics and higher specific signal within clinically convenient times after administration. They also can be synthesized in radiolabeled form more easily and may offer a shorter path to regulatory approval. Coenen et al., 2010; Cho et al., 2012; Reilly et al., 2015.

FAP-α is a type II transmembrane serine protease of the prolyl oligopeptidase family, which are distinguished by their ability to cleave the post-proline peptide bond. It has been shown to play a role in cancer by modifying bioactive signaling peptides through this enzymatic activity. Kelly, 2005; Edosada et al., 2006. FAP-α exists as a homodimer to carry out its enzymatic function. Inhibitors selectively targeting FAP-α have been reported. Lo et al., 2009; Tsai et al., 2010; Ryabtsova et al., 2012; Poplawski et al., 2013; Jansen et al., 2013; Jansen et al., 2014. DPPIV is FAP's closest homolog with over 50% sequence similarity and over 70% similarity in the catalytic region. Juillerat-Jeanneret et al., 2017. Prolyl endopeptidase (PREP) is phylogenetically related to FAP and, similar to FAP, cleaves the post-proline peptide bond of its substrates. Jambunathan et al., 2012. Therefore, it is essential to establish the specificity of the imaging compounds for FAP over DPPIV and PREP. The FAP-α selective inhibitors could potentially provide the solution for direct optical agents or radioisotope for FAP-α targeted imaging and therapy.

The presently disclosed subject matter provides an FAP-α selective targeting moiety feasible to be modified with optical dye, radiometal chelation complex, and other radioisotope prosthetic groups. It provides a platform for imaging and radiotherapy targeting FAP-α. In some instances, linkers are chosen such that the FAP pharmacophore and labeled prosthetic group, e.g., a prosthetic group labeled with 18F, are separated with hydrophilic amino-acid based linkers. The hydrophilic linkers decrease non-specific binding and improve pharmacokinetics of the radiotracers

### 5.2.3 Significance for Nuclear Imaging and Radiotherapy.

Radionuclide molecular imaging including PET is the most mature molecular imaging technique without tissue penetration limitations. Due to its advantages of high sensitivity and quantifiability, radionuclide molecular imaging plays an important role in clinical and preclinical research. Youn and Hong, 2012; Chen et al., 2014.

Many radionuclides, primarily β- and alpha emitters, have been investigated for targeted radioimmunotherapy and include both radiohalogens and radiometals (Table 1). The highly potent and specific binding moiety targeting FAP-α enables its nuclear imaging and radiotherapy. Herein, the first synthesis of nuclear imaging and radiotherapy agents based on this dual-targeting moiety to FAP-α for nuclear imaging is described.

### 5.3 Experimental Section

### 5.3.1 Synthesis of the Ligands:

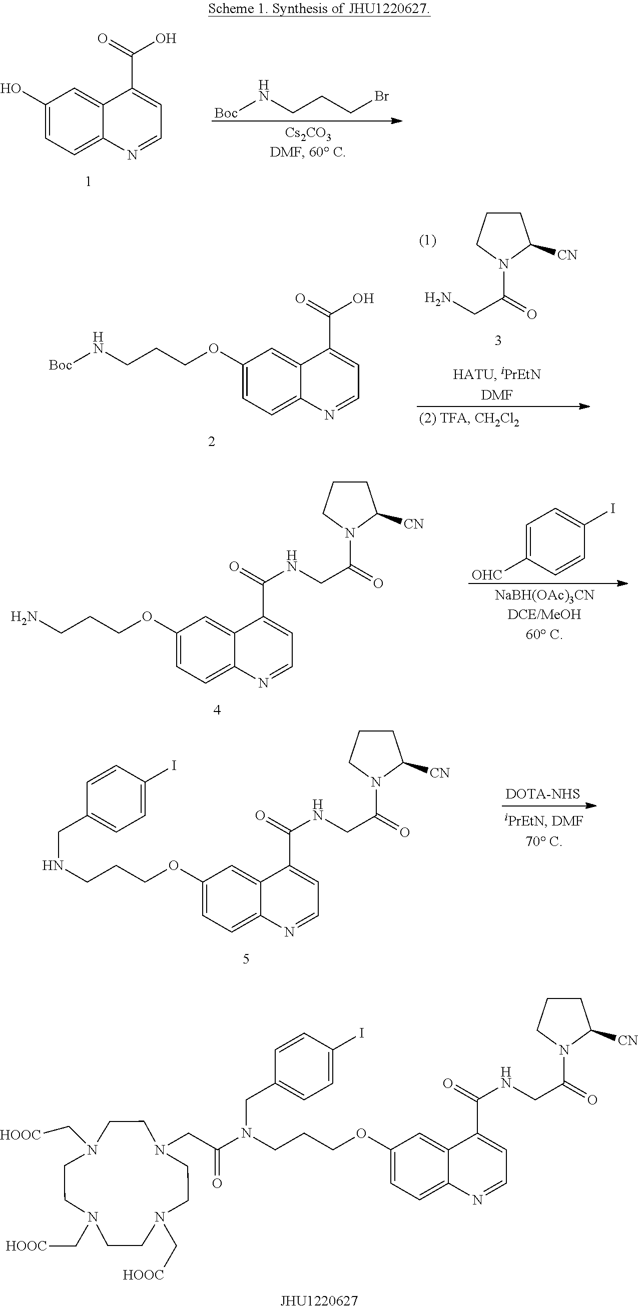

6-(3-((tert-butoxycarbonyl)amino)propoxy)quinoline-4-carboxylic acid (2): To a stirred solution of 6-Hydroxyquinoline-4-carboxylic acid (1) (300 mg, 1.586 mmol, 1 eq.) and cesium carbonate (1.55 g, 4.758 mmol, 3 eq.) in 5 mL DMF, tert-butyl (3-bromopropyl) carbamate (944 mg, 3.965 mmol, 2.5 eq.) was added and the mixture allowed to stir overnight at 60° C. The reaction was cooled to room temperature and diluted with 2 mL acetonitrile and 5 mL water, followed by addition of 600 μL of 12M NaOH. After stirring at room temperature for 30 min, the reaction mixture was loaded onto a 30 g C18 cartridge (Biotage Sfar). The product was purified with a MeCN/water/TFA gradient (5/100/0.1 to 60/10/0.1). 312 mg of product 2 was obtained with a yield of 90%. ¹H-NMR (400 MHZ, CD₃OD): δ 8.80 (d, J=3.2 Hz, 1H), 8.28 (s, 1H), 8.04-7.98 (comp, 2H), 7.51 (d, J=7.2 Hz, 1H), 4.19 (t, J=7.6 Hz, 2H), 3.29-3.26 (comp, 2H), 2.04 (t, J=7.6 Hz, 2H), 1.42 (s, 9H). ESI-MS: m/z calculated for C₁₈H₂₃N₂O₅ [M+H]⁺ 347.4; found 347.1.

(S)-6-(3-aminopropoxy)-N-(2-(2-cyanopyrrolidin-1-yl)-2-oxoethyl)quinoline-4-carboxamide (4): To a stirred solution of 2 (184 mg, 0.53 mmol, 1.0 eq.), 3 (122 mg, 0.795 mmol, 1.5 eq.) and HATU (302 mg, 0.795 mmol, 1.5 eq.) in DMF (3 mL) was added DIPEA (231 μL, 1.325 mmol, 2.5 eq.) at room temperature. The reaction mixture was stirred overnight at room temperature. After removal of solvent under vacuum, 2 mL of TFA/CH₂Cl₂ (1:1) was added and stirred for 30 mins. Reaction mixture was concentrated and loaded onto a 30 g C18 cartridge (Biotage Sfar). The product was purified with a MeCN/water/TFA gradient (5/100/0.1 to 60/10/0.1). After lyophilization, 120 mg of product 4 was obtained with a yield of 74%. NMR (400 MHZ, CD₃OD): δ 8.93 (d, J=4.0 Hz, 1H), 8.17 (d, J=2.0 Hz, 1H), 8.10 (d, J=7.2 Hz, 1H), 7.81 (d, J=4.0 Hz, 1H), 7.67 (dd, J=7.6, 2.0 Hz, 1H), 4.84-4.78 (m, 1H), 4.47-4.38 (comp, 2H), 4.38-4.28 (comp, 2H), 3.86-3.78 (m, 1H), 3.70-3.62 (m, 1H), 3.24-3.18 (comp, 2H), 2.39-2.18 (comp, 6H). ESI-MS: m/z calculated for C₂₀H₂₄N₅O₃ [M+H]⁺ 382.4; found 382.2.

(S)—N-(2-(2-cyanopyrrolidin-1-yl)-2-oxoethyl)-6-(3-((4-iodobenzyl)amino)propoxy)quinoline-4-carboxamide (5): To a solution of 4 (40 mg, 0.105 mmol, 1.0 eq.) and 4-iodobenzaldehyde (27 mg, 0.115 mmol, 1.1 eq.) in 2 mL 1,2-dichloroethane/CH₃OH (1:1), sodium triacetoxyborohydride (76 mg, 0.314 mmol, 3 eq.) was added and stirred at 60° C. for 48 h. After cooling to room temperature, the reaction mixture was purified by silica gel flash chromatography eluting with 1% to 15% CH₃OH in CH₂Cl. 23 mg of product 4 was obtained in 50% yield. NMR (400 MHZ, CD₃OD): δ 8.80-8.75 (m, 1H), 8.02-7.96 (m, 2H), 7.80 (d, J=6.00 Hz, 2H), 7.58 (d, J=3.60 Hz, 1H), 7.45 (dd, J=7.20, 1.6 Hz, 1H), 7.26 (d, J=6.40 Hz, 2H), 4.81-4.75 (m, 1H), 4.40-4.34 (comp, 2H), 4.34-4.28 (comp, 2H), 4.15 (s, 2H), 3.86-3.77 (m, 1H), 3.70-3.61 (m, 1H), 3.28-3.21 (comp, 2H), 2.38-2.17 (comp, 6H). ESI-MS: m/z calculated for C₂₇H₂₈IN₅O₃ [M+H]⁺ 598.5; found 598.1.

Compound JHU1220627: To a stirred solution of 5 (7.5 mg, 11.41 μmol, 1.0 eq.) and DOTA-NHS-ester (10.4 mg, 13.69 μmol, 1.2 eq.) in DMF (250 μL) was added DIPEA (20 μL, 228.13 μmol, 20 eq.) at room temperature. The reaction mixture was stirred at 70° C. for 2 h and another 1.2 eq. of DOTA-NHS ester was added and stirred overnight. Solvent was then evaporated off and re-dissolved in acetonitrile/water (1:1) and pH adjusted to approximately 3 by addition of TFA. The product was purified by preparative HPLC chromatography using Phenomenex 21.2 mm×250 mm Luna, λ 254 nm, 10 um PREP C18 (2) column and isocratic eluent 30% Acetonitrile/70% water/0.1% TFA, at a flow rate of 10 mL/min. After lyophilization, 6.2 mg of JHU1220627 was obtained as a white solid in 55% yield. ESI-MS: m/z calculated for C₄₃H₅₅IN₉O₁₀ [M+H]⁺ 984.9; found 984.3.

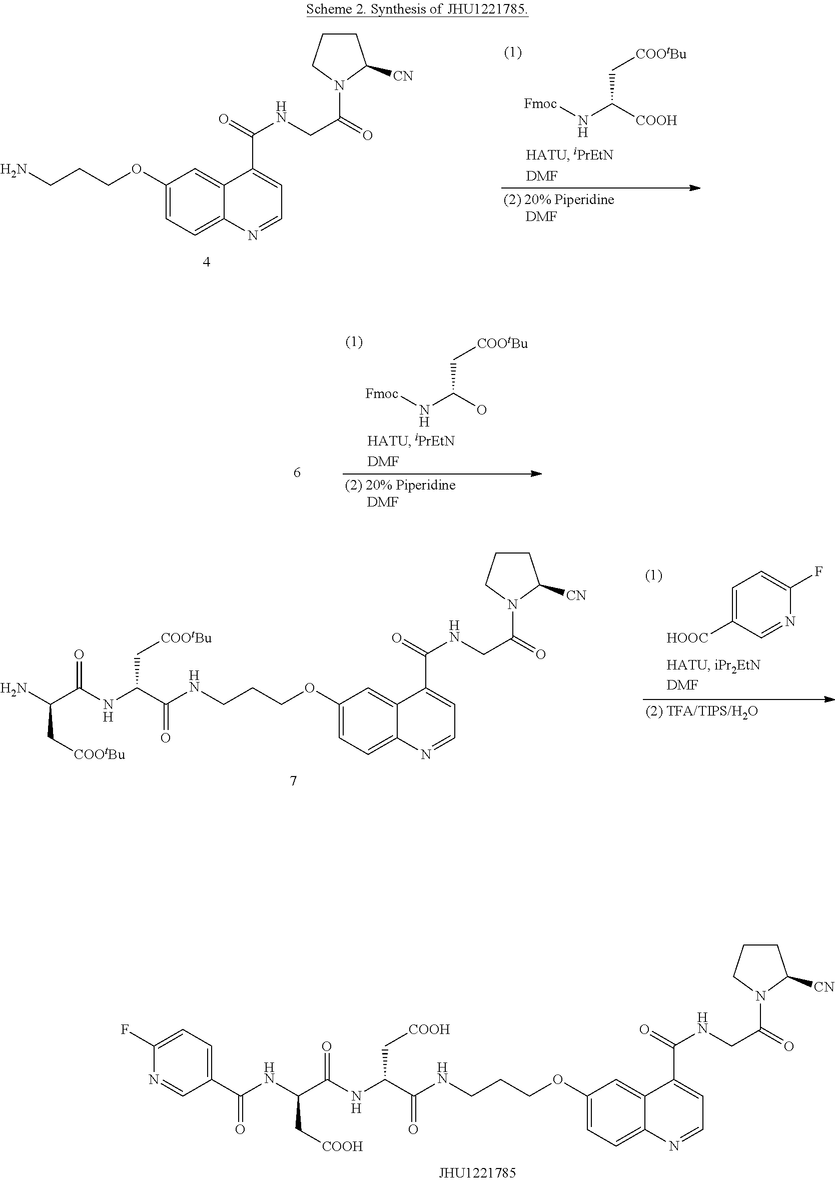

Compound 7: To a stirring solution of 4 (120 mg, 0.3146 mmol, 1.0 eq.), Fmoc-D-Asp (t-Bu)-OH (388 mg, 0.9438 mmol, 3 eq.) and HATU (359 mg, 0.9438 mmol, 3 eq.) in DMF (3 mL), DIPEA (219 μL, 1.2584 mmol, 4 eq.) was added and the mixture allowed to stir overnight at room temperature. After evaporating off the solvent, the mixture was stirred in 20% piperidine in DMF for 30 min. The reaction mixture was then diluted with EtOAc and washed with water (3×) and brine. The combined organics was dried over anhydrous Na₂SO₄, filtered, concentrated and dried under vacuum. The crude intermediate was then dissolved in 4 mL DMF, and Fmoc-D-Asp(t-Bu)-OH (388 mg, 0.9438 mmol, 3 eq.), HATU (359 mg, 0.9438 mmol, 3 eq.), DIPEA (219 μL, 1.2584 mmol, 4 eq.) were added and stirred at room temperature for 24 h. Solvent was then evaporated off and the mixture stirred in 20% piperidine in DMF for 30 min. The reaction mixture was then diluted with EtOAc and washed with water (3×) and brine. The combined organics were dried over anhydrous Na₂SO₄, filtered, concentrated and purified by silica gel flash chromatography, eluting with 1% to 10% CH₃OH in CH₂Cl₂. 138 mg of product 7 was obtained in 61% yield. ESI-MS: m/z calculated for C₃₆H₅₀N₇O₉ [M+H]⁺ 724.8; found 724.4.

Compound JHU1221785: To a stirring solution of 7 (25 mg, 34.54 μmol, 1.0 eq.), 6-fluoronicitinic acid (14.6 mg, 103.62 μmol, 3 eq.) and HATU (39.4 mg, 103.62 μmol, 3 eq.) in DMF (1 mL), DIPEA (24 μL, 135.15 μmol, 4 eq.) was added and the mixture allowed to stir overnight at room temperature. The reaction mixture was then diluted with EtOAc and washed with water (3×) and brine. The combined organics was dried over anhydrous Na₂SO₄, filtered and concentrated. 2 mL of TFA/CH₂Cl₂ (1:1) was added and stirred for 30 min. The mixture was then concentrated and loaded onto a 12 g C18 cartridge (Biotage Sfar). The product was purified with a MeCN/water/TFA gradient (5/100/0.1 to 50/10/0.1). After lyophilization, 10 mg of JHU1221785 was obtained as a white solid in 40% yield. NMR (400 MHZ, CD₃OD): δ 9.00-8.95 (m, 1H), 8.62 (d, J=13.2 Hz, 1H), 8.39-8.26 (m, 1H), 8.18-8.13 (m, 1H), 8.13-8.08 (m, 1H), 7.98-7.92 (m, 1H), 7.79-7.72 (m, 1H), 7.15-7.08 (m, 1H), 4.77-4.83 (m, 1H), 4.72-4.68 (m, 1H), 4.40 (s, 2H), 4.37-4.26 (comp, 2H), 3.86-3.77 (m, 1H), 3.70-3.60 (m, 1H), 3.50-3.41 (comp, 2H), 2.98-2.66 (comp, 5H), 2.37-2.26 (comp, 2H), 2.26-2.17 (comp, 2H), 2.15-2.05 (comp, 2H). ESI-MS: m/z calculated for C₃₄H₃₆FN₈O₁₀ [M+H]⁺ 735.7; found 735.3.

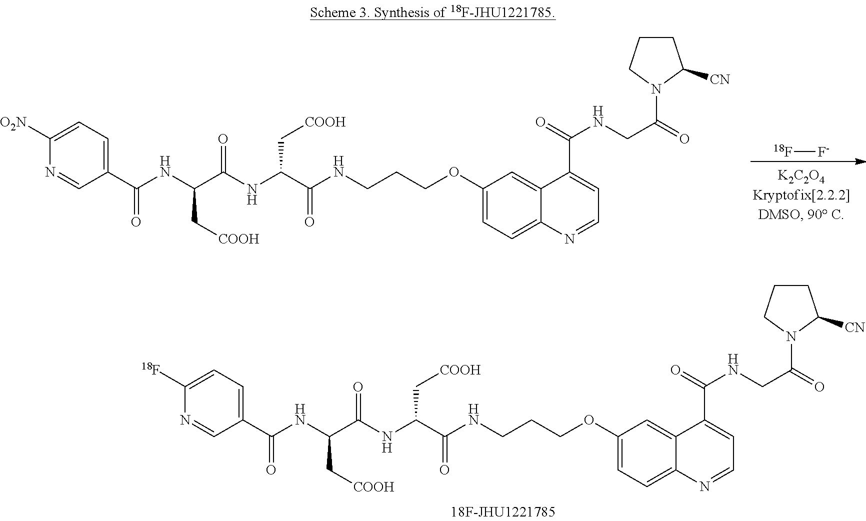

Methyl (6-hydroxyquinoline-4-carbonyl)glycinate (8): 6-Hydroxyquinoline-4-carboxylic acid (1) (200 mg, 1.05 mmol, 1.0 eq.), methyl glycinate HCl salt (200 mg, 1.58 mmol, 1.5 eq.) and HATU (603 mg, 1.58 mmol, 1.5 eq.) were dissolved in 5 mL anhydrous DMF. To the solution, DIPEA (0.46 mL, 2.64 mmol, 2.5 eq.) was added. The reaction was stirred at room temperature for 6 h. After the solvent was removed under vacuum, the mixture was loaded onto a 25 g C18 cartridge and the product was purified with a MeCN/water/TFA gradient (0/100/0.1 to 90/10/0.1). After lyophilization, the product 2 was obtained as a yellow powder with a yield of 76% (209 mg). ¹H-NMR (400 MHZ, CD₃OD): δ 8.69 (s, 1H), 7.94 (d, J=7.92 Hz, 1H), 7.57-7.51 (m, 3H), 7.42-7.37 (m, 1H), 4.21 (s, 2H), 3.81 (s, 3H). ¹³C-NMR (100 MHZ, CD₃OD): δ 172.4, 160.9, 145.1, 143.7, 129.7, 129.4, 128.3, 121.8, 119.6, 112.4, 109.1, 56.8, 44.8. ESI-MS: m/z calculated for C₁₃H₁₃N₂O₄ [M+H]⁺ 261.3; found 261.0.

Methyl (6-(3-((tert-butoxycarbonyl)amino)propoxy)quinoline-4-carbonyl)glycinate (9): Methyl (6-hydroxyquinoline-4-carbonyl)glycinate (8) (100 mg, 0.38 mmol, 1.0 eq.), 3-(Boc-amino) propyl bromide (230 mg, 0.96 mmol, 2.5 eq.) were dissolved in 4 mL anhydrous DMF. Cs₂CO₃ (376 mg, 1.15 mmol, 3.0 eq.) was added to the solution and the reaction was stirred at room temperature overnight. After filtration, the solvent was removed under vacuum and the remaining mixture was loaded onto a 25 g C18 cartridge. The product was purified with a MeCN/water/TFA gradient (0/100/0.1 to 90/10/0.1). After lyophilization, the product 3 was obtained as a yellow powder with a yield of 54% (86 mg). ¹H-NMR (400 MHZ, CDCl₃): δ 8.68-8.37 (m, 2H), 8.02 (d, J=9.1 Hz, 1H), 7.80 (s, 1H), 7.72-7.64 (m, 1H), 7.40 (d, J=9.1 Hz, 1H), 4.94 (br s, 1H), 4.41-4.31 (m, 2H), 4.27-4.18 (m, 2H), 3.85 (s, 3H), 3.44-3.30 (m, 2H), 2.13-2.00 (m, 2H), 1.43 (s, 9H). ¹³C NMR (100 MHz, CDCl₃): δ 170.1, 167.2, 158.4, 144.7, 142.3, 128.4, 126.1, 124.7, 119.1, 103.7, 79.5, 60.4, 52.5, 41.4, 37.7, 29.3, 28.4. ESI-MS: m/z calculated for C₂₁H₂₈N₃O₆ [M+H]⁺ 418.5; found 418.1.

tert-Butyl(S)-(3-((4-((2-(2-cyanopyrrolidin-1-yl)-2-oxoethyl)carbamoyl)quinolin-6-yl)oxy)propyl)carbamate (11): Compound 9 (145 mg, 0.347 mmol, 1.0 eq.) and LiOH (58.4 mg, 1.39 mmol, 4.0 eq.) was stirred in 5 mL of H₂O/THF (1/1) for 6 hours. After most of the THF was removed under vacuum, the mixture was loaded onto a 25 g C18 cartridge and eluted with a MeCN/water/TFA gradient (0/100/0.1 to 90/10/0.1) to remove the salts. After removal of solvent and drying, the product 10 was obtained in quantitative yield. To a stirred solution of 10 (144 mg, 0.357 mmol, 1.0 eq.) in anhydrous DMF (4 mL) was added(S)-pyrrolidine-2-carbonitrile (56.8 mg, 0.428 mmol, 1.2 eq.), HATU (163 mg, 0.428 mmol, 1.2 eq.) and DIPEA (125 μL, 0.714 mmol, 2.0 eq.). After 6 hours, the solvent was removed under vacuum and the crude mixture was loaded onto a 25 g C18 cartridge (Silicycle, Canada). The product was purified with a MeCN/water/TFA gradient (0/100/0.1 to 90/10/0.1). After lyophilization, the product 11 was obtained with a yield of 80% (135 mg). ¹H NMR (400 MHZ, CDCl₃): δ 8.73 (s, 1H), 7.95 (d, J=10.2 Hz, 1H), 7.68 (br s, 1H), 7.63-7.56 (m, 1H), 7.56-7.48 (m, 1H), 7.38-7.29 (m, 1H), 5.27 (br s, 1H), 4.84-4.72 (m, 1H), 4.46-4.35 (m, 1H), 4.33-4.20 (m, 1H), 4.17-4.09 (m, 2H), 3.78-3.64 (m, 1H), 3.59-3.46 (m, 1H), 3.36 (s, 2H), 2.38-2.17 (m, 4H), 1.42 (s, 9H), 1.35-1.27 (m, 2H). ¹³C NMR (100 MHz, CDCl₃): δ 167.6, 167.5, 157.9, 156.2, 146.3, 130.2, 125.7, 123.7, 119.3, 118.0, 103.3, 79.0, 65.9, 46.8, 45.7, 42.2, 37.6, 29.8, 29.3, 28.4, 25.1. ESI-MS: m/z calculated for C₂₅H₃₂N₅O₅ [M+H]⁺ 482.6; found 482.2.

(S)-6-(3-aminopropoxy)-N-(2-(2-cyanopyrrolidin-1-yl)-2-oxoethyl)quinoline-4-carboxamide (4): Compound 11 (100 mg, 0.207 mmol.) was treated with a 1 mL solution of TFA/methylene chloride (1/1) for 2 h. The solvent was removed under vacuum, and the crude mixture was loaded onto a 12 g C18 cartridge (Silicycle, Canada). The product was purified with a MeCN/water/TFA gradient (0/100/0.1 to 90/10/0.1). After lyophilization, product 6 was obtained with a quantitative yield. ¹H NMR (500 MHZ, D₂O): δ 9.05 (dd, J=2.0, 5.5 Hz, 1H), 8.24 (dd, J=2.0, 9.0 Hz, 1H), 8.13 (dd, J=2.0, 5.5 Hz, 1H), 7.93-7.80 (m, 2H), 4.84 (t, J=4.5 Hz, 2H), 4.58-4.32 (m, 4H), 3.87-3.78 (m, 1H), 3.67 (q, J=8.0 Hz, 1H), 3.29 (t, J=6.0 Hz, 2H), 2.45-2.12 (m, 6H), 1.34 (d, J=6.0 Hz, 1H). ESI-MS: m/z calculated for C₂₀H₂₃N₅O₃ [M+H]⁺ 382.4; found 382.2.

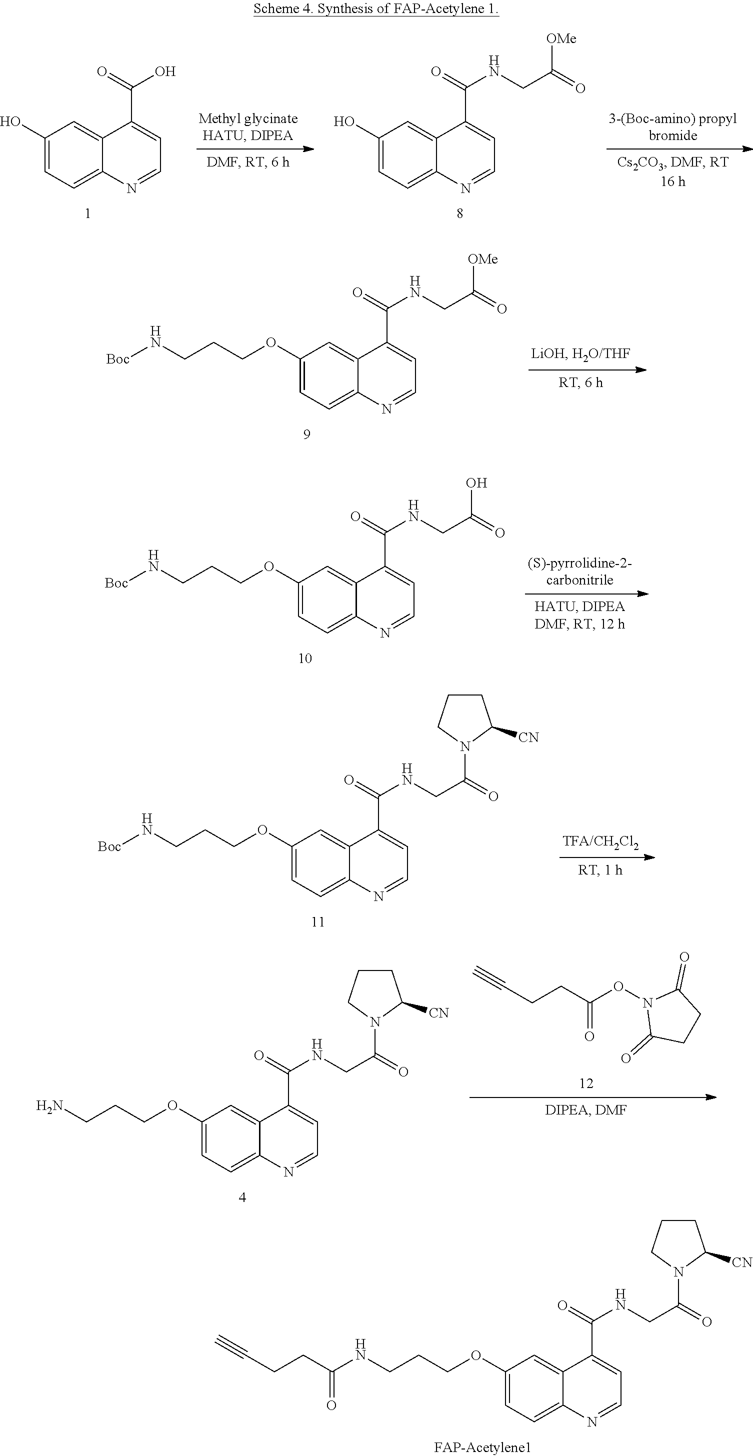

(S)—N-(2-(2-cyanopyrrolidin-1-yl)-2-oxoethyl)-6-(3-(pent-4-ynamido)propoxy)quinoline-4-carboxamide (FAP-Acetylene1): To the stirred solution of 4 (10 mg, 0.026 mmol, 1.0 eq.) in

DMF (1 mL) was added 2,5-dioxopyrrolidin-1-yl pent-4-ynoate (12) (5.12 mg, 0.026 mmol, 1.0 eq.) and DIPEA (13.5 μL, 0.078 mmol, 3.0 eq.). After 2 hours, the solvent was removed under vacuum and the crude mixture was loaded onto a 12 g C18 cartridge (Silicycle, Canada). The product was purified with a MeCN/water/TFA gradient (0/100/0.1 to 90/10/0.1). After lyophilization, 8.6 mg of FAP-Acetylene1 was obtained with a yield of 71%. ¹H NMR (500 MHz, DMSO-d6): δ 9.05 (t, J=5.5 Hz, 1H), 8.84 (d, J=4.5 Hz, 1H), 8.03-7.95 (m, 2H), 7.86 (d, J=2.0 Hz, 1H), 7.56 (d, J=4.0 Hz, 1H), 7.49 (dd, J=2.5, 9.0 Hz, 1H), 4.85-4.78 (m, 1H), 4.17 (t, J=6.0 Hz, 2H), 2.92-2.86 (m, 2H), 2.81 (s, 1H), 2.74 (t, J=2.5 Hz, 1H), 2.37-2.31 (m, 3H), 2.29-2.23 (m, 3H), 2.22-2.13 (m, 2H), 2.12-1.99 (m, 3H), 1.97-1.89 (m, 2H); ESI-MS: m/z calculated for C₂₅H₂₈N₅O₄ [M+H]⁺ 462.2; found 462.1.

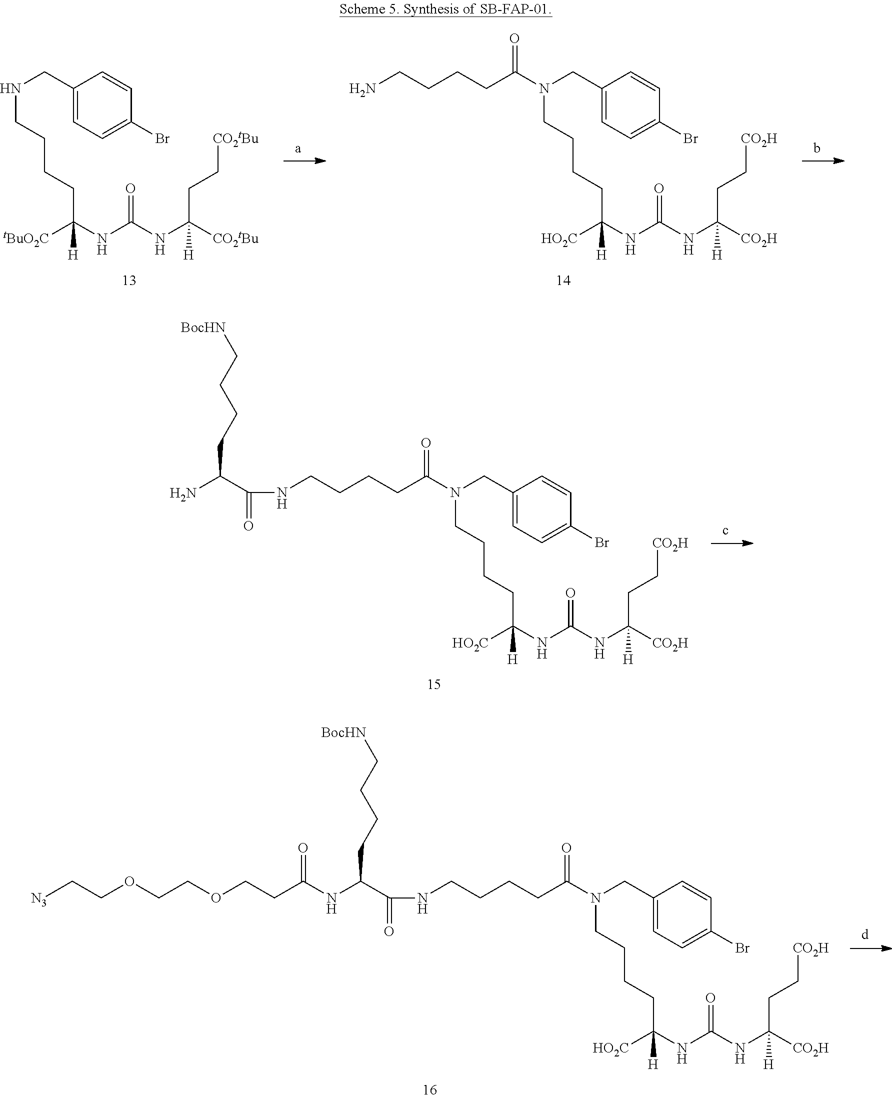 [SMILES: CC(C)(C)OC(=O)CC[C@H](NC(=O)N[C@@H](CCCCNCc1ccc(Br)cc1)C(=O)OC(C)(C)C)C(=O)OC(C)(C)C ; NCCCCC(=O)N(CCCC[C@H](NC(=O)N[C@@H](CCC(=O)O)C(=O)O)C(=O)O)Cc1ccc(Br)cc1 ; CC(C)(C)OC(=O)NCCCC[C@H](N)C(=O)NCCCCC(=O)N(CCCC[C@H](NC(=O)N[C@@H](CCC(=O)O)C(=O)O)C(=O)O)Cc1ccc(Br)cc1 ; CC(C)(C)OC(=O)NCCCC[C@H](NC(=O)CCOCCOCCN=[N+]=[N-])C(=O)NCCCCC(=O)N(CCCC[C@H](NC(=O)N[C@@H](CCC(=O)O)C(=O)O)C(=O)O)Cc1ccc(Br)cc1]

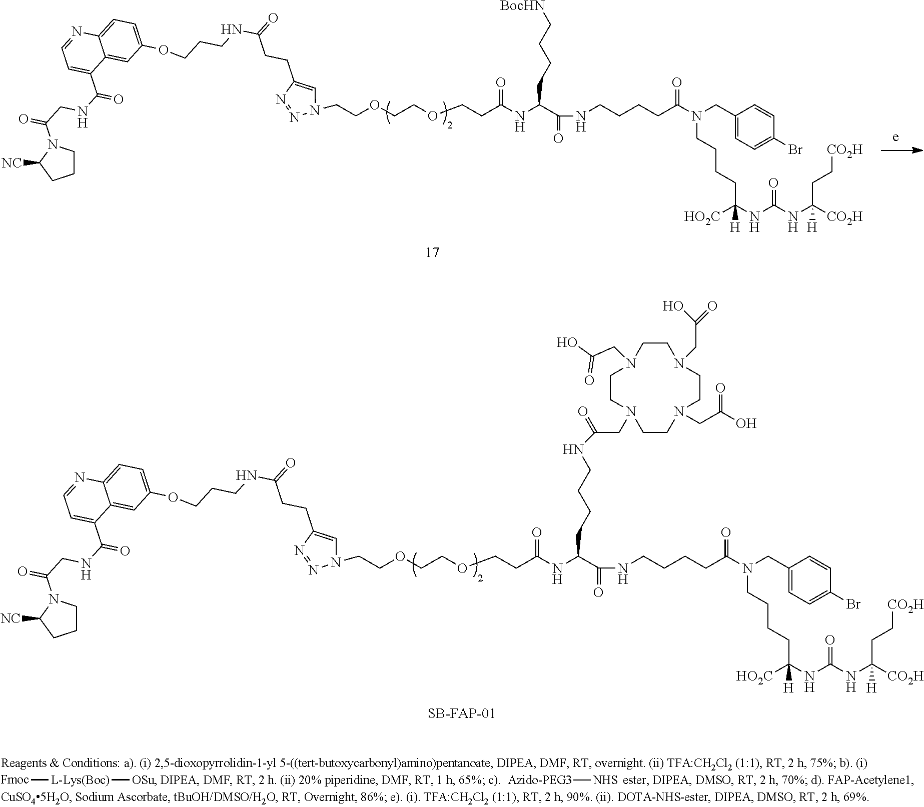

(((S)-5-(5-Amino-N-(4-bromobenzyl)pentanamido)-1-carboxypentyl)carbamoyl)-L-glutamic acid (14): To a stirred solution of 13 (214 mg, 0.326 mmol, 1.0 eq.) and 2,5-dioxopyrrolidin-1-yl 5-((tert-butoxycarbonyl)amino) pentanoate (102.5 mg, 0.326 mmol, 1.0 eq.) in DMF (2 mL) was added DIPEA (170 μL, 0.978 mmol, 3.0 eq.) at room temperature. The reaction mixture was stirred for 24 h and concentrated to get crude product. To the above crude was added 2 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 14 (143 mg, 75%) as a white solid. H¹-NMR (500 MHz, D₂O): δ 7.55 (d, J=8.5 Hz, 1H), 7.51 (d, J=8.5 Hz, 1H), 7.12 (dd, J=2.5, 8.0 Hz, 2H), 4.59 (s, 1H), 4.51 (s, 1H), 4.29-4.22 (m, 1H), 4.19-4.11 (m, 1H), 3.39-3.29 (m, 2H), 3.05-2.88 (m, 2H), 2.59-2.38 (m, 4H), 2.22-2.10 (m, 1H), 2.02-1.90 (m, 1H), 1.82-1.46 (m, 8H), 1.39-1.24 (m, 2H).

(10S,23S,27S)-10-Amino-18-(4-bromobenzyl)-2,2-dimethyl-4,11,17,25-tetraoxo-3-oxa-5,12,18,24,26-pentaazanonacosane-23,27,29-tricarboxylic acid (15): To a stirred solution of 14 (139 mg, 0.236 mmol, 1.0 eq.) and Fmoc-L-Lys (Boc)-OSu (133.9 mg, 0.236 mmol, 1.0 eq.) in DMF (2 mL) was added DIPEA (250 μL, 1.42 mmol, 6.0 eq.) at room temperature. The reaction mixture was stirred for 2 h and concentrated to get crude product. To the above crude was added 2 mL of 20% piperidine in DMF (3 mL) at room temperature and mixture was stirred for 1 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 15 (125 mg, 65%) as a white solid. H¹-NMR (500 MHz, DMSO-d₆): δ 8.43-8.32 (m, 1H), 8.08 (s, 2H), 7.93 (d, J=7.5 Hz, 1H), 7.74 (d, J=7.5 Hz, 1H), 7.55 (d, J=8.0 Hz, 1H), 7.41 (d, J=8.0 Hz, 1H), 6.74 (t, J=5.0 Hz, 1H), 6.40-6.26 (m, 2H), 4.52 (s, 1H), 4.44 (s, 1H), 4.14-3.98 (m, 2H), 3.23-2.97 (m, 5H), 2.93-2.82 (m, 2H), 2.37 (t, J=7.0 Hz, 1H), 2.31-2.15 (m, 3H), 1.96-1.43 (m, 14H), 1.35 (s, 9H), 1.29-1.15 (m, 4H); [M+H]⁺ calcd for C₃₅H₅₆BrN₆O₁₁, 815.3192; found, 815.3184.

(11S,24S,28S)-1-Azido-19-(4-bromobenzyl)-11-(4-((tert-butoxycarbonyl)amino)butyl)-9,12,18,26-tetraoxo-3,6-dioxa-10,13,19,25,27-pentaazatriacontane-24,28,30-tricarboxylic acid (16): To a stirred solution of 15 (47.3 mg, 0.058 mmol, 1.0 eq.) and Azido-PEG3-NHS ester (20 mg, 0.058 mmol, 1.0 eq.) in DMSO (100μ) was added DIPEA (80 μL, 0.464 mmol, 8.0 eq.) at room temperature. The reaction mixture was stirred for 2 h and concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 11 (42 mg, 70%) as a white solid. ESI-MS: m/z calculated for C₄₄H₇₁BrN₉O₁₅ [M+H]⁺ 1044.4; found 1044.3.

Compound 17: To a stirred solution of FAP-Acetylene1 (6 mg, 0.0129 mmol, 1.0 eq.) in t-BuOH:H2O (1:2, 300 μL) was added CuSO₄.5H₂O (0.486 mg, 0.0019 mmol, 0.15 eq.) and sodium ascorbate (1.15 mg, 0.0058 mmol, 0.45 eq.). To the above reaction mixture was added solution of product 16 (13.5 mg, 0.0129 mmol, 1.0 eq.) in DMSO (100 μL) and stirred for overnight at room temperature. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 17 (16.7 mg, 86%) as a white solid. ESI-MS: m/z calculated for C₆₉H₉₈BrN₁₄O₁₉ [M+H]⁺ 1505.6; found 1505.4.

Compound SB-FAP-01: To the compound 17 (9 mg, 0.0059 mmol) was added 1 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded free amine in quantitative yield. To a stirred solution of amine (3.8 mg, 0.0027 mmol, 1.0 eq.) and DOTA-NHS-ester (3.08 mg, 0.004 mmol, 1.5 eq.) in DMSO (50 μL) was added DIPEA (4.7 μL, 0.027 mmol, 10.0 eq.) at room temperature. The reaction mixture was stirred for 2 h and crude was purified by preparative RP-HPLC chromatography using 0.1% TFA in H2O and 0.1% TFA in acetonitrile as eluents followed by lyophilization afforded compound SB-FAP-01 (3.3 mg, 69%) as a white solid. RP-HPLC purification was achieved using Agilent System, λ 254 nm, 250 mm×10 mm Phenomenex Luna C18 column, solvent gradient: 90% H2O (0.1% TFA) and 10% ACN (0.1% TFA), reaching 90% of ACN in 25 min at a flow rate of 5 mL/min, product eluted at 13.8 min]. HRMS (ESI) m/z: [M+H]⁺ calcd for C₈₀H₁₁₄BrN₁₈O₂₄, 1789.7456; found, 1789.7442.

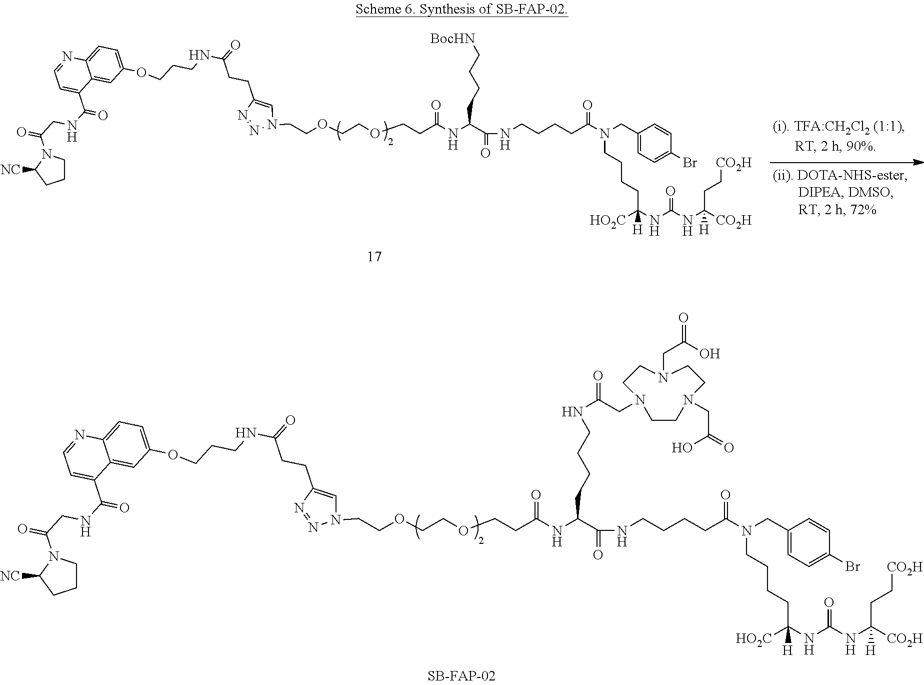

Compound SB-FAP-02: To the compound 17 (8 mg, 0.0053 mmol) was added 1 mL of TFA/CH₂Cl₂ (1:1) at room temperature and mixture was stirred for 2 h. Reaction mixture was concentrated to get the crude which was loaded onto a 12 g C18 cartridge (Silicycle, Canada).

The product was purified with a MeCN/water/TFA gradient (0/100/0.1 to 90/10/0.1). After lyophilization, afforded free amine in quantitative yield. To a stirred solution of amine (6.7 mg, 0.000.0047 mmol, 1.0 eq.) and NOTA-NHS-ester (3.46 mg, 0.0052 mmol, 1.1 eq.) in DMSO (50 μL) was added triethylamine (6.6 μL, 0.047 mmol, 10.0 eq.) at room temperature. The reaction mixture was stirred for 2 h and crude was purified by preparative RP-HPLC chromatography using 0.1% TFA in H₂O and 0.1% TFA in acetonitrile as eluents followed by lyophilization afforded compound SB-FAP-02 (5.8 mg, 72%) as a white solid. [RP-HPLC purification was achieved using Agilent System, λ 254 nm, 250 mm×10 mm Phenomenex Luna C18 column, solvent gradient: 98% H₂O (0.1% TFA) and 2% ACN (0.1% TFA), reaching 50% of ACN in 0 to 20 min, 90% of ACN in 20 to 25 min at a flow rate of 5 mL/min, product eluted at 16.8 min]. ¹H-NMR (500 MHz, DMSO-d6): δ 12.11 (Broad singlet, 5H), 9.04 (t, J=5.5 Hz, 1H), 8.84-8.80 (m, 1H), 8.19-8.10 (m, 1H), 8.03-7.90 (m, 3H), 7.59-7.43 (m, 4H), 7.19-7.10 (m, 2H), 6.37-6.25 (m, 2H), 4.82 (s, 2H), 4.51 (m, 2H), 4.43 (s, 2H), 4.26-4.02 (m, 14H), 3.79-3.66 (m, 6H), 3.64-3.56 (m, 6H), 3.44 (d, J=16.5, 8H), 3.29-2.94 (m, 11H), 2.86-2.79 (m, 2H), 2.42 (t, J=7.0, 2H), 2.39-2.30 (m, 3H), 2.28-2.16 (m, 4H), 2.12-2.01 (m, 2H), 1.96-1.86 (m, 2H), 1.73-1.17 (m, 20H); HRMS (ESI) m/z: [M+H]⁺ calcd for C₇₆H₁₀₉BrN₁₇O₂₂, 1690.7129; found, 1690.7110.

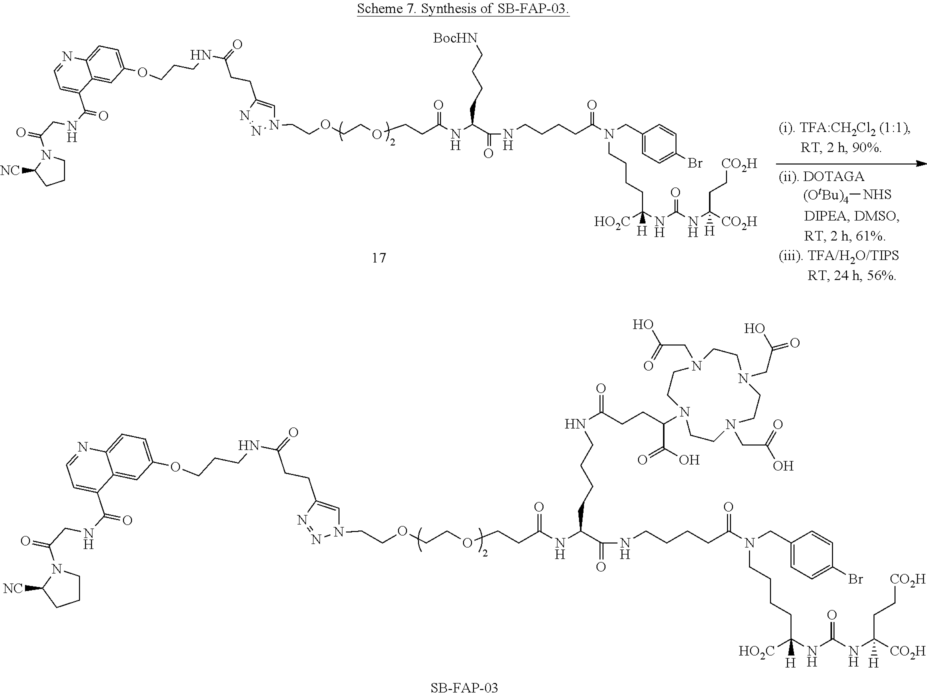

Compound SB-FAP-03: To the compound 17 (8 mg, 0.0053 mmol) was added 1 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded free amine in quantitative yield (6.7 mg). To a stirred solution of amine (6.0 mg, 0.0042 mmol, 1.0 eq.) and DOTAGA (^{t}Bu) 4-NHS-ester (4.08 mg, 0.0064 mmol, 1.2 eq.) in DMF (100 μL) was added diisopropylethylamine (5.9 μL, 0.042 mmol, 10.0 eq.) at room temperature. The reaction mixture was stirred for 2 h and crude was purified by preparative RP-HPLC chromatography to afford product (5.4 mg, 61%) as a white solid. To the stirred solution of above product was added cocktail of TFA/H₂O/TIPS (950 μL/25 μL/25 μL) at room temperature. The reaction mixture was stirred for 24 h and the crude was purified by preparative RP-HPLC chromatography using 0.1% TFA in H₂O and 0.1% TFA in acetonitrile as eluents followed by lyophilization afforded compound SB-FAP-03 (2.7 mg, 56%) as a white solid. HRMS (ESI) m/z: [M+H]⁺ calcd for C₈₃H₁₂₀BrN₁₈O₂₆, 1863.7812; found, 1863.7799.

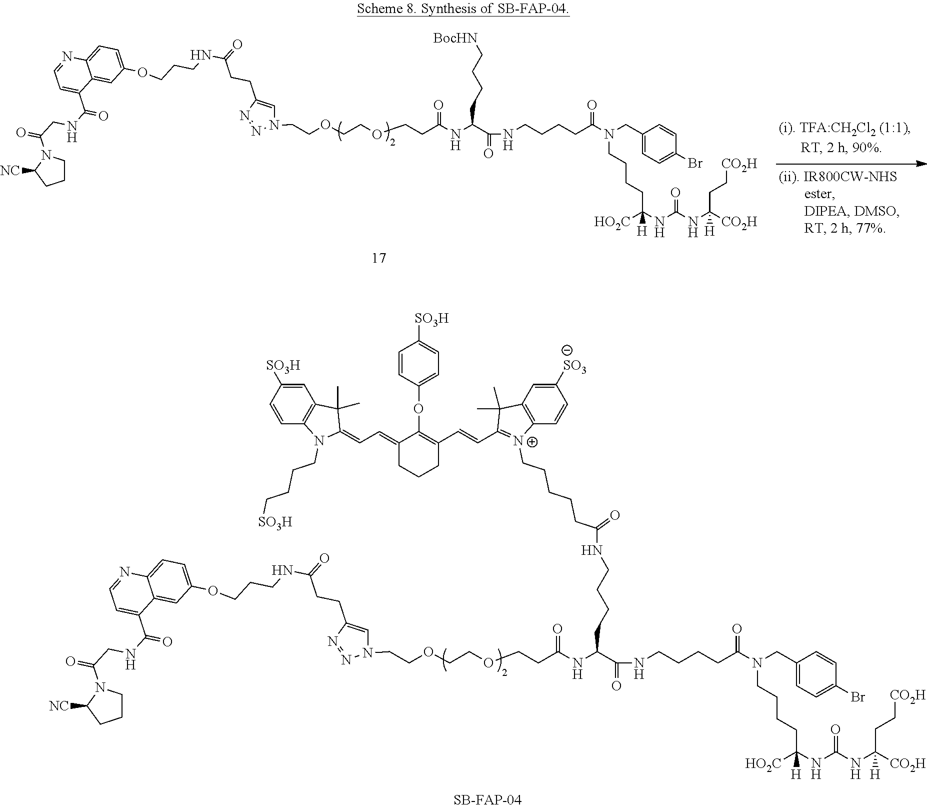

Compound SB-FAP-04: To the compound 17 (8 mg, 0.0053 mmol) was added 1 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded free amine in quantitative yield. To a stirred solution of amine (1.9 mg, 0.00137 mmol, 1.5 eq.) and IR800CW-NHS ester (1.07 mg, 0.000917 mmol, 1.0 eq.) in DMSO (20 μL) was added DIPEA (2.0 μL, 0.0137 mmol, 15.0 eq.) at room temperature. The reaction mixture was stirred for 2 h and crude was purified by preparative RP-HPLC chromatography using 20 mM triethylammoniumacetate buffer and acetonitrile as eluents followed by lyophilization afforded compound SB-FAP-04 (1.6 mg, 77%) as a green solid. HRMS (ESI) m/z: [M/2]⁺ calcd for C₁₁₀H₁₄₃BrN₁₆O₃₁S₄, 1195.409171; found, 1195.4080.

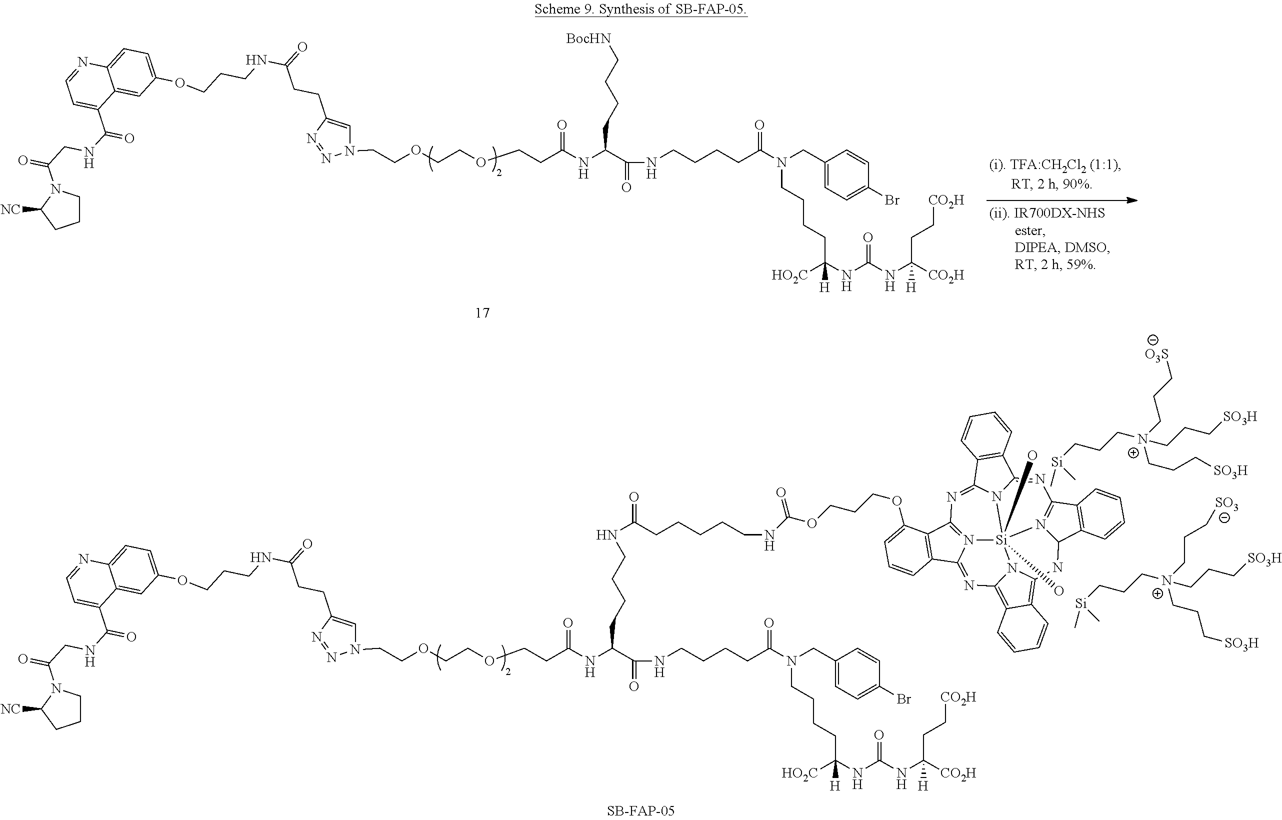

Compound SB-FAP-05: To the compound 17 (8 mg, 0.0053 mmol) was added 1 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded free amine in quantitative yield. To a stirred solution of amine (1.43 mg, 1.01 μmol, 2.0 eq.) and IR800CW-NHS ester (1.0 mg, 0.51 μmol, 1.0 eq.) in DMSO (20μ L) was added DIPEA (2.7 μL, 15.3 □mol, 30.0 eq.) at room temperature. The reaction mixture was stirred for 2 h and crude was purified by preparative RP-HPLC chromatography using 20 mM triethylammoniumacetate buffer and acetonitrile as eluents followed by lyophilization afforded compound SB-FAP-05 (0.95 mg, 59%) as a green solid.

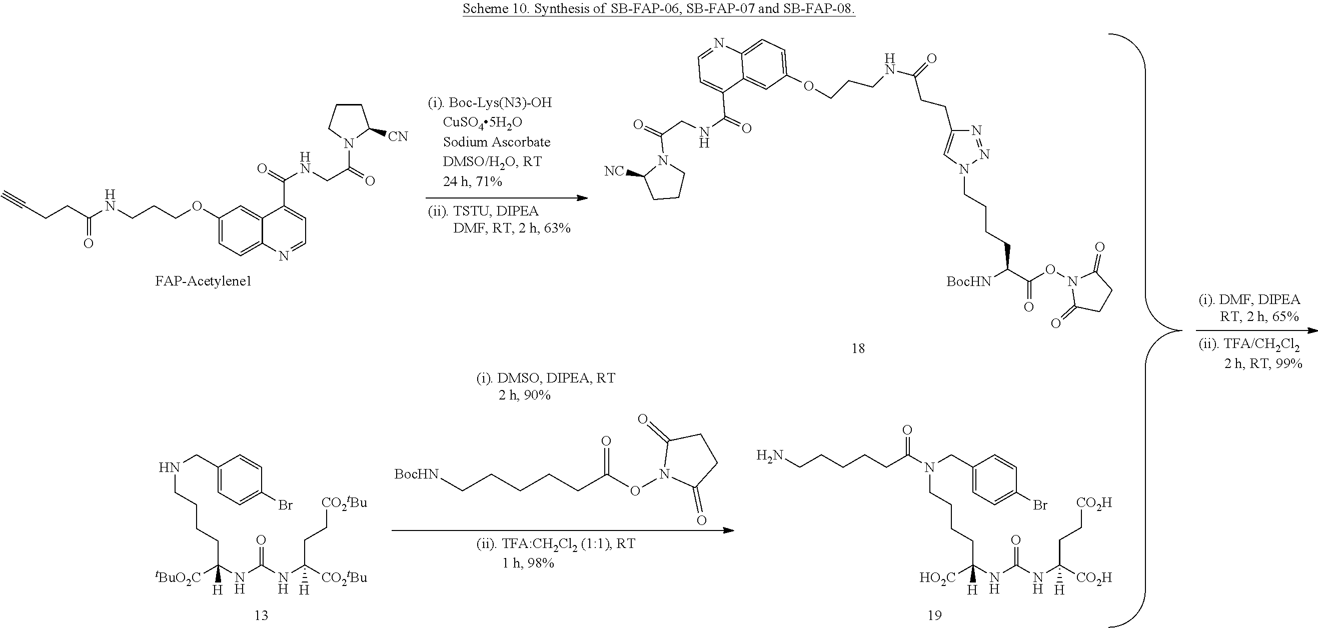 [SMILES: C#CCCC(=O)NCCCOc1ccc2nccc(C(=O)NCC(=O)N3CCC[C@H]3C#N)c2c1 ; CC(C)(C)OC(=O)N[C@@H](CCCCn1cc(CCC(=O)NCCCOc2ccc3nccc(C(=O)NCC(=O)N4CCC[C@H]4C#N)c3c2)nn1)C(=O)ON1C(=O)CCC1=O ; CC(C)(C)OC(=O)CC[C@H](NC(=O)N[C@@H](CCCCNCc1ccc(Br)cc1)C(=O)OC(C)(C)C)C(=O)OC(C)(C)C ; CC(C)(C)OC(=O)NCCCCCC(=O)ON1C(=O)CCC1=O ; NCCCCCC(=O)N(CCCC[C@H](NC(=O)N[C@@H](CCC(=O)O)C(=O)O)C(=O)O)Cc1ccc(Br)cc1]

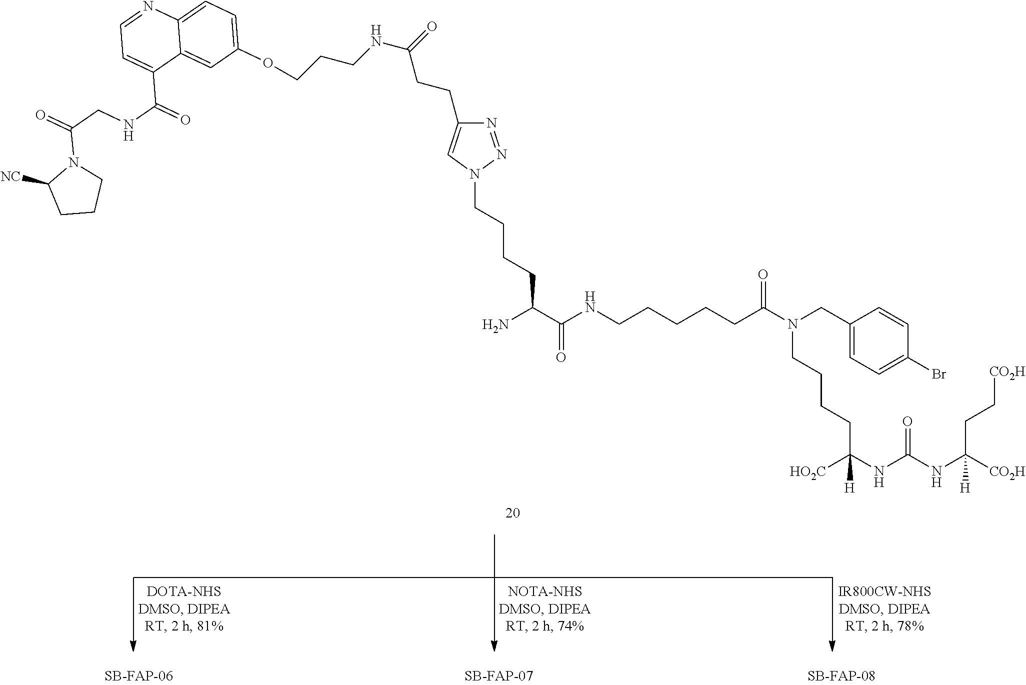 [SMILES: N#C[C@@H]1CCCN1C(=O)CNC(=O)c1ccnc2ccc(OCCCNC(=O)CCc3cn(CCCC[C@H](N)C(=O)NCCCCCC(=O)N(CCCC[C@H](NC(=O)N[C@@H](CCC(=O)O)C(=O)O)C(=O)O)Cc4ccc(Br)cc4)nn3)cc12]

2,5-Dioxopyrrolidin-1-yl(S)-2-((tert-butoxycarbonyl)amino)-6-(4-(3-((3-((4-((2-((S)-2-cyanopyrrolidin-1-yl)-2-oxoethyl)carbamoyl)quinolin-6-yl)oxy)propyl)amino)-3-oxopropyl)-1H-1,2,3-triazol-1-yl) hexanoate (18): To a stirred solution of FAP-Acetylene1 (10 mg, 0.0216 mmol, 1.0 eq.) and Boc-Lys(N3)-OH in DMSO:H₂O (1:1, 400 μL) was added freshly prepared solution of CuSO₄·5H₂O (0.81 mg, 3.24 μmol, 0.15 eq.) and sodium ascorbate (1.93 mg, 9.74 μmol, 0.45 eq.). Reaction mixture was stirred for overnight at room temperature and concentrated to get the crude which was loaded onto a 12 g C18 cartridge (Silicycle, Canada). The product was purified with a MeCN/water/TFA gradient (0/100/0.1 to 90/10/0.1). After lyophilization, afforded triazole product (11.3 mg, 71%) as a white solid. To the stirred solution of triazole product (8.5 mg, 0.0115 mmol, 1.0 eq.) in dimethylformamide (500 μL) added TSTU (5.23 mg, 0.0173 mmol, 1.5 eq.) and diisopropylethylamine (6.0 μL, 0.034 mmol, 3.0 eq.) at room temperature under nitrogen atmosphere. The reaction mixture was stirred for 2 h and concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 18 (6.0 mg, 63%) as a white solid.

(((S)-5-(6-Amino-N-(4-bromobenzyl) hexanamido)-1-carboxypentyl)carbamoyl)-L-glutamic acid (19): To a stirred solution of 13 (365 mg, 0.556 mmol, 1.0 eq.) and 2,5-dioxopyrrolidin-1-yl 6-((tert-butoxycarbonyl)amino) hexanoate (174.7 mg, 0.556 mmol, 1.0 eq.) in DMF (2 mL) was added DIPEA (233 μL, 1.669 mmol, 3.0 eq.) at room temperature. The reaction mixture was stirred for 24 h and concentrated to get crude product. To the above crude was added 2 mL of TFA/CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 19 (360 mg, 98%) as a white solid.

Compound 20: To a stirred solution of 18 (6.9 mg, 8.3 μmol, 1.0 eq.) and 19 (7.47 mg, 12.4 μmol, 1.5 eq.) in DMF was added DIPEA (13.7 μL, 99.6 μmol, 12.0 eq.) at room temperature. The reaction mixture was stirred for 2 h and concentrated to get crude product. To the above crude was added 1 mL of TFA:CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 15 (7.0 mg, 98%) as a white solid.

Compound SB-FAP-06: To a stirred solution of amine 20 (3.81 mg, 3.13 μmol, 1.0 eq.) and DOTA-NHS-ester (2.62 mg, 3.44 μmol, 1.1 eq.) in DMSO (50 μL) was added DIPEA (5.2 μL, 37.6 μmol, 12.0 eq.) at room temperature. The reaction mixture was stirred for 2 h and crude was purified by preparative RP-HPLC chromatography using 0.1% TFA in H₂O and 0.1% TFA in acetonitrile as eluents followed by lyophilization afforded compound SB-FAP-06 (4.0 mg, 81%) as a white solid.

Compound SB-FAP-07: To a stirred solution of amine 20 (3.4 mg, 2.79 μmol, 1.0 eq.) and NOTA-NHS-ester (2.2 mg, 3.35 μmol, 1.2 eq.) in DMSO (65μ) was added DIPEA (4.7 μL, 33.5 μmol, 12.0 eq.) at room temperature. The reaction mixture was stirred for 2 h and crude was purified by preparative RP-HPLC chromatography using 0.1% TFA in H₂O and 0.1% TFA in acetonitrile as eluents followed by lyophilization afforded compound SB-FAP-06 (3.1 mg, 74%) as a white solid.

Compound SB-FAP-08: To a stirred solution of amine 20 (1.35 mg, 1.11 μmol, 1.3 eq.) and IR800CW-NHS-ester (1.0 mg, 0.85 μmol, 1.0 eq.) in DMSO (25 μL) was added DIPEA (1.8 μL, 12.86 μmol, 15.0 eq.) at room temperature. The reaction mixture was stirred for 2 h and crude was purified by preparative RP-HPLC chromatography using 20 mM triethylammonium acetate buffer in acetonitrile as eluents followed by lyophilization afforded compound SB-FAP-08 (0.85 mg, 78%) as a green solid.

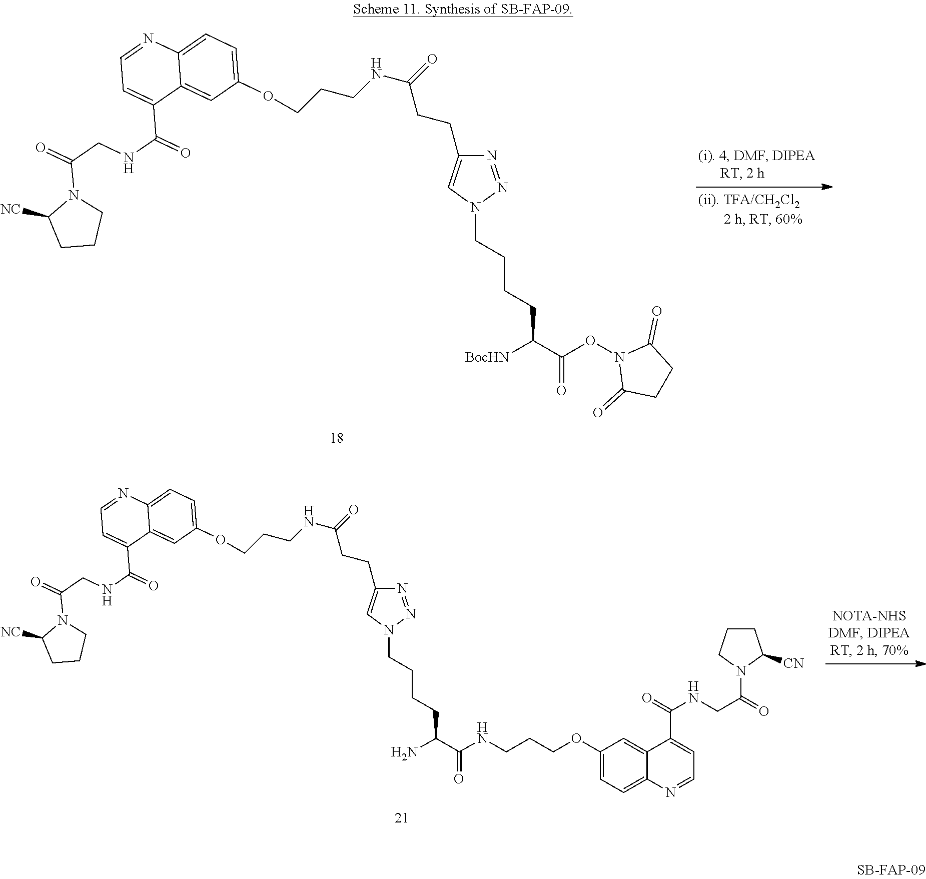 [SMILES: CC(C)(C)OC(=O)N[C@@H](CCCCn1cc(CCC(=O)NCCCOc2ccc3nccc(C(=O)NCC(=O)N4CCC[C@H]4C#N)c3c2)nn1)C(=O)ON1C(=O)CCC1=O ; N#C[C@@H]1CCCN1C(=O)CNC(=O)c1ccnc2ccc(OCCCNC(=O)CCc3cn(CCCC[C@H](N)C(=O)NCCCOc4ccc5nccc(C(=O)NCC(=O)N6CCC[C@H]6C#N)c5c4)nn3)cc12]

Compound 21: To a stirred solution of 18 (6.0 mg, 7.22 μmol, 1.0 eq.) and 4 (2.75 mg, 7.22 μmol, 1.0 eq.) in DMF (300 μL) was added DIPEA (7.5 μL, 943.3 μmol, 6.0 eq.) at room temperature. The reaction mixture was stirred for 2 h and concentrated to get crude product. To the above crude was added 1 mL of TFA:CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 21 (4.3 mg, 60%) as a white solid.

Compound SB-FAP-09: To a stirred solution of amine 21 (3.12 mg, 3.12 μmol, 1.0 eq.) and NOTA-NHS-ester (2.3 mg, 3.44 μmol, 1.1 eq.) in DMSO (75 μL) was added DIPEA (4.35 μL, 31.2 μmol, 10.0 eq.) at room temperature. The reaction mixture was stirred for 2 h and crude was purified by preparative RP-HPLC chromatography using 0.1% TFA in H₂O and 0.1% TFA in acetonitrile as eluents followed by lyophilization afforded compound SB-FAP-09 (2.8 mg, 70%) as a white solid.

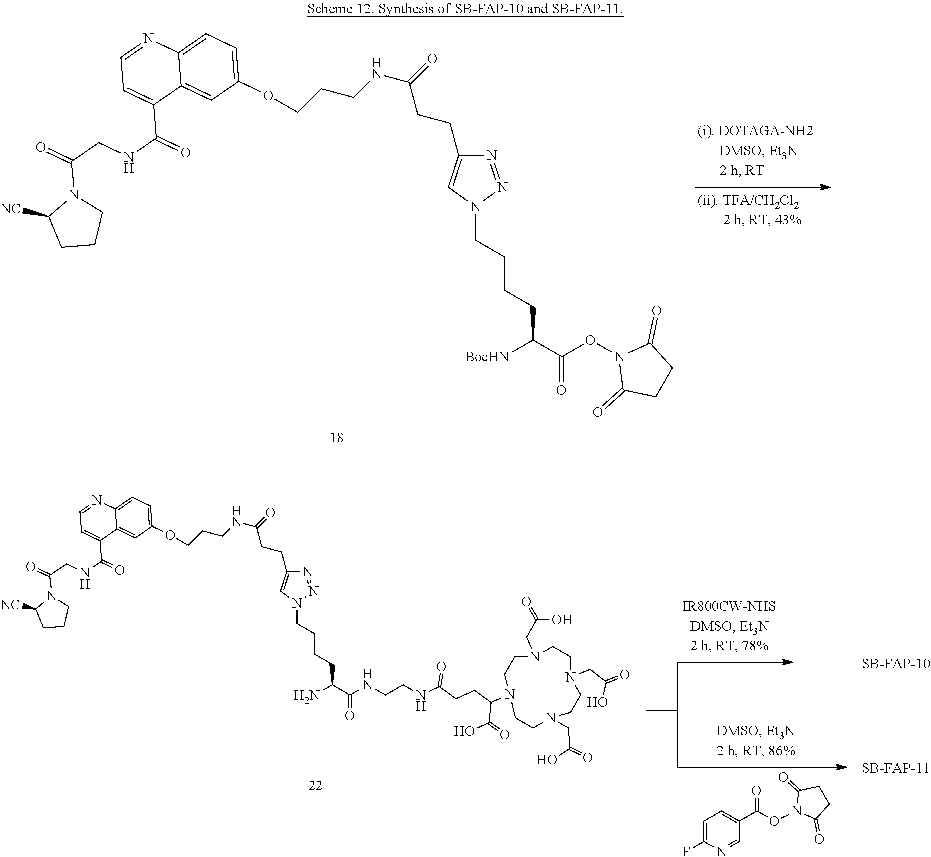 [SMILES: CC(C)(C)OC(=O)N[C@@H](CCCCn1cc(CCC(=O)NCCCOc2ccc3nccc(C(=O)NCC(=O)N4CCC[C@H]4C#N)c3c2)nn1)C(=O)ON1C(=O)CCC1=O ; N#C[C@@H]1CCCN1C(=O)CNC(=O)c1ccnc2ccc(OCCCNC(=O)CCc3cn(CCCC[C@H](N)C(=O)NCCNC(=O)CCC(C(=O)O)N4CCN(CC(=O)O)CCN(CC(=O)O)CCN(CC(=O)O)CC4)nn3)cc12 ; O=C(ON1C(=O)CCC1=O)c1ccc(F)nc1]

2,2′,2″-(10-(4-((2-((S)-2-amino-6-(4-(3-((3-((4-((2-((S)-2-cyanopyrrolidin-1-yl)-2-oxoethyl)carbamoyl)quinolin-6-yl)oxy)propyl)amino)-3-oxopropyl)-1H-1,2,3-triazol-1-yl)hexanamido)ethyl)amino)-1-carboxy-4-oxobutyl)-1,4,7,10-tetraazacyclododecane-1,4,7-triyl)triacetic acid (22): To a stirred solution of 13 (10.8 mg, 12.99 μmol, 1.0 eq.) and DOTAGA-NH₂ (6.74 mg, 12.9 μmol, 1.0 eq.) in DMSO (100 μL) was added triethylamine (21.7 μL, 156.0 μmol, 12.0 eq.) at room temperature. The reaction mixture was stirred for 2 h and concentrated to get crude product. To the above crude was added 1 mL of TFA:CH₂Cl₂ (1:1) at room temperature and mixture was stirred for 2 h. Reaction mixture was concentrated to get the crude which was loaded onto a 12 g C18 cartridge (Silicycle, Canada). The product was purified with a MeCN/water/TFA gradient (0/100/0.1 to 90/10/0.1). After lyophilization, afforded compound 22 (4.3 mg, 43%) as a white solid.

1-(6-(((2S)-1-((2-(4-carboxy-4-(4,7,10-tris(carboxymethyl)-1,4,7,10-tetraazacyclododecan-1-yl)butanamido)ethyl)amino)-6-(4-(3-((3-((4-((2-((S)-2-cyanopyrrolidin-1-yl)-2-oxoethyl)carbamoyl)quinolin-6-yl)oxy)propyl)amino)-3-oxopropyl)-1H-1,2,3-triazol-1-yl)-1-oxohexan-2-yl)amino)-6-oxohexyl)-2-((E)-2-((E)-3-(2-((E)-3,3-dimethyl-5-sulfo-1-(4-sulfobutyl)indolin-2-ylidene)ethylidene)-2-(4-sulfophenoxy)cyclohex-1-en-1-yl)vinyl)-3,3-dimethyl-3H-indol-1-ium-5-sulfonate (SB-FAP-10): To a stirred solution of amine 22 (1.63 mg, 1.44 μmol, 1.2 eq.) and IR800CW-NHS-ester (1.4 mg, 1.20 μmol, 1.0 eq.) in DMSO (25 μL) was added triethylamine (3.4 μL, 24.0 μmol, 20.0 eq.) at room temperature. The reaction mixture was stirred for 2 h and crude was purified by preparative RP-HPLC chromatography using 20 mM triethylammonium acetate buffer in acetonitrile as eluents followed by lyophilization afforded compound SB-FAP-08 (0.92 mg, 78%) as a green solid.

2,2′,2″-(10-(1-Carboxy-4-((2-((S)-6-(4-(3-((3-((4-((2-((S)-2-cyanopyrrolidin-1-yl)-2-oxoethyl)carbamoyl)quinolin-6-yl)oxy)propyl)amino)-3-oxopropyl)-1H-1,2,3-triazol-1-yl)-2-(6-fluoronicotinamido)hexanamido)ethyl)amino)-4-oxobutyl)-1,4,7,10-tetraazacyclododecane-1,4,7-triyl)triacetic acid (SB-FAP-11): To a stirred solution of amine 22 (5.9 mg, 5.20 μmol, 1.0 eq.) and 2,5-dioxopyrrolidin-1-yl 6-fluoronicotinate (1.5 mg, 6.20 μmol, 1.2 eq.) in DMSO (50 μL) was added triethylamine (7.2 μL, 52.0 μmol, 10.0 eq.) at room temperature. The reaction mixture was stirred for 2 h and crude was purified by preparative RP-HPLC chromatography using 0.1% TFA in H₂O and 0.1% TFA in acetonitrile as eluents followed by lyophilization afforded compound SB-FAP-11 (2.8 mg, 70%) as a white solid.

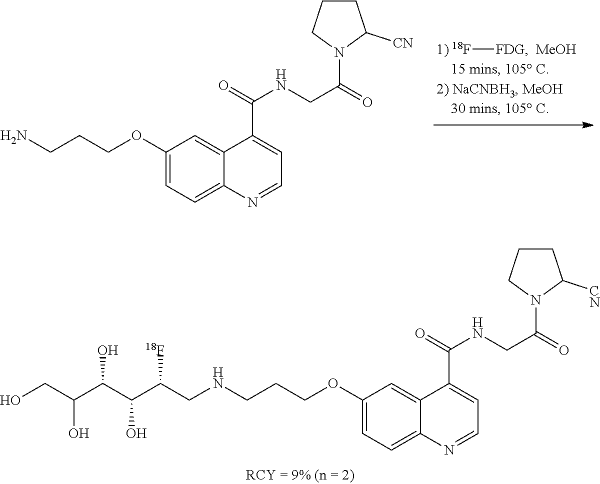

### Example 6

### Representative FAP-α Inhibitors

Representative FAP-α inhibitors and representative biological data are provided in Table 3.

TABLE 3
Representative FAP-α Inhibitors

| | | | | | | FAP | FAP |
|---|---|---|---|---|---|---|---|
| | | | MW | DPPIV | PREP | IC₅₀ | Ki |
| No | Structure | Code | (g/mol) | IC₅₀ | IC₅₀ | (nM) | (nM) |
| 1 | 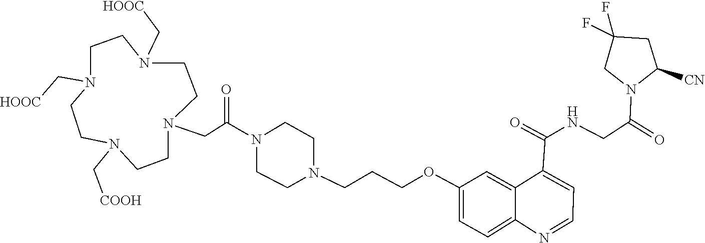 [SMILES: N#C[C@@H]1CC(F)(F)CN1C(=O)CNC(=O)c1ccnc2ccc(OCCCN3CCN(C(=O)CN4CCN(CC(=O)O)CCN(CC(=O)O)CCN(CC(=O)O)CC4)CC3)cc12] | FAPI-04 (Reference) | 872.93 | 8.81 | 796 nM | 0.75 | 0.15 |
| 2 | 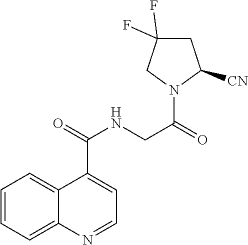 [SMILES: N#C[C@@H]1CC(F)(F)CN1C(=O)CNC(=O)c1ccnc2ccccc12] | UAMC- 01110 (Reference) | 344.32 | 7.47 μM | 96.93 nM | 0.57 | 0.11 |
| 3 | 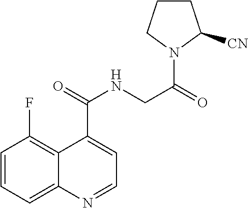 [SMILES: N#C[C@@H]1CCCN1C(=O)CNC(=O)c1ccnc2cccc(F)c12] | JHU1219438 | 326.33 | >20 μM | 95.84 nM | 0.912 | 0.1824 |
| 4 | 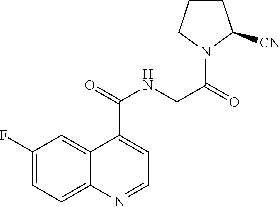 [SMILES: N#C[C@@H]1CCCN1C(=O)CNC(=O)c1ccnc2ccc(F)cc12] | JHU1220450 | 326.33 | >20 μM | 48.02 nM | 0.119 | 0.0239 |
| 5 | 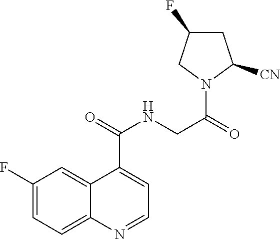 [SMILES: N#C[C@@H]1C[C@H](F)CN1C(=O)CNC(=O)c1ccnc2ccc(F)cc12] | JHU1220471 | 344.32 | 2.53 μM | 11.51 nM | 0.7335 | 0.1467 |
| 6 | 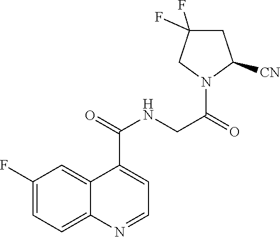 [SMILES: N#C[C@@H]1CC(F)(F)CN1C(=O)CNC(=O)c1ccnc2ccc(F)cc12] | JHU1220466 | 362.31 | 3.50 μM | 46.97 nM | 0.3097 | 0.062 |
| 7 | 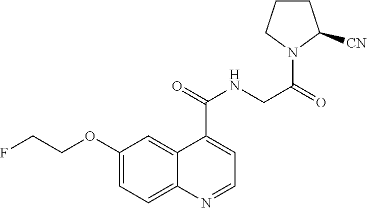 [SMILES: N#C[C@@H]1CCCN1C(=O)CNC(=O)c1ccnc2ccc(OCCF)cc12] | JHU1220470 | 370.38 | >20 μM | 1410 nM | 0.343 | 0.0686 |
| 8 | 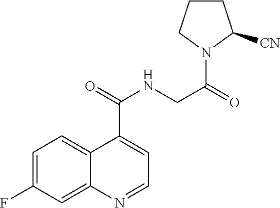 [SMILES: N#C[C@@H]1CCCN1C(=O)CNC(=O)c1ccnc2cc(F)ccc12] | JHU1220501 | 326.33 | >20 μM | 522.0 nM | 2.164 | 0.433 |
| 9 | 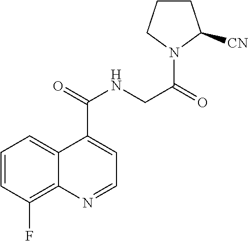 [SMILES: N#C[C@@H]1CCCN1C(=O)CNC(=O)c1ccnc2c(F)cccc12] | JHU1220502 | 326.33 | >20 μM | 84.46 nM | 5.044 | 1.009 |
| 10 | 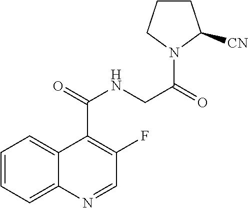 [SMILES: N#C[C@@H]1CCCN1C(=O)CNC(=O)c1c(F)cnc2ccccc12] | JHU1220503 | 326.33 | >20 μM | 688.0 nM | 222.8 | 44.56 |
| 11 | 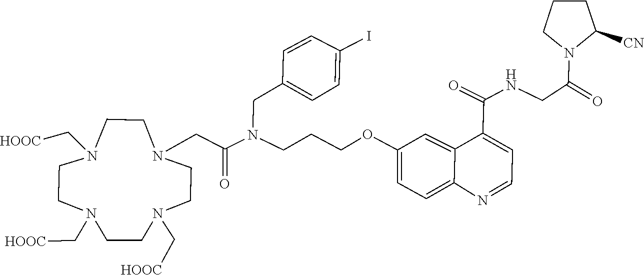 [SMILES: N#C[C@@H]1CCCN1C(=O)CNC(=O)c1ccnc2ccc(OCCCN(Cc3ccc(I)cc3)C(=O)CN3CCN(CC(=O)O)CCN(CC(=O)O)CCN(CC(=O)O)CC3)cc12] | JHU1220627 | 983.86 | 836.0 μM | >2.00 mM | 1.41 | 0.28 |
| 12 | 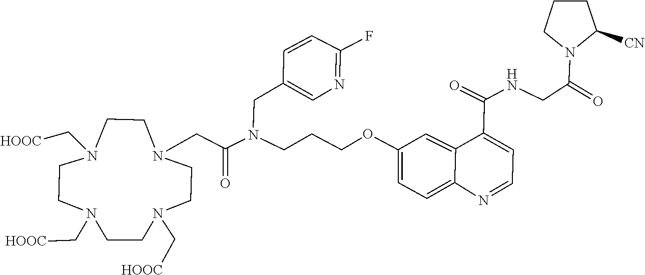 [SMILES: N#C[C@@H]1CCCN1C(=O)CNC(=O)c1ccnc2ccc(OCCCN(Cc3ccc(F)nc3)C(=O)CN3CCN(CC(=O)O)CCN(CC(=O)O)CCN(CC(=O)O)CC3)cc12] | JHU1221709 | 876.94 | 12.59 μM | 21.95 μM | 2.09 | 0.42 |
| 13 | 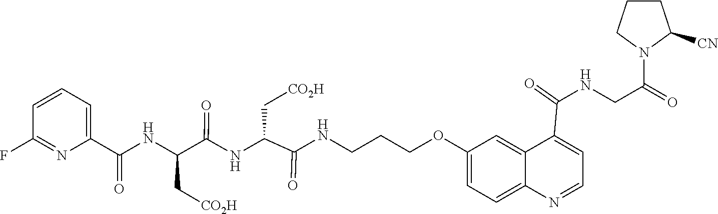 [SMILES: N#C[C@@H]1CCCN1C(=O)CNC(=O)c1ccnc2ccc(OCCCNC(=O)[C@@H](CC(=O)O)NC(=O)[C@@H](CC(=O)O)NC(=O)c3cccc(F)n3)cc12] | JHU1221708 | 734.70 | 7.91 μM | 6.91 μM | 1.19 | 0.24 |
| 14 | 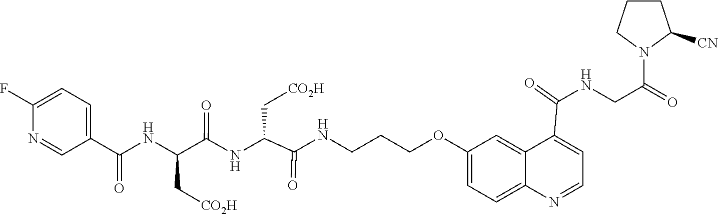 [SMILES: N#C[C@@H]1CCCN1C(=O)CNC(=O)c1ccnc2ccc(OCCCNC(=O)[C@@H](CC(=O)O)NC(=O)[C@@H](CC(=O)O)NC(=O)c3ccc(F)nc3)cc12] | JHU1221785 | 734.70 | 17.73 μM | 86.57 μM | 1.31 | 0.26 |
| 15 | 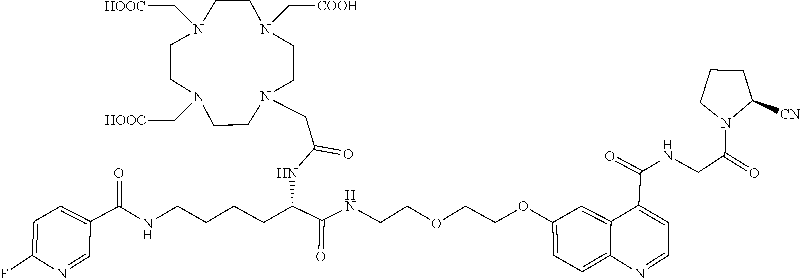 [SMILES: N#C[C@@H]1CCCN1C(=O)CNC(=O)c1ccnc2ccc(OCCOCCNC(=O)[C@H](CCCCNC(=O)c3ccc(F)nc3)NC(=O)CN3CCN(CC(=O)O)CCN(CC(=O)O)CCN(CC(=O)O)CC3)cc12] | JHU1221803 | 1049.13 | 6.22 μM | 1.09 μM | 1.40 | 0.28 |
| 16 | 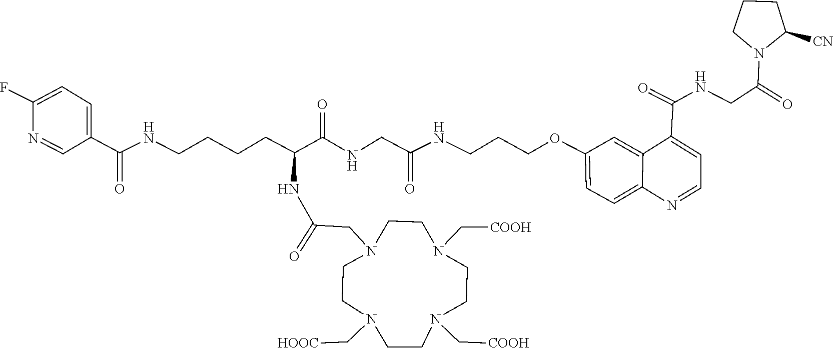 [SMILES: N#C[C@@H]1CCCN1C(=O)CNC(=O)c1ccnc2ccc(OCCCNC(=O)CNC(=O)[C@H](CCCCNC(=O)c3ccc(F)nc3)NC(=O)CN3CCN(CC(=O)O)CCN(CC(=O)O)CCN(CC(=O)O)CC3)cc12] | JHU1221804 | 1076.15 | 8.31 μM | 1.71 μM | 1.01 | 0.20 |
| 17 | 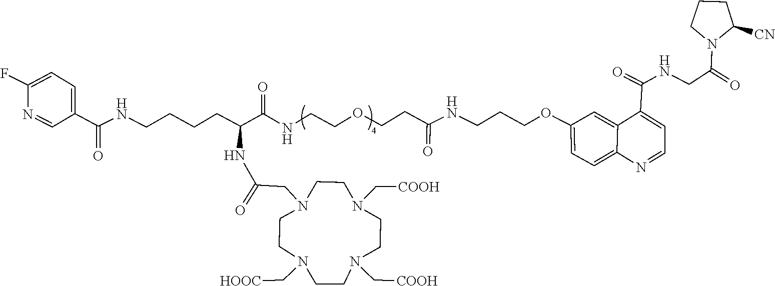 | JHU1221823 | 1266.39 | 7.47 μM | 0.24 μM | 0.59 | 0.12 |
| 18 | 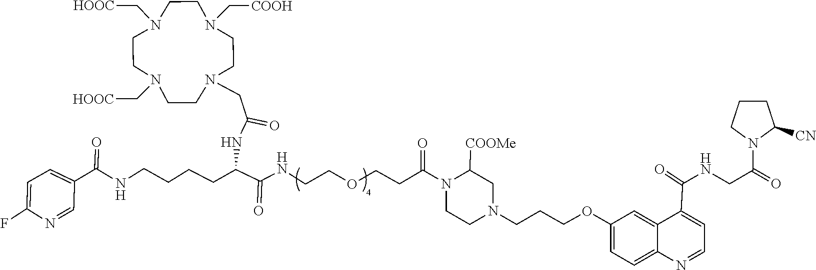 | JHU1221850 | 1393.54 | | | | |
| 19 | 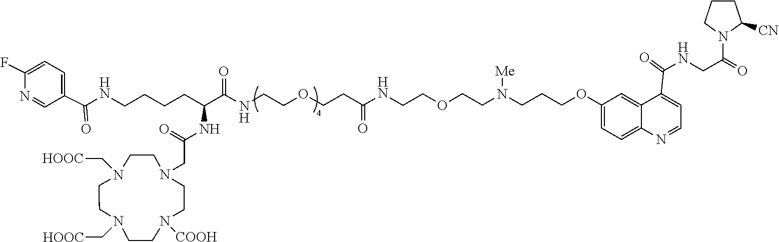 | JHU1222870 | 1367.54 | | | | |
| 20 | 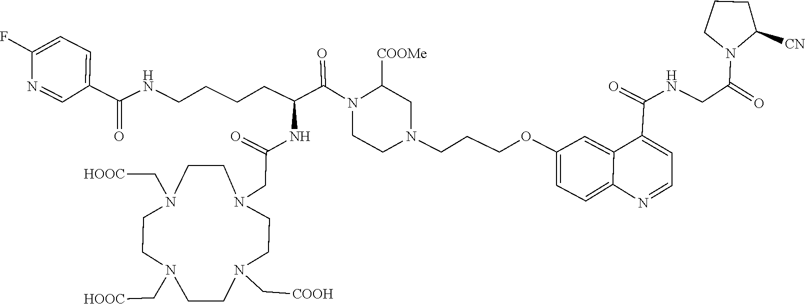 [SMILES: COC(=O)C1CN(CCCOc2ccc3nccc(C(=O)NCC(=O)N4CCC[C@H]4C#N)c3c2)CCN1C(=O)[C@H](CCCCNC(=O)c1ccc(F)nc1)NC(=O)CN1CCN(CC(=O)O)CCN(CC(=O)O)CCN(CC(=O)O)CC1] | JHU1222909 | 1146.25 | | | | |
| 21 | 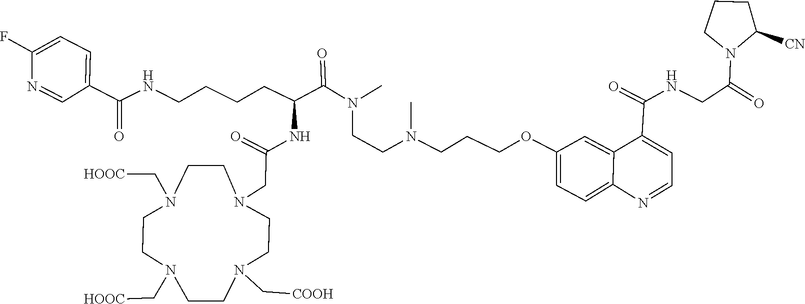 [SMILES: CN(CCCOc1ccc2nccc(C(=O)NCC(=O)N3CCC[C@H]3C#N)c2c1)CCN(C)C(=O)[C@H](CCCCNC(=O)c1ccc(F)nc1)NC(=O)CN1CCN(CC(=O)O)CCN(CC(=O)O)CCN(CC(=O)O)CC1] | JHU1222910 | 1090.23 | | | | |
| 22 | 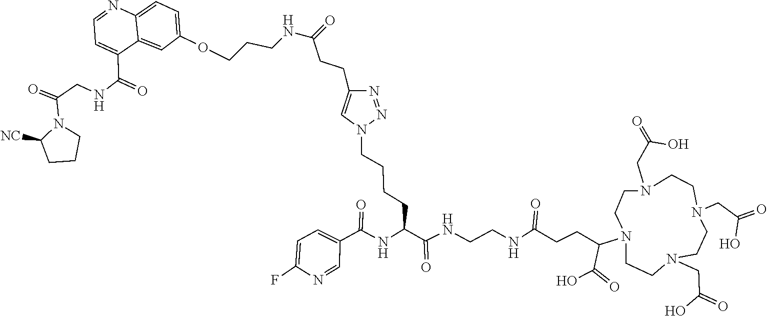 [SMILES: N#C[C@@H]1CCCN1C(=O)CNC(=O)c1ccnc2ccc(OCCCNC(=O)CCc3cn(CCCC[C@H](NC(=O)c4ccc(F)nc4)C(=O)NCCNC(=O)CCC(C(=O)O)N4CCN(CC(=O)O)CCN(CC(=O)O)CCN(CC(=O)O)CC4)nn3)cc12] | SB-FAP-11 | 1257.35 | 9.48 μM | 0.51 μM | 0.35 | 0.07 |
| 23 | 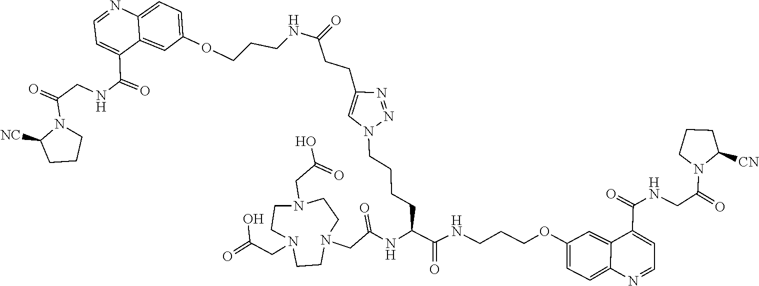 | SB-FAP-09 | 1282.43 | 2.05 μM | 0.63 μM | 0.48 | 0.1 |
| 24 | 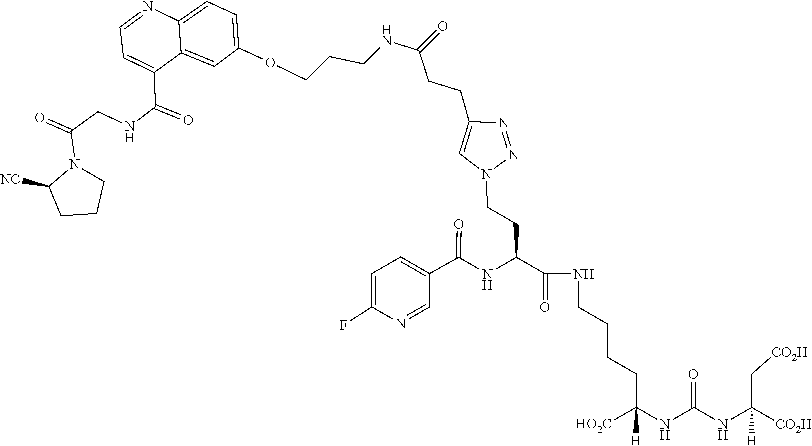 | SRI-07-52 | 1015.39 | | | | |
| 25 | 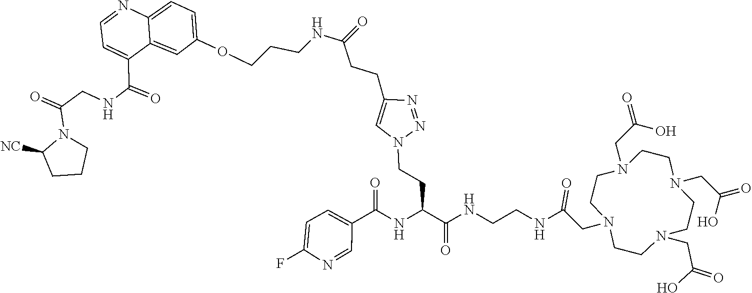 | SRI-07-56 | 1156.52 | | | | |
| 26 | 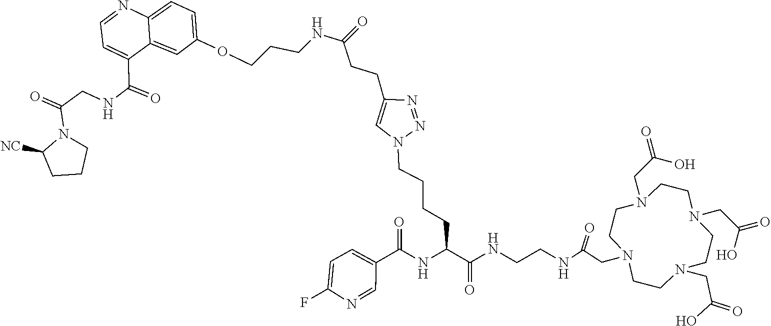 | SRI-08-35 | 1184.55 | | | | |
| 27 | 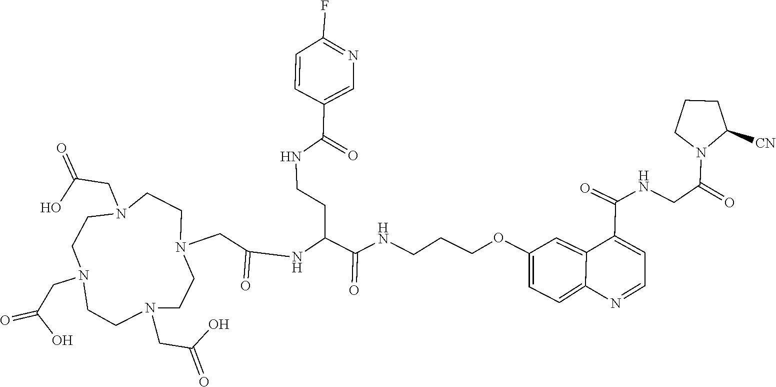 [SMILES: N#C[C@@H]1CCCN1C(=O)CNC(=O)c1ccnc2ccc(OCCCNC(=O)C(CCNC(=O)c3ccc(F)nc3)NC(=O)CN3CCN(CC(=O)O)CCN(CC(=O)O)CCN(CC(=O)O)CC3)cc12] | SRI-08-15 | 990.44 | | | | |
| 28 | 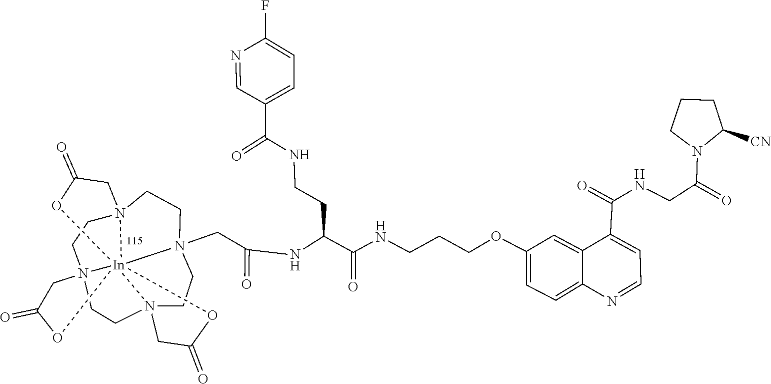 | SRI-08-51 | 1102.32 | | | | |
| 29 | 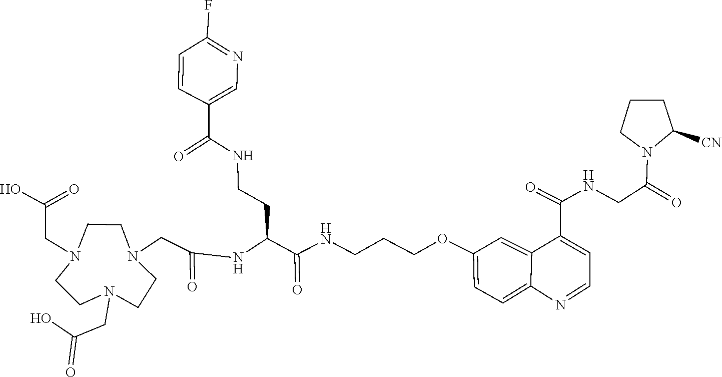 [SMILES: N#C[C@@H]1CCCN1C(=O)CNC(=O)c1ccnc2ccc(OCCCNC(=O)[C@H](CCNC(=O)c3ccc(F)nc3)NC(=O)CN3CCN(CC(=O)O)CCN(CC(=O)O)CC3)cc12] | SRI-08-64 | 889.39 | | | | |
| 30 | 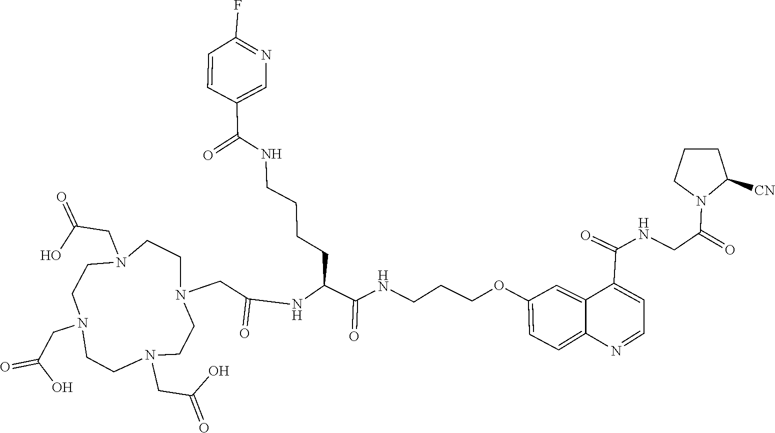 [SMILES: N#C[C@@H]1CCCN1C(=O)CNC(=O)c1ccnc2ccc(OCCCNC(=O)[C@H](CCCCNC(=O)c3ccc(F)nc3)NC(=O)CN3CCN(CC(=O)O)CCN(CC(=O)O)CCN(CC(=O)O)CC3)cc12] | SRI-08-62 | 1018.47 | | | | |
| 31 | 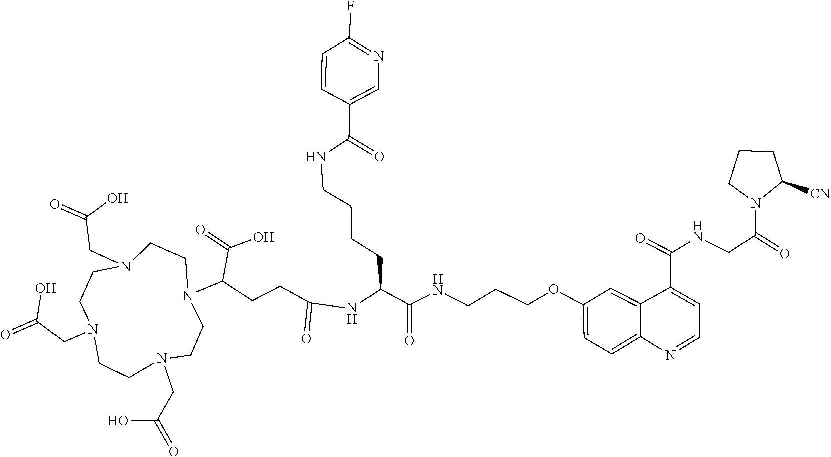 [SMILES: N#C[C@@H]1CCCN1C(=O)CNC(=O)c1ccnc2ccc(OCCCNC(=O)[C@H](CCCCNC(=O)c3ccc(F)nc3)NC(=O)CCC(C(=O)O)N3CCN(CC(=O)O)CCN(CC(=O)O)CCN(CC(=O)O)CC3)cc12] | SRI-08-66 | 1090.49 | | | | |
| 32 | 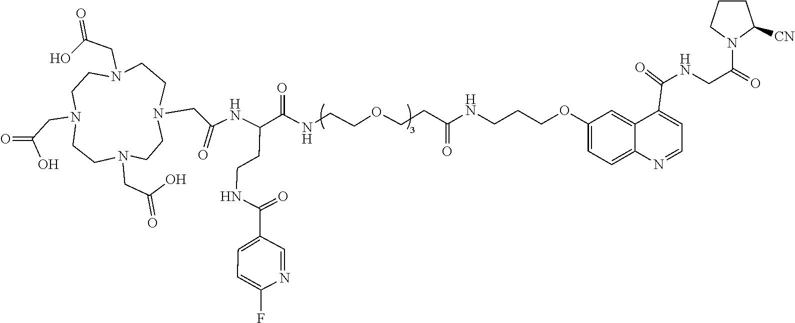 | SRI-08-27 | 1193.55 | | | | |
| 33 | 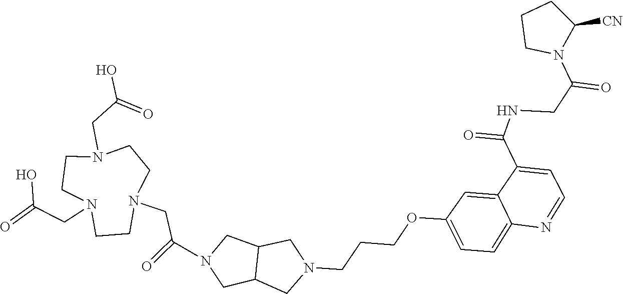 [SMILES: N#C[C@@H]1CCCN1C(=O)CNC(=O)c1ccnc2ccc(OCCCN3CC4CN(C(=O)CN5CCN(CC(=O)O)CCN(CC(=O)O)CC5)CC4C3)cc12] | SRI-07-86 | 761.39 | | | | |
| 34 | 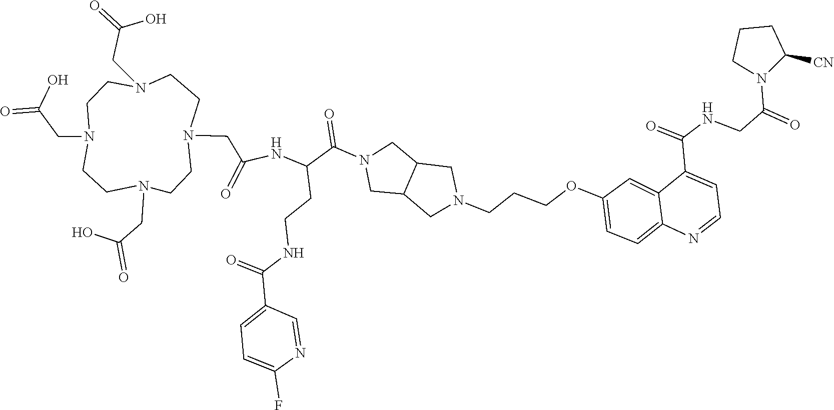 [SMILES: N#C[C@@H]1CCCN1C(=O)CNC(=O)c1ccnc2ccc(OCCCN3CC4CN(C(=O)C(CCNC(=O)c5ccc(F)nc5)NC(=O)CN5CCN(CC(=O)O)CCN(CC(=O)O)CCN(CC(=O)O)CC5)CC4C3)cc12] | SRI-08-37 | 1085.51 | | | | |
| | 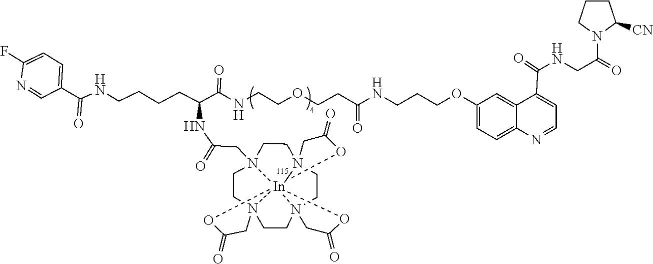 | | 1378.19 | | | | |
| | 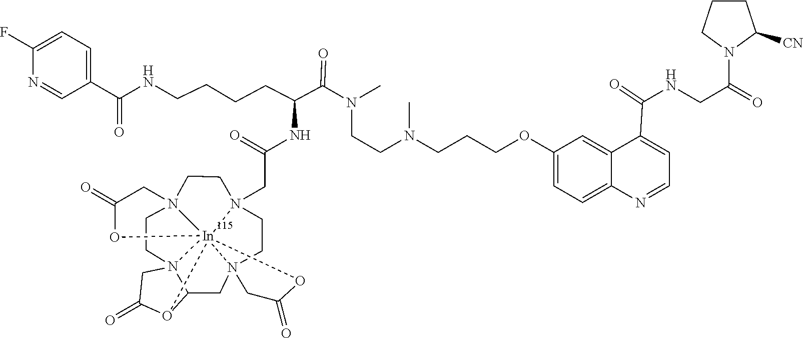 | | 1202.02 | | | | |
| | 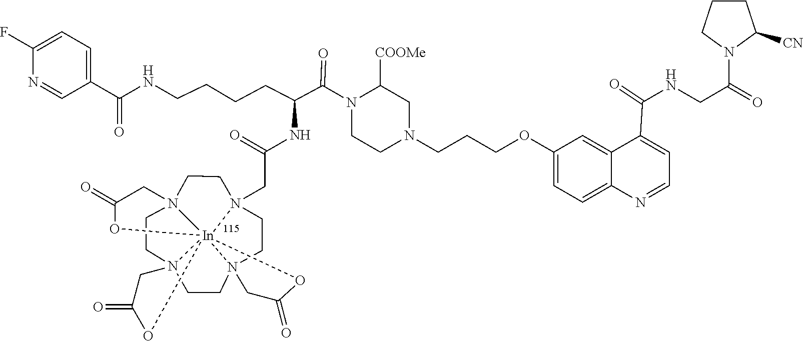 | | 1258.04 | | | | |
| | 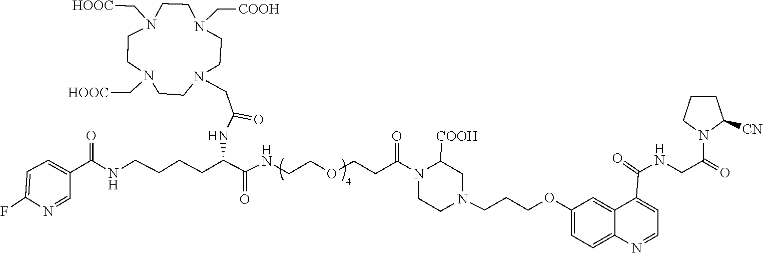 | | 1379.51 | | | | |
| | 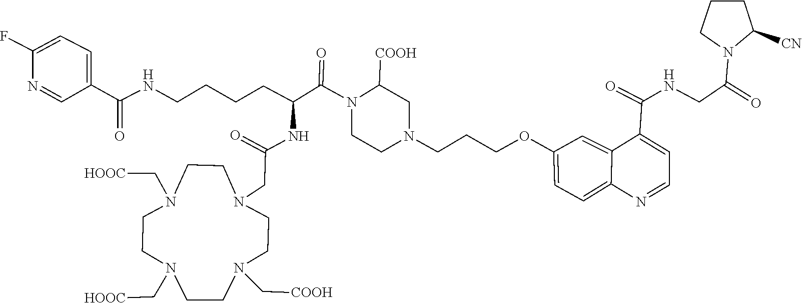 [SMILES: N#C[C@@H]1CCCN1C(=O)CNC(=O)c1ccnc2ccc(OCCCN3CCN(C(=O)[C@H](CCCCNC(=O)c4ccc(F)nc4)NC(=O)CN4CCN(CC(=O)O)CCN(CC(=O)O)CCN(CC(=O)O)CC4)C(C(=O)O)C3)cc12] | | 1132.22 | | | | |
| | 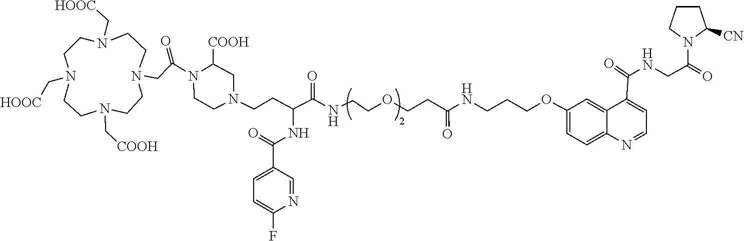 | | 1249.32 | | | | |
| | 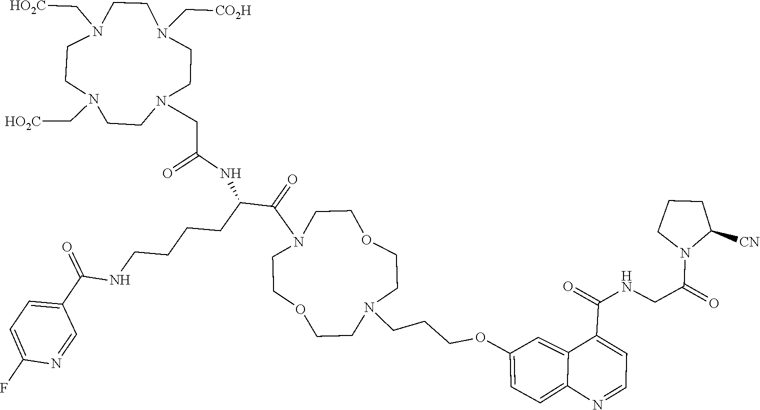 [SMILES: N#C[C@@H]1CCCN1C(=O)CNC(=O)c1ccnc2ccc(OCCCN3CCOCCN(C(=O)[C@H](CCCCNC(=O)c4ccc(F)nc4)NC(=O)CN4CCN(CC(=O)O)CCN(CC(=O)O)CCN(CC(=O)O)CC4)CCOCC3)cc12] | | 1176.32 | | | | |
| | 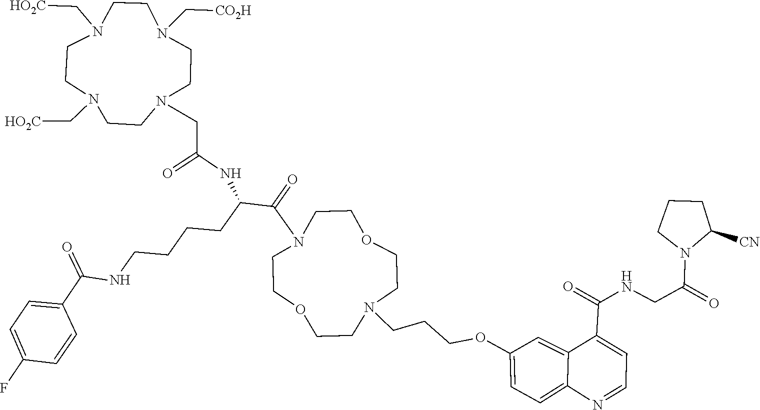 [SMILES: N#C[C@@H]1CCCN1C(=O)CNC(=O)c1ccnc2ccc(OCCCN3CCOCCN(C(=O)[C@H](CCCCNC(=O)c4ccc(F)cc4)NC(=O)CN4CCN(CC(=O)O)CCN(CC(=O)O)CCN(CC(=O)O)CC4)CCOCC3)cc12] | | | | | | |
| | 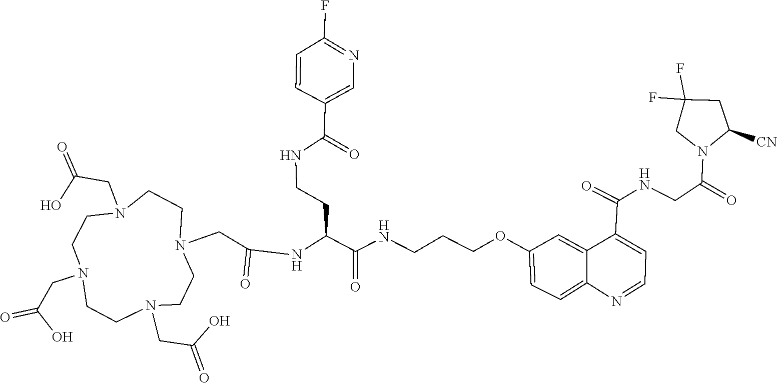 [SMILES: N#C[C@@H]1CC(F)(F)CN1C(=O)CNC(=O)c1ccnc2ccc(OCCCNC(=O)[C@H](CCNC(=O)c3ccc(F)nc3)NC(=O)CN3CCN(CC(=O)O)CCN(CC(=O)O)CCN(CC(=O)O)CC3)cc12] | | 1026.42 | | | | |
| | 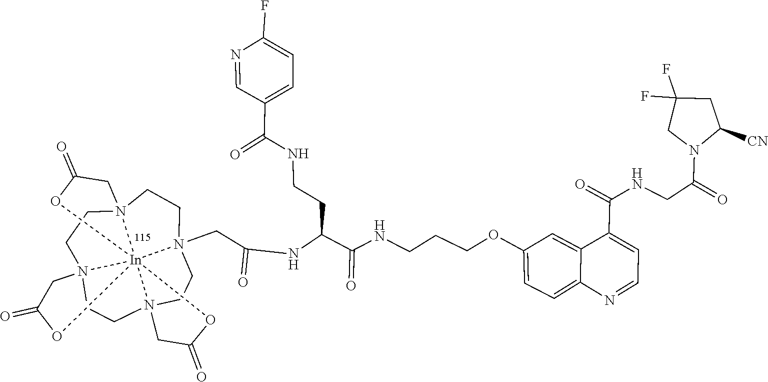 | | 1138.30 | | | | |
| | 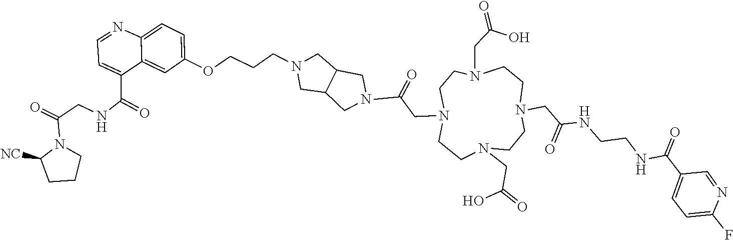 [SMILES: N#C[C@@H]1CCCN1C(=O)CNC(=O)c1ccnc2ccc(OCCCN3CC4CN(C(=O)CN5CCN(CC(=O)O)CCN(CC(=O)NCCNC(=O)c6ccc(F)nc6)CCN(CC(=O)O)CC5)CC4C3)cc12] | | 1027.50 | | | | |
| | 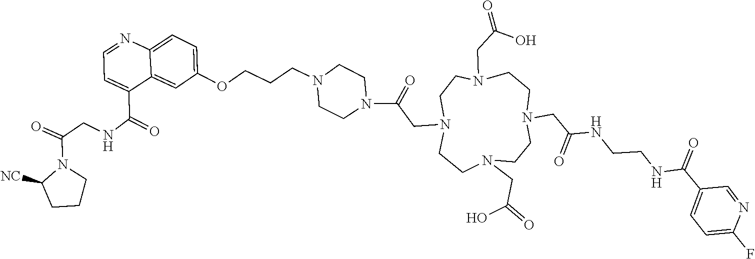 [SMILES: N#C[C@@H]1CCCN1C(=O)CNC(=O)c1ccnc2ccc(OCCCN3CCN(C(=O)CN4CCN(CC(=O)O)CCN(CC(=O)NCCNC(=O)c5ccc(F)nc5)CCN(CC(=O)O)CC4)CC3)cc12] | | 1001.49 | | | | |
| | 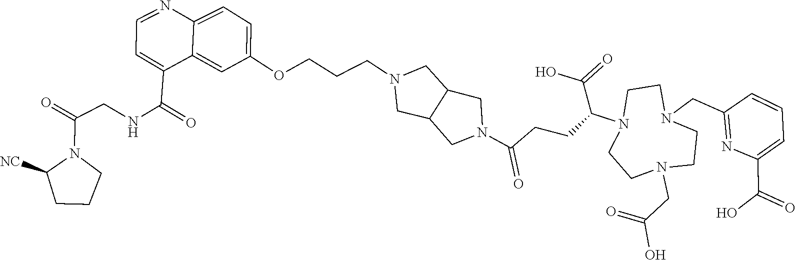 [SMILES: N#C[C@@H]1CCCN1C(=O)CNC(=O)c1ccnc2ccc(OCCCN3CC4CN(C(=O)CC[C@H](C(=O)O)N5CCN(CC(=O)O)CCN(Cc6cccc(C(=O)O)n6)CC5)CC4C3)cc12] | | 911.03 | | | | |
| | 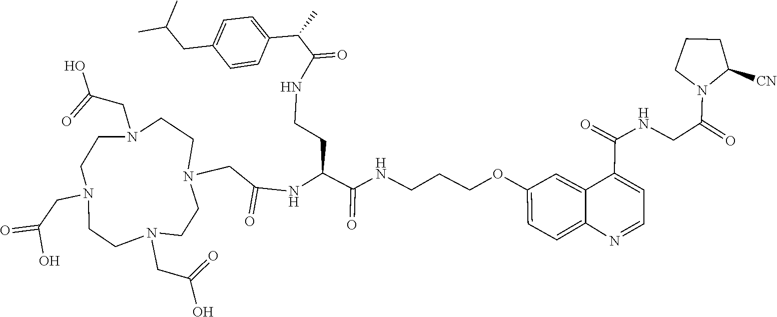 [SMILES: CC(C)Cc1ccc([C@H](C)C(=O)NCC[C@H](NC(=O)CN2CCN(CC(=O)O)CCN(CC(=O)O)CCN(CC(=O)O)CC2)C(=O)NCCCOc2ccc3nccc(C(=O)NCC(=O)N4CCC[C@H]4C#N)c3c2)cc1] | | 1056.23 | | | | |

### Example 7

### Representative FAP-α/PSMA Dual Inhibitors

Representative FAP-α/PSMA dual inhibitors and representative biological data are provided in Table 4.

TABLE 4
FAP-α/PSMA Dual Inhibitors

| | | | | | | FAP | FAP | PSMA | PSMA |
|---|---|---|---|---|---|---|---|---|---|
| | | | MW | DPPIV | PREP | IC₅₀ | Ki | IC₅₀ | Ki |
| No | Structure | Code | (g/mol) | IC₅₀ | IC₅₀ | (nM) | (nM) | (nM) | (nM) |
| i | 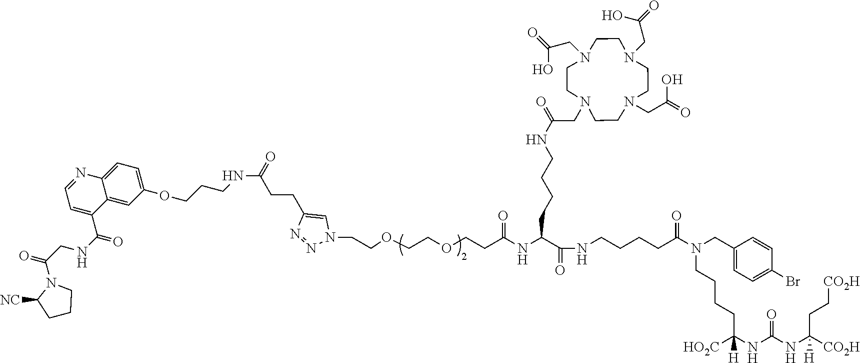 | SB- FAP- 01 | 1789.74 | 16.90 μM | 0.35 μM | 1.79 | 0.36 | 83.16 | 16.63 |
| ii | 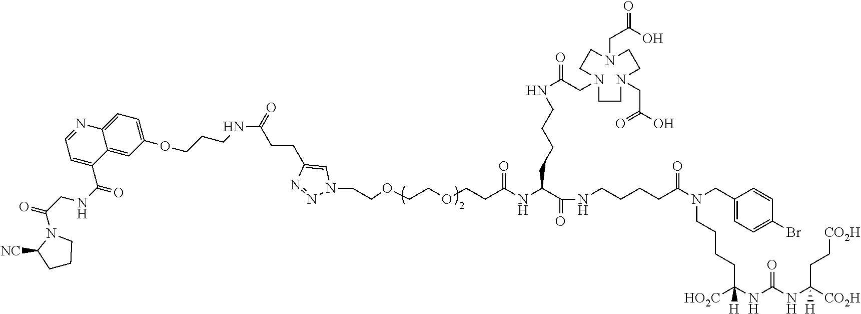 | SB- FAP- 02 | 1690.71 | 8.81 μM | 0.58 μM | 1.53 | 0.31 | 90.00 | 18.10 |
| iii | 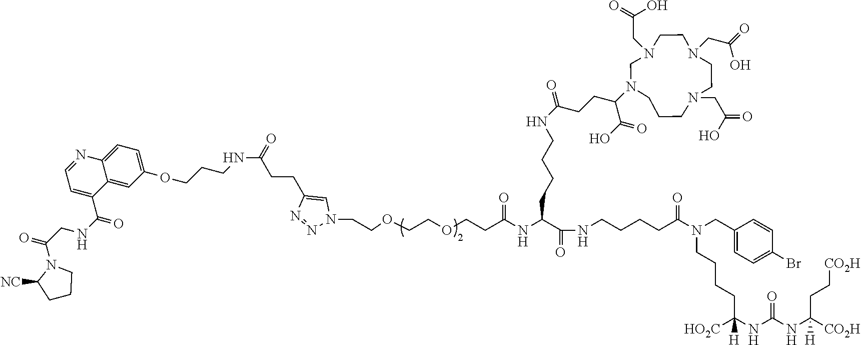 | SB- FAP- 03 | 1862.77 | 18.38 μM | 1.37 μM | 3.42 | 0.68 | 14.47 | 2.89 |
| iv | 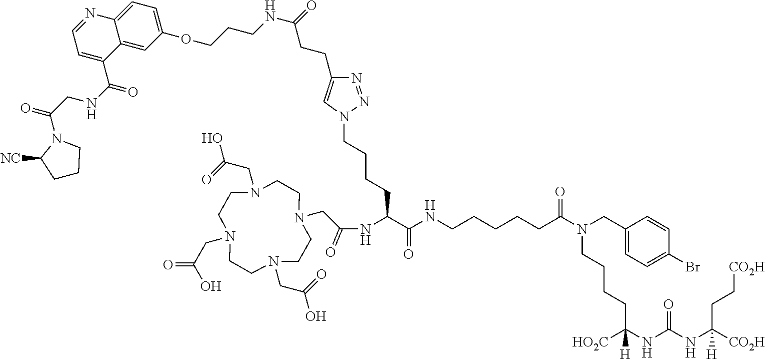 [SMILES: N#C[C@@H]1CCCN1C(=O)CNC(=O)c1ccnc2ccc(OCCCNC(=O)CCc3cn(CCCC[C@H](NC(=O)CN4CCN(CC(=O)O)CCN(CC(=O)O)CCN(CC(=O)O)CC4)C(=O)NCCCCCC(=O)N(CCCC[C@H](NC(=O)N[C@@H](CCC(=O)O)C(=O)O)C(=O)O)Cc4ccc(Br)cc4)nn3)cc12] | SB- FAP- 06 | 1603.60 | 14.09 μM | 0.37 μM | 1.84 | 0.37 | 4.71 | 0.94 |
| v | 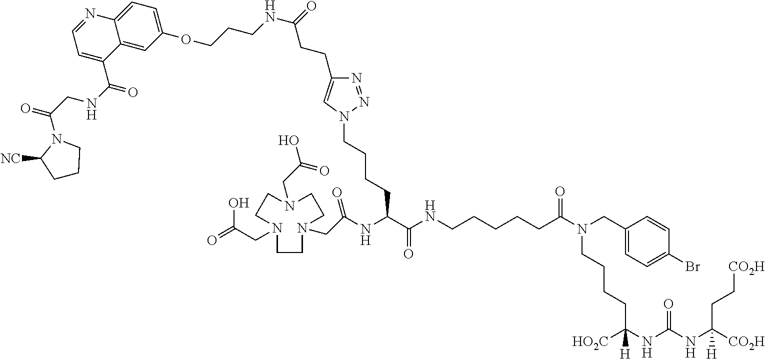 [SMILES: N#C[C@@H]1CCCN1C(=O)CNC(=O)c1ccnc2ccc(OCCCNC(=O)CCc3cn(CCCC[C@H](NC(=O)CN4CCN(CC(=O)O)CCN(CC(=O)O)CC4)C(=O)NCCCCCC(=O)N(CCCC[C@H](NC(=O)N[C@@H](CCC(=O)O)C(=O)O)C(=O)O)Cc4ccc(Br)cc4)nn3)cc12] | SB- FAP- 07 | 1502.49 | 11.67 μM | 1.07 μM | 0.65 | 0.13 | 39.61 | 7.92 |
| vi | 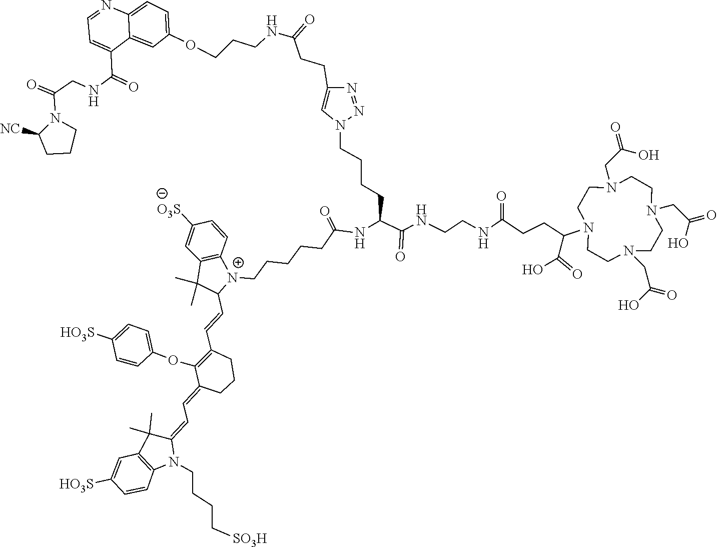 | SB- FAP- 10 | 2117.79 | 11.94 μM | 2.53 μM | 3.43 | 0.69 | N/A | N/A |
| vii | 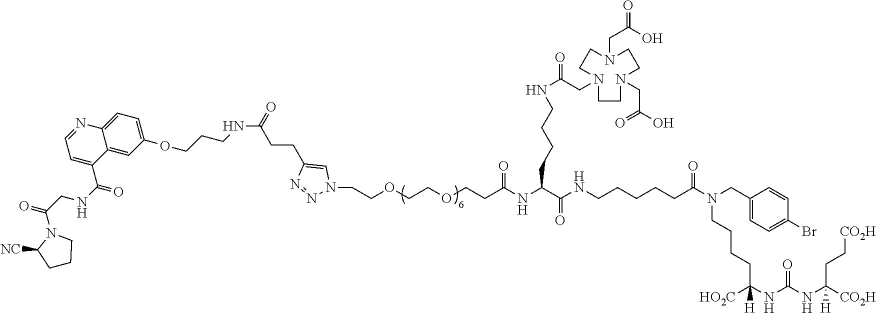 | SRI- 07- 31 | 1837.89 | 5.61 μM | 2.73 μM | 2.88 | 0.58 | 181.40 | 36.82 |
| viii | 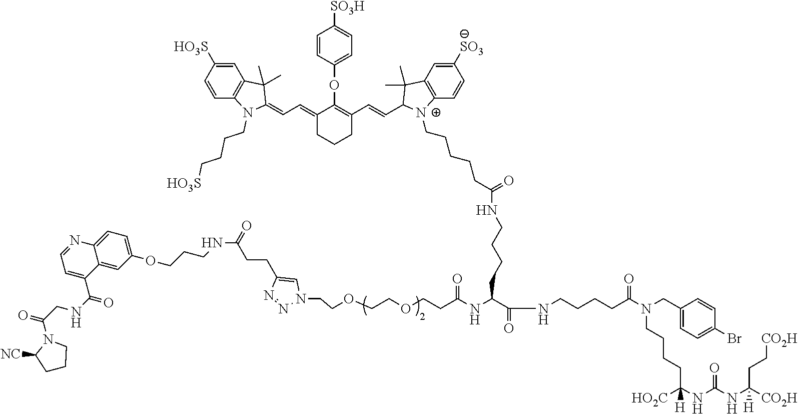 | SB- FAP- 04 | 2388.80 | 4.93 μM | 1.27 μM | 3.76 | 0.75 | 26.62 | 5.33 |
| ix | 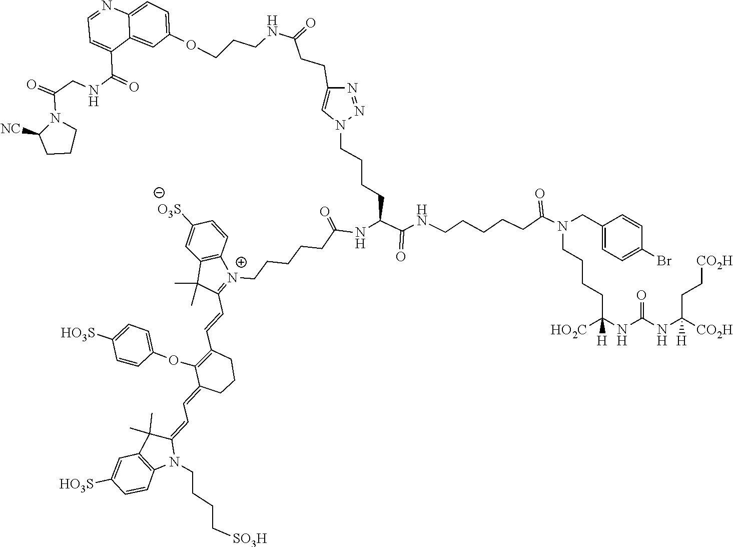 | SB- FAP- 08 | 2202.35 | 26.97 μM | 1.76 μM | 4.67 | 0.93 | 23.30 | 4.66 |
| x | 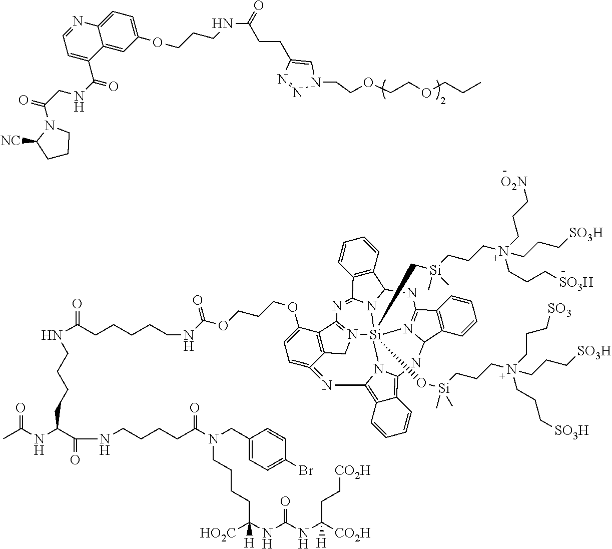 | SB- FAP- 05 | 3111.98 | 4.90 μM | 2.28 μM | 2.92 | 0.58 | 18.54 | 3.71 |
| xi | 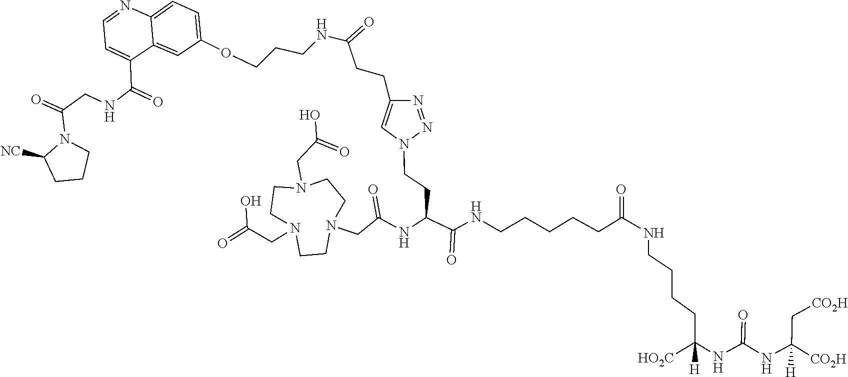 | SRI- 07- 42 | 1291.39 | 9.91 μM | 11.24 μM | 2.23 | 0.45 | 4.71 (μM) | 0.94 (μM) |

### Example 8

### Biodistribution ¹¹¹In-SRI-08-15 and Related Compounds

Tissue biodistribution of ¹¹¹In-SRI-8-15 and related compounds is provided in Table 5 (n=4, time-point 1 h). As provided in Table 5, ¹¹¹In-SRI-08-15, by virtue of placement of a metal chelator in the DOTA ring, unexpectedly enables very high uptake in U87 cells. Unlike [¹⁸F]FAPI-74, Al[¹⁸F]F chemistry is not used to introduce ¹⁸F. In contrast, the presently disclosed compounds are prepared in one step from a trimethylammonium precursor at very high specific activity.

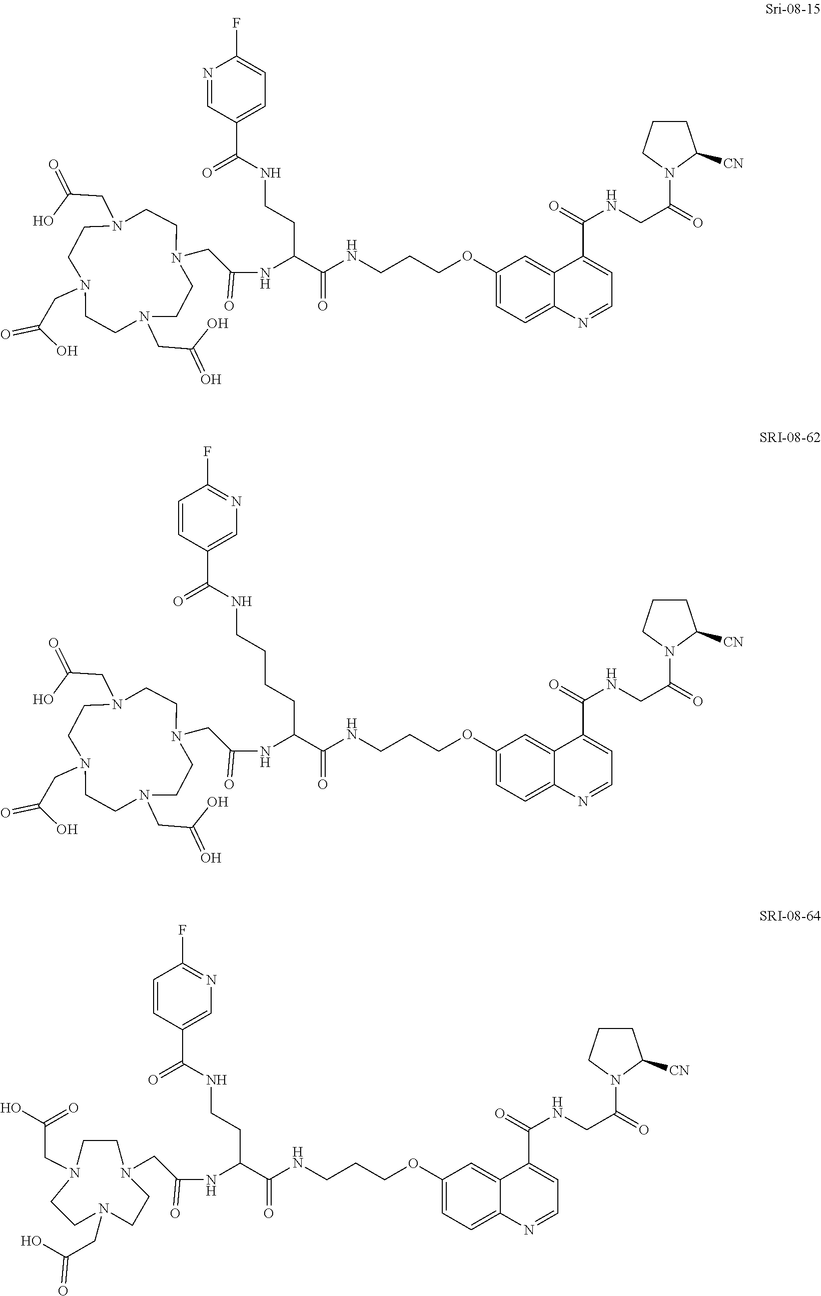 [SMILES: N#C[C@@H]1CCCN1C(=O)CNC(=O)c1ccnc2ccc(OCCCNC(=O)C(CCNC(=O)c3ccc(F)nc3)NC(=O)CN3CCN(CC(=O)O)CCN(CC(=O)O)CCN(CC(=O)O)CC3)cc12 ; N#C[C@@H]1CCCN1C(=O)CNC(=O)c1ccnc2ccc(OCCCNC(=O)C(CCCCNC(=O)c3ccc(F)nc3)NC(=O)CN3CCN(CC(=O)O)CCN(CC(=O)O)CCN(CC(=O)O)CC3)cc12 ; N#C[C@@H]1CCCN1C(=O)CNC(=O)c1ccnc2ccc(OCCCNC(=O)C(CCNC(=O)c3ccc(F)nc3)NC(=O)CN3CCN(CC(=O)O)CCN(CC(=O)O)CC3)cc12]

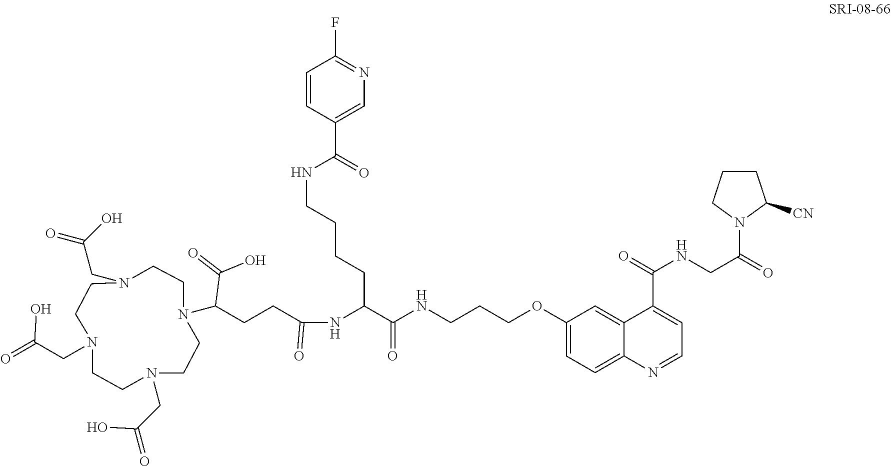 [SMILES: N#C[C@@H]1CCCN1C(=O)CNC(=O)c1ccnc2ccc(OCCCNC(=O)C(CCCCNC(=O)c3ccc(F)nc3)NC(=O)CCC(C(=O)O)N3CCN(CC(=O)O)CCN(CC(=O)O)CCN(CC(=O)O)CC3)cc12]

TABLE 5
Biodistribution ¹¹¹In-SRI-08-15 and Related Compounds

| | ¹¹¹In- | ¹¹¹In- | ¹¹¹In- | ¹¹¹In- |
|---|---|---|---|---|
| | SRI-08-64 | SRI-08-62 | SRI-08-66 | SRI-08-15 |
| Blood | 17.14 ± 3.58 | 7.37 ± 1.24 | 9.89 ± 0.93 | 4.23 ± 0.47 |
| Heart | 5.35 ± 1.53 | 2.99 ± 0.23 | 3.63 ± 0.30 | 1.69 ± 0.08 |
| Lung | 10.11 ± 4.23 | 4.47 ± 0.66 | 5.76 ± 0.46 | 3.41 ± 0.15 |
| Liver | 10.40 ± 2.46 | 3.40 ± 0.39 | 3.47 ± 0.76 | 3.41 ± 0.12 |
| Stomach | 3.00 ± 0.71 | 1.74 ± 0.07 | 1.78 ± 0.27 | 1.55 ± 0.12 |
| Pancreas | 12.60 ± 1.66 | 9.00 ± 1.56 | 5.99 ± 2.32 | 5.84 ± 0.75 |
| Spleen | 4.53 ± 1.60 | 2.33 ± 0.72 | 2.30 ± 0.36 | 1.38 ± 0.09 |
| Fat | 0.98 ± 0.46 | 0.67 ± 0.06 | 0.65 ± 0.08 | 0.63 ± 0.08 |
| Kidney | 7.82 ± 0.96 | 3.26 ± 0.33 | 4.17 ± 0.42 | 2.66 ± 0.11 |
| Muscle | 2.48 ± 0.51 | 2.29 ± 0.28 | 1.95 ± 0.31 | 2.53 ± 0.14 |
| Sm Intest | 7.13 ± 2.60 | 5.58 ± 0.93 | 5.64 ± 0.49 | 3.84 ± 0.60 |
| Salivary | 10.45 ± 4.40 | 8.16 ± 1.09 | 7.26 ± 0.67 | 9.42 ± 0.63 |
| Lacrimal | 8.98 ± 3.46 | 5.71 ± 0.76 | 4.63 ± 0.38 | 5.57 ± 0.91 |
| Bladder | 5.01 ± 1.98 | 2.50 ± 0.67 | 7.26 ± 5.86 | 6.78 ± 2.45 |
| Bone | 5.79 ± 1.98 | 5.29 ± 1.18 | 4.58 ± 0.51 | 6.45 ± 0.39 |
| U87 | 8.85 ± 2.85 | 10.11 ± 1.23 | 7.06 ± 0.53 | 18.66 ± 6.73 |

### Example 9

### Biodistribution of Representative Compounds

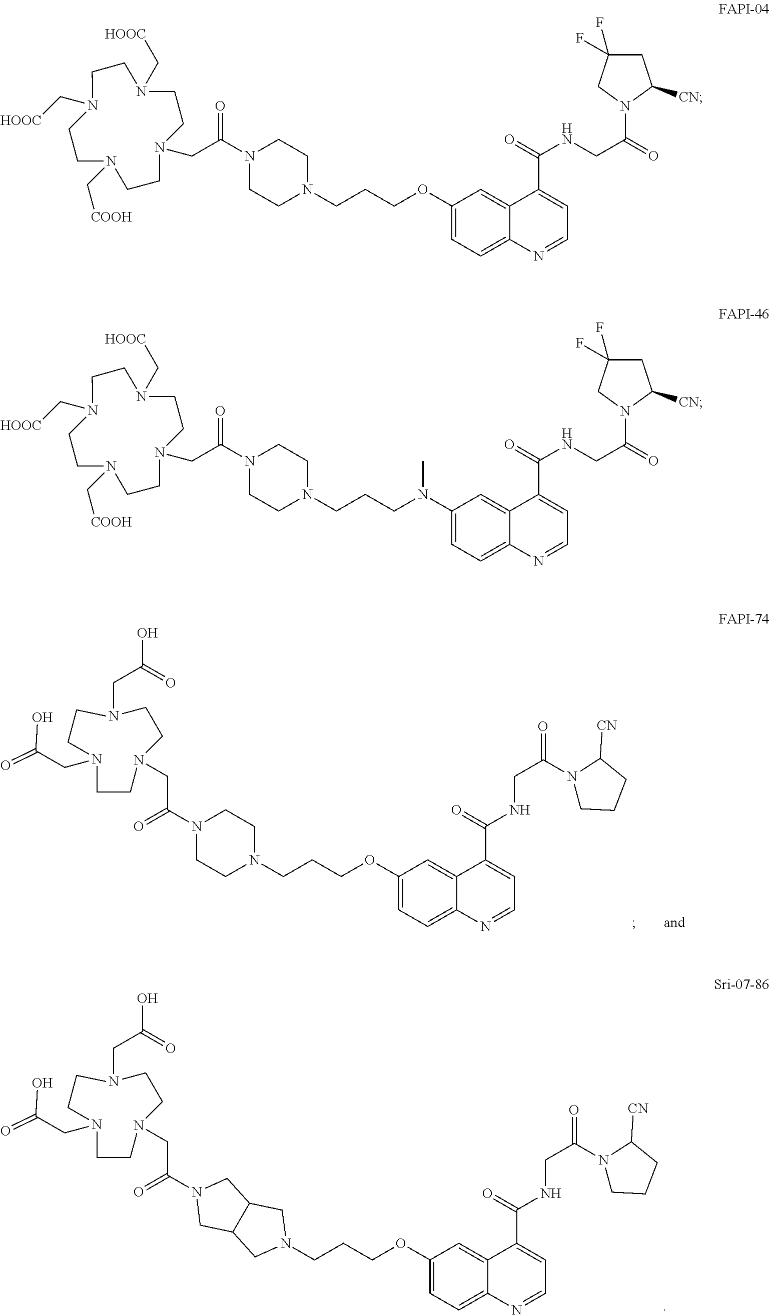 [SMILES: N#C[C@@H]1CC(F)(F)CN1C(=O)CNC(=O)c1ccnc2ccc(OCCCN3CCN(C(=O)CN4CCN(CC(=O)O)CCN(CC(=O)O)CCN(CC(=O)O)CC4)CC3)cc12 ; CN(CCCN1CCN(C(=O)CN2CCN(CC(=O)O)CCN(CC(=O)O)CCN(CC(=O)O)CC2)CC1)c1ccc2nccc(C(=O)NCC(=O)N3CC(F)(F)C[C@H]3C#N)c2c1 ; N#CC1CCCN1C(=O)CNC(=O)c1ccnc2ccc(OCCCN3CCN(C(=O)CN4CCN(CC(=O)O)CCN(CC(=O)O)CC4)CC3)cc12 ; N#CC1CCCN1C(=O)CNC(=O)c1ccnc2ccc(OCCCN3CC4CN(C(=O)CN5CCN(CC(=O)O)CCN(CC(=O)O)CC5)CC4C3)cc12]

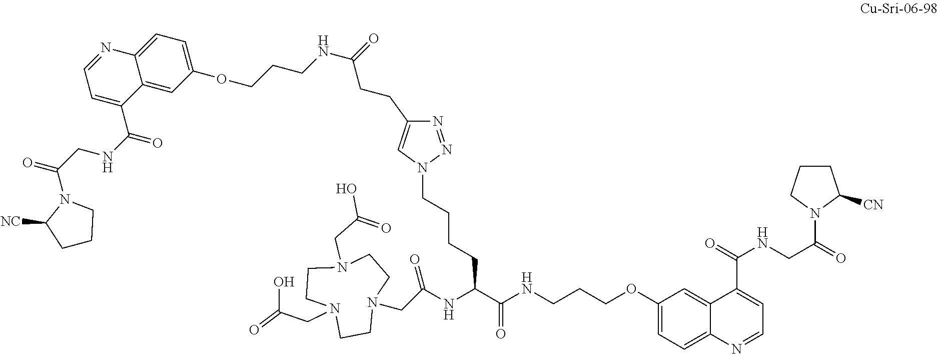 [SMILES: N#C[C@@H]1CCCN1C(=O)CNC(=O)c1ccnc2ccc(OCCCNC(=O)CCc3cn(CCCC[C@H](NC(=O)CN4CCN(CC(=O)O)CCN(CC(=O)O)CC4)C(=O)NCCCOc4ccc5nccc(C(=O)NCC(=O)N6CCC[C@H]6C#N)c5c4)nn3)cc12]

Head-to-head biodistribution study (% ID/g) (n=5/group). Dose 20 μCi. ⁶⁴Cu-Sri-07-86 and ⁶⁴Cu-FAPI-74 at 1 h time-point. Note ⁶⁴Cu-FAPI-04 contains a DOTA group still showed low liver uptake, most likely related to the piperazine group next to DOTA, acting as a co-ligand. The DOTA chelated compound exhibited >10% ID/g liver uptake.

| | | | ⁶⁴Cu-FAPI-04 |
|---|---|---|---|
| | ⁶⁴Cu-Sri-07-86 | ⁶⁴Cu-FAPI-74 | (n = 3) |
| Blood | 2.54 ± 0.90 | 2.48 ± 0.87 | 3.43 ± 0.86 |
| Heart | 1.04 ± 0.28 | 1.16 ± 0.28 | 1.85 ± 0.46 |
| Lung | 2.02 ± 0.93 | 2.48 ± 0.64 | 3.03 ± 0.76 |
| Liver | 1.97 ± 0.36 | 1.69 ± 0.83 | 2.56 ± 0.85 |
| Stomach | 1.26 ± 0.45 | 1.29 ± 0.45 | 2.02 ± 0.77 |
| Pancreas | 1.89 ± 0.57 | 2.09 ± 1.12 | 2.35 ± 0.72 |
| Spleen | 1.13 ± 0.42 | 1.23 ± 0.41 | 1.46 ± 0.58 |
| Fat | 0.31 ± 0.08 | 0.48 ± 0.43 | 0.56 ± 0.10 |
| Kidney | 4.00 ± 1.28 | 3.83 ± 2.31 | 2.79 ± 0.46 |
| Muscle | 1.65 ± 1.10 | 2.05 ± 0.84 | 2.16 ± 0.58 |
| Small Intestine | 3.15 ± 1.34 | 3.80 ± 2.06 | 3.18 ± 0.77 |
| Salivary Gland | 2.77 ± 0.53 | 4.42 ± 0.55 | 10.08 ± 2.00 |
| Lacrimal gland | 3.53 ± 2.93 | 5.17 ± 1.51 | 9.26 ± 4.86 |
| Bladder | 5.55 ± 4.26 | 3.51 ± 0.30 | 6.38 ± 1.44 |
| Bone | 2.84 ± 0.83 | 7.94 ± 0.78 | 7.51 ± 2.99 |
| U87 | 6.55 ± 1.41 | 11.42 ± 1.11 | 9.53 ± 2.09 |

| Tumor | | | |
|---|---|---|---|
| weight | ⁶⁴Cu-Sri-07-86 | ⁶⁴Cu-FAPI-74 | ⁶⁴Cu-FAPI-04 |
| | 0.41 | 0.46 | 0.05 |
| | 0.46 | 0.18 | 0.03 |
| | 0.35 | 0.43 | 0.04 |
| | 0.41 | 0.12 | |
| | 0.39 | 0.71 | |
| Average | 0.40 ± 0.04 | 0.38 ± 0.24 | 0.04 ± 0.01 |
| wt ± StDev | | | |

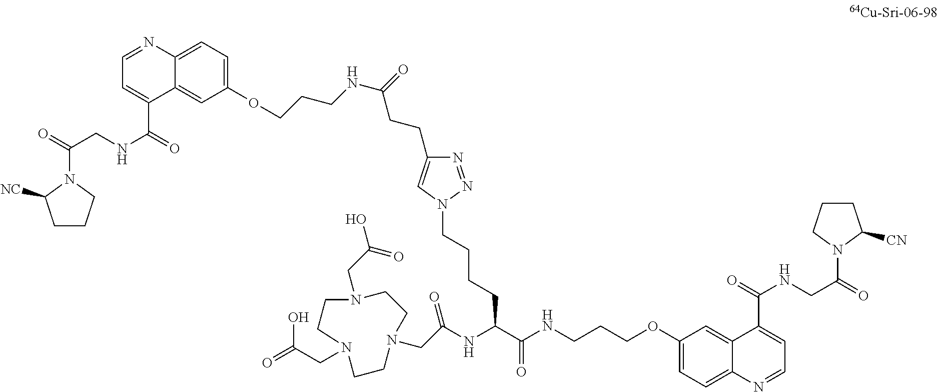 [SMILES: N#C[C@@H]1CCCN1C(=O)CNC(=O)c1ccnc2ccc(OCCCNC(=O)CCc3cn(CCCC[C@H](NC(=O)CN4CCN(CC(=O)O)CCN(CC(=O)O)CC4)C(=O)NCCCOc4ccc5nccc(C(=O)NCC(=O)N6CCC[C@H]6C#N)c5c4)nn3)cc12]

Compound Name: ⁶⁴Cu-Sri-06-98. Dose: 200 μCi, n=3 per group. Note the position of triazole group and liver uptake.

| | | 2 h + 10 nmol | |
|---|---|---|---|
| | 2 h | Sri-06-98 | 4 h |
| Blood | 3.49 ± 0.18 | 0.27 ± 0.08 | 2.54 ± 0.37 |
| Heart | 3.70 ± 0.22 | 0.17 ± 0.01 | 2.40 ± 0.12 |
| Lung | 2.62 ± 0.11 | 0.49 ± 0.01 | 2.04 ± 0.10 |
| Liver | 2.80 ± 0.31 | 0.68 ± 0.16 | 2.68 ± 0.08 |
| Stomach | 2.09 ± 1.00 | 0.31 ± 0.02 | 1.60 ± 0.36 |
| Pancreas | 4.59 ± 2.38 | 0.21 ± 0.04 | 4.54 ± 0.62 |
| Spleen | 1.51 ± 0.14 | 0.25 ± 0.01 | 1.14 ± 0.16 |
| Fat | 0.47 ± 0.02 | 0.07 ± 0.02 | 0.50 ± 0.12 |
| Kidney | 2.00 ± 0.52 | 1.57 ± 0.00 | 1.67 ± 0.03 |
| Muscle | 2.46 ± 0.20 | 0.09 ± 0.02 | 2.13 ± 0.14 |
| Small Intestine | 2.97 ± 0.33 | 0.54 ± 0.08 | 2.69 ± 0.42 |
| Salivary Glands | 8.88 ± 1.37 | 0.34 ± 0.02 | 5.92 ± 0.57 |
| Lacrimal Gland | 5.07 ± 2.20 | 0.16 ± 0.02 | 4.54 ± 0.44 |
| Bladder | 1.63 ± 0.23 | 5.61 ± 7.96 | 5.56 ± 1.53 |
| Bone | 2.91 ± 0.36 | 0.59 ± 0.65 | 2.69 ± 0.14 |
| U87 | 7.64 ± 2.12 | 2.70 ± 1.00 | 11.15 ± 1.49 |

tumor weight
⁶⁴Cu-Sri-06-98
0.1644
0.1942
0.2037
0.1172
0.044
0.1097
0.1556
0.2547
0.25 ± 0.17

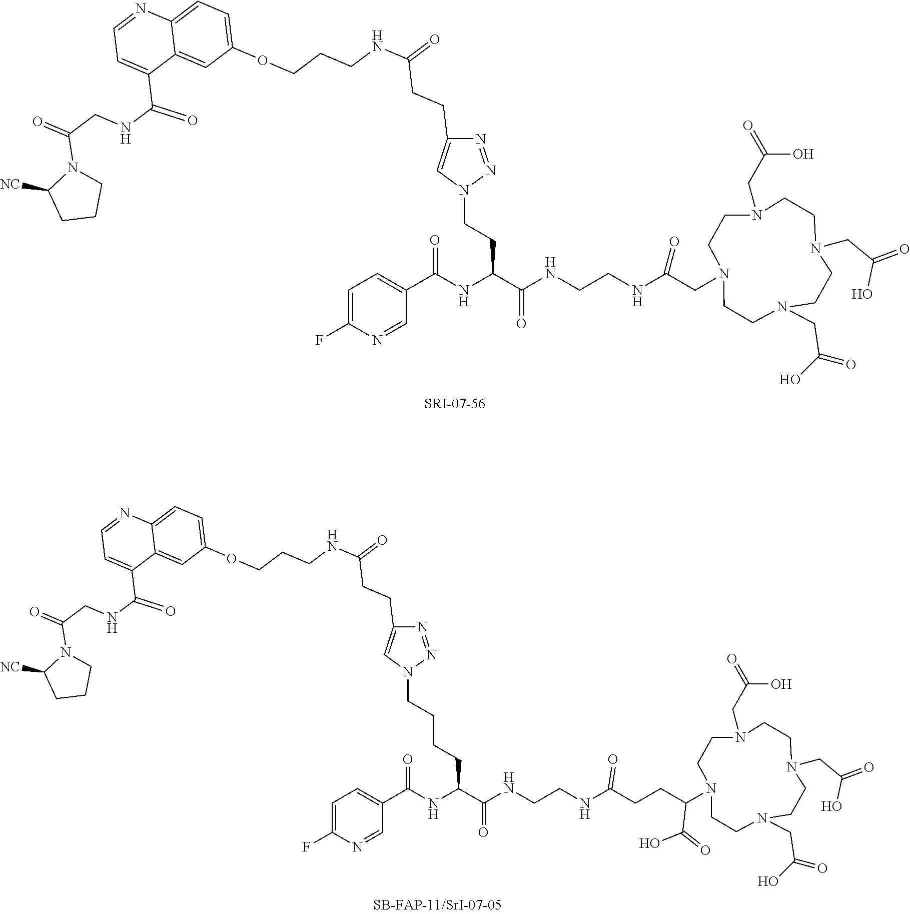 [SMILES: N#C[C@@H]1CCCN1C(=O)CNC(=O)c1ccnc2ccc(OCCCNC(=O)CCc3cn(CC[C@H](NC(=O)c4ccc(F)nc4)C(=O)NCCNC(=O)CN4CCN(CC(=O)O)CCN(CC(=O)O)CCN(CC(=O)O)CC4)nn3)cc12 ; N#C[C@@H]1CCCN1C(=O)CNC(=O)c1ccnc2ccc(OCCCNC(=O)CCc3cn(CCCC[C@H](NC(=O)c4ccc(F)nc4)C(=O)NCCNC(=O)CCC(C(=O)O)N4CCN(CC(=O)O)CCN(CC(=O)O)CCN(CC(=O)O)CC4)nn3)cc12]

Compound Name: ⁶⁴Cu-SRI-06-57, ⁶⁴Cu-FAPI-04, ⁶⁴Cu-07-56 and ⁶⁴Cu-07-05

Dose: 20 μCi, n=3 per group, Tumor model: U87 in NSG mice. Note the position of the triazole group and DOTA.

| | Group 2 | | | | 64Cu-Sr- | |
|---|---|---|---|---|---|---|
| | ⁶⁴Cu-SRI- | | ⁶⁴Cu-Sri07- | ⁶⁴Cu-Sri- | 07-05 | ⁶⁴Cu-07-05 |
| | 06-57 | ⁶⁴Cu- | 56 Control | 07-56 G2 | Control | G2 (DOTA) |
| | G3 | FAPI-04 | (DOTAGA) | (DOTAGA) | (DOTA) | (n = 2) |
| Blood | 2.29 ± 0.17 | 3.43 ± 0.86 | 9.73 ± 1.42 | 2.10 ± 0.29 | 8.46 ± 0.87 | 2.18 ± 0.51 |
| Heart | 1.58 ± 0.17 | 1.85 ± 0.46 | 5.31 ± 0.65 | 1.87 ± 0.15 | 4.86 ± 2.75 | 3.21 ± 0.63 |
| Lung | 2.71 ± 0.40 | 3.03 ± 0.76 | 6.95 ± 1.11 | 4.15 ± 0.14 | 8.31 ± 2.94 | 5.40 ± 0.06 |
| Liver | 4.27 ± 0.53 | 2.56 ± 0.85 | 13.25 ± 1.40 | 8.01 ± 1.34 | 9.96 ± 1.43 | 12.99 ± 5.14 |
| Stomach | 2.17 ± 0.12 | 2.02 ± 0.77 | 3.99 ± 0.32 | 4.40 ± 0.17 | 5.63 ± 2.03 | 4.29 ± 0.28 |
| Pancreas | 1.02 ± 0.72 | 2.35 ± 0.72 | 10.15 ± 0.85 | 2.65 ± 0.06 | 5.32 ± 2.97 | 4.24 ± 0.47 |
| Spleen | 1.83 ± 0.14 | 1.46 ± 0.58 | 3.60 ± 0.37 | 2.09 ± 0.23 | 3.21 ± 1.53 | 2.85 ± 0.41 |
| Fat | 1.03 ± 0.39 | 0.56 ± 0.10 | 0.98 ± 0.28 | 0.33 ± 0.05 | 0.37 ± 0.15 | 0.73 ± 0.18 |
| Muscle | 1.52 ± 0.20 | 2.79 ± 0.46 | 2.18 ± 0.11 | 1.16 ± 0.22 | 1.70 ± 0.79 | 1.90 ± 0.31 |
| Kidney | 9.05 ± 0.88 | 2.16 ± 0.58 | 4.54 ± 0.51 | 4.75 ± 1.45 | 4.99 ± 1.40 | 4.31 ± 0.47 |
| Sm. | 3.01 ± 0.08 | 3.18 ± 0.77 | 8.08 ± 1.61 | 5.93 ± 0.98 | 8.58 ± 2.20 | 8.85 ± 2.55 |
| Intest. | | | | | | |
| Salivary | 4.64 ± 0.26 | 10.08 ± 2.00 | 7.92 ± 0.81 | 4.01 ± 0.46 | 5.16 ± 1.99 | 7.40 ± 0.15 |
| Lacrimal | 3.32 ± 0.36 | 9.26 ± 4.86 | 8.01 ± 1.50 | 2.27 ± 0.74 | 4.67 ± 1.78 | 4.51 ± 0.25 |
| Bladder | 6.81 ± 3.65 | 6.38 ± 1.44 | 1.43 ± 0.41 | 1.81 ± 0.37 | 14.26 ± 8.93 | 3.62 ± 0.04 |
| Bone | 3.32 ± 0.74 | 7.51 ± 2.99 | 3.83 ± 0.13 | 4.90 ± 0.53 | 4.02 ± 1.17 | 6.42 ± 3.81 |
| U87 | 7.79 ± 1.12 | 9.53 ± 2.09 | 4.01 ± 0.84 | 9.23 ± 1.57 | 3.43 ± 0.92 | 8.91 ± 3.90 |

### Tumor Weight:

| Group 2 | ⁶⁴Cu- | ⁶⁴Cu-07-56 | ⁶⁴Cu-07- | ⁶⁴Cu-07-05 | ⁶⁴Cu-07- | Group 2 | |
|---|---|---|---|---|---|---|---|
| ⁶⁴Cu-SRI- | FAPI- | Control | 56 G2 | Control | 05 G2 | ⁶⁴Cu-SRI- | ⁶⁴Cu- |
| 06-57 | 04 | (DOTAGA) | (DOTAGA | (DOTA) | (DOTA) | 06-57 | FAPI-04 |
| 0.05 | 0.05 | 0.03 | 0.05 | 0.07 | 0.18 | 0.11 | 0.28 |
| 0.05 | 0.03 | 0.06 | 0.07 | 0.02 | 0.28 | 0.09 | 0.01 |
| 0.05 | 0.04 | 0.08 | 0.03 | 0.02 | 0.16 | 0.06 | 0.02 |
| 0.05 ± 0.00 | 0.04 ± 0.01 | 0.06 ± 0.02 | 0.05 ± 0.01 | 0.04 ± 0.02 | 0.21 ± 0.05 | 0.09 ± 0.02 | 0.10 ± 0.12 |

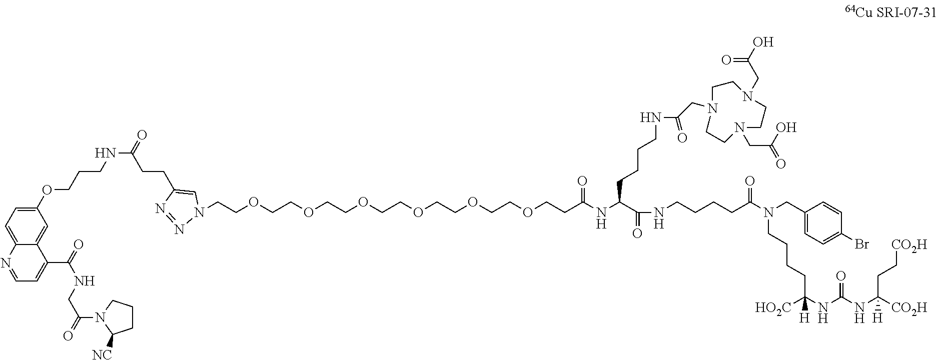 [SMILES: N#C[C@@H]1CCCN1C(=O)CNC(=O)c1ccnc2ccc(OCCCNC(=O)CCc3cn(CCOCCOCCOCCOCCOCCOCCC(=O)N[C@@H](CCCCNC(=O)CN4CCN(CC(=O)O)CCN(CC(=O)O)CC4)C(=O)NCCCCC(=O)N(CCCC[C@H](NC(=O)N[C@@H](CCC(=O)O)C(=O)O)C(=O)O)Cc4ccc(Br)cc4)nn3)cc12]

Dose: 20 μCi, n=3 at 1 h and n=4 at 2 and 4 h per group, Tumor model: SK-MEL-24 in NSG mice

| Animal #: | 1 h | 2 h | 4 h |
|---|---|---|---|
| Blood | 9.73 ± 0.29 | 6.36 ± 0.89 | 3.29 ± 0.07 |
| Heart | 4.36 ± 0.23 | 2.92 ± 0.27 | 1.87 ± 0.26 |
| Lung | 7.30 ± 1.16 | 5.76 ± 0.57 | 4.55 ± 0.51 |
| Liver | 5.30 ± 0.77 | 5.82 ± 0.72 | 6.03 ± 1.00 |
| Stomach | 3.29 ± 1.04 | 3.20 ± 0.85 | 2.23 ± 0.58 |
| Panc | 9.98 ± 0.99 | 6.66 ± 0.38 | 4.48 ± 0.88 |
| Spleen | 3.06 ± 0.46 | 2.56 ± 0.40 | 2.69 ± 0.44 |
| Fat | 0.80 ± 0.31 | 0.91 ± 0.83 | 0.49 ± 0.14 |
| Kidney | 8.08 ± 1.48 | 8.05 ± 1.27 | 7.26 ± 3.43 |
| Muscle | 2.99 ± 0.18 | 2.24 ± 0.42 | 1.32 ± 0.22 |
| Sm in | 6.52 ± 0.85 | 5.44 ± 1.52 | 3.54 ± 0.57 |
| Sal gl | 12.60 ± 2.46 | 9.121.51 ± | 4.47 ± 0.89 |
| Lac gl | 9.20 ± 1.74 | 7.00 ± 2.22 | 3.01 ± 1.94 |
| Bladder | 3.53 ± 0.52 | 4.76 ± 2.68 | 4.63 ± 1.72 |
| Bone | 6.00 ± 0.27 | 5.61 ± 1.73 | 3.42 ± 0.92 |
| SKMEL24 | 13.25 ± 1.32 | 12.48 ± 1.03 | 9.82 ± 3.39 |

### Tumor Weight:

| 1 h | 2 h | 4 h |
|---|---|---|
| 0.05 | 0.02 | 0.19 |
| 0.07 | 0.21 | 0.16 |
| 0.03 | 0.04 | 0.16 |
| | 0.06 | 0.13 |
| 0.05 | 0.08 | 0.16 |

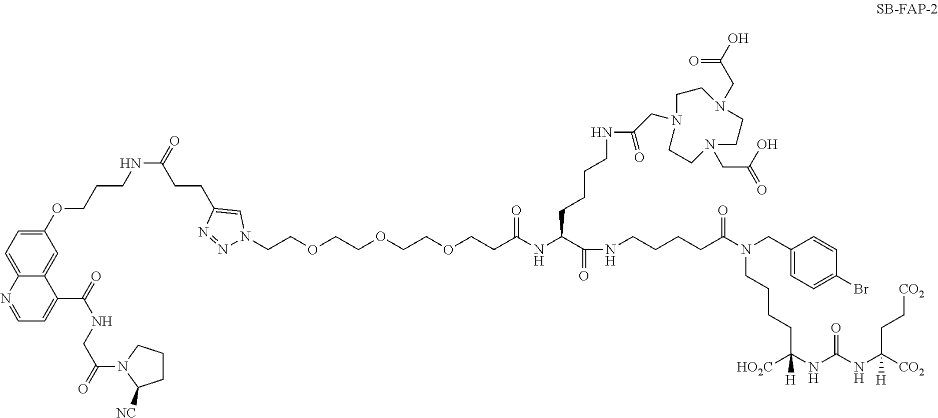

Tissue biodistribution data of ⁶⁴Cu-Sri-06-57 (SB-FAP-2) and ⁶⁴Cu-FAPI-04 in male NSG mice bearing U87 xenografts on the upper flank (Data are % ID/g, expressed as mean±SD) (n=3-4) at 2 h.

| | ⁶⁴Cu-FP-L1 | |
|---|---|---|
| | Group 3 | ⁶⁴Cu-FAPI-04 |
| Blood | 1.87 ± 0.24 | 3.43 ± 1.06 |
| Heart | 1.36 ± 0.28 | 1.85 ± 0.56 |
| Lung | 2.80 ± 0.68 | 3.03 ± 0.93 |
| Liver | 4.05 ± 0.53 | 2.56 ± 1.04 |
| Stomach | 1.89 ± 0.10 | 2.02 ± 0.94 |
| Pancreas | 0.99 ± 0.18 | 2.35 ± 0.88 |
| Spleen | 1.39 ± 0.10 | 1.46 ± 0.71 |
| Fat | 0.35 ± 0.20 | 0.56 ± 0.12 |
| Muscle | 1.11 ± 0.23 | 2.79 ± 0.56 |
| Kidney | 7.26 ± 2.83 | 2.16 ± 0.71 |
| Sm. Intestine | 2.32 ± 0.79 | 3.18 ± 0.95 |
| Salivary glands | 3.10 ± 0.51 | 10.08 ± 2.45 |
| Lacrimal glands | 4.74 ± 1.57 | 9.26 ± 5.95 |
| Bladder | 6.16 ± 4.37 | 6.38 ± 1.76 |
| Bone | 2.02 ± 0.79 | 7.51 ± 3.66 |
| U87 | 10.83 ± 1.02 | 9.53 ± 2.55 |

Tissue Biodistribution Data of ⁶⁴Cu-Sri-06-57 (SB-FAP-2), n=3-4

| | | 1 h | | 2 h | | |
|---|---|---|---|---|---|---|
| | | block ± | | Nude | | |
| | | 10 nmol | | mice | | |
| | 1 h | FAPI-04 | 2 h (n = 4) | (n = 4) | 4 h | 24 h |
| Blood | 10.12 ± 3.23 | 1.38 ± 0.19 | 9.93 ± 2.13 | 6.50 ± 1.05 | 3.91 ± 0.73 | 0.91 ± 0.23 |
| Heart | 3.42 ± 2.73 | 0.94 ± 0.35 | 4.62 ± 0.68 | 2.77 ± 0.25 | 2.13 ± 0.17 | 1.19 ± 0.34 |
| Lung | 5.32 ± 1.42 | 2.63 ± 0.27 | 5.65 ± 1.54 | 4.37 ± 0.48 | 3.31 ± 0.26 | 2.07 ± 0.40 |
| Liver | 5.35 ± 0.43 | 6.19 ± 1.06 | 6.15 ± 0.65 | 4.16 ± 0.66 | 5.52 ± 0.39 | 4.21 ± 1.00 |
| Stomach | 2.51 ± 0.25 | 1.96 ± 0.07 | 2.19 ± 0.33 | 2.02 ± 0.22 | 2.02 ± 0.30 | 1.27 ± 0.14 |
| Pancreas | 7.17 ± 1.19 | 1.01 ± 0.10 | 5.98 ± 0.58 | 5.04 ± 0.36 | 3.19 ± 0.11 | 0.72 ± 0.15 |
| Spleen | 2.20 ± 0.54 | 1.55 ± 0.07 | 2.68 ± 0.62 | 1.88 ± 0.35 | 2.05 ± 0.13 | 1.67 ± 0.32 |
| Fat | 0.77 ± 0.12 | 0.31 ± 0.04 | 0.66 ± 0.52 | 0.86 ± 0.03 | 0.53 ± 0.10 | 0.23 ± 0.10 |
| Kidney | 21.58 ± 5.33 | 6.09 ± 0.18 | 26.85 ± 7.39 | 11.66 ± 0.95 | 16.57 ± 2.68 | 2.45 ± 0.32 |
| Muscle | 2.29 ± 0.08 | 0.38 ± 0.05 | 2.10 ± 0.13 | 1.80 ± 0.26 | 1.08 ± 0.10 | 0.23 ± 0.04 |
| Sm.in | 4.22 ± 1.78 | 3.00 ± 0.22 | 6.12 ± 1.91 | 4.33 ± 1.13 | 4.26 ± 0.33 | 2.16 ± 0.29 |
| Sal.gl | 12.01 ± 3.62 | 2.00 ± 0.19 | 8.96 ± 1.47 | 5.60 ± 0.94 | 4.80 ± 0.82 | 0.92 ± 0.14 |
| Lac.gl | 5.47 ± 4.13 | 1.48 ± 0.45 | 8.11 ± 1.24 | 5.44 ± 0.78 | 3.72 ± 0.72 | 0.87 ± 0.14 |
| Bladder | 2.41 ± 0.62 | 1.22 ± 0.15 | 2.68 ± 0.83 | 1.80 ± 0.24 | 1.72 ± 0.33 | 0.57 ± 0.13 |
| Bone | 4.17 ± 0.13 | 0.72 ± 0.08 | 5.36 ± 1.89 | 3.51 ± 0.45 | 2.39 ± 0.37 | 0.48 ± 0.08 |
| Tumor | 12.04 ± 2.39 | 4.37 ± 0.70 | 14.65 ± 3.68 | 7.25 ± 1.24 | 13.81 ± 1.78 | 3.25 ± 0.05 |

### Tumor Size (NSG Mice, U87 Tumor)

| | 1 h | | | |
|---|---|---|---|---|
| 1 h | block | 2 h | 4 h | 24 h |
| 0.31 | 0.57 | 0.51 | 0.52 | 0.52 |
| 0.44 | 0.43 | 0.24 | 0.46 | 0.38 |
| 0.52 | 0.23 | 0.21 | 0.11 | 0.54 |
| | | 0.37 | | |

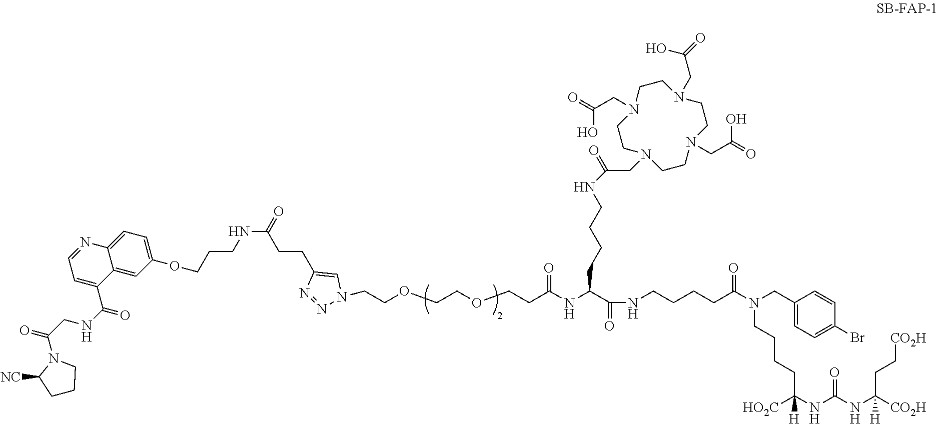

Biodistribution study using ⁵⁵Co-SB-FAP-01 (DOTA) AT 2 h (n=4), NCA. In MB-MDA-231 NSG mice (U87 tumors were not available), dose: ˜10 μCi.

| | | 2 h % ID/g | |
|---|---|---|---|
| | | (block) | At 1 h |
| | 2 h % | (FAPI-04) | nude mice |
| (N = 4) | ID/g | 30 nmol | SB-FAP-2 |
| Blood | 3.05 ± 0.35 | 0.300.05 | 8.47 ± 0.16 |
| Heart | 1.25 ± 0.08 | 0.16 ± 0.02 | 3.46 ± 0.07 |
| Lung | 2.41 ± 0.24 | 0.48 ± 0.04 | 5.16 ± 0.04 |
| Liver | 1.67 ± 0.31 | 0.33 ± 0.02 | 6.74 ± 0.16 |
| Stomach | 2.05 ± 0.46 | 0.20 ± 0.01 | 2.72 ± 0.30 |
| Pancreas | 4.47 ± 0.36 | 0.22 ± 0.03 | 4.95 ± 0.24 |
| Spleen | 2.20 ± 0.45 | 0.63 ± 0.10 | 2.38 ± 0.04 |
| Fat | 0.95 ± 0.26 | .14 ± 0.03 | 0.61 ± 0.31 |
| Kidney | 41.36 ± 5.68 | 20.04 ± 2.26 | 12.14 ± 1.39 |
| Muscle | 2.31 ± 0.23 | 0.08 ± 0.02 | 1.59 ± 0.07 |
| Small Intestine | 1.65 ± 0.32 | 0.22 ± 0.06 | 6.69 ± 0.82 |
| Salivary gland | 5.18 ± 0.53 | 0.30 ± 0.04 | 7.75 ± 0.99 |
| Lacrimal gland | 5.41 ± 0.74 | 0.22 ± 0.16 | 6.61 ± 1.23 |
| Bladder | 4.86 ± 1.55 | 1.62 ± 0.66 | 1.82 ± 0.26 |
| Bone | 4.96 ± 0.43 | 0.23 ± 0.04 | 3.34 ± 0.32 |
| MDA-MB-231 | 8.55 ± 3.56 | 0.51 ± 0.06 | 3.60 ± 0.32 |

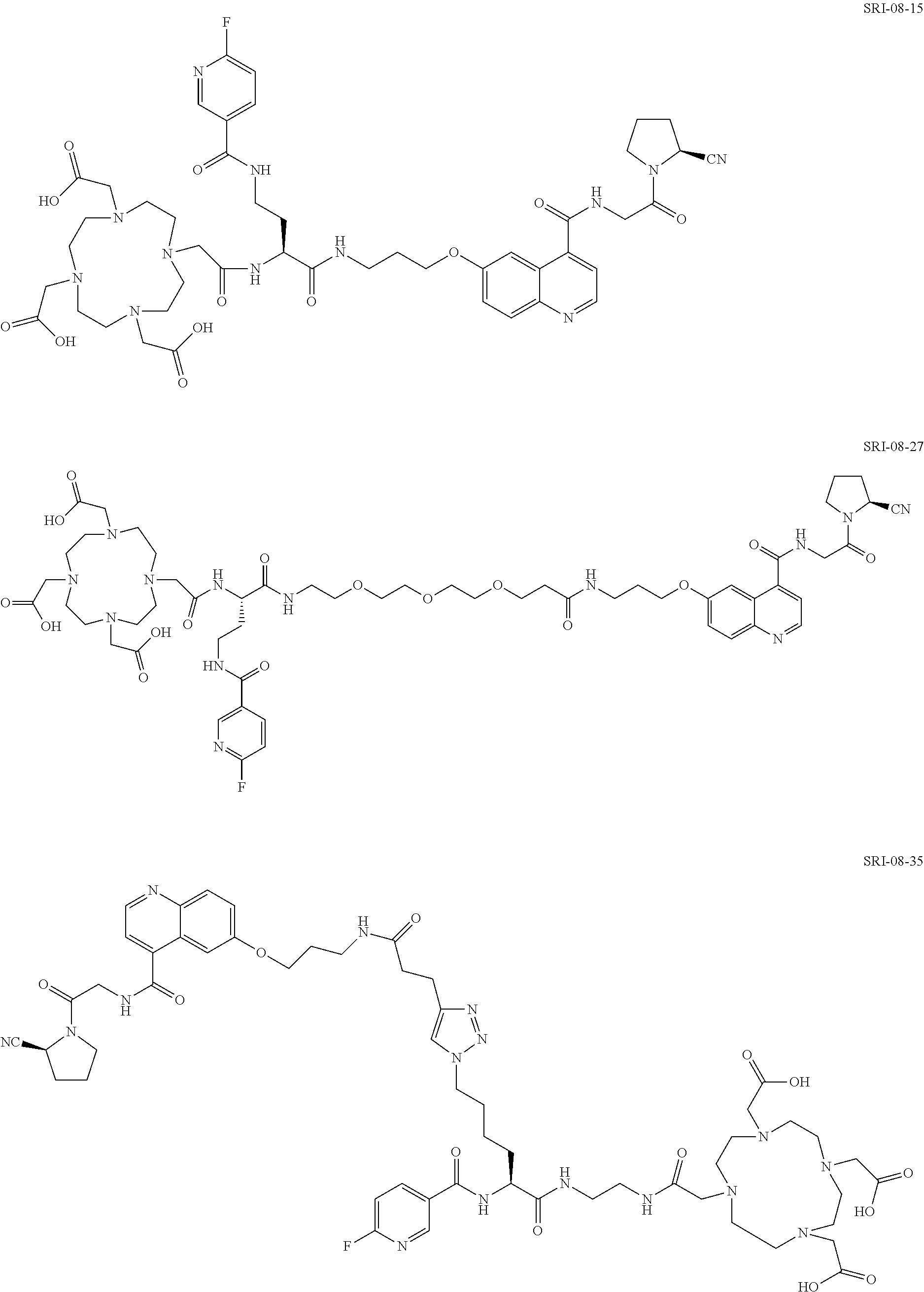 [SMILES: N#C[C@@H]1CCCN1C(=O)CNC(=O)c1ccnc2ccc(OCCCNC(=O)[C@H](CCNC(=O)c3ccc(F)nc3)NC(=O)CN3CCN(CC(=O)O)CCN(CC(=O)O)CCN(CC(=O)O)CC3)cc12 ; N#C[C@@H]1CCCN1C(=O)CNC(=O)c1ccnc2ccc(OCCCNC(=O)CCOCCOCCOCCNC(=O)[C@H](CCNC(=O)c3ccc(F)nc3)NC(=O)CN3CCN(CC(=O)O)CCN(CC(=O)O)CCN(CC(=O)O)CC3)cc12 ; N#C[C@@H]1CCCN1C(=O)CNC(=O)c1ccnc2ccc(OCCCNC(=O)CCc3cn(CCCC[C@H](NC(=O)c4ccc(F)nc4)C(=O)NCCNC(=O)CN4CCN(CC(=O)O)CCN(CC(=O)O)CCN(CC(=O)O)CC4)nn3)cc12]

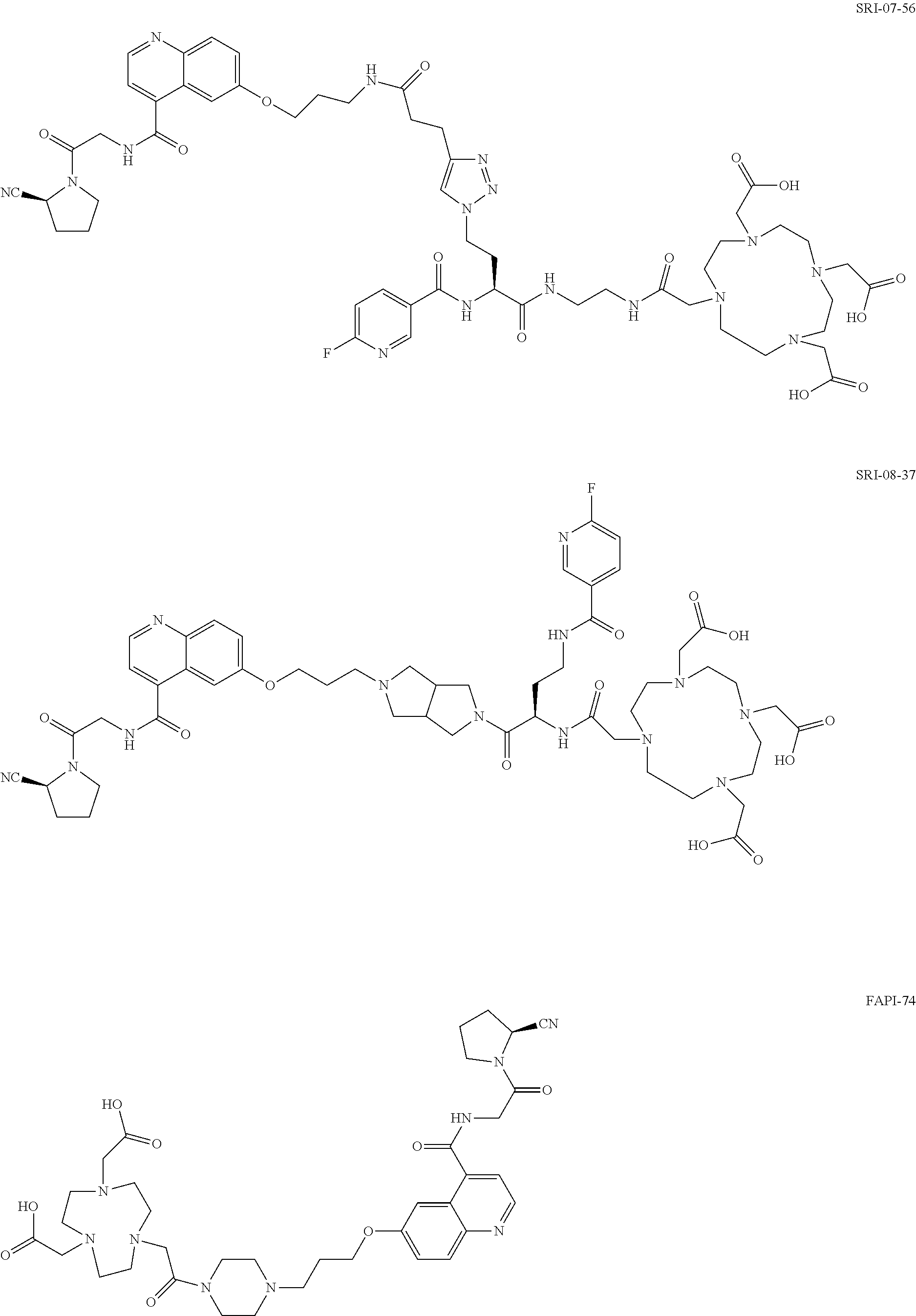 [SMILES: N#C[C@@H]1CCCN1C(=O)CNC(=O)c1ccnc2ccc(OCCCNC(=O)CCc3cn(CC[C@H](NC(=O)c4ccc(F)nc4)C(=O)NCCNC(=O)CN4CCN(CC(=O)O)CCN(CC(=O)O)CCN(CC(=O)O)CC4)nn3)cc12 ; N#C[C@@H]1CCCN1C(=O)CNC(=O)c1ccnc2ccc(OCCCN3CC4CN(C(=O)[C@@H](CCNC(=O)c5ccc(F)nc5)NC(=O)CN5CCN(CC(=O)O)CCN(CC(=O)O)CCN(CC(=O)O)CC5)CC4C3)cc12 ; N#C[C@@H]1CCCN1C(=O)CNC(=O)c1ccnc2ccc(OCCCN3CCN(C(=O)CN4CCN(CC(=O)O)CCN(CC(=O)O)CC4)CC3)cc12]

| | FAPI-74 | Sri-08-15 | Sri-08-35 | Sri-08-27 | Sri-07-56 | Sri-08-37 |
|---|---|---|---|---|---|---|
| Blood | 5.43 ± 0.55 | 3.92 ± 0.39 | 15.40 ± 1.08 | 12.65 ± 2.31 | 20.47 ± 8.29 | 4.59 ± 0.50 |
| Heart | 2.40 ± 0.16 | 1.97 ± 0.34 | 7.04 ± 0.93 | 5.39 ± 1.11 | 7.16 ± 3.22 | 2.24 ± 1.02 |
| Lung | 4.14 ± 0.39 | 2.90 ± 0.24 | 9.40 ± 1.61 | 7.76 ± 1.31 | 10.70 ± 4.41 | 2.96 ± 0.38 |
| Liver | 2.48 ± 0.39 | 2.73 ± 0.64 | 6.13 ± 0.67 | 5.15 ± 0.75 | 6.87 ± 2.54 | 2.02 ± 0.13 |
| Stomach | 2.52 ± 0.28 | 1.42 ± 0.56 | 2.17 ± 0.30 | 1.83 ± 0.66 | 3.73 ± 1.63 | 1.85 ± 0.19 |
| Pancreas | 13.26 ± 1.54 | 7.33 ± 1.73 | 10.46 ± 2.88 | 7.20 ± 3.00 | 10.62 ± 3.62 | 6.76 ± 1.47 |
| Spleen | 1.83 ± 0.49 | 1.59 ± 0.57 | 3.08 ± 0.27 | 2.59 ± 0.49 | 4.21 ± 1.74 | 1.45 ± 0.17 |
| Fat | 1.06 ± 0.18 | 0.92 ± 0.21 | 0.56 ± 0.20 | 0.45 ± 0.16 | 0.49 ± 0.23 | 1.04 ± 0.55 |
| Kidney | 3.83 ± 0.40 | 2.65 ± 0.27 | 5.47 ± 0.73 | 3.98 ± 0.66 | 5.16 ± 2.02 | 2.82 ± 0.06 |
| Muscle | 3.76 ± 0.55 | 3.03 ± 0.63 | 1.94 ± 0.74 | 1.55 ± 0.43 | 2.06 ± 0.95 | 2.74 ± 0.18 |
| Sm | 4.06 ± 0.62 | 3.85 ± 0.77 | 8.20 ± 2.20 | 6.28 ± 2.63 | 9.55 ± 3.91 | 3.36 ± 0.76 |
| Intest | | | | | | |
| Saliv. | 13.25 ± 3.07 | 8.95 ± 0.98 | 7.49 ± 1.17 | 6.40 ± 1.30 | 7.59 ± 3.44 | 11.70 ± 1.32 |
| Gland | | | | | | |
| Lacrim | 12.44 ± 1.85 | 8.01 ± 1.01 | 5.27 ± 1.52 | 4.62 ± 1.02 | 4.32 ± 1.56 | 9.11 ± 2.71 |
| Gland | | | | | | |
| Bladder | 2.37 ± 0.06 | 1.67 ± 0.32 | 1.59 ± 0.32 | 1.18 ± 0.13 | 7.14 ± 3.21 | 4.28 ± 4.14 |
| Bone | 8.17 ± 0.71 | 5.97 ± 1.02 | 3.75 ± 0.43 | 2.83 ± 0.36 | 4.47 ± 1.71 | 6.37 ± 0.98 |
| U87 | 21.00 ± 5.73 | 20.41 ± 3.28 | 7.84 ± 1.95 | 6.56 ± 1.76 | 5.67 ± 2.42 | 20.43 ± 1.73 |

### Example 10

### Synthesis of FAP-triamine Compounds

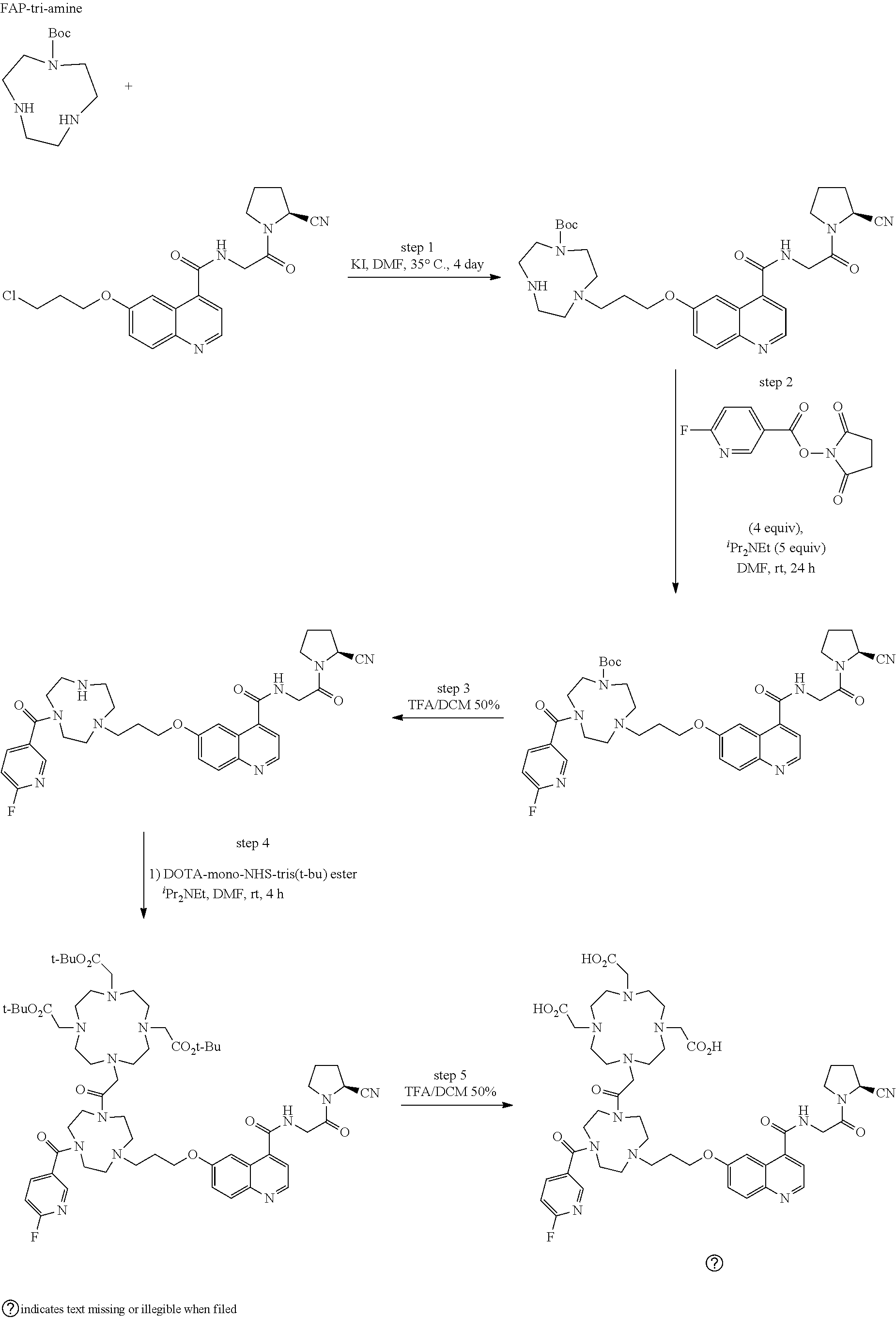

### Example 11

### Introduction of DOTA Chelator of Lysine

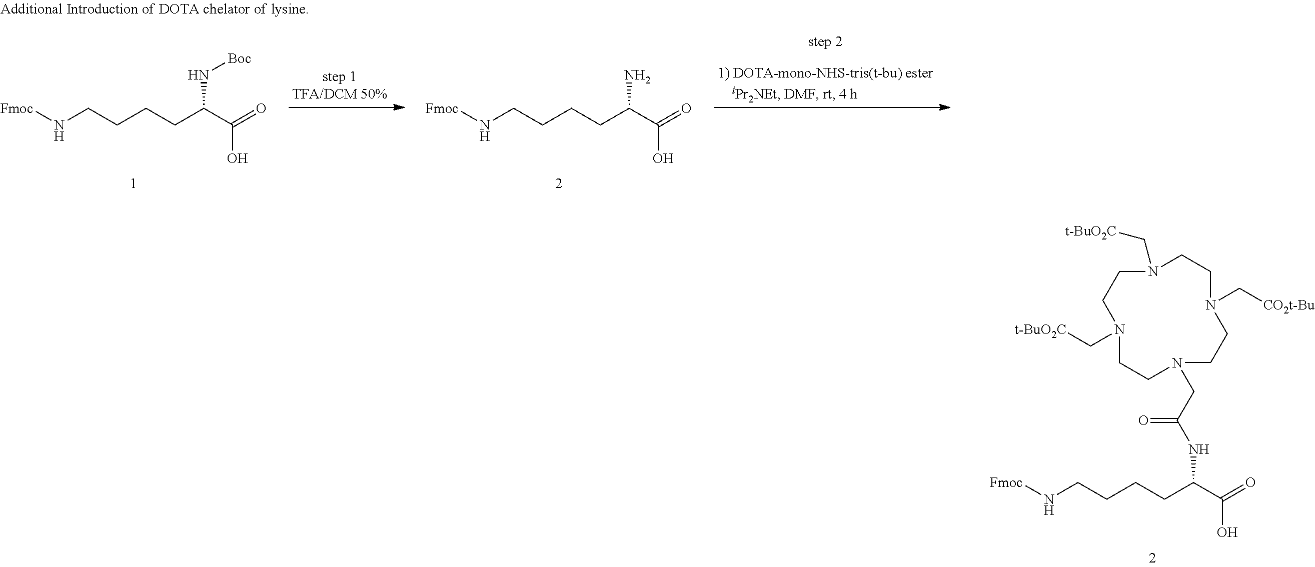

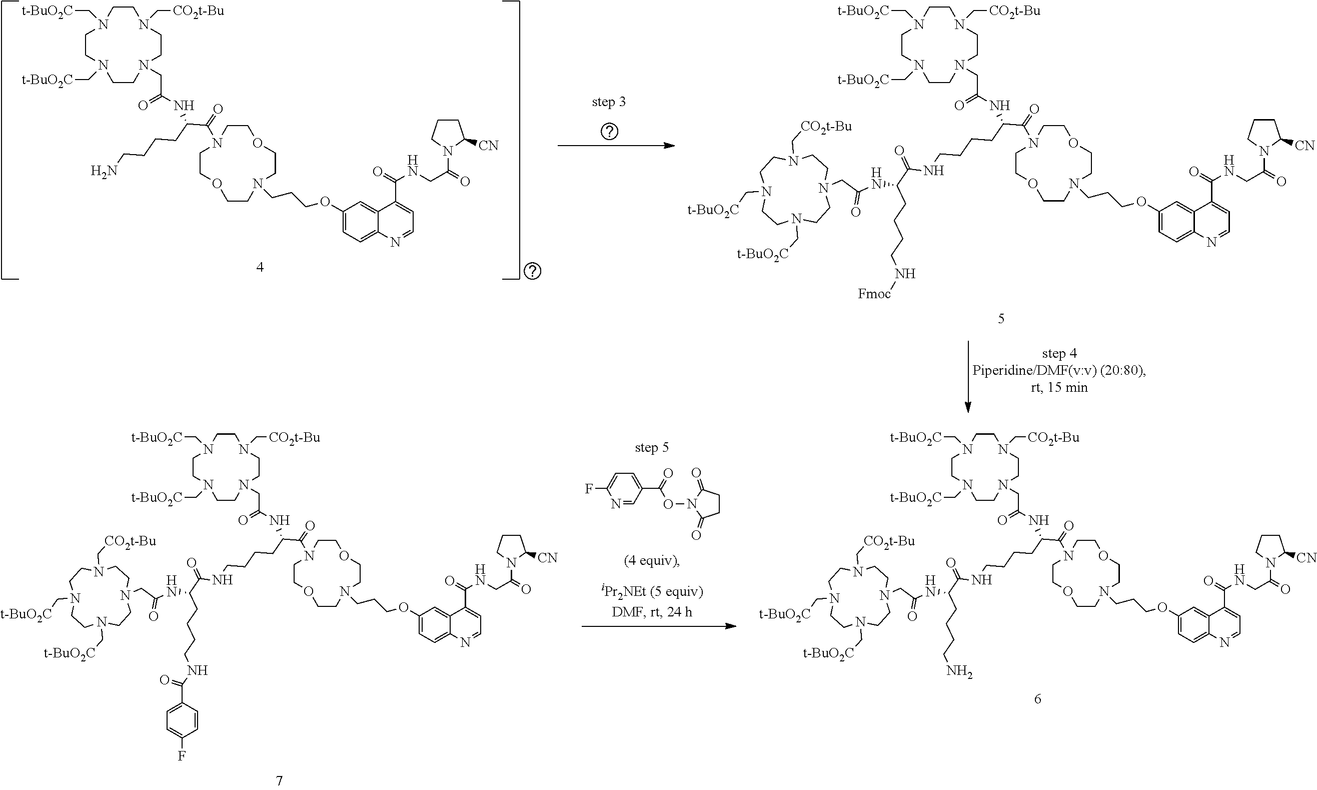

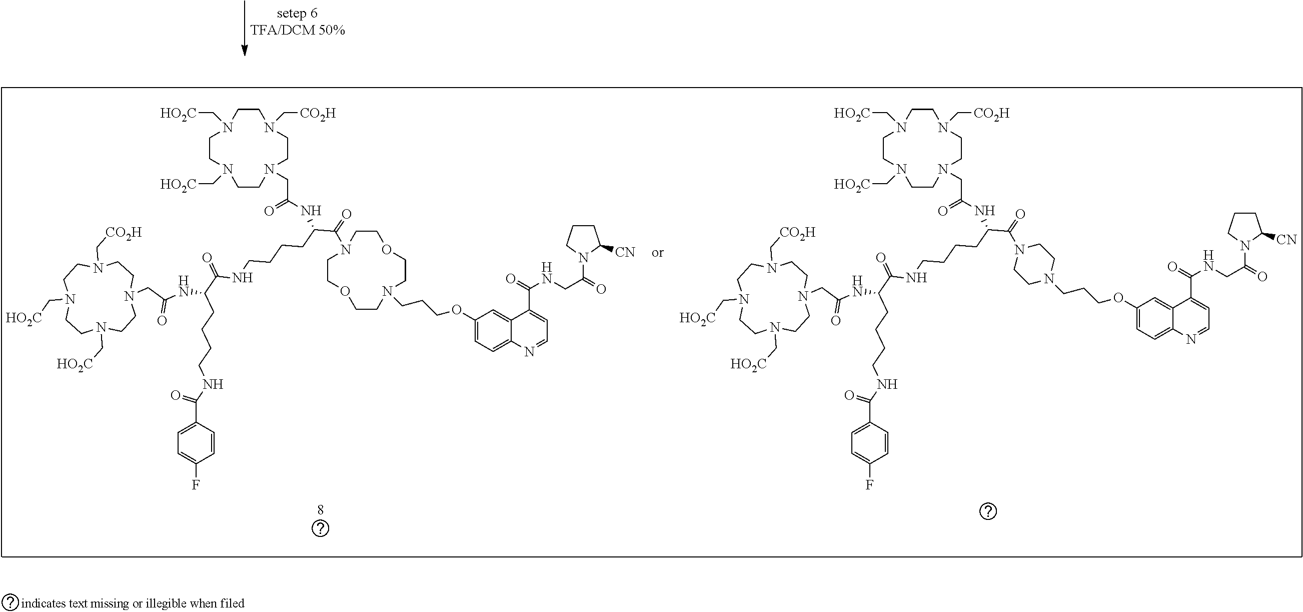 [SMILES: N#C[C@@H]1CCCN1C(=O)CNC(=O)c1ccnc2ccc(OCCCN3CCOCCN(C(=O)[C@H](CCCCNC(=O)[C@H](CCCCNC(=O)c4ccc(F)cc4)NC(=O)CN4CCN(CC(=O)O)CCN(CC(=O)O)CCN(CC(=O)O)CC4)NC(=O)CN4CCN(CC(=O)O)CCN(CC(=O)O)CCN(CC(=O)O)CC4)CCOCC3)cc12 ; N#C[C@@H]1CCCN1C(=O)CNC(=O)c1ccnc2ccc(OCCCN3CCN(C(=O)[C@H](CCCCNC(=O)[C@H](CCCCNC(=O)c4ccc(F)cc4)NC(=O)CN4CCN(CC(=O)O)CCN(CC(=O)O)CCN(CC(=O)O)CC4)NC(=O)CN4CCN(CC(=O)O)CCN(CC(=O)O)CCN(CC(=O)O)CC4)CC3)cc12]

### Example 12

### Sc-¹⁸F Labeled FAPI

Below is a scheme for the Sc-18F labeled FAPI and representative compounds.

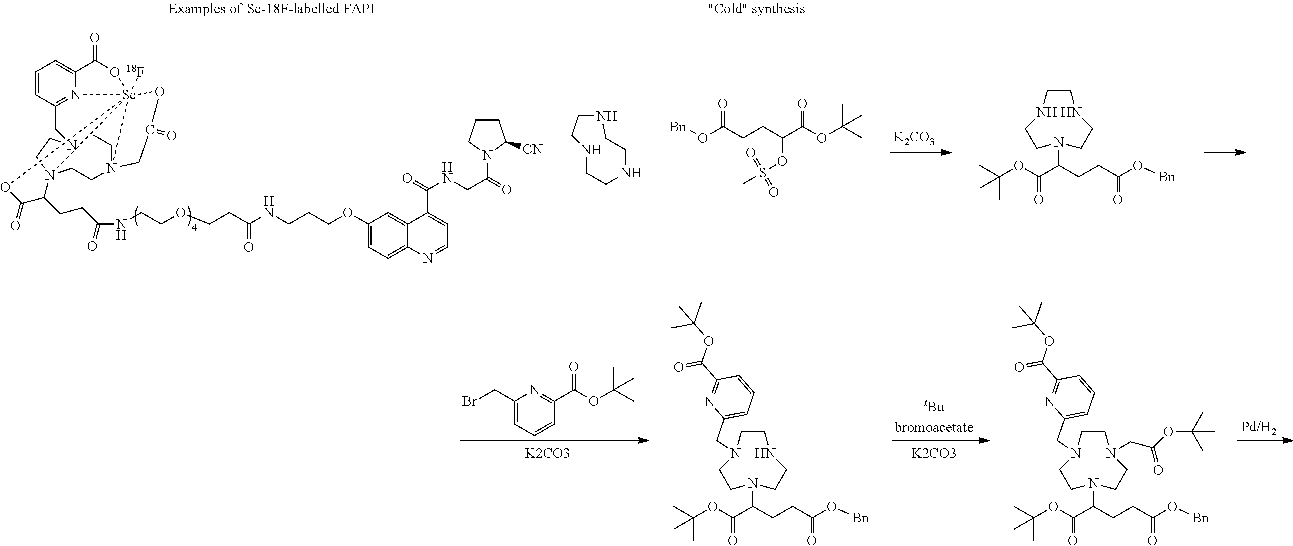

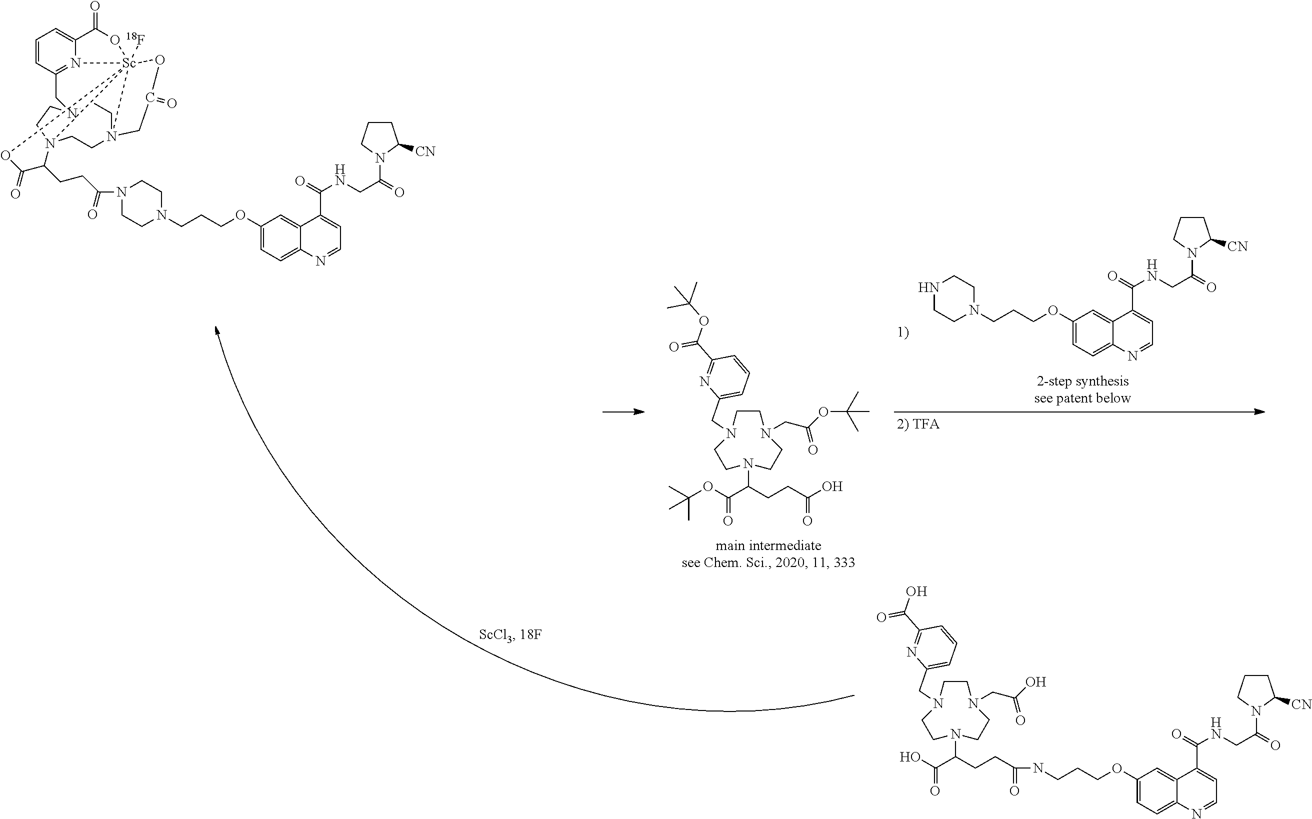

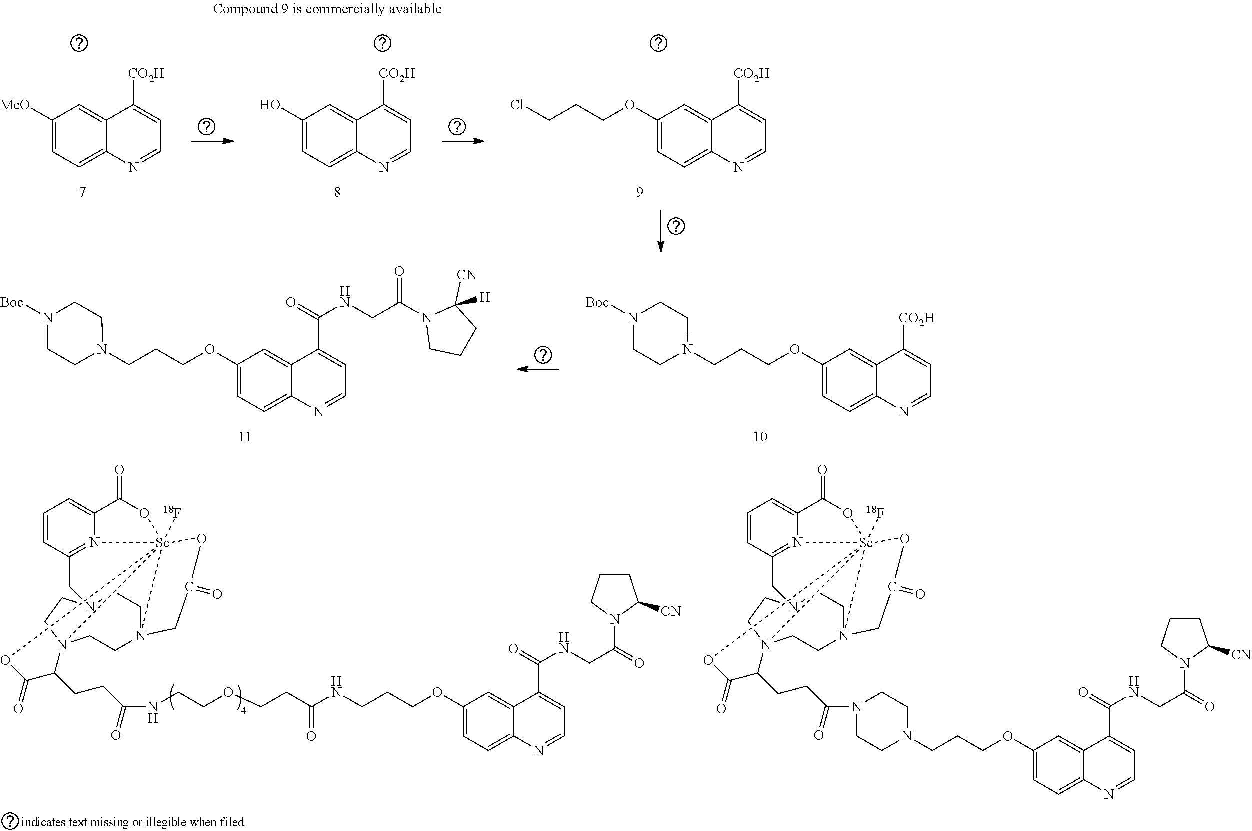

### Example 13

### Biodistribution of Representative Compounds

Biodistribution ([¹⁸F]JHU122047)

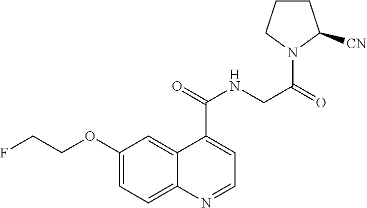 [SMILES: N#C[C@@H]1CCCN1C(=O)CNC(=O)c1ccnc2ccc(OCCF)cc12]

Injected dose: 26 μCi per mouse
Blocker: UAMC-01110
Vehicle: 12% DMSO in saline
Time: 1 hr post injection of the tracer (Blocker and vehicle was injected (i.p.) 15 min prior to the tracer injection
One of the blocker mouse has aberrant uptake in small and large intestine (highlighted)
[¹⁸F]JHU122047 + Vehicle

| | 1 | 2 | | | | |
|---|---|---|---|---|---|---|
| Animal #: | 28 | 27.7 | Compound: | % ID/g | | |
| Body Wt .: | % ID/g | % ID/g | Tissue | Mean | | Std. |
| Blood | 0.38 | 0.34 | Blood | 0.36 | ± | 0.03 |
| Heart | 0.35 | 0.35 | Heart | 0.35 | ± | 0.00 |
| Lung | 0.34 | 0.37 | Lung | 0.36 | ± | 0.02 |
| Liver | 0.67 | 0.59 | Liver | 0.68 | ± | 0.02 |
| Spleen | 0.26 | 0.27 | Spleen | 0.26 | ± | 0.01 |
| Pancreas | 0.33 | 0.32 | Pancreas. | 0.33 | ± | 0.00 |
| Stomach | 0.34 | 0.30 | Stomach | 0.32 | ± | 0.03 |
| Small intestine | 1.35 | 3.60 | Small intestine | 2.48 | ± | 1.59 |
| Large intestine | 0.42 | 1.57 | Large intestine | 0.99 | ± | 0.81 |
| Fat | 0.06 | 0.16 | Fat | 0.11 | ± | 0.07 |
| Muscle | 0.31 | 0.37 | Muscle | 0.34 | ± | 0.04 |
| Salivary gland | 0.63 | 0.47 | Salivary glend | 0.58 | ± | 0.15 |
| Brain | 0.14 | 0.15 | Brain | 0.14 | ± | 0.01 |
| Kidney | 1.94 | 0.56 | Kidney | 1.25 | ± | 0.97 |
| Bledder | 1.76 | 0.50 | Bladder | 1.13 | ± | 0.89 |
| Tumor | 2.20 | 1.72 | Tumor | 1.96 | ± | 0.34 |

[¹⁸F]JHU122047 + Blocker

| | 3 | 4 | | | | |
|---|---|---|---|---|---|---|
| Animal #: | 22.7 | 23.1 | Compound: | % ID/g | | |
| Body Wt.: | % ID/g | % ID/g | Tissue | Mean | | Std. |
| Blood | 0.28 | 0.08 | Blood | 0.12 | ± | 0.15 |
| Heart | 0.26 | 0.51 | Heart | 0.38 | ± | 0.17 |
| Lung | 0.25 | 0.41 | Lung | 0.33 | ± | 0.11 |
| Liver | 0.45 | 0.87 | Liver | 0.66 | ± | 0.30 |
| Spleen | 0.19 | 0.37 | Spleen | 0.28 | ± | 0.18 |
| Pancreas | 0.16 | 0.29 | Pancreas | 0.23 | ± | 0.09 |
| Stomach | 0.41 | 0.59 | Stomach | 0.50 | ± | 0.12 |
| Small intestine | 1.12 | 15.94 | Small intestine | 8.53 | ± | 10.48 |
| Large intestine | 0.70 | 3.68 | Large intestine | 2.19 | ± | 2.11 |
| Fat | 0.05 | 0.11 | Fat | 0.08 | ± | 0.04 |
| Muscle | 0.20 | 0.35 | Muscle | 0.27 | ± | 0.11 |
| Salivary gland | 0.20 | 0.36 | Salivary gland | 0.28 | ± | 0.11 |
| Brain | 0.17 | 0.22 | Brain | 0.19 | ± | 0.04 |
| Kidney | 0.35 | 0.85 | Kidney | 0.60 | ± | 0.35 |
| Bledder | 1.78 | 2.43 | Bledder | 2.10 | ± | 0.46 |
| Tumor | 0.32 | 0.59 | Tumor | 0.45 | ± | 0.19 |

Biodistribution ([¹⁸F]JHU1221785)

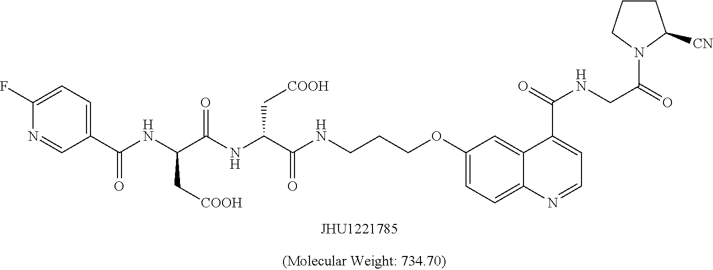 [SMILES: N#C[C@@H]1CCCN1C(=O)CNC(=O)c1ccnc2ccc(OCCCNC(=O)[C@@H](CC(=O)O)NC(=O)[C@@H](CC(=O)O)NC(=O)c3ccc(F)nc3)cc12]

Injected dose: 20 μCi per mouse (SA: 240.5 GBq/μmol; 6500 mCi/μmol)
Blocker: Cold FAPI-04 (10 nmole, 6500-fold access)
Time: 30 and 60post injection of the tracer (Blocker was injected simultaneously with the
[¹⁸F]JHU1221785, 30 min

| | 22.2 | 22.6 | | % ID/g | | |
|---|---|---|---|---|---|---|
| Body Wt.: | % ID/g | % ID/g | Tissue | Mean | | Std. |
| Blood | 5.98 | 3.90 | Blood | 4.94 | ± | 1.47 |
| Heart | 2.51 | 2.01 | Heart | 2.26 | ± | 0.35 |
| Lung | 2.77 | 2.53 | Lung | 2.65 | ± | 0.17 |
| Liver | 6.56 | 8.08 | Liver | 7.32 | ± | 1.08 |
| Spleen | 1.03 | 0.97 | Spleen | 1.00 | ± | 0.04 |
| Pancrees | 1.92 | 2.71 | Pancreas | 2.31 | ± | 0.56 |
| Stomach | 0.72 | 0.92 | Stomach | 0.82 | ± | 0.14 |
| Small intestine | 2.60 | 2.38 | Small intestine | 2.49 | ± | 0.16 |
| Large intestine | 0.58 | 0.86 | Large intestine | 0.72 | ± | 0.20 |
| Fat | 0.21 | 0.19 | Fat | 0.20 | ± | 0.01 |
| Muscle | 0.81 | 1.12 | Muscle | 0.96 | ± | 0.22 |
| Salivery gland | 2.81 | 3.05 | Salivary gland | 2.93 | ± | 0.17 |
| Brain | 0.14 | 0.15 | Brain | 0.14 | ± | 0.01 |
| Kidney | 1.60 | 3.33 | Kidney | 2.47 | ± | 1.23 |
| Bladder | 0.93 | 4.69 | Bladder | 2.81 | ± | 2.66 |
| Tumor | 1.14 | 1.34 | Tumor | 1.24 | ± | 0.14 |
| Femur | 2.78 | 2.44 | | 2.61 | ± | 0.24 |

[¹⁸F]JHU1221785, 60 min

| | 25.5 | 24.5 | | % ID/g | | |
|---|---|---|---|---|---|---|
| Body Wt.: | % ID/g | % ID/g | Tissue | Mean | | Std. |
| Blood | 2.56 | 4.57 | Blood | 3.57 | ± | 1.42 |
| Heart | 1.38 | 1.73 | Heart | 1.56 | ± | 0.25 |
| Lung | 1.70 | 2.65 | Lung | 2.18 | ± | 0.67 |
| Liver | 7.41 | 8.27 | Liver | 7.84 | ± | 0.61 |
| Spleen | 0.78 | 1.09 | Spleen | 0.93 | ± | 0.22 |
| Pancreas | 1.96 | 2.17 | Pancreas | 2.07 | ± | 0.15 |
| Stomach | 0.60 | 0.81 | Stomach | 0.71 | ± | 0.15 |
| Small intestine | 1.65 | 2.01 | Small intestine | 1.83 | ± | 0.26 |
| Large intestine | 0.61 | 0.78 | Large intestine | 0.69 | ± | 0.12 |
| Fat | 0.22 | 0.21 | Fat | 0.22 | ± | 0.01 |
| Muscle | 1.06 | 0.89 | Muscle | 0.98 | ± | 0.12 |
| Salivery gland | 2.17 | 4.73 | Salivary gland | 3.45 | ± | 1.80 |
| Brain | 0.09 | 0.12 | Brain | 0.10 | ± | 0.08 |
| Kidney | 0.79 | 1.04 | Kidney | 0.91 | ± | 0.18 |
| Bladder | 0.76 | 0.92 | Bladder | 0.84 | ± | 0.12 |
| Tumor | 2.59 | 1.47 | Tumor | 2.23 | ± | 1.07 |
| Femur | 2.06 | 2.07 | | 2.07 | ± | 0.01 |

[¹⁸F]JHU1221785 + Blocker, 60 min

| | 22.7 | 23.1 | | % ID/g | | |
|---|---|---|---|---|---|---|
| Body Wt.: | % ID/g | % ID/g | Tissue | Mean | | Std. |
| Blood | 0.11 | 9.26 | Blood | 4.68 | ± | 6.47 |
| Heart | 0.06 | 0.07 | Heart | 0.07 | ± | 0.01 |
| Lung | 0.13 | 0.20 | Lung | 0.17 | ± | 0.05 |
| Liver | 9.42 | 10.36 | Liver | 9.89 | ± | 0.67 |
| Spleen | 0.08 | 0.25 | Spleen | 0.16 | ± | 0.12 |
| Pancreas | 0.05 | 0.22 | Pancreas | 0.13 | ± | 0.12 |
| Stomach | 0.09 | 0.23 | Stomach | 0.16 | ± | 0.10 |
| Small intestine | 0.88 | 6.19 | Small intestine | 3.54 | ± | 3.75 |
| Large intestine | 0.08 | 0.33 | Large intestine | 0.20 | ± | 0.18 |
| Fat | 0.03 | 0.21 | Fat | 0.12 | ± | 0.13 |
| Muscle | 0.04 | 0.07 | Muscle | 0.06 | ± | 0.02 |
| Salivery gland | 0.04 | 0.06 | Salivary gland | 0.05 | ± | 0.01 |
| Brain | 0.02 | 0.04 | Brain | 0.03 | ± | 0.01 |
| Kidney | 0.41 | 0.51 | Kidney | 0.46 | ± | 0.07 |
| Bladder | 0.23 | 0.67 | Bladder | 0.45 | ± | 0.31 |
| Tumor | 0.08 | 0.11 | Tumor | 0.10 | ± | 0.02 |
| Femur | 1.52 | 1.50 | | 1.51 | ± | 0.01 |

Biodistribution ([¹⁸F]JHU1221823)

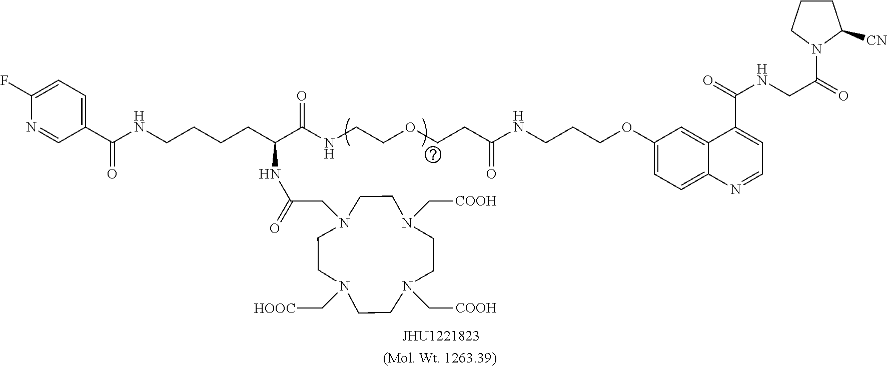

Injected dose: 2 μCi per mouse
Blocker: Cold FAPI-04
Time: 30 and 60 min post injection of the tracer (Blocker was injected simultaneously with tracer)
[¹⁸F]JHU1221823, 30 min

| | 22 | 24.8 | | % ID/g | | |
|---|---|---|---|---|---|---|
| Body Wt.: | % ID/g | % ID/g | Tissue | Mean | | Std. |
| Blood | 11.89 | 9.72 | Blood | 10.81 | ± | 1.54 |
| Heart | 5.25 | 4.76 | Heart | 5.01 | ± | 0.34 |
| Lung | 6.92 | 5.83 | Lung | 6.38 | ± | 0.78 |
| Liver | 2.58 | 2.30 | Liver | 2.44 | ± | 0.20 |
| Spleen | 2.08 | 2.12 | Spleen | 2.08 | ± | 0.05 |
| Pancreas | 3.58 | 2.88 | Pancreas | 3.13 | ± | 0.35 |
| Stomach | 1.84 | 1.21 | Stomach | 1.52 | ± | 0.45 |
| Small intestine | 3.08 | 2.48 | Small intestine | 2.78 | ± | 0.42 |
| Large intestine | 1.61 | 2.67 | Large intestine | 2.14 | ± | 0.75 |
| Fat | 0.41 | 0.43 | Fat | 0.42 | ± | 0.01 |
| Muscle | 1.74 | 1.71 | Muscle | 1.73 | ± | 0.02 |
| Salivery gland | 7.35 | 9.57 | Salivary gland | 8.51 | ± | 1.84 |
| Brain | 0.26 | 0.31 | Brain | 0.29 | ± | 0.04 |
| Kidney | 3.37 | 2.96 | Kidney | 3.17 | ± | 0.28 |
| Bladder | 1.53 | 3.45 | Bladder | 2.49 | ± | 1.36 |
| Tumor | 2.90 | 3.32 | Tumor | 3.11 | ± | 0.30 |
| Femur | 3.95 | 3.45 | Femur | 3.50 | ± | 0.07 |

[¹⁸F]JHU1221823, 60 min

| | 25.5 | 24.5 | | % ID/g | | |
|---|---|---|---|---|---|---|
| Body Wt.: | % ID/g | % ID/g | Tissue | Mean | | Std. |
| Blood | 9.50 | 10.76 | Blood | 10.03 | ± | 1.03 |
| Heart | 4.67 | 4.58 | Heart | 4.52 | ± | 0.21 |
| Lung | 5.36 | 6.75 | Lung | 6.05 | ± | 0.98 |
| Liver | 2.49 | 2.75 | Liver | 2.62 | ± | 0.19 |
| Spleen | 2.25 | 2.45 | Spleen | 2.35 | ± | 0.14 |
| Pancreas | 5.44 | 3.95 | Pancreas | 3.70 | ± | 0.36 |
| Stomach | 1.68 | 1.57 | Stomach | 1.68 | ± | 0.07 |
| Small intestine | 8.18 | 3.18 | Small intestine | 5.18 | ± | 0.00 |
| Large intestine | 1.70 | 5.45 | Large intestine | 5.58 | ± | 2.65 |
| Fat | 0.53 | 0.58 | Fat | 0.56 | ± | 0.04 |
| Muscle | 1.76 | 1.75 | Muscle | 1.75 | ± | 0.00 |
| Salivery gland | 9.14 | 8.79 | Salivary gland | 8.97 | ± | 0.25 |
| Brain | 0.40 | 0.50 | Brain | 0.45 | ± | 0.07 |
| Kidney | 2.76 | 2.75 | Kidney | 2.75 | ± | 0.02 |
| Bladder | 3.64 | 7.58 | Bladder | 5.51 | ± | 2.64 |
| Tumor | 5.22 | 7.17 | Tumor | 6.20 | ± | 1.88 |
| Femur | 3.21 | 3.87 | Femur | 3.54 | ± | 0.47 |

[¹⁸F]JHU1221823 + Blocker, 60 min

| | 22.7 | 23.1 | | % ID/g | | |
|---|---|---|---|---|---|---|
| Body Wt.: | % ID/g | % ID/g | Tissue | Mean | | Std. |
| Blood | 0.22 | 0.28 | Blood | 0.25 | ± | 0.04 |
| Heart | 0.22 | 0.33 | Heart | 0.28 | ± | 0.08 |
| Lung | 0.54 | 0.89 | Lung | 0.72 | ± | 0.25 |
| Liver | 0.35 | 0.51 | Liver | 0.44 | ± | 0.12 |
| Spleen | 0.29 | 0.38 | Spleen | 0.51 | ± | 0.03 |
| Pancreas | 0.27 | 0.46 | Pancreas | 0.37 | ± | 0.13 |
| Stomach | 0.28 | 0.40 | Stomach | 0.34 | ± | 0.08 |
| Small intestine | 0.91 | 1.06 | Small intestine | 0.99 | ± | 0.11 |
| Large intestine | 0.41 | 0.52 | Large intestine | 0.47 | ± | 0.07 |
| Fat | 0.15 | 0.27 | Fat | 0.21 | ± | 0.08 |
| Muscle | 0.25 | 0.61 | Muscle | 0.48 | ± | 0.25 |
| Salivery gland | 0.37 | 0.48 | Salivary gland | 0.42 | ± | 0.08 |
| Brain | 0.06 | 0.11 | Brain | 0.09 | ± | 0.04 |
| Kidney | 1.85 | 1.69 | Kidney | 1.77 | ± | 0.11 |
| Bladder | 2.62 | 2.04 | Bladder | 2.33 | ± | 0.41 |
| Tumor | 0.89 | 1.19 | Tumor | 1.04 | ± | 0.21 |
| Femur | 0.98 | 2.58 | Femur | 1.78 | ± | 1.18 |

Biodistribution ([¹⁸F]SB-FAP-09)
Injected dose: 20 μCi per mouse (SA: 19.05 GBq/μmol; 515.01 mCi/μmol)
Blocker: FAPI-04 (10 nmole, 257.7 fold access)
Time: 1 hr post injection of the tracer (Blocker and vehicle was injected (i.p.) 15 min prior to the tracer injection
One of the blocker mouse has aberrant uptake in small and large intestine (highlighted)
[¹⁸F]SB-FAP-09

| | 23.2 | 22.7 | | % ID/g | | |
|---|---|---|---|---|---|---|
| Body Wt.: | % ID/g | % ID/g | Tissue | Mean | | Std. |
| Blood | 5.93 | 5.79 | Blood | 5.86 | ± | 0.10 |
| Heart | 2.70 | 2.65 | Heart | 2.67 | ± | 0.04 |
| Lung | 3.40 | 3.30 | Lung | 3.35 | ± | 0.07 |
| Liver | 4.93 | 4.52 | Liver | 4.76 | ± | 0.29 |
| Spleen | 1.92 | 2.83 | Spleen | 2.38 | ± | 0.64 |
| Pancreas | 4.60 | 3.58 | Pancreas | 4.09 | ± | 0.72 |
| Stomach | 0.83 | 1.09 | Stomach | 0.96 | ± | 0.19 |
| Small intestine | 1.41 | 1.46 | Small intestine | 1.44 | ± | 0.04 |
| Large intestine | 1.23 | 1.08 | Large intestine | 1.15 | ± | 0.11 |
| Fat | 0.30 | 0.24 | Fat | 0.27 | ± | 0.04 |
| Muscle | 1.86 | 1.50 | Muscle | 1.68 | ± | 0.26 |
| Salivery gland | 6.81 | 3.89 | Salivary gland | 5.35 | ± | 2.07 |
| Brain | 0.19 | 0.18 | Brain | 0.19 | ± | 0.01 |
| Kidney | 1.39 | 1.36 | Kidney | 1.37 | ± | 0.02 |
| Bladder | 1.16 | 1.53 | Bladder | 1.35 | ± | 0.27 |
| Tumor | 3.12 | 5.36 | Tumor | 4.24 | ± | 1.59 |

[¹⁸F]SB-FAP-09 + Blocker

| | 22.7 | 23.1 | | % ID/g | | |
|---|---|---|---|---|---|---|
| Body Wt.: | % ID/g | % ID/g | Tissue | Mean | | Std. |
| Blood | 0.27 | 0.33 | Blood | 0.30 | ± | 0.04 |
| Heart | 0.21 | 0.21 | Heart | 0.21 | ± | 0.00 |
| Lung | 0.86 | 0.62 | Lung | 0.74 | ± | 0.17 |
| Liver | 3.99 | 5.79 | Liver | 4.89 | ± | 1.27 |
| Spleen | 2.43 | 2.20 | Spleen | 2.31 | ± | 0.16 |
| Pancreas | 0.18 | 0.21 | Pancreas | 0.19 | ± | 0.02 |
| Stomach | 0.22 | 0.29 | Stomach | 0.26 | ± | 0.05 |
| Small intestine | 0.43 | 0.28 | Small intestine | 0.35 | ± | 0.11 |
| Large intestine | 0.27 | 0.30 | Large intestine | 0.28 | ± | 0.03 |
| Fat | 0.10 | 0.05 | Fat | 0.07 | ± | 0.04 |
| Muscle | 0.48 | 0.17 | Muscle | 0.33 | ± | 0.22 |
| Salivery gland | 0.26 | 0.37 | Salivary gland | 0.32 | ± | 0.08 |
| Brain | 0.05 | 0.06 | Brain | 0.06 | ± | 0.00 |
| Kidney | 0.80 | 0.90 | Kidney | 0.85 | ± | 0.07 |
| Bladder | 0.86 | 0.37 | Bladder | 0.61 | ± | 0.34 |
| Tumor | 1.14 | 0.77 | Tumor | 0.95 | ± | 0.26 |

Biodistribution ([¹⁸F]SB-FAP-09). 2nd
Injected dose: 20 μCi per mouse (SA: 102.7 GBq/μmol; 2775.7 mCi/μmol)
Blocker: FAPI-04 (10 nmole, 1387-fold access)
Time: 15, 30, 60, and 120 min post injection of the tracer (Blocker was injected simultaneously with the tracer)
[¹⁸F]SB-FAP-09, 15 min

| | 22.7 | 23.1 | | % ID/g | | |
|---|---|---|---|---|---|---|
| Body Wt.: | % ID/g | % ID/g | Tissue | Mean | | Std. |
| Blood | 2.18 | 2.36 | Blood | 2.27 | ± | 0.13 |
| Heart | 1.69 | 1.93 | Heart | 1.81 | ± | 0.17 |
| Lung | 1.71 | 1.80 | Lung | 1.76 | ± | 0.06 |
| Liver | 1.54 | 1.87 | Liver | 1.70 | ± | 0.23 |
| Spleen | 1.04 | 1.34 | Spleen | 1.19 | ± | 0.21 |
| Pancreas | 2.21 | 1.95 | Pancreas | 2.08 | ± | 0.18 |
| Stomach | 0.87 | 0.93 | Stomach | 0.90 | ± | 0.05 |
| Small intestine | 1.40 | 1.73 | Small intestine | 1.57 | ± | 0.23 |
| Large intestine | 1.27 | 1.46 | Large intestine | 1.37 | ± | 0.13 |
| Fat | 0.36 | 0.29 | Fat | 0.33 | ± | 0.05 |
| Muscle | 1.75 | 2.09 | Muscle | 1.92 | ± | 0.25 |
| Salivery gland | 2.94 | 3.25 | Salivary gland | 3.09 | ± | 0.22 |
| Brain | 0.13 | 0.13 | Brain | 0.13 | ± | 0.00 |
| Kidney | 2.46 | 2.60 | Kidney | 2.53 | ± | 0.10 |
| Bladder | 2.99 | 2.43 | Bladder | 2.71 | ± | 0.40 |
| Tumor | 2.88 | 3.37 | Tumor | 3.18 | ± | 0.34 |
| Femur | 11.17 | 13.83 | Femur | 12.50 | ± | 1.88 |

[¹⁸F]SB-FAP-09, 30 min

| | 22.1 | 28.7 | | % ID/g | | |
|---|---|---|---|---|---|---|
| Body Wt.: | % ID/g | % ID/g | Tissue | Mean | | Std. |
| Blood | 1.99 | 1.86 | Blood | 1.92 | ± | 0.09 |
| Heart | 1.21 | 1.37 | Heart | 1.29 | ± | 0.11 |
| Lung | 1.23 | 1.26 | Long | 1.25 | ± | 0.02 |
| Liver | 1.02 | 1.03 | Liver | 1.03 | ± | 0.01 |
| Spleen | 0.80 | 0.91 | Spleen | 0.86 | ± | 0.08 |
| Pancreas | 1.39 | 1.99 | Pancreas | 1.69 | ± | 0.43 |
| Stomach | 0.77 | 0.67 | Stomach | 0.72 | ± | 0.07 |
| Small intestine | 0.90 | 1.06 | Small intestine | 0.98 | ± | 0.11 |
| Large intestine | 0.86 | 0.99 | Large intestine | 0.92 | ± | 0.09 |
| Fat | 0.18 | 0.32 | Fat | 0.25 | ± | 0.10 |
| Muscle | 1.50 | 1.43 | Muscle | 1.47 | ± | 0.05 |
| Salivery gland | 2.43 | 3.05 | Salivary gland | 2.74 | ± | 0.44 |
| Brain | 0.17 | 0.76 | Brain | 0.46 | ± | 0.42 |
| Kidney | 1.26 | 1.31 | Kidney | 1.29 | ± | 0.03 |
| Bladder | 8.39 | 4.00 | Bladder | 6.20 | ± | 3.11 |
| Tumor | 5.74 | 3.04 | Tumor | 4.39 | ± | 1.91 |
| Femur | 26.68 | 12.07 | Femur | 19.38 | ± | 10.33 |

[¹⁸F]SB-FAP-09, 60 min

| | 22.5 | 24.2 | | % ID/g | | |
|---|---|---|---|---|---|---|
| Body Wt.: | % ID/g | % ID/g | Tissue | Mean | | Std. |
| Blood | 1.25 | 1.25 | Blood | 1.25 | ± | 0.00 |
| Heart | 0.79 | 0.91 | Heart | 0.85 | ± | 0.08 |
| Lung | 0.96 | 0.78 | Lung | 0.87 | ± | 0.13 |
| Liver | 0.60 | 0.55 | Liver | 0.57 | ± | 0.04 |
| Spleen | 0.63 | 0.46 | Spleen | 0.54 | ± | 0.12 |
| Pancreas | 1.94 | 2.16 | Pancreas | 2.05 | ± | 0.16 |
| Stomach | 0.38 | 0.47 | Stomach | 0.42 | ± | 0.07 |
| Small intestine | 0.70 | 0.69 | Small intestine | 0.70 | ± | 0.01 |
| Large intestine | 0.71 | 0.94 | Large intestine | 0.82 | ± | 0.16 |
| Fat | 0.47 | 0.80 | Fat | 0.38 | ± | 0.12 |
| Muscle | 1.79 | 0.98 | Muscle | 1.38 | ± | 0.57 |
| Salivery gland | 2.62 | 3.08 | Salivary gland | 2.85 | ± | 0.32 |
| Brain | 0.10 | 0.16 | Brain | 0.13 | ± | 0.04 |
| Kidney | 0.77 | 0.75 | Kidney | 0.76 | ± | 0.01 |
| Bladder | 6.35 | 1.89 | Bladder | 4.12 | ± | 3.15 |
| Tumor | 4.92 | 4.21 | Tumor | 4.56 | ± | 0.50 |
| Femur | 17.80 | 15.88 | Femur | 16.84 | ± | 1.36 |

[¹⁸F]SB-FAP-09, 60 min + Blocker

| | 25.5 | 24.5 | | % ID/g | | |
|---|---|---|---|---|---|---|
| Body Wt.: | % ID/g | % ID/g | Tissue | Mean | | Std. |
| Blood | 0.28 | 0.22 | Blood | 0.25 | ± | 0.04 |
| Heart | 0.18 | 0.22 | Heart | 0.20 | ± | 0.03 |
| Lung | 0.30 | 0.24 | Lung | 0.27 | ± | 0.05 |
| Liver | 0.28 | 0.33 | Liver | 0.30 | ± | 0.04 |
| Spleen | 0.13 | 0.16 | Spleen | 0.15 | ± | 0.02 |
| Pancreas | 0.14 | 0.11 | Pancreas | 0.13 | ± | 0.02 |
| Stomach | 0.17 | 0.24 | Stomach | 0.21 | ± | 0.05 |
| Small intestine | 0.26 | 0.19 | Small intestien | 0.23 | ± | 0.05 |
| Large intestine | 0.19 | 0.27 | Large intestine | 0.23 | ± | 0.05 |
| Fat | 0.04 | 0.03 | Fat | 0.03 | ± | 0.01 |
| Muscle | 0.16 | 0.66 | Muscle | 0.41 | ± | 0.35 |
| Salivery gland | 0.20 | 0.20 | Salivary gland | 0.20 | ± | 0.00 |
| Brain | 0.09 | 0.08 | Brain | 0.08 | ± | 0.01 |
| Kidney | 0.81 | 0.57 | Kidney | 0.69 | ± | 0.17 |
| Bladder | 3.02 | 1.75 | Bladder | 2.38 | ± | 0.90 |
| Tumor | 0.99 | 0.64 | Tumor | 0.81 | ± | 0.25 |
| Femur | 24.81 | 27.15 | Femur | 25.98 | ± | 1.65 |

[¹⁸F]SB-FAP-09, 120 min

| | 20.6 | 20.6 | | % ID/g | | |
|---|---|---|---|---|---|---|
| Body Wt.: | % ID/g | % ID/g | Tissue | Mean | | Std. |
| Blood | 1.61 | 1.38 | Blood | 1.49 | ± | 0.16 |
| Heart | 0.68 | 0.79 | Heart | 0.73 | ± | 0.08 |
| Lung | 0.90 | 1.08 | Lung | 0.99 | ± | 0.13 |
| Liver | 0.58 | 0.58 | Liver | 0.58 | ± | 0.00 |
| Spleen | 0.39 | 0.30 | Spleen | 0.34 | ± | 0.06 |
| Pancreas | 1.45 | 2.03 | Pancreas | 1.74 | ± | 0.41 |
| Stomach | 0.47 | 0.43 | Stomach | 0.45 | ± | 0.03 |
| Small intestine | 0.54 | 0.45 | Small intestine | 0.49 | ± | 0.06 |
| Large intestine | 1.14 | 1.70 | Large intestine | 1.42 | ± | 0.40 |
| Fat | 0.17 | 0.23 | Fat | 0.20 | ± | 0.05 |
| Muscle | 0.71 | 0.79 | Muscle | 0.75 | ± | 0.06 |
| Salivery gland | 2.99 | 2.53 | Salivary gland | 2.76 | ± | 0.33 |
| Brain | 0.11 | 0.10 | Brain | 0.10 | ± | 0.01 |
| Kidney | 3.17 | 0.46 | Kidney | 1.82 | ± | 1.92 |
| Bladder | 1.98 | 0.60 | Bladder | 1.29 | ± | 0.97 |
| Tumor | 5.83 | 3.96 | Tumor | 4.89 | ± | 1.32 |
| Femur | 32.67 | 31.51 | Femur | 32.09 | ± | 0.82 |

Biodistribution (Laurence 2^{nd})
Injected dose: 20 μCi per mouse (SA: GBq/μmol; mCi/μmol)
Blocker: FAPI-04 (10 nmole, -fold access)
Time: 15, 30, and 60post injection of the tracer (Blocker was injected simultaneously with the tracer)
[¹⁸F]SB-FAP-09, 15 min

| | 1 | 2 | | | | |
|---|---|---|---|---|---|---|
| Aimal #: | 21.3 | 23.5 | Compound: | % ID/g | | |
| Body Wt.: | % ID/g | % ID/g | Tissue | Mean | | Std. |
| Blood | 2.00 | 2.04 | Blood | 2.02 | ± | 0.03 |
| Heart | 1.97 | 2.00 | Heart | 1.99 | ± | 0.03 |
| Lung | 1.81 | 2.13 | Lung | 1.97 | ± | 0.23 |
| Liver | 1.97 | 2.02 | Liver | 2.00 | ± | 0.03 |
| Spleen | 1.29 | 1.42 | Spleen | 1.35 | ± | 0.09 |
| Pancreas | 0.72 | 0.63 | Pancreas | 0.68 | ± | 0.06 |
| Stomach | 1.29 | 1.22 | Stomach | 1.26 | ± | 0.05 |
| Small intestine | 1.59 | 1.47 | Small intestine | 1.53 | ± | 0.08 |
| Large intestine | 1.32 | 1.46 | Large intestine | 1.39 | ± | 0.10 |
| Fat | 0.23 | 0.43 | Fat | 0.33 | ± | 0.14 |
| Muscle | 1.62 | 1.13 | Muscle | 1.38 | ± | 0.35 |
| Salivery gland | 1.14 | 1.30 | Salivary gland | 1.22 | ± | 0.11 |
| Brain | 0.28 | 0.17 | Brain | 0.23 | ± | 0.08 |
| Kidney | 3.54 | 3.94 | Kidney | 3.74 | ± | 0.28 |
| Bladder | 2.51 | 1.93 | Bladder | 2.22 | ± | 0.41 |
| Tumor | 1.79 | 1.26 | Tumor | 1.52 | ± | 0.37 |
| Femur | 18.11 | 15.55 | | 16.83 | ± | 1.81 |

[¹⁸F]SB-FAP-09, 30 min

| | 5 | 6 | | | | |
|---|---|---|---|---|---|---|
| Aimal #: | 22.7 | 23.1 | Compound: | % ID/g | | |
| Body Wt.: | % ID/g | % ID/g | Tissue | Mean | | Std. |
| Blood | 0.62 | 0.75 | Blood | 0.68 | ± | 0.09 |
| Heart | 0.67 | 0.88 | Heart | 0.77 | ± | 0.14 |
| Lung | 0.58 | 0.85 | Lung | 0.71 | ± | 0.19 |
| Liver | 0.53 | 0.74 | Liver | 0.63 | ± | 0.15 |
| Spleen | 0.48 | 0.60 | Spleen | 0.54 | ± | 0.09 |
| Pancreas | 0.22 | 0.25 | Pancreas | 0.24 | ± | 0.02 |
| Stomach | 0.51 | 0.51 | Stomach | 0.51 | ± | 0.00 |
| Small intestine | 0.51 | 0.57 | Small intestine | 0.54 | ± | 0.04 |
| Large intestine | 0.48 | 0.50 | Large intestine | 0.49 | ± | 0.01 |
| Fat | 0.05 | 0.13 | Fat | 0.09 | ± | 0.06 |
| Muscle | 1.13 | 0.54 | Muscle | 0.84 | ± | 0.42 |
| Salivery gland | 0.26 | 0.42 | Salivary gland | 0.34 | ± | 0.11 |
| Brain | 0.27 | 0.13 | Brain | 0.20 | ± | 0.10 |
| Kidney | 1.08 | 1.69 | Kidney | 1.38 | ± | 0.43 |
| Bladder | 5.15 | 3.83 | Bladder | 4.49 | ± | 0.93 |
| Tumor | 0.56 | 3.48 | Tumor | 2.02 | ± | 2.07 |
| Femur | 26.62 | 22.86 | | 24.74 | ± | 2.66 |

[¹⁸F]SB-FAP-09, 60 min

| | 9 | 10 | | | | |
|---|---|---|---|---|---|---|
| Aimal #: | 25.5 | 24.5 | Compound: | % ID/g | | |
| Body Wt.: | % ID/g | % ID/g | Tissue | Mean | | Std. |
| Blood | 0.15 | 0.17 | Blood | 0.16 | ± | 0.02 |
| Heart | 0.15 | 0.17 | Heart | 0.16 | ± | 0.02 |
| Lung | 0.19 | 0.16 | Lung | 0.17 | ± | 0.02 |
| Liver | 0.13 | 0.14 | Liver | 0.14 | ± | 0.01 |
| Spleen | 0.11 | 0.12 | Spleen | 0.12 | ± | 0.01 |
| Pancreas | 0.05 | 0.05 | Pancreas | 0.05 | ± | 0.00 |
| Stomach | 0.09 | 0.11 | Stomach | 0.10 | ± | 0.02 |
| Small intestine | 0.11 | 0.11 | Small intestine | 0.11 | ± | 0.00 |
| Large intestine | 0.11 | 0.15 | Large intestine | 0.13 | ± | 0.03 |
| Fat | 0.01 | 0.02 | Fat | 0.02 | ± | 0.00 |
| Muscle | 0.56 | 0.11 | Muscle | 0.33 | ± | 0.32 |
| Salivery gland | 0.07 | 0.08 | Salivary gland | 0.07 | ± | 0.01 |
| Brain | 0.19 | 0.09 | Brain | 0.14 | ± | 0.08 |
| Kidney | 0.26 | 0.31 | Kidney | 0.28 | ± | 0.03 |
| Bladder | 0.35 | 0.49 | Bladder | 0.42 | ± | 0.10 |
| Tumor | N/A | 0.15 | Tumor | 0.15 | ± | N/A |
| Femur | 21.53 | 25.67 | | 23.60 | ± | 2.93 |

[¹⁸F]SB-FAP-09, 60 min + Blocker

| | 13 | 14 | | | | |
|---|---|---|---|---|---|---|
| Aimal #: | 25.9 | 24.1 | Compound: | % ID/g | | |
| Body Wt.: | % ID/g | % ID/g | Tissue | Mean | | Std. |
| Blood | 0.19 | 0.25 | Blood | 0.22 | ± | 0.04 |
| Heart | 0.20 | 0.24 | Heart | 0.22 | ± | 0.03 |
| Lung | 0.19 | 0.57 | Lung | 0.38 | ± | 0.27 |
| Liver | 0.16 | 0.27 | Liver | 0.22 | ± | 0.07 |
| Spleen | 0.15 | 0.18 | Spleen | 0.16 | ± | 0.02 |
| Pancreas | 0.06 | 0.10 | Pancreas | 0.08 | ± | 0.03 |
| Stomach | 0.12 | 0.17 | Stomach | 0.15 | ± | 0.04 |
| Small intestine | 0.12 | 0.19 | Small intestine | 0.16 | ± | 0.05 |
| Large intestine | 0.15 | 0.17 | Large intestine | 0.16 | ± | 0.01 |
| Fat | 0.07 | 0.02 | Fat | 0.05 | ± | 0.04 |
| Muscle | 0.60 | 0.55 | Muscle | 0.57 | ± | 0.03 |
| Salivery gland | 0.12 | 0.12 | Salivary gland | 0.12 | ± | 0.00 |
| Brain | 0.63 | 0.11 | Brain | 0.37 | ± | 0.37 |
| Kidney | 0.53 | 0.43 | Kidney | 0.49 | ± | 0.09 |
| Bladder | 0.65 | 0.36 | Bladder | 0.51 | ± | 0.19 |
| Tumor | 0.17 | 0.21 | Tumor | 0.19 | ± | 0.03 |
| Femur | 22.88 | 26.88 | | 24.88 | ± | 2.83 |

Biodistribution ([⁶⁸Ga]FAPI04)
Injected dose: 20 μCi per mouse (SA: 3.33 GBq/μmol; 90 mCi/μmol)
Blocker: Cold FAPI-04 (10 nmole, 45-fold access)
Time: 30and 60post injection of the tracer (Blocker was injected simultaneously with the tracer)
[⁶⁸Ga]FAPI-04, 30 min

| | 24 | 24.1 | | % ID/g | | |
|---|---|---|---|---|---|---|
| Body Wt.: | % ID/g | % ID/g | Tissue | Mean | | Std. |
| Blood | 0.89 | 1.03 | Blood | 0.96 | ± | 0.10 |
| Heart | 0.34 | 0.39 | Heart | 0.37 | ± | 0.03 |
| Lung | 0.88 | 1.10 | Lung | 0.99 | ± | 0.16 |
| Liver | 0.49 | 0.43 | Liver | 0.46 | ± | 0.04 |
| Spleen | 0.25 | 0.29 | Spleen | 0.27 | ± | 0.03 |
| Pancreas | 0.25 | 0.31 | Pancreas | 0.28 | ± | 0.04 |
| Stomach | 0.29 | 0.40 | Stomach | 0.34 | ± | 0.08 |
| Small intestine | 0.27 | 0.34 | Small intestine | 0.31 | ± | 0.05 |
| Large intestine | 0.37 | 0.45 | Large intestine | 0.41 | ± | 0.06 |
| Fat | 0.13 | 0.12 | Fat | 0.13 | ± | 0.01 |
| Muscle | 0.18 | 0.25 | Muscle | 0.22 | ± | 0.05 |
| Salivery gland | 0.34 | 0.37 | Salivary gland | 0.35 | ± | 0.02 |
| Brain | 0.01 | 0.02 | Brain | 0.01 | ± | 0.01 |
| Kidney | 1.97 | 2.94 | Kidney | 2.45 | ± | 0.69 |
| Bladder | 1.83 | 15.32 | Bladder | 8.58 | ± | 9.54 |
| Tumor | 1.35 | 1.14 | Tumor | 1.24 | ± | 0.15 |
| Femur | 0.19 | 0.31 | Femur | 0.23 | ± | 0.11 |

[⁶⁸Ga]FAPI-04, 60 min

| | 25.5 | 24.5 | | % ID/g | | |
|---|---|---|---|---|---|---|
| Body Wt.: | % ID/g | % ID/g | Tissue | Mean | | Std. |
| Blood | 0.18 | 0.24 | Blood | 0.21 | ± | 0.05 |
| Heart | 0.10 | 0.10 | Heart | 0.10 | ± | 0.00 |
| Lung | 0.26 | 0.24 | Lung | 0.25 | ± | 0.01 |
| Liver | 0.22 | 0.20 | Liver | 0.21 | ± | 0.01 |
| Spleen | 0.09 | 0.11 | Spleen | 0.10 | ± | 0.01 |
| Pancreas | 0.08 | 0.10 | Pancreas | 0.09 | ± | 0.02 |
| Stomach | 0.26 | 0.12 | Stomach | 0.19 | ± | 0.10 |
| Small intestine | 0.12 | 0.12 | Small intestine | 0.12 | ± | 0.00 |
| Large intestine | 0.14 | 0.16 | Large intestine | 0.15 | ± | 0.01 |
| Fat | 0.06 | 0.05 | Fat | 0.05 | ± | 0.00 |
| Muscle | 0.09 | 0.09 | Muscle | 0.09 | ± | 0.00 |
| Salivery gland | 0.16 | 0.26 | Salivary gland | 0.21 | ± | 0.07 |
| Brain | 0.03 | 0.10 | Brain | 0.07 | ± | 0.05 |
| Kidney | 1.11 | 1.09 | Kidney | 1.10 | ± | 0.01 |
| Bladder | 0.53 | 11.63 | Bladder | 6.08 | ± | 7.85 |
| Tumor | 0.57 | 0.96 | Tumor | 0.76 | ± | 0.27 |
| Femur | 0.28 | 0.50 | Femur | 0.39 | ± | 0.16 |

[⁶⁸Ga]FAPI-04 + Blocker, 60 min

| | 22.7 | 23.1 | | % ID/g | | |
|---|---|---|---|---|---|---|
| Body Wt.: | % ID/g | % ID/g | Tissue | Mean | | Std. |
| Blood | 0.11 | 0.16 | Blood | 0.13 | ± | 0.03 |
| Heart | 0.13 | 0.08 | Heart | 0.10 | ± | 0.03 |
| Lung | 0.21 | 0.18 | Lung | 0.20 | ± | 0.02 |
| Liver | 0.20 | 0.19 | Liver | 0.19 | ± | 0.01 |
| Spleen | 0.09 | 0.10 | Spleen | 0.09 | ± | 0.01 |
| Pancreas | 0.07 | 0.06 | Pancreas | 0.06 | ± | 0.01 |
| Stomach | 0.07 | 0.12 | Stomach | 0.10 | ± | 0.03 |
| Small intestine | 0.07 | 0.09 | Small intestine | 0.08 | ± | 0.01 |
| Large intestine | 0.09 | 0.11 | Large intestine | 0.10 | ± | 0.01 |
| Fat | 0.17 | 0.05 | Fat | 0.11 | ± | 0.09 |
| Muscle | 0.06 | 0.07 | Muscle | 0.07 | ± | 0.01 |
| Salivery gland | 0.11 | 0.09 | Salivary gland | 0.10 | ± | 0.01 |
| Brain | 0.02 | 0.02 | Brain | 0.02 | ± | 0.00 |
| Kidney | 1.04 | 1.10 | Kidney | 1.07 | ± | 0.04 |
| Bladder | 0.35 | 0.41 | Bladder | 0.38 | ± | 0.04 |
| Tumor | 0.48 | 0.43 | Tumor | 0.46 | ± | 0.04 |
| Femur | 0.06 | 0.13 | Femur | 0.10 | ± | 0.05 |

Biodistribution ([¹⁸F]JHU1221785)

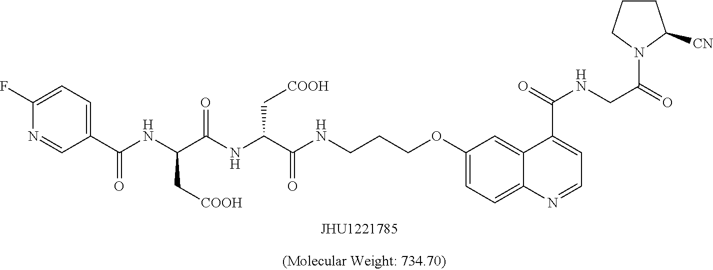 [SMILES: N#C[C@@H]1CCCN1C(=O)CNC(=O)c1ccnc2ccc(OCCCNC(=O)[C@@H](CC(=O)O)NC(=O)[C@@H](CC(=O)O)NC(=O)c3ccc(F)nc3)cc12]

Injected dose: 20 μCi per mouse (SA: 240.5 GBq/μmol; 6500 mCi/μmol)
Blocker: Cold FAPI-04 (10 nmole, 6500-fold access)
Time: 30 and 60post injection of the tracer (Blocker was injected simultaneously with the tracer)
[¹⁸F]HU1221785, 30 min

| | 22.2 | 22.6 | | % ID/g | | |
|---|---|---|---|---|---|---|
| Body Wt.: | % ID/g | % ID/g | Tissue | Mean | | Std. |
| Blood | 5.98 | 3.90 | Blood | 4.94 | ± | 1.47 |
| Heart | 2.51 | 2.01 | Heart | 2.26 | ± | 0.35 |
| Lung | 2.77 | 2.53 | Lung | 2.65 | ± | 0.17 |
| Liver | 6.56 | 8.08 | Liver | 7.32 | ± | 1.08 |
| Spleen | 1.03 | 0.97 | Spleen | 1.00 | ± | 0.04 |
| Pancreas | 1.92 | 2.71 | Pancreas | 2.31 | ± | 0.56 |
| Stomach | 0.72 | 0.92 | Stomach | 0.82 | ± | 0.14 |
| Small intestine | 2.60 | 2.38 | Small intestine | 2.49 | ± | 0.16 |
| Large intestine | 0.58 | 0.86 | Large intestine | 0.72 | ± | 0.20 |
| Fat | 0.21 | 0.19 | Fat | 0.20 | ± | 0.01 |
| Muscle | 0.81 | 1.12 | Muscle | 0.96 | ± | 0.22 |
| Salivery gland | 2.81 | 3.05 | Salivary gland | 2.93 | ± | 0.17 |
| Brain | 0.14 | 0.15 | Brain | 0.14 | ± | 0.01 |
| Kidney | 1.60 | 3.33 | Kidney | 2.47 | ± | 1.23 |
| Bladder | 0.93 | 4.69 | Bladder | 2.81 | ± | 2.66 |
| Tumor | 1.14 | 1.14 | Tumor | 1.24 | ± | 0.14 |
| Femur | 2.78 | 2.44 | Femur | 2.61 | ± | 0.24 |

[¹⁸F]JHU1221785, 60 min

| | 25.5 | 24.5 | | % ID/g | | |
|---|---|---|---|---|---|---|
| Body Wt.: | % ID/g | % ID/g | Tissue | Mean | | Std. |
| Blood | 2.56 | 4.57 | Blood | 3.57 | ± | 1.42 |
| Heart | 1.38 | 1.73 | Heart | 1.56 | ± | 0.25 |
| Lung | 1.70 | 2.65 | Lung | 2.18 | ± | 0.67 |
| Liver | 7.41 | 8.27 | Liver | 7.84 | ± | 0.61 |
| Spleen | 0.78 | 1.09 | Spleen | 0.93 | ± | 0.22 |
| Pancreas | 1.96 | 2.17 | Pancreas | 2.07 | ± | 0.15 |
| Stomach | 0.60 | 0.81 | Stomach | 0.71 | ± | 0.15 |
| Small intestine | 1.65 | 2.01 | Small intestine | 1.83 | ± | 0.26 |
| Large intestine | 0.61 | 0.78 | Large intestine | 0.69 | ± | 0.12 |
| Fat | 0.22 | 0.12 | Fat | 0.22 | ± | 0.01 |
| Muscle | 1.03 | 0.69 | Muscle | 0.98 | ± | 0.12 |
| Salivery gland | 2.17 | 4.73 | Salivary gland | 3.45 | ± | 1.80 |
| Brain | 0.09 | 0.12 | Brain | 0.10 | ± | 0.03 |
| Kidney | 0.79 | 1.04 | Kidney | 0.91 | ± | 0.18 |
| Bladder | 0.76 | 0.92 | Bladder | 0.84 | ± | 0.12 |
| Tumor | 2.99 | 1.47 | Tumor | 2.23 | ± | 1.07 |
| Femur | 2.06 | 2.07 | Femur | 2.07 | ± | 0.01 |

[¹⁸F]JHU1221785 + Blocker, 60 min

| | 22.7 | 23.1 | | % ID/g | | |
|---|---|---|---|---|---|---|
| Body Wt.: | % ID/g | % ID/g | Tissue | Mean | | Std. |
| Blood | 0.11 | 9.26 | Blood | 4.68 | ± | 6.47 |
| Heart | 0.06 | 0.07 | Heart | 0.07 | ± | 0.01 |
| Lung | 0.13 | 0.20 | Lung | 0.17 | ± | 0.05 |
| Liver | 9.42 | 10.36 | Liver | 9.69 | ± | 0.67 |
| Spleen | 0.08 | 0.25 | Spleen | 0.16 | ± | 0.12 |
| Pancreas | 0.05 | 0.22 | Pancreas | 0.13 | ± | 0.12 |
| Stomach | 0.09 | 0.23 | Stomach | 0.16 | ± | 0.10 |
| Small intestine | 0.88 | 6.19 | Small intestine | 3.54 | ± | 3.75 |
| Large intestine | 0.08 | 0.33 | Large intestine | 0.20 | ± | 0.18 |
| Fat | 0.03 | 0.21 | Fat | 0.12 | ± | 0.13 |
| Muscle | 0.04 | 0.07 | Muscle | 0.06 | ± | 0.02 |
| Salivery gland | 0.04 | 0.06 | Salivary gland | 0.05 | ± | 0.01 |
| Brain | 0.02 | 0.04 | Brain | 0.03 | ± | 0.01 |
| Kidney | 0.41 | 0.51 | Kidney | 0.46 | ± | 0.07 |
| Bladder | 0.23 | 0.67 | Bladder | 0.43 | ± | 0.31 |
| Tumor | 0.08 | 0.11 | Tumor | 0.10 | ± | 0.02 |
| Femur | 1.52 | 1.50 | Femur | 1.51 | ± | 0.01 |

Biodistribution ([¹⁸F]FAP-FDG)
Injected dose: 20 μCi per mouse (SA: GBq/μmol; mCi/μmol)
Blocker; Cold FAPI-04 (10 nmole, access)
Time: 60 min post injection of the tracer (Blocker was injected simultaneously with the tracer)
[¹⁸F]JHU1221785, 60 min

| | 25.5 | 24.5 | | % ID/g | | |
|---|---|---|---|---|---|---|
| Body Wt.: | % ID/g | % ID/g | Tissue | Mean | | Std. |
| Blood | 0.21 | 0.25 | Blood | 0.23 | ± | 0.03 |
| Heart | 23.96 | 16.00 | Heart | 19.98 | ± | 5.62 |
| Lung | 2.15 | 2.85 | Lung | 2.50 | ± | 0.49 |
| Liver | 0.55 | 0.70 | Liver | 0.63 | ± | 0.11 |
| Spleen | 1.20 | 1.38 | Spleen | 1.29 | ± | 0.13 |
| Pancreas | 1.25 | 1.42 | Pancreas | 1.33 | ± | 0.12 |
| Stomach | 2.04 | 3.39 | Stomach | 2.72 | ± | 0.96 |
| Small intestine | 1.21 | 0.91 | Small intestine | 1.06 | ± | 0.21 |
| Large intestine | 4.03 | 4.32 | Large intestine | 4.17 | ± | 0.20 |
| Fat | 3.29 | 1.13 | Fat | 2.21 | ± | 1.53 |
| Muscle | 7.65 | 4.71 | Muscle | 6.18 | ± | 2.08 |
| Salivery gland | 6.70 | 3.91 | Salivary gland | 5.31 | ± | 1.97 |
| Brain | 5.31 | 6.18 | Brain | 5.74 | ± | 0.61 |
| Kidney | 1.13 | 2.01 | Kidney | 1.57 | ± | 0.62 |
| Bladder | 2.55 | 2.49 | Bladder | 2.52 | ± | 0.04 |
| Tumor | 4.12 | 5.91 | Tumor | 5.02 | ± | 1.27 |
| Femur | 2.28 | 2.68 | Femur | 2.48 | ± | 0.28 |

[¹⁸F]JHU1221785 + Blocker, 60 min

| | 22.2 | 22.6 | | % ID/g | | |
|---|---|---|---|---|---|---|
| Body Wt.: | % ID/g | % ID/g | Tissue | Mean | | Std. |
| Blood | 0.20 | 0.25 | Blood | 0.22 | ± | 0.03 |
| Heart | 11.97 | 17.79 | Heart | 14.88 | ± | 4.11 |
| Lung | 2.25 | 2.08 | Lung | 2.17 | ± | 0.12 |
| Liver | 0.38 | 0.55 | Liver | 0.47 | ± | 0.12 |
| Spleen | 0.92 | 1.68 | Spleen | 1.30 | ± | 0.54 |
| Pancreas | 1.20 | 0.82 | Pancreas | 1.01 | ± | 0.27 |
| Stomach | 3.50 | 2.02 | Stomach | 2.76 | ± | 1.05 |
| Small intestine | 0.61 | 0.92 | Small intestine | 0.77 | ± | 0.22 |
| Large intestine | 3.51 | 3.22 | Large intestine | 3.37 | ± | 0.20 |
| Fat | 0.63 | 0.26 | Fat | 0.44 | ± | 0.26 |
| Muscle | 6.83 | 6.64 | Muscle | 6.73 | ± | 0.13 |
| Salivery gland | 5.34 | 2.19 | Salivary gland | 3.76 | ± | 2.23 |
| Brain | 4.18 | 4.67 | Brain | 4.43 | ± | 0.34 |
| Kidney | 0.95 | 0.87 | Kidney | 0.91 | ± | 0.05 |
| Bladder | 1.45 | 2.74 | Bladder | 2.10 | ± | 0.91 |
| Tumor | 3.33 | 3.97 | Tumor | 3.65 | ± | 0.45 |
| Femur | 1.33 | 1.67 | Femur | 1.50 | ± | 0.24 |

Biodistribution ([¹⁸F]Sri-07-56)
Injected dose: 2 μCi per mouse (3780 mCi/μmol)
Blocker: Cold FAPI-04 (10 nmol)
Time: 60 min post injection of the tracer (Blocker was injected simultaneously with the tracer)
[¹⁸F]Sri-07-56, 60 min

| | 22.7 | 23.1 | | % ID/g | | |
|---|---|---|---|---|---|---|
| Body Wt.: | % ID/g | % ID/g | Tissue | Mean | | Std. |
| Blood | 10.48 | 7.56 | Blood | 9.02 | ± | 2.06 |
| Heart | 4.72 | 3.07 | Heart | 3.90 | ± | 1.17 |
| Lung | 5.06 | 3.66 | Lung | 4.36 | ± | 0.99 |
| Liver | 2.82 | 2.25 | Liver | 2.54 | ± | 0.40 |
| Spleen | 2.19 | 1.64 | Spleen | 1.91 | ± | 0.39 |
| Pancreas | 2.39 | 1.96 | Pancreas | 2.18 | ± | 0.30 |
| Stomach | 1.05 | 0.67 | Stomach | 0.86 | ± | 0.26 |
| Small intestine | 2.06 | 1.40 | Small intestine | 1.73 | ± | 0.46 |
| Large intestine | 0.90 | 0.95 | Large intestine | 0.93 | ± | 0.04 |
| Fat | 0.38 | 1.40 | Fat | 0.89 | ± | 0.72 |
| Muscle | 0.74 | 1.06 | Muscle | 0.90 | ± | 0.23 |
| Salivery gland | 4.40 | 2.70 | Salivary gland | 3.55 | ± | 1.20 |
| Brain | 0.28 | 0.16 | Brain | 0.22 | ± | 0.09 |
| Kidney | 2.24 | 1.96 | Kidney | 2.10 | ± | 0.20 |
| Bladder | 0.86 | 1.12 | Bladder | 0.99 | ± | 0.18 |
| Tumor | 1.86 | 1.70 | Tumor | 1.78 | ± | 0.11 |
| Femur | 1.63 | 1.01 | Femur | 1.32 | ± | 0.44 |

[¹⁸F]Sri-07-56 + Blocker, 60 min

| | 21.3 | 25.4 | | % ID/g | | |
|---|---|---|---|---|---|---|
| Body Wt.: | % ID/g | % ID/g | Tissue | Mean | | Std. |
| Blood | 0.09 | 0.08 | Blood | 0.08 | ± | 0.01 |
| Heart | 0.05 | 0.05 | Heart | 0.05 | ± | 0.00 |
| Lung | 0.12 | 0.22 | Lung | 0.17 | ± | 0.07 |
| Liver | 0.08 | 0.10 | Liver | 0.09 | ± | 0.01 |
| Spleen | 0.04 | 0.02 | Spleen | 0.03 | ± | 0.01 |
| Pancreas | 0.02 | 0.09 | Pancreas | 0.04 | ± | 0.07 |
| Stomach | 0.10 | 0.03 | Stomach | 0.06 | ± | 0.05 |
| Small intestine | 0.19 | 0.15 | Small intestine | 0.02 | ± | 0.24 |
| Large intestine | 0.00 | 0.99 | Large intestine | 0.49 | ± | 0.70 |
| Fat | 0.04 | 0.05 | Fat | 0.01 | ± | 0.07 |
| Muscle | 0.15 | 0.15 | Muscle | 0.00 | ± | 0.21 |
| Salivery gland | 0.17 | 0.05 | Salivary gland | 0.11 | ± | 0.08 |
| Brain | 0.04 | 0.04 | Brain | 0.00 | ± | 0.06 |
| Kidney | 0.69 | 0.72 | Kidney | 0.71 | ± | 0.02 |
| Bladder | 0.75 | 0.49 | Bladder | 0.62 | ± | 0.18 |
| Tumor | 0.35 | 0.36 | Tumor | 0.36 | ± | 0.00 |
| Femur | 0.21 | 0.15 | Femur | 0.18 | ± | 0.04 |

Biodistribution ([¹⁸F]Sri-07-52)
Injected dose: ~100 μCi per mouse
Blocker: Cold FAPI-04 (10 nmol)

| Time point | Specific activity |
|---|---|
| 0.5 h (n = 2) | Tracer baseline |
| 1 h (n = 2) | Tracer baseline |
| 2 h (n = 2) | Tracer baseline |
| 2 h (n = 2) | Tracer + 0.25 nmol carrier/dose |
| 2 h (n = 2) | Tracer + 0.5 nmol carrier/dose |
| 2 h (n = 2) | Tracer + FAPI blocker (1 mg/kg) |

[¹⁸F]Sri-07-52, 30 min

| | 23.5 | 20.4 | | % ID/g | | |
|---|---|---|---|---|---|---|
| Body Wt.: | % ID/g | % ID/g | Tissue | Mean | | Std. |
| Blood | 9.03 | 11.04 | Blood | 10.03 | ± | 1.42 |
| Heart | 4.65 | 4.52 | Heart | 4.58 | ± | 0.09 |
| Lung | 6.53 | 5.82 | Lung | 6.18 | ± | 0.50 |
| Liver | 3.03 | 3.36 | Liver | 3.20 | ± | 0.23 |
| Spleen | 2.06 | 1.99 | Spleen | 2.03 | ± | 0.05 |
| Pancreas | 6.54 | 5.18 | Pancreas | 5.86 | ± | 0.96 |
| Stomach | 1.72 | 1.71 | Stomach | 1.71 | ± | 0.01 |
| Small intestine | 4.18 | 4.54 | Small intestine | 4.36 | ± | 0.26 |
| Large intestine | 5.82 | 1.74 | Large intestine | 3.78 | ± | 2.88 |
| Fat | 0.36 | 0.64 | Fat | 0.50 | ± | 0.20 |
| Muscle | 2.07 | 2.12 | Muscle | 2.09 | ± | 0.04 |
| Salivery gland | 11.28 | 16.41 | Salivary gland | 13.84 | ± | 3.63 |
| Brain | 0.24 | 0.19 | Brain | 0.21 | ± | 0.03 |
| Kidney | 2.62 | 2.80 | Kidney | 2.71 | ± | 0.13 |
| Bladder | 1.47 | 2.50 | Bladder | 1.98 | ± | 0.73 |
| Tumor | 5.57 | 4.40 | Tumor | 4.99 | ± | 0.89 |
| Femur | 2.15 | 2.91 | Femur | 2.53 | ± | 0.54 |

Biodistribution ([¹⁸F]Sri-07-52)
Injected dose: 20 μCi per mouse
Blocker: Cold FAPI-04 (10 nmol)
[18F]Sri-07-52, 60 min

| | 22.7 | 23.1 | | % ID/g | | |
|---|---|---|---|---|---|---|
| Body Wt.: | % ID/g | % ID/g | Tissue | Mean | | Std. |
| Blood | 2.58 | 2.21 | Blood | 2.39 | ± | 0.26 |
| Heart | 1.36 | 1.42 | Heart | 1.39 | ± | 0.04 |
| Lung | 1.60 | 1.32 | Lung | 1.46 | ± | 0.19 |
| Liver | 1.11 | 1.04 | Liver | 1.07 | ± | 0.05 |
| Spleen | 0.72 | 0.77 | Spleen | 0.74 | ± | 0.03 |
| Pancreas | 3.34 | 3.27 | Pancreas | 3.30 | ± | 0.05 |
| Stomach | 0.66 | 0.69 | Stomach | 0.68 | ± | 0.02 |
| Small intestine | 1.56 | 1.57 | Small intestine | 1.57 | ± | 0.00 |
| Large intestine | 1.11 | 1.21 | Large intestine | 1.16 | ± | 0.07 |
| Fat | 0.53 | 0.29 | Fat | 0.41 | ± | 0.17 |
| Muscle | 1.43 | 1.38 | Muscle | 1.40 | ± | 0.08 |
| Salivery gland | 4.35 | 5.90 | Salivary gland | 5.13 | ± | 1.10 |
| Brain | 0.08 | 0.11 | Brain | 0.09 | ± | 0.02 |
| Kidney | 1.18 | 1.23 | Kidney | 1.21 | ± | 0.03 |
| Bladder | 1.90 | 1.61 | Bladder | 1.75 | ± | 0.21 |
| Tumor | 2.44 | 2.10 | Tumor | 2.27 | ± | 0.24 |
| Femur | 1.91 | 2.29 | Femur | 2.10 | ± | 0.27 |

[¹⁸F]Sri-07-52, 120 min

| | 23.5 | 20.4 | | % ID/g | | |
|---|---|---|---|---|---|---|
| Body Wt.: | % ID/g | % ID/g | Tissue | Mean | | Std. |
| Blood | 4.70 | 6.19 | Blood | 5.44 | ± | 1.05 |
| Heart | 3.20 | 3.21 | Heart | 3.20 | ± | 0.01 |
| Lung | 2.65 | 3.65 | Lung | 3.15 | ± | 0.70 |
| Liver | 2.28 | 2.43 | Liver | 2.33 | ± | 0.14 |
| Spleen | 1.54 | 1.49 | Spleen | 1.51 | ± | 0.04 |
| Pancreas | 3.00 | 4.96 | Pancreas | 3.98 | ± | 1.38 |
| Stomach | 1.00 | 1.23 | Stomach | 1.11 | ± | 0.16 |
| Small intestine | 2.55 | 2.72 | Small intestine | 2.64 | ± | 0.12 |
| Large intestine | 0.85 | 1.30 | Large intestine | 1.08 | ± | 0.32 |
| Fat | 0.74 | 0.38 | Fat | 0.56 | ± | 0.25 |
| Muscle | 1.58 | 1.47 | Muscle | 1.53 | ± | 0.08 |
| Salivery gland | 6.20 | 8.21 | Salivary gland | 7.20 | ± | 1.42 |
| Brain | 0.32 | 0.13 | Brain | 0.22 | ± | 0.13 |
| Kidney | 1.66 | 3.77 | Kidney | 2.71 | ± | 1.49 |
| Bladder | 0.97 | 2.35 | Bladder | 1.66 | ± | 0.98 |
| Tumor | 1.67 | 2.25 | Tumor | 1.96 | ± | 0.41 |
| Femur | 1.99 | 2.35 | Femur | 2.17 | ± | 0.26 |

[¹⁸F]Sri-07-52 + Carrier, 120 min

| | 25.5 | 24.5 | | % ID/g | | |
|---|---|---|---|---|---|---|
| Body Wt.: | % ID/g | % ID/g | Tissue | Mean | | Std. |
| Blood | 0.66 | 0.73 | Blood | 0.70 | ± | 0.05 |
| Heart | 0.33 | 0.32 | Heart | 0.32 | ± | 0.01 |
| Lung | 0.47 | 0.49 | Lung | 0.48 | ± | 0.01 |
| Liver | 0.42 | 0.40 | Liver | 0.41 | ± | 0.01 |
| Spleen | 0.24 | 0.22 | Spleen | 0.23 | ± | 0.02 |
| Pancreas | 0.77 | 0.86 | Pancreas | 0.81 | ± | 0.06 |
| Stomach | 0.27 | 0.29 | Stomach | 0.28 | ± | 0.01 |
| Small intestine | 0.43 | 0.74 | Small intestine | 0.58 | ± | 0.22 |
| Large intestine | 0.26 | 4.31 | Large intestine | 2.28 | ± | 2.87 |
| Fat | 0.13 | 0.46 | Fat | 0.30 | ± | 0.23 |
| Muscle | 0.59 | 0.67 | Muscle | 0.63 | ± | 0.06 |
| Salivery gland | 1.82 | 1.99 | Salivary gland | 1.91 | ± | 0.12 |
| Brain | 0.03 | 0.08 | Brain | 0.06 | ± | 0.03 |
| Kidney | 0.56 | 0.68 | Kidney | 0.62 | ± | 0.08 |
| Bladder | 0.84 | 0.79 | Bladder | 0.82 | ± | 0.03 |
| Tumor | 3.72 | 3.41 | Tumor | 3.57 | ± | 0.22 |
| Femur | 1.70 | 1.76 | Femur | 1.73 | ± | 0.05 |

[¹⁸F]Sri-07-52 + Blocker, 120 min

| | 23.5 | 22.2 | | % ID/g | | |
|---|---|---|---|---|---|---|
| Body Wt.: | % ID/g | % ID/g | Tissue | Mean | | Std. |
| Blood | 0.01 | 0.01 | Blood | 0.01 | ± | 0.00 |
| Heart | 0.00 | 0.00 | Heart | 0.00 | ± | 0.00 |
| Lung | 0.01 | 0.31 | Lung | 0.16 | ± | 0.21 |
| Liver | 0.02 | 0.05 | Liver | 0.03 | ± | 0.02 |
| Spleen | 0.01 | 0.10 | Spleen | 0.06 | ± | 0.07 |
| Pancreas | 0.09 | 0.10 | Pancreas | 0.09 | ± | 0.01 |
| Stomach | 0.07 | 0.26 | Stomach | 0.17 | ± | 0.13 |
| Small intestine | 0.15 | 9.58 | Small intestine | 4.86 | ± | 6.66 |
| Large intestine | 4.40 | 0.11 | Large intestine | 2.26 | ± | 3.03 |
| Fat | 0.00 | 0.03 | Fat | 0.01 | ± | 0.02 |
| Muscle | 0.02 | 0.02 | Muscle | 0.02 | ± | 0.00 |
| Salivery gland | 0.01 | 0.03 | Salivary gland | 0.02 | ± | 0.01 |
| Brain | 0.01 | 0.29 | Brain | 0.15 | ± | 0.20 |
| Kidney | 0.14 | 0.01 | Kidney | 0.08 | ± | 0.09 |
| Bladder | 0.11 | 1.40 | Bladder | 0.75 | ± | 0.91 |
| Tumor | 0.17 | 0.03 | Tumor | 0.10 | ± | 0.10 |
| Femur | 0.06 | 0.01 | | 0.03 | ± | 0.04 |

Biodistribution ([¹⁸F]JHU1221823)

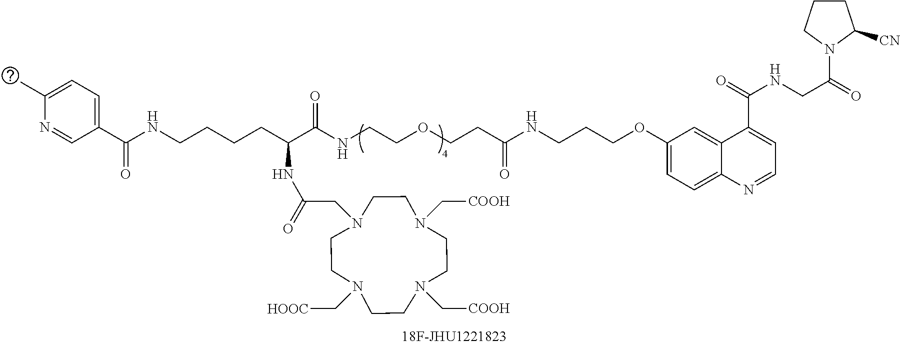

Injected dose: 20 μCi per mouse (SA: 7200 mCi/μmol)
Blocker: Cold FAPI-04 (10 nmol)
Carrier: 0.5 nmol/mouse
[¹⁸F]JHU1221823, 60 min

| | 22.7 | 23.1 | | % ID/g | | |
|---|---|---|---|---|---|---|
| Body Wt.: | % ID/g | % ID/g | Tissue | Mean | | Std. |
| Blood | 9.28 | 9.80 | Blood | 9.54 | ± | 0.37 |
| Heart | 4.31 | 4.98 | Heart | 4.65 | ± | 0.48 |
| Lung | 5.53 | 5.91 | Lung | 5.72 | ± | 0.27 |
| Liver | 2.68 | 3.29 | Liver | 2.99 | ± | 0.43 |
| Spleen | 1.86 | 2.43 | Spleen | 2.15 | ± | 0.40 |
| Pancreas | 1.62 | 2.66 | Pancreas | 2.14 | ± | 0.73 |
| Stomach | 0.84 | 1.45 | Stomach | 1.14 | ± | 0.43 |
| Small intestine | 2.59 | 3.46 | Small intestine | 3.02 | ± | 0.62 |
| Large intestine | 1.29 | 1.69 | Large intestine | 1.49 | ± | 0.29 |
| Fat | 0.47 | 1.68 | Fat | 1.07 | ± | 0.86 |
| Muscle | 1.79 | 1.78 | Muscle | 1.78 | ± | 0.01 |
| Salivery gland | 7.44 | 6.43 | Salivary gland | 6.93 | ± | 0.71 |
| Brain | 0.23 | 0.22 | Brain | 0.22 | ± | 0.01 |
| Kidney | 2.70 | 2.77 | Kidney | 2.73 | ± | 0.05 |
| Bladder | 6.41 | 2.64 | Bladder | 4.53 | ± | 2.66 |
| Tumor | 4.35 | 7.05 | Tumor | 5.71 | ± | 1.92 |
| Femur | 2.55 | 3.11 | Femur | 2.83 | ± | 0.40 |

[¹⁸F]JHU1221823, 120 min

| | 23.7 | 22.4 | | % ID/g | | |
|---|---|---|---|---|---|---|
| Body Wt.: | % ID/g | % ID/g | Tissue | Mean | | Std. |
| Blood | 5.77 | 6.78 | Blood | 6.28 | ± | 0.72 |
| Heart | 3.18 | 3.79 | Heart | 3.49 | ± | 0.43 |
| Lung | 3.13 | 4.16 | Lung | 3.65 | ± | 0.73 |
| Liver | 1.94 | 2.42 | Liver | 2.18 | ± | 0.34 |
| Spleen | 1.49 | 1.67 | Spleen | 1.58 | ± | 0.13 |
| Pancreas | 4.03 | 5.32 | Pancreas | 4.67 | ± | 0.91 |
| Stomach | 1.11 | 1.37 | Stomach | 1.24 | ± | 0.18 |
| Small intestine | 1.88 | 2.21 | Small intestine | 2.04 | ± | 0.23 |
| Large intestine | 1.40 | 1.36 | Large intestine | 1.38 | ± | 0.03 |
| Fat | 0.97 | 1.93 | Fat | 1.45 | ± | 0.68 |
| Muscle | 1.18 | 1.15 | Muscle | 1.16 | ± | 0.02 |
| Salivery gland | 5.17 | 11.05 | Salivary gland | 8.11 | ± | 4.16 |
| Brain | 0.19 | 0.17 | Brain | 0.18 | ± | 0.01 |
| Kidney | 1.61 | 2.24 | Kidney | 1.93 | ± | 0.44 |
| Bladder | 2.63 | 1.21 | Bladder | 1.92 | ± | 1.01 |
| Tumor | 2.88 | 2.94 | Tumor | 2.91 | ± | 0.04 |
| Femur | 1.71 | 1.91 | | 1.61 | ± | 0.14 |

[¹⁸F]JHU1221823 + Carrier, 60 min

| | 25.5 | 24.5 | | % ID/g | | |
|---|---|---|---|---|---|---|
| Body Wt.: | % ID/g | % ID/g | Tissue | Mean | | Std. |
| Blood | 0.13 | 0.15 | Blood | 0.14 | ± | 0.01 |
| Heart | 0.05 | 0.08 | Heart | 0.06 | ± | 0.02 |
| Lung | 0.26 | 0.40 | Lung | 0.33 | ± | 0.10 |
| Liver | 0.21 | 0.18 | Liver | 0.20 | ± | 0.03 |
| Spleen | 0.07 | 0.09 | Spleen | 0.08 | ± | 0.01 |
| Pancreas | 0.11 | 0.14 | Pancreas | 0.13 | ± | 0.03 |
| Stomach | 0.06 | 0.08 | Stomach | 0.07 | ± | 0.02 |
| Small intestine | 0.10 | 0.14 | Small intestine | 0.12 | ± | 0.03 |
| Large intestine | 4.42 | 0.07 | Large intestine | 2.25 | ± | 3.07 |
| Fat | 0.05 | 0.07 | Fat | 0.06 | ± | 0.01 |
| Muscle | 0.03 | 0.13 | Muscle | 0.08 | ± | 0.07 |
| Salivery gland | 0.17 | 0.30 | Salivary gland | 0.23 | ± | 0.09 |
| Brain | 0.01 | 0.02 | Brain | 0.01 | ± | 0.01 |
| Kidney | 1.02 | 1.13 | Kidney | 1.07 | ± | 0.08 |
| Bladder | 0.19 | 0.24 | Bladder | 0.22 | ± | 0.03 |
| Tumor | 0.66 | 1.37 | Tumor | 1.01 | ± | 0.50 |
| Femur | 0.33 | 0.52 | Femur | 0.42 | ± | 0.14 |

[¹⁸F]JHU1221823 + Blocker, 60 min

| | 22.2 | 22 | | % ID/g | | |
|---|---|---|---|---|---|---|
| Body Wt.: | % ID/g | % ID/g | Tissue | Mean | | Std. |
| Blood | 0.15 | 0.21 | Blood | 0.18 | ± | 0.04 |
| Heart | 0.07 | 0.13 | Heart | 0.10 | ± | 0.04 |
| Lung | 0.24 | 0.44 | Lung | 0.34 | ± | 0.14 |
| Liver | 0.16 | 0.27 | Liver | 0.22 | ± | 0.08 |
| Spleen | 0.08 | 0.16 | Spleen | 0.12 | ± | 0.05 |
| Pancreas | 0.15 | 0.17 | Pancreas | 0.16 | ± | 0.02 |
| Stomach | 0.09 | 0.10 | Stomach | 0.10 | ± | 0.01 |
| Small intestine | 0.14 | 0.35 | Small intestine | 0.25 | ± | 0.15 |
| Large intestine | 0.11 | 0.17 | Large intestine | 0.14 | ± | 0.04 |
| Fat | 0.07 | 0.08 | Fat | 0.08 | ± | 0.01 |
| Muscle | 0.29 | 0.25 | Muscle | 0.27 | ± | 0.03 |
| Salivery gland | 0.22 | 0.27 | Salivary gland | 0.24 | ± | 0.03 |
| Brain | 0.02 | 0.03 | Brain | 0.02 | ± | 0.01 |
| Kidney | 1.12 | 2.23 | Kidney | 1.68 | ± | 0.78 |
| Bladder | 0.36 | 4.97 | Bladder | 2.67 | ± | 3.26 |
| Tumor | 0.83 | 0.93 | Tumor | 0.88 | ± | 0.07 |
| Femur | 0.22 | 0.36 | Femur | 0.29 | ± | 0.10 |

Biodistribution ([¹⁸F]FAPI-74)
Injected dose: 20 μCi per mouse (SA: 378.4 mCi/umol)
Blocker: Cold FAPI-04 (10 nmol)
NUDE, 60 min

| | 22 | 20.9 | | % ID/g | | |
|---|---|---|---|---|---|---|
| Body Wt.: | % ID/g | % ID/g | Tissue | Mean | | Std. |
| Blood | 0.90 | 1.09 | Blood | 0.99 | ± | 0.13 |
| Heart | 0.39 | 0.48 | Heart | 0.43 | ± | 0.06 |
| Lung | 0.72 | 0.82 | Lung | 0.77 | ± | 0.07 |
| Liver | 0.45 | 0.63 | Liver | 0.54 | ± | 0.13 |
| Spleen | 0.25 | 0.33 | Spleen | 0.29 | ± | 0.06 |
| Pancreas | 0.72 | 0.62 | Pancreas | 0.67 | ± | 0.07 |
| Stomach | 0.33 | 0.40 | Stomach | 0.37 | ± | 0.05 |
| Small intestine | 0.36 | 0.41 | Small intestine | 0.38 | ± | 0.04 |
| Large intestine | 0.36 | 0.30 | Large intestine | 0.33 | ± | 0.04 |
| Fat | 0.06 | 0.21 | Fat | 0.13 | ± | 0.11 |
| Muscle | 0.60 | 1.03 | Muscle | 0.82 | ± | 0.31 |
| Salivery gland | 1.24 | 1.45 | Salivary gland | 1.35 | ± | 0.15 |
| Brain | 0.04 | 0.06 | Brain | 0.05 | ± | 0.02 |
| Kidney | 1.77 | 1.90 | Kidney | 1.83 | ± | 0.09 |
| Bladder | 0.57 | 1.27 | Bladder | 0.92 | ± | 0.50 |
| Tumor | 3.18 | 4.81 | Tumor | 3.99 | ± | 1.15 |
| Femur | 2.01 | 2.46 | Femur | 2.24 | ± | 0.31 |

NOD/SCID, 60 min

| | 26.3 | 24.1 | | % ID/g | | |
|---|---|---|---|---|---|---|
| Body Wt.: | % ID/g | % ID/g | Tissue | Mean | | Std. |
| Blood | 0.70 | 0.89 | Blood | 0.80 | ± | 0.13 |
| Heart | 0.83 | 0.46 | Heart | 0.39 | ± | 0.09 |
| Lung | 0.66 | 0.74 | Lung | 0.70 | ± | 0.05 |
| Liver | 2.04 | 1.47 | Liver | 1.75 | ± | 0.41 |
| Spleen | 0.18 | 0.23 | Spleen | 0.20 | ± | 0.04 |
| Pancreas | 0.65 | 1.05 | Pancreas | 0.85 | ± | 0.28 |
| Stomach | 0.36 | 0.40 | Stomach | 0.38 | ± | 0.03 |
| Small intestine | 0.49 | 0.57 | Small intestine | 0.53 | ± | 0.06 |
| Large intestine | 0.37 | 0.47 | Large intestine | 0.42 | ± | 0.07 |
| Fat | 2.12 | 0.16 | Fat | 1.14 | ± | 1.38 |
| Muscle | 5.95 | 1.10 | Muscle | 3.52 | ± | 3.43 |
| Salivery gland | 1.17 | 1.22 | Salivary gland | 1.19 | ± | 0.03 |
| Brain | 0.10 | 0.07 | Brain | 0.08 | ± | 0.03 |
| Kidney | 1.53 | 1.71 | Kidney | 1.62 | ± | 0.12 |
| Bladder | 28.51 | 15.16 | Bladder | 21.84 | ± | 9.44 |
| Tumor | 5.47 | 18.17 | Tumor | 9.32 | ± | 5.44 |
| Femur | 1.75 | 2.05 | Femur | 1.90 | ± | 0.21 |

NUDE, 60 min + Blocker

| | 22.7 | 23.1 | | % ID/g | | |
|---|---|---|---|---|---|---|
| Body Wt.: | % ID/g | % ID/g | Tissue | Mean | | Std. |
| Blood | 0.07 | 0.04 | Blood | 0.05 | ± | 0.02 |
| Heart | 0.05 | 0.03 | Heart | 0.04 | ± | 0.01 |
| Lung | 0.17 | 0.12 | Lung | 0.15 | ± | 0.04 |
| Liver | 0.19 | 0.18 | Liver | 0.19 | ± | 0.01 |
| Spleen | 0.09 | 0.04 | Spleen | 0.07 | ± | 0.03 |
| Pancreas | 0.04 | 0.02 | Pancreas | 0.03 | ± | 0.01 |
| Stomach | 0.07 | 0.04 | Stomach | 0.05 | ± | 0.02 |
| Small intestine | 0.10 | 0.15 | Small intestine | 0.12 | ± | 0.04 |
| Large intestine | 0.06 | 0.05 | Large intestine | 0.06 | ± | 0.01 |
| Fat | 0.03 | 0.02 | Fat | 0.03 | ± | 0.00 |
| Muscle | 0.05 | 0.02 | Muscle | 0.04 | ± | 0.03 |
| Salivery gland | 0.05 | 0.06 | Salivary gland | 0.05 | ± | 0.01 |
| Brain | 0.01 | 0.02 | Brain | 0.02 | ± | 0.01 |
| Kidney | 1.15 | 0.74 | Kidney | 0.94 | ± | 0.29 |
| Bladder | 0.45 | 0.27 | Bladder | 0.36 | ± | 0.13 |
| Tumor | 0.09 | 0.07 | Tumor | 0.08 | ± | 0.02 |
| Femur | 0.52 | 0.36 | Femur | 0.44 | ± | 0.11 |

NOD/SCID, 60 min + Blocker

| | 22.7 | 23.1 | | % ID/g | | |
|---|---|---|---|---|---|---|
| Body Wt.: | % ID/g | % ID/g | Tissue | Mean | | Std. |
| Blood | 0.18 | 0.19 | Blood | 0.19 | ± | 0.01 |
| Heart | 0.11 | 0.15 | Heart | 0.13 | ± | 0.03 |
| Lung | 0.27 | 0.49 | Lung | 0.38 | ± | 0.16 |
| Liver | 1.74 | 1.65 | Liver | 1.70 | ± | 0.06 |
| Spleen | 0.10 | 0.15 | Spleen | 0.13 | ± | 0.04 |
| Pancreas | 0.07 | 0.19 | Pancreas | 0.10 | ± | 0.04 |
| Stomach | 0.18 | 0.17 | Stomach | 0.17 | ± | 0.01 |
| Small intestine | 0.48 | 0.45 | Small intestine | 0.47 | ± | 0.02 |
| Large intestine | 0.18 | 0.19 | Large intestine | 0.18 | ± | 0.00 |
| Fat | 0.07 | 0.10 | Fat | 0.08 | ± | 0.02 |
| Muscle | 0.07 | 0.12 | Muscle | 0.09 | ± | 0.03 |
| Salivery gland | 0.12 | 0.14 | Salivary gland | 0.13 | ± | 0.02 |
| Brain | 0.02 | 0.08 | Brain | 0.02 | ± | 0.01 |
| Kidney | 1.61 | 1.54 | Kidney | 1.57 | ± | 0.05 |
| Bladder | 12.70 | 11.31 | Bladder | 12.01 | ± | 0.99 |
| Tumor | 0.21 | 0.23 | Tumor | 0.22 | ± | 0.01 |
| Femur | 0.87 | 0.43 | Femur | 0.40 | ± | 0.04 |

NUDE, 120 min

| | 25.5 | 24.5 | | % ID/g | | |
|---|---|---|---|---|---|---|
| Body Wt.: | % ID/g | % ID/g | Tissue | Mean | | Std. |
| Blood | 0.66 | 0.56 | Blood | 0.61 | ± | 0.07 |
| Heart | 0.34 | 0.28 | Heart | 0.31 | ± | 0.04 |
| Lung | 0.49 | 0.56 | Lung | 0.53 | ± | 0.05 |
| Liver | 0.45 | 0.42 | Liver | 0.44 | ± | 0.02 |
| Spleen | 0.22 | 0.22 | Spleen | 0.22 | ± | 0.00 |
| Pancreas | 0.53 | 0.34 | Pancreas | 0.43 | ± | 0.14 |
| Stomach | 0.27 | 0.25 | Stomach | 0.26 | ± | 0.01 |
| Small intestine | 0.35 | 0.31 | Small intestine | 0.33 | ± | 0.03 |
| Large intestine | 0.34 | 0.41 | Large intestine | 0.37 | ± | 0.05 |
| Fat | 0.13 | 0.10 | Fat | 0.12 | ± | 0.02 |
| Muscle | 0.49 | 0.45 | Muscle | 0.47 | ± | 0.03 |
| Salivery gland | 1.02 | 0.78 | Salivary gland | 0.90 | ± | 0.17 |
| Brain | 0.05 | 0.06 | Brain | 0.06 | ± | 0.00 |
| Kidney | 1.50 | 1.43 | Kidney | 1.46 | ± | 0.06 |
| Bladder | 1.31 | 1.80 | Bladder | 1.55 | ± | 0.35 |
| Tumor | 2.65 | 3.51 | Tumor | 3.08 | ± | 0.61 |
| Femur | 2.27 | 2.55 | Femur | 2.41 | ± | 0.20 |

NOD/SCID, 120 min

| | 25.5 | 24.5 | | % ID/g | | |
|---|---|---|---|---|---|---|
| Body Wt.: | % ID/g | % ID/g | Tissue | Mean | | Std. |
| Blood | 0.40 | 0.47 | Blood | 0.43 | ± | 0.05 |
| Heart | 0.20 | 0.29 | Heart | 0.24 | ± | 0.06 |
| Lung | 0.36 | 0.57 | Lung | 0.47 | ± | 0.15 |
| Liver | 1.11 | 0.88 | Liver | 1.00 | ± | 0.16 |
| Spleen | 0.26 | 0.36 | Spleen | 0.31 | ± | 0.07 |
| Pancreas | 0.38 | 0.77 | Pancreas | 0.57 | ± | 0.27 |
| Stomach | 0.22 | 0.35 | Stomach | 0.28 | ± | 0.09 |
| Small intestine | 0.38 | 0.95 | Small intestine | 0.67 | ± | 0.40 |
| Large intestine | 0.30 | 0.45 | Large intestine | 0.37 | ± | 0.11 |
| Fat | 0.09 | 0.18 | Fat | 0.11 | ± | 0.04 |
| Muscle | 0.25 | 0.35 | Muscle | 0.30 | ± | 0.07 |
| Salivery gland | 0.74 | 0.62 | Salivary gland | 0.58 | ± | 0.08 |
| Brain | 0.04 | 0.04 | Brain | 0.04 | ± | 0.00 |
| Kidney | 1.51 | 1.58 | Kidney | 1.55 | ± | 0.05 |
| Bladder | 3.86 | | Bladder | 3.86 | ± | |
| Tumor | 3.13 | | Tumor | 3.13 | ± | |
| Femur | 1.59 | | Femur | 1.59 | ± | |

Gamma counter error for the last three samples
Biodistribution ([¹⁸F]JHU1221823)

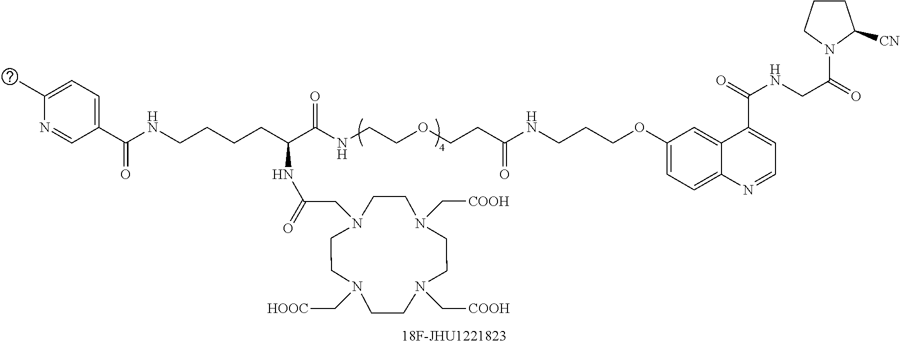

Injected dose: 20 μCi per mouse
Carrier: 0.25, 0.1, 0.05, and 0.01 nmol/mouse
60 min post injection
[¹⁸F]JHU1221823 + 0.25 nmole

| | 20.2 | 21.8 | | % ID/g | | |
|---|---|---|---|---|---|---|
| Body Wt.: | % ID/g | % ID/g | Tissue | Mean | | Std. |
| Blood | 1.76 | 2.03 | Blood | 1.90 | ± | 0.19 |
| Heart | 0.91 | 0.91 | Heart | 0.91 | ± | 0.00 |
| Lung | 1.17 | 1.57 | Lung | 1.37 | ± | 0.29 |
| Liver | 0.73 | 0.77 | Liver | 0.75 | ± | 0.03 |
| Spleen | 0.44 | 0.48 | Spleen | 0.46 | ± | 0.03 |
| Pancreas | 2.11 | 1.42 | Pancreas | 1.77 | ± | 0.49 |
| Stomach | 0.61 | 0.77 | Stomach | 0.69 | ± | 0.12 |
| Small intestine | 0.75 | 1.07 | Small intestine | 0.91 | ± | 0.23 |
| Large intestine | 0.62 | 0.68 | Large intestine | 0.65 | ± | 0.04 |
| Fat | 0.65 | 0.37 | Fat | 0.51 | ± | 0.19 |
| Muscle | 1.32 | 1.19 | Muscle | 1.25 | ± | 0.09 |
| Salivery gland | 3.78 | 2.85 | Salivary gland | 3.31 | ± | 0.66 |
| Brain | 0.08 | 0.08 | Brain | 0.08 | ± | 0.00 |
| Kidney | 1.70 | 2.15 | Kidney | 1.92 | ± | 0.32 |
| Bladder | 3.04 | 1.98 | Bladder | 2.51 | ± | 0.75 |
| Tumor | 3.93 | 3.11 | Tumor | 3.52 | ± | 0.57 |
| Femur | 3.97 | 4.56 | Femur | 4.26 | ± | 0.42 |

[¹⁸F]JHU1221823 + 0.1 nmole

| | 22.7 | 23.1 | | % ID/g | | |
|---|---|---|---|---|---|---|
| Body Wt.: | % ID/g | % ID/g | Tissue | Mean | | Std. |
| Blood | 3.88 | 4.32 | Blood | 4.10 | ± | 0.32 |
| Heart | 1.80 | 1.87 | Heart | 1.84 | ± | 0.05 |
| Lung | 2.31 | 2.83 | Lung | 2.57 | ± | 0.36 |
| Liver | 1.01 | 1.30 | Liver | 1.16 | ± | 0.21 |
| Spleen | 0.91 | 1.01 | Spleen | 0.96 | ± | 0.07 |
| Pancreas | 4.18 | 5.17 | Pancreas | 4.67 | ± | 0.70 |
| Stomach | 0.89 | 1.12 | Stomach | 1.00 | ± | 0.16 |
| Small intestine | 1.66 | 1.63 | Small intestine | 1.64 | ± | 0.02 |
| Large intestine | 2.91 | 1.79 | Large intestine | 2.35 | ± | 0.79 |
| Fat | 2.17 | 1.13 | Fat | 1.65 | ± | 0.74 |
| Muscle | 1.69 | 2.25 | Muscle | 1.97 | ± | 0.40 |
| Salivery gland | 7.28 | 9.35 | Salivary gland | 8.31 | ± | 1.46 |
| Brain | 0.08 | 0.17 | Brain | 0.12 | ± | 0.06 |
| Kidney | 2.03 | 2.00 | Kidney | 2.01 | ± | 0.03 |
| Bladder | 1.62 | 1.87 | Bladder | 1.75 | ± | 0.18 |
| Tumor | 5.48 | 4.73 | Tumor | 5.08 | ± | 0.49 |
| Femur | 3.44 | 4.00 | Femur | 3.72 | ± | 0.40 |

[¹⁸F]JHU1221823 + 0.05 nmole

| | 25.5 | 24.5 | | % ID/g | | |
|---|---|---|---|---|---|---|
| Body Wt.: | % ID/g | % ID/g | Tissue | Mean | | Std. |
| Blood | 7.42 | 7.67 | Blood | 7.55 | ± | 0.18 |
| Heart | 3.03 | 3.04 | Heart | 3.03 | ± | 0.01 |
| Lung | 4.05 | 4.16 | Lung | 4.10 | ± | 0.08 |
| Liver | 1.92 | 2.34 | Liver | 2.13 | ± | 0.30 |
| Spleen | 1.37 | 1.62 | Spleen | 1.50 | ± | 0.18 |
| Pancreas | 4.01 | 6.69 | Pancreas | 5.35 | ± | 1.90 |
| Stomach | 1.07 | 1.48 | Stomach | 1.27 | ± | 0.29 |
| Small intestine | 1.46 | 2.18 | Small intestine | 1.82 | ± | 0.51 |
| Large intestine | 1.48 | 1.58 | Large intestine | 1.53 | ± | 0.07 |
| Fat | 0.94 | 0.61 | Fat | 0.78 | ± | 0.24 |
| Muscle | 1.47 | 2.20 | Muscle | 1.83 | ± | 0.52 |
| Salivery gland | 10.22 | 11.63 | Salivary gland | 10.93 | ± | 1.00 |
| Brain | 0.20 | 0.21 | Brain | 0.21 | ± | 0.01 |
| Kidney | 2.02 | 2.55 | Kidney | 2.29 | ± | 0.38 |
| Bladder | 6.14 | 2.43 | Bladder | 4.29 | ± | 2.63 |
| Tumor | 2.59 | 4.26 | Tumor | 3.42 | ± | 1.18 |
| Femur | 2.83 | 3.67 | Femur | 3.25 | ± | 0.60 |

Biodistribution ([¹⁸F]JHU1221823

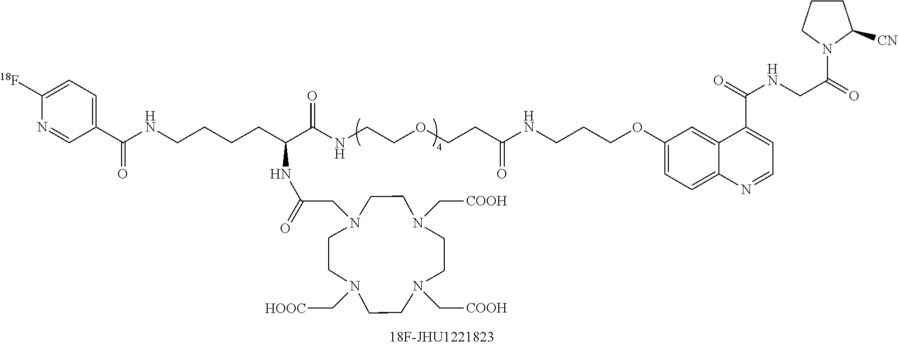

Injected dose: 20 μCi per mouse (SA: 7200 mCi/μmol)
Blocker: Cold FAPI-04 (10 nmol)
Carrier: 0.5 nmol/mouse
[¹⁸F]JHU1221823 + 0.01 nmole

| | 22.3 | 21.7 | | % ID/g | | |
|---|---|---|---|---|---|---|
| Body Wt.: | % ID/g | % ID/g | Tissue | Mean | | Std. |
| Blood | 11.19 | 11.39 | Blood | 11.29 | ± | 0.14 |
| Heart | 4.74 | 4.50 | Heart | 4.62 | ± | 0.17 |
| Lung | 6.08 | 6.21 | Lung | 6.15 | ± | 0.09 |
| Liver | 2.78 | 3.40 | Liver | 3.09 | ± | 0.44 |
| Spleen | 1.88 | 2.13 | Spleen | 2.01 | ± | 0.18 |
| Pancreas | 4.01 | 4.14 | Pancreas | 4.07 | ± | 0.09 |
| Stomach | 1.33 | 1.65 | Stomach | 1.49 | ± | 0.22 |
| Small intestine | 2.22 | 2.17 | Small intestine | 2.20 | ± | 0.03 |
| Large intestine | 1.60 | 1.63 | Large intestine | 1.62 | ± | 0.02 |
| Fat | 0.23 | 0.44 | Fat | 0.34 | ± | 0.15 |
| Muscle | 2.03 | 1.73 | Muscle | 1.88 | ± | 0.21 |
| Salivery gland | 7.47 | 7.48 | Salivary gland | 7.47 | ± | 0.00 |
| Brain | 0.32 | 0.28 | Brain | 0.30 | ± | 0.02 |
| Kidney | 2.82 | 3.11 | Kidney | 2.96 | ± | 0.21 |
| Bladder | 1.82 | 2.18 | Bladder | 2.00 | ± | 0.26 |
| Tumor | 1.88 | 2.86 | Tumor | 2.37 | ± | 0.70 |
| Femur | 3.05 | 4.01 | | 3.53 | ± | 0.68 |

[¹⁸F]JHU1221823

| | 22.7 | 23.1 | | % ID/g | | |
|---|---|---|---|---|---|---|
| Body Wt.: | % ID/g | % ID/g | Tissue | Mean | | Std. |
| Blood | 9.28 | 9.80 | Blood | 9.54 | ± | 0.37 |
| Heart | 4.31 | 4.98 | Heart | 4.65 | ± | 0.48 |
| Lung | 5.58 | 5.91 | Lung | 5.72 | ± | 0.27 |
| Liver | 2.68 | 3.29 | Liver | 2.99 | ± | 0.43 |
| Spleen | 1.85 | 2.43 | Spleen | 2.15 | ± | 0.40 |
| Pancreas | 1.62 | 2.66 | Pancreas | 2.14 | ± | 0.73 |
| Stomach | 0.84 | 1.45 | Stomach | 1.14 | ± | 0.43 |
| Small intestine | 2.59 | 3.46 | Small intestine | 3.02 | ± | 0.62 |
| Large intestine | 1.29 | 1.69 | Large intestine | 1.49 | ± | 0.29 |
| Fat | 0.47 | 1.68 | Fat | 1.07 | ± | 0.86 |
| Muscle | 1.79 | 1.78 | Muscle | 1.78 | ± | 0.01 |
| Salivery gland | 7.44 | 6.43 | Salivary gland | 6.93 | ± | 0.71 |
| Brain | 0.23 | 0.22 | Brain | 0.22 | ± | 0.01 |
| Kidney | 2.70 | 2.77 | Kidney | 2.73 | ± | 0.05 |
| Bladder | 6.41 | 2.64 | Bladder | 4.53 | ± | 2.66 |
| Tumor | 4.35 | 7.0+ | Tumor | 5.71 | ± | 1.92 |
| Femur | 2.55 | 3.11 | Femur | 2.83 | ± | 0.40 |

Biodistribution ([¹⁸F]JHU1221823)

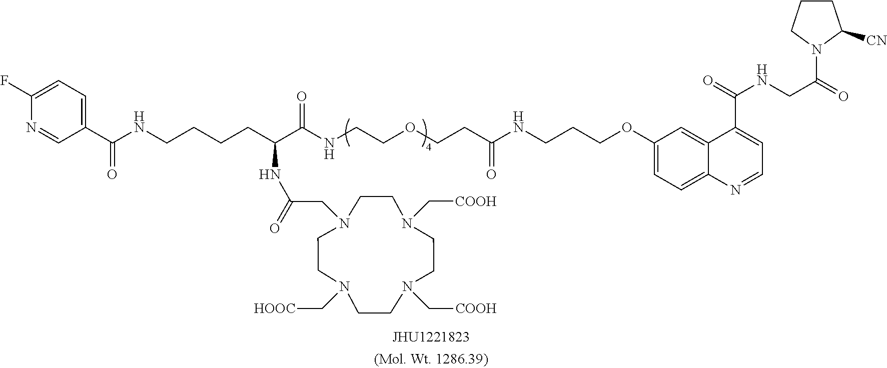

Injected dose: 5 μCi per mouse
Baseline (~5000 mCi/μmol), Carrier 350 (350 mCi/μmol), Carrier 700 (mCi/μmol)
Time: 60 min post injection of the tracer
Tested in Nude and NOD/SCID model with U87 tumor
<Nude mice>
[¹⁸F]JHU1221823, Baseline

| | 23 | 22.2 | | % ID/g | | |
|---|---|---|---|---|---|---|
| Body Wt.: | % ID/g | % ID/g | Tissue | Mean | | Std. |
| Blood | 10.83 | 12.08 | Blood | 11.46 | ± | 0.88 |
| Heart | 5.09 | 5.98 | Heart | 5.54 | ± | 0.63 |
| Lung | 6.48 | 7.46 | Lung | 6.97 | ± | 0.69 |
| Liver | 3.56 | 3.06 | Liver | 3.31 | ± | 0.36 |
| Spleen | 2.17 | 1.96 | Spleen | 2.06 | ± | 0.15 |
| Pancreas | 3.42 | 3.30 | Pancreas | 3.36 | ± | 0.09 |
| Stomach | 1.14 | 1.32 | Stomach | 1.23 | ± | 0.13 |
| Small intestine | 2.47 | 2.93 | Small intestine | 2.70 | ± | 0.33 |
| Large intestine | 1.38 | 1.42 | Large intestine | 1.40 | ± | 0.03 |
| Fat | 1.10 | 0.89 | Fat | 0.99 | ± | 0.15 |
| Muscle | 1.44 | 2.06 | Muscle | 1.75 | ± | 0.44 |
| Salivery gland | 6.35 | 7.47 | Salivary gland | 6.91 | ± | 0.79 |
| Brain | 0.22 | 0.24 | Brain | 0.23 | ± | 0.01 |
| Kidney | 2.76 | 3.06 | Kidney | 2.91 | ± | 0.21 |
| Bladder | 2.02 | 2.74 | Bladder | 2.38 | ± | 0.51 |
| Tumor | 2.14 | 2.52 | Tumor | 2.33 | ± | 0.27 |
| Femur | 1.98 | 2.84 | Femur | 2.41 | ± | 0.61 |

[¹⁸F]JHU1221823, Carrier 350

| | 22.7 | 23.1 | | % ID/g | | |
|---|---|---|---|---|---|---|
| Body Wt.: | % ID/g | % ID/g | Tissue | Mean | | Std. |
| Blood | 6.11 | 4.56 | Blood | 5.84 | ± | 1.10 |
| Heart | 2.83 | 2.18 | Heart | 2.50 | ± | 0.46 |
| Lung | 3.70 | 3.20 | Lung | 3.45 | ± | 0.36 |
| Liver | 2.06 | 1.75 | Liver | 1.90 | ± | 0.22 |
| Spleen | 1.45 | 1.12 | Spleen | 1.29 | ± | 0.23 |
| Pancreas | 5.80 | 6.78 | Pancreas | 6.29 | ± | 0.69 |
| Stomach | 1.60 | 1.53 | Stomach | 1.56 | ± | 0.05 |
| Small intestine | 2.29 | 2.54 | Small intestine | 2.42 | ± | 0.17 |
| Large intestine | 1.92 | 1.61 | Large intestine | 1.76 | ± | 0.22 |
| Fat | 0.54 | 1.24 | Fat | 0.89 | ± | 0.50 |
| Muscle | 2.58 | 2.23 | Muscle | 2.41 | ± | 0.25 |
| Salivery gland | 10.81 | 11.50 | Salivary gland | 11.15 | ± | 0.49 |
| Brain | 0.16 | 0.12 | Brain | 0.14 | ± | 0.03 |
| Kidney | 2.25 | 1.88 | Kidney | 2.07 | ± | 0.26 |
| Bladder | 1.62 | 1.85 | Bladder | 1.73 | ± | 0.16 |
| Tumor | 2.01 | 2.53 | Tumor | 2.27 | ± | 0.37 |
| Femur | 3.83 | 4.47 | Femur | 4.15 | ± | 0.45 |

[¹⁸F]JHU1221823, Carroer 700

| | 25.5 | 24.5 | | % ID/g | | |
|---|---|---|---|---|---|---|
| Body Wt.: | % ID/g | % ID/g | Tissue | Mean | | Std. |
| Blood | 10.55 | 9.33 | Blood | 9.94 | ± | 0.86 |
| Heart | 4.92 | 4.38 | Heart | 4.65 | ± | 0.38 |
| Lung | 6.05 | 5.08 | Lung | 5.57 | ± | 0.69 |
| Liver | 2.76 | 2.85 | Liver | 2.81 | ± | 0.06 |
| Spleen | 1.75 | 1.70 | Spleen | 1.72 | ± | 0.03 |
| Pancreas | 3.40 | 4.43 | Pancreas | 3.91 | ± | 0.73 |
| Stomach | 1.18 | 1.57 | Stomach | 1.37 | ± | 0.28 |
| Small intestine | 2.37 | 1.98 | Small intestine | 2.18 | ± | 0.28 |
| Large intestine | 1.45 | 1.79 | Large intestine | 1.62 | ± | 0.24 |
| Fat | 0.41 | 0.56 | Fat | 0.49 | ± | 0.10 |
| Muscle | 2.06 | 2.07 | Muscle | 2.07 | ± | 0.01 |
| Salivery gland | 9.08 | 7.18 | Salivary gland | 8.13 | ± | 1.34 |
| Brain | 0.21 | 0.19 | Brain | 0.20 | ± | 0.02 |
| Kidney | 2.54 | 2.52 | Kidney | 2.53 | ± | 0.02 |
| Bladder | 2.15 | 2.30 | Bladder | 2.23 | ± | 0.11 |
| Tumor | 1.81 | 2.22 | Tumor | 2.01 | ± | 0.29 |
| Femur | 2.93 | 3.18 | Femur | 3.06 | ± | 0.17 |

[¹⁸F]JHU1221823, Baseline

| | 22.4 | 20.1 | | % ID/g | | |
|---|---|---|---|---|---|---|
| Body Wt.: | % ID/g | % ID/g | Tissue | Mean | | Std. |
| Blood | 11.98 | 10.01 | Blood | 10.99 | ± | 1.40 |
| Heart | 7.41 | 6.48 | Heart | 6.95 | ± | 0.66 |
| Lung | 7.27 | 6.79 | Lung | 7.03 | ± | 0.34 |
| Liver | 4.04 | 4.72 | Liver | 4.38 | ± | 0.48 |
| Spleen | 2.33 | 1.99 | Spleen | 2.16 | ± | 0.24 |
| Pancreas | 6.82 | 9.52 | Pancreas | 8.17 | ± | 1.91 |
| Stomach | 1.66 | 1.97 | Stomach | 1.81 | ± | 0.22 |
| Small intestine | 3.79 | 3.54 | Small intestine | 3.66 | ± | 0.17 |
| Large intestine | 1.66 | 2.70 | Large intestine | 2.18 | ± | 0.73 |
| Fat | 0.53 | 0.71 | Fat | 0.62 | ± | 0.12 |
| Muscle | 1.14 | 2.13 | Muscle | 1.64 | ± | 0.69 |
| Salivery gland | 11.69 | 12.09 | Salivary gland | 11.89 | ± | 0.28 |
| Brain | 0.23 | 0.21 | Brain | 0.22 | ± | 0.01 |
| Kidney | 3.14 | 3.67 | Kidney | 3.41 | ± | 0.37 |
| Bladder | 3.22 | 10.62 | Bladder | 6.92 | ± | 5.24 |
| Tumor | 1.17 | 5.00 | Tumor | 3.08 | ± | 2.71 |
| Femur | 1.95 | 2.78 | | 2.37 | ± | 0.59 |

<NOD/SCID>
[¹⁸F]JHU1221823, Carrier 350

| | 20 | 20 | | % ID/g | | |
|---|---|---|---|---|---|---|
| Body Wt.: | % ID/g | % ID/g | Tissue | Mean | | Std. |
| Blood | 3.99 | 4.29 | Blood | 4.14 | ± | 0.21 |
| Heart | 2.25 | 2.02 | Heart | 2.14 | ± | 0.16 |
| Lung | 2.51 | 2.98 | Lung | 2.74 | ± | 0.33 |
| Liver | 1.71 | 1.81 | Liver | 1.76 | ± | 0.07 |
| Spleen | 1.60 | 1.01 | Spleen | 1.80 | ± | 0.42 |
| Pancreas | 10.66 | 9.69 | Pancreas | 10.18 | ± | 0.69 |
| Stomach | 1.47 | 1.47 | Stomach | 1.47 | ± | 0.00 |
| Small intestine | 2.86 | 1.91 | Small intestine | 2.29 | ± | 0.53 |
| Large intestine | 2.18 | 1.97 | Large intestine | 2.08 | ± | 0.15 |
| Fat | 0.89 | 0.82 | Fat | 0.85 | ± | 0.05 |
| Muscle | 2.02 | 2.04 | Muscle | 2.03 | ± | 0.01 |
| Salivery gland | 9.63 | 9.35 | Salivary gland | 9.49 | ± | 0.20 |
| Brain | 0.10 | 0.09 | Brain | 0.09 | ± | 0.01 |
| Kidney | 2.00 | 2.27 | Kidney | 2.13 | ± | 0.19 |
| Bladder | 2.09 | 6.57 | Bladder | 4.33 | ± | 3.17 |
| Tumor | 8.22 | 9.06 | Tumor | 8.64 | ± | 0.59 |
| Femur | 3.67 | 3.18 | Femur | 3.43 | ± | 0.34 |

[¹⁸F]JHU1221823, Carrier 700

| | 21.6 | 25.8 | | % ID/g | | |
|---|---|---|---|---|---|---|
| Body Wt.: | % ID/g | % ID/g | Tissue | Mean | | Std. |
| Blood | 5.82 | | Blood | 5.82 | ± | |
| Heart | 3.48 | | Heart | 3.48 | ± | |
| Lung | 3.66 | | Lung | 3.56 | ± | |
| Liver | 2.36 | | Liver | 2.36 | ± | |
| Spleen | 1.46 | | Spleen | 1.46 | ± | |
| Pancreas | 13.03 | | Pancreas | 13.03 | ± | |
| Stomach | 1.64 | | Stomach | 1.64 | ± | |
| Small intestine | 3.48 | | Small intestine | 3.48 | ± | |
| Large intestine | 2.26 | | Large intestine | 2.26 | ± | |
| Fat | 0.90 | | Fat | 0.90 | ± | |
| Muscle | 1.99 | | Muscle | 1.99 | ± | |
| Salivery gland | 11.31 | | Salivary gland | 11.31 | ± | |
| Brain | 0.16 | | Brain | 0.16 | ± | |
| Kidney | 2.45 | | Kidney | 2.45 | ± | |
| Bladder | 8.20 | | Bladder | 8.20 | ± | |
| Tumor | 7.79 | | Tumor | 7.79 | ± | |
| Femur | 2.41 | | Femur | 2.41 | ± | |

Biodistribution ([¹⁸F]Sri-08-51)
Injected dose: 25 μCi per mouse (SA: 1966 mCi/umol)
Chelated with cold indium
Blocker: Cold FAPI-04 (10 nmol)
Tested in both Nude and NOD/SCID models with U87 at 1 hr post injection
Nude Model
[¹⁸F]Sri-08-51 + Blocker, 60 min

| | 24.9 | 26 | | % ID/g | | |
|---|---|---|---|---|---|---|
| Body Wt.: | % ID/g | % ID/g | Tissue | Mean | | Std. |
| Blood | 0.10 | 0.10 | Blood | 0.10 | ± | 0.00 |
| Heart | 0.05 | 0.05 | Heart | 0.05 | ± | 0.00 |
| Lung | 0.21 | 0.22 | Lung | 0.21 | ± | 0.00 |
| Liver | 1.88 | 2.01 | Liver | 1.95 | ± | 0.10 |
| Spleen | 0.08 | 0.08 | Spleen | 0.08 | ± | 0.00 |
| Pancreas | 0.05 | 0.05 | Pancreas | 0.05 | ± | 0.00 |
| Stomach | 0.08 | 0.15 | Stomach | 0.11 | ± | 0.05 |
| Small intestine | 3.50 | 2.97 | Small intestine | 3.24 | ± | 0.38 |
| Large intestine | 0.08 | 0.11 | Large intestine | 0.09 | ± | 0.02 |
| Fat | 0.03 | 0.01 | Fat | 0.02 | ± | 0.01 |
| Muscle | 0.07 | 0.04 | Muscle | 0.05 | ± | 0.02 |
| Salivery gland | 0.07 | 0.08 | Salivary gland | 0.08 | ± | 0.00 |
| Brain | 0.01 | 0.01 | Brain | 0.01 | ± | 0.00 |
| Kidney | 1.19 | 1.05 | Kidney | 1.12 | ± | 0.10 |
| Bladder | 0.22 | 0.74 | Bladder | 0.48 | ± | 0.37 |
| Tumor | 0.17 | 0.18 | Tumor | 0.18 | ± | 0.01 |
| Femur | 0.12 | 0.12 | Femur | 0.12 | ± | 0.00 |

[¹⁸F]Sri-08-51, 60 min

| | 22.7 | 23.1 | | % ID/g | | |
|---|---|---|---|---|---|---|
| Body Wt.: | % ID/g | % ID/g | Tissue | Mean | | Std. |
| Blood | 3.06 | 3.20 | Blood | 3.13 | ± | 0.10 |
| Heart | 1.35 | 1.52 | Heart | 1.43 | ± | 0.12 |
| Lung | 2.03 | 2.25 | Lung | 2.14 | ± | 0.16 |
| Liver | 1.81 | 1.93 | Liver | 1.87 | ± | 0.09 |
| Spleen | 0.90 | 1.02 | Spleen | 0.96 | ± | 0.08 |
| Pancreas | 3.94 | 3.77 | Pancreas | 3.85 | ± | 0.12 |
| Stomach | 1.06 | 1.16 | Stomach | 1.11 | ± | 0.07 |
| Small intestine | 1.93 | 1.63 | Small intestine | 1.78 | ± | 0.21 |
| Large intestine | 1.20 | 1.30 | Large intestine | 1.25 | ± | 0.08 |
| Fat | 0.52 | 0.77 | Fat | 0.64 | ± | 0.18 |
| Muscle | 1.86 | 1.93 | Muscle | 1.90 | ± | 0.05 |
| Salivery gland | 7.74 | 8.71 | Salivary gland | 8.22 | ± | 0.69 |
| Brain | 0.09 | 0.08 | Brain | 0.09 | ± | 0.01 |
| Kidney | 1.69 | 1.72 | Kidney | 1.70 | ± | 0.02 |
| Bladder | 1.18 | 2.99 | Bladder | 2.09 | ± | 1.28 |
| Tumor | 10.87 | 13.08 | Tumor | 11.97 | ± | 1.56 |
| Femur | 4.44 | 3.86 | Femur | 4.15 | ± | 0.41 |

NOD/SCID Model
[¹⁸F]Sri-08-51 + Blocker, 60 min

| | 25.5 | 24.5 | | % ID/g | | |
|---|---|---|---|---|---|---|
| Body Wt.: | % ID/g | % ID/g | Tissue | Mean | | Std. |
| Blood | 0.22 | 0.40 | Blood | 0.31 | ± | 0.13 |
| Heart | 0.11 | 0.25 | Heart | 0.18 | ± | 0.10 |
| Lung | 0.32 | 0.61 | Lung | 0.47 | ± | 0.21 |
| Liver | 4.02 | 8.14 | Liver | 6.08 | ± | 2.91 |
| Spleen | 0.14 | 0.32 | Spleen | 0.23 | ± | 0.13 |
| Pancreas | 0.08 | 0.17 | Pancreas | 0.13 | ± | 0.07 |
| Stomach | 0.25 | 0.45 | Stomach | 0.35 | ± | 0.15 |
| Small intestine | 0.94 | 2.12 | Small intestine | 1.53 | ± | 0.84 |
| Large intestine | 0.19 | 0.35 | Large intestine | 0.27 | ± | 0.11 |
| Fat | 0.06 | 0.06 | Fat | 0.06 | ± | 0.00 |
| Muscle | 0.07 | 0.49 | Muscle | 0.28 | ± | 0.30 |
| Salivery gland | 0.45 | 0.26 | Salivary gland | 0.35 | ± | 0.14 |
| Brain | 0.02 | 1.02 | Brain | 0.52 | ± | 0.70 |
| Kidney | 1.42 | 1.40 | Kidney | 1.41 | ± | 0.01 |
| Bladder | 13.35 | 61.94 | Bladder | 37.64 | ± | 34.36 |
| Tumor | 1.34 | 0.91 | Tumor | 1.13 | ± | 0.30 |
| Femur | 0.88 | 0.38 | Femur | 0.88 | ± | 0.00 |

[¹⁸F]Sri-08-51, 60 min

| | 21 | 21.4 | | % ID/g | | |
|---|---|---|---|---|---|---|
| Body Wt.: | % ID/g | % ID/g | Tissue | Mean | | Std. |
| Blood | 4.18 | 5.83 | Blood | 5.01 | ± | 1.17 |
| Heart | 1.74 | 2.39 | Heart | 2.07 | ± | 0.45 |
| Lung | 2.77 | 2.76 | Lung | 2.77 | ± | 0.00 |
| Liver | 3.23 | 4.21 | Liver | 3.72 | ± | 0.69 |
| Spleen | 1.27 | 1.71 | Spleen | 1.49 | ± | 0.31 |
| Pancreas | 3.99 | 4.82 | Pancreas | 4.40 | ± | 0.59 |
| Stomach | 1.65 | 1.75 | Stomach | 1.70 | ± | 0.06 |
| Small intestine | 2.09 | 2.09 | Small intestine | 2.09 | ± | 0.00 |
| Large intestine | 1.91 | 1.87 | Large intestine | 1.89 | ± | 0.03 |
| Fat | 2.81 | 0.45 | Fat | 1.63 | ± | 1.67 |
| Muscle | 2.84 | 2.83 | Muscle | 2.84 | ± | 0.01 |
| Salivery gland | 12.48 | 14.32 | Salivary gland | 13.40 | ± | 1.30 |
| Brain | 0.26 | 0.25 | Brain | 0.25 | ± | 0.01 |
| Kidney | 2.10 | 2.19 | Kidney | 2.15 | ± | 0.07 |
| Bladder | 44.77 | 23.08 | Bladder | 33.92 | ± | 15.34 |
| Tumor | 16.64 | 15.98 | Tumor | 16.31 | ± | 0.46 |
| Femur | 4.83 | 5.16 | | 5.00 | ± | 0.23 |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Allinen M, Beroukhim R, Cai L, Brennan C, Lahti-Domenici J, Huang H, Porter D, Hu M, Chin L, Richardson A, Schnitt S, Sellers W R, Polyak K. Molecular characterization of the tumor microenvironment in breast cancer. Cancer Cell. 2004 July; 6 (1): 17-32.
Bae S, Park C W, Son H K, Ju H K, Paik D, Jeon C J, Koh G Y, Kim J, Kim H. Fibroblast activation protein alpha identifies mesenchymal stromal cells from human bone marrow. Br J Haematol. 2008 September; 142 (5): 827-30.
Bandari R P, Carmack T L, Malhotra A, Watkinson L, Fergason Cantrell E A, Lewis M R, et al. Development of heterobivalent theranostic probes having high affinity/selectivity for the GRPR/PSMA. Journal of Medicinal Chemistry. 2021; 64:2151-66.
Banerjee et al., Preclinical Evaluation of 203/212Pb-Labeled Low-Molecular-Weight Compounds for Targeted Radiopharmaceutical Therapy of Prostate Cancer, J. Nucl. Med., 61 (1), 80-88 (2020).
Banerjee S R, Kumar V, Lisok A, Chen J, Minn I, Brummet M, et al. 177Lu-labeled low-molecular-weight agents for PSMA-targeted radiopharmaceutical therapy. Eur J Nucl Med Mol Imaging. 2019; 46:2545-57.
Banerjee S R, Pullambhatla M, Byun Y, Nimmagadda S, Foss C A, Green G, et al. Sequential SPECT and Optical Imaging of Experimental Models of Prostate Cancer with a Dual Modality Inhibitor of the Prostate-Specific Membrane Antigen. Angewandte Chemie International Edition. 2011; 50:9167-70.
Bejarano L, Jordão MJC, Joyce JA. Therapeutic targeting of the tumor microenvironment. Cancer Discovery. 2021; 11:933-59.
Brennen W N, Isaacs J T, Denmeade S R. Rationale behind targeting fibroblast activation protein-expressing carcinoma-associated fibroblasts as a novel chemotherapeutic strategy. Molecular Cancer Therapeutics. 2012; 11:257-66.
Busek P, Balaziova E, Matrasova I, Hilser M, Tomas R, Syrucek M, et al. Fibroblast activation protein alpha is expressed by transformed and stromal cells and is associated with mesenchymal features in glioblastoma. Tumor Biology. 2016; 37:13961-71.
Chang S S, Reuter V E, Heston W D W, Bander N H, Grauer L S, Gaudin P B. Five different anti-prostate-specific membrane antigen (PSMA) antibodies confirm PSMA expression in tumor-associated neovasculature. Cancer Res. 1999; 59:3192.
Chen Z Y, Wang Y X, Lin Y, Zhang J S, Yang F, Zhou Q L, Liao Y Y. Advance of molecular imaging technology and targeted imaging agent in imaging and therapy. Biomed Res Int. 2014; 2014:819324. PMCID: PMC3943245.
Cheng Y, Prusoff W H. Relationship between the inhibition constant (K1) and the concentration of inhibitor which causes 50 percent inhibition (I50) of an enzymatic reaction. Biochem Pharmacol. 1973; 22:3099-108.
Cho et al., Biodistribution, tumor detection, and radiation dosimetry of 18F-DCFBC, a low-molecular-weight inhibitor of prostate-specific membrane antigen, in patients with metastatic prostate cancer. Journal of nuclear medicine: official publication, Society of Nuclear Medicine. 2012; 53:1883-1891.
Cho, S Y, et al., Biodistribution, tumor detection, and radiation dosimetry of 18F-DCFBC, a low-molecular-weight inhibitor of prostate-specific membrane antigen, in patients with metastatic prostate cancer. Journal of nuclear medicine: official publication, Society of Nuclear Medicine. 2012; 53:1883-1891.
Coenen H H, Elsinga P H, Iwata R, Kilbourn M R, Pillai M R, Rajan M G, Wagner H N Jr. Zaknun J J. Fluorine-18 radiopharmaceuticals beyond [18F]FDG for use in oncology and neurosciences. Nuclear medicine and biology. 2010; 37:727-740.
Cohen A, Larson S M, Old L J, et al, Antibody targeting in metastatic colon cancer: a phase I study of monoclonal antibody F19 against a cell-surface protein of reactive tumor stromal fibroblasts. J Clin Oncol. 1994 June; 12 (6): 1193-203.
Cohen S J, Alpaugh R K, Palazzo I, Meropol N J, Rogatko A, Xu Z, et al. Fibroblast activation protein and its relationship to clinical outcome in pancreatic adenocarcinoma. Pancreas. 2008; 37:154-8.
Conway R E, Joiner K, Patterson A, Bourgeois D, Rampp R, Hannah B C, et al. Prostate specific membrane antigen produces pro-angiogenic laminin peptides downstream of matrix metalloprotease-2. Angiogenesis. 2013; 16:847-60.
Delgado-Bellido D, Serrano-Saenz S, Fernández-Cortés M, Oliver F J. Vasculogenic mimicry signaling revisited: focus on non-vascular VE-cadherin. Molecular Cancer. 2017; 16:65.
Eder M, Schäfer M, Bauder-Wüst U, Haberkorn U, Eisenhut M, Kopka K. Preclinical evaluation of a bispecific low-molecular heterodimer targeting both PSMA and GRPR for improved PET imaging and therapy of prostate cancer. The Prostate. 2014; 74:659-68.
Edosada C Y, Quan C, Tran T, Pham V, Wiesmann C, Fairbrother W, Wolf B B. Peptide substrate profiling defines fibroblast activation protein as an endopeptidase of strict Gly (2)-Pro(1)-cleaving specificity. FEBS Lett. 2006 Mar. 6; 580 (6): 1581-6.
Errarte P, Guarch R, Pulido R, Blanco L, Nunes-Xavier C E, Beitia M, et al. The Expression of Fibroblast Activation Protein in Clear Cell Renal Cell Carcinomas Is Associated with Synchronous Lymph Node Metastases. PloS one. 2016; 11: e0169105.
Fischer E, Chaitanya K, Wuest T, et al. Radioimmunotherapy of fibroblast activation protein positive tumors by rapidly internalizing antibodies. Clin Cancer Res. 2012; 18:6208-6218.
Fitzgerald A A, Weiner L M. The role of fibroblast activation protein in health and malignancy. Cancer and Metastasis Reviews. 2020; 39:783-803.
Franco O E, Shaw A K, Strand D W, Hayward S W. Cancer associated fibroblasts in cancer pathogenesis. Semin Cell Dev Biol. 2010 February; 21 (1): 33-9.
Garin-Chesa P, Old L J, Rettig W J. Cell surface glycoprotein of reactive stromal fibroblasts as a potential antibody target in human epithelial cancers. Proc Natl Acad Sci USA. 1990 September; 87 (18): 7235-9. PMCID: PMC54718.
He M, Henderson M, Muth S, Murphy A, Zheng L. Preclinical mouse models for immunotherapeutic and non-immunotherapeutic drug development for pancreatic ductal adenocarcinoma. Annals of Pancreatic Cancer. 2020; 3.
Herrmann K, Schwaiger M, Lewis J S, Solomon S B, McNeil B J, Baumann M, et al. Radiotheranostics: a roadmap for future development. The Lancet Oncology. 2020; 21: e146-e56.
Hintz H M, Gallant J P, Vander Griend D J, Coleman I M, Nelson P S, LeBeau A M. Imaging fibroblast activation protein alpha improves diagnosis of metastatic prostate cancer with positron emission tomography. Clinical Cancer Research. 2020; 26:4882-91.
Hofman M S, Emmett L, Sandhu S, Iravani A, Joshua A M, Goh J C, et al. [177Lu]Lu-PSMA-617 versus cabazitaxel in patients with metastatic castration-resistant prostate cancer (TheraP): a randomised, open-label, phase 2 trial. The Lancet. 2021; 397:797-804.
Hofman M S, Lawrentschuk N, Francis R J, Tang C, Vela I, Thomas P, et al. Prostate-specific membrane antigen PET-CT in patients with high-risk prostate cancer before curativeintent surgery or radiotherapy (proPSMA): a prospective, randomised, multicentre study. The Lancet. 2020; 395:1208-16.
Huang, X.; Bennett, M.; Thorpe, P. E. Prostate 2004, 61, 1.
Imlimthan S, Moon E S, Rathke H, Afshar-Oromieh A, Rösch F, Rominger A, et al. New frontiers in cancer imaging and therapy based on radiolabeled fibroblast activation protein inhibitors: A rational review and current progress. Pharmaceuticals. 2021; 14:1023.
International PCT Patent Application No. WO2010/108125 A2 for PSMA-Targeting Compounds and Uses Thereof, to Pomper et al., published Sep. 23, 2010.
International PCT Patent Application No. WO2018232280 for PSMA Targeted Fluorescent Agents for Image Guided Surgery, to Pomper et al., published Dec. 20, 2018.
International PCT Patent Application No. WO2019/154886 for FAP Inhibitor, to Haberkorn et al., published Aug. 15, 2019.
International PCT Patent Publication No. WO 2013/107820 for Novel FAP Inhibitors to Jansen et al., published Jul. 25, 2013.
Isik E G, Has-Simsek D, Sanli O, Sanli Y, Kuyumcu S. Fibroblast activation protein-targeted pet imaging of metastatic castration-resistant prostate cancer compared with ⁶⁸Ga-PSMA and 18F-FDG PET/CT. Clinical Nuclear Medicine. 2021.
Jambunathan K, Watson D S, Endsley A N, Kodukula K, Galande A K, Comparative analysis of the substrate preferences of two Post-proline cleaving endopeptidases, prolyl Oligopeptidase and fibroblast activation protein a, FEBS Lett. 2012; 586 (16): 2507-2512,
Jansen K, Heirbaut L, Cheng J D, Joossens J, Ryabtsova O, Cos P, Maes L, Lambeir A M, De Meester I, Augustyns K, Van der Veken P. Selective Inhibitors of Fibroblast Activation Protein (FAP) with a (4-Quinolinoyl)-glycyl-2-cyanopyrrolidine Scaffold. ACS Med Chem Lett. 2013 Mar. 18; 4 (5): 491-6.
Jansen K, Heirbaut L, Verkerk R, Cheng J D, Joossens J, Cos P, et al. Extended structure-activity relationship and pharmacokinetic investigation of (4-Quinolinoyl)glycyl-2-cyanopyrrolidine Inhibitors of Fibroblast Activation Protein (FAP). Journal of Medicinal Chemistry. 2014; 57:3053-74.
Juillerat-Jeanneret L, Tafelmeyer P, Golshayan D, Fibroblast activation protein-alpha in fibrogenic disorders and cancer: more than a prolyl-specific peptidase? Expert Opin. Ther. Targets 2017, 21:977-991.
Kalluri R. The biology and function of fibroblasts in cancer. Nature Reviews Cancer. 2016; 16:582-98.
Keane F M, Yao T-W, Seelk S, Gall M G, Chowdhury S, Poplawski S E, et al. Quantitation of fibroblast activation protein (FAP)-specific protease activity in mouse, baboon and human fluids and organs. FEBS Open Bio. 2014; 4:43-54.
Kelly T. Fibroblast activation protein-alpha and dipeptidyl peptidase IV (CD26): cell-surface proteases that activate cell signaling and are potential targets for cancer therapy. Drug Resist Updat. 2005 February-April; 8 (1-2): 51-8.
Kesch C, Yirga L, Dendl K, Handke A, Darr C, Krafft U, et al. High fibroblast-activation-protein expression in castration-resistant prostate cancer supports the use of FAPI-molecular theranostics. European Journal of Nuclear Medicine and Molecular Imaging. 2021; 49:385-9.
Kessel K, Seifert R, Weckesser M, Boegemann M, Huss S, Kratochwil C, et al. Prostate-specific membrane antigen and fibroblast activation protein distribution in prostate cancer: preliminary data on immunohistochemistry and PET imaging. Annals of Nuclear Medicine. 2021.
Kraman M, Bambrough P J, Arnold J N, Roberts E W, Magiera L, Jones J O, Gopinathan A, Tuveson D A, Fearon D T. Suppression of antitumor immunity by stromal cells expressing fibroblast activation protein-alpha. Science. 2010 Nov. 5; 330 (6005): 827-30.
Kratochwil C, Flechsig P, Lindner T, Abderrahim L, Altmann A, Mier W, et al. 68Ga-FAPI PET/CT: Tracer uptake in 28 different kinds of cancer. Journal of Nuclear Medicine. 2019; 60:801-5.
Krishnaraju V S, Kumar R, Mittal B R, Sharma V, Singh H, Nada R, et al. Differentiating benign and malignant pancreatic masses: Ga-68 PSMA PET/CT as a new diagnostic avenue. European Radiology. 2021; 31:2199-208.
Laverman P, van der Geest T, Terry S Y, Gerrits D, Walgreen B, Helsen M M, Nayak T K, Freimoser-Grundschober A, Waldhauer I, Hosse R J, Moessner E, Umana P, Klein C, Oyen W J, Koenders M I, Boerman O C. Immuno-PET and Immuno-SPECT of Rheumatoid Arthritis with Radiolabeled Anti-Fibroblast Activation Protein Antibody Correlates with Severity of Arthritis. J Nucl Med. 2015 May; 56 (5): 778-83.
Ling X, Cutler C S, Anderson C J. The radiopharmaceutical chemistry of the radioisotopes of copper. In: Lewis J S, Windhorst A D, Zeglis B M, editors. Radiopharmaceutical Chemistry. Cham: Springer International Publishing; 2019. p. 335-58.
Liu, H.; Moy, P.; Kim, S.; Xia, Y.; Rajasekaran, A.; Navarro, V.; Knudsen, B.; Bander, N. H. Cancer Res. 1997, 57, 3629.
Lo P C, Chen J, Stefflova K, Warren M S, Navab R, Bandarchi B, Mullins S, Tsao M, Cheng J D, Zheng G. Photodynamic molecular beacon triggered by fibroblast activation protein on cancer-associated fibroblasts for diagnosis and treatment of epithelial cancers. J Med Chem. 2009 Jan. 22; 52 (2): 358-68.
Loktev A, Lindner T, Burger E-M, Altmann A, Giesel F, Kratochwil C, et al. Development of fibroblast activation protein-targeted radiotracers with improved tumor retention. Journal of Nuclear Medicine. 2019; 60:1421-9.
López J I, Errarte P, Erramuzpe A, Guarch R, Cortés J M, Angulo J C, et al. Fibroblast activation protein predicts prognosis in clear cell renal cell carcinoma. Human Pathology. 2016; 54:100-5.
Luo H, Hernandez R, Hong H, Graves S A, Yang Y, England C G, et al. Noninvasive brain cancer imaging with a bispecific antibody fragment, generated via click chemistry. Proceedings of the National Academy of Sciences. 2015; 112:12806-11.
Meester I, Augustyns K, Van der Veken P. Acylated Gly-(2-cyano) pyrrolidines as inhibitors of fibroblast activation protein (FAP) and the issue of FAP/prolyl oligopeptidase (PREP)-selectivity. Bioorg Med Chem Lett. 2012 May 15; 22 (10): 3412-7.
Meyer A R, Carducci M A, Denmeade S R, Markowski M C, Pomper M G, Pierorazio P M, et al. Improved identification of patients with oligometastatic clear cell renal cell carcinoma with PSMA-targeted 18F-DCFPyL PET/C T. Annals of Nuclear Medicine. 2019; 33:617-23.
Mona C E, Benz M R, Hikmat F, Grogan T R, Lückerath K, Razmaria A, et al. Correlation of 68Ga-FAPi-46 PET biodistribution with FAP expression by immunohistochemistry in patients with solid cancers: a prospective translational exploratory study. Journal of Nuclear Medicine. 2021: jnumed. 121.262426.
Nimmagadda S, Pullambhatla M, Chen Y, Parsana P, Lisok A, Chatterjee S, et al. Low-level endogenous PSMA expression in nonprostatic tumor xenografts is sufficient for in vivo tumor targeting and imaging. Journal of Nuclear Medicine. 2018; 59:486-93.
Olszewski R T, Bukhari N, Zhou J, Kozikowski A P, Wroblewski J T, Shamimi-Noori S, et al. NAAG peptidase inhibition reduces locomotor activity and some stereotypes in the PCP model of schizophrenia via group II mGluR. Journal of Neurochemistry. 2004; 89:876-85.
Paschalis A, Sheehan B, Riisnaes R, Rodrigues D N, Gurel B, Bertan C, et al. Prostate-specific membrane antigen heterogeneity and DNA repair defects in prostate cancer. European Urology. 2019; 76:469-78.
Pereira B A, Vennin C, Papanicolaou M, Chambers C R, Herrmann D, Morton J P, et al. CAF Subpopulations: A New Reservoir of Stromal Targets in Pancreatic Cancer. Trends in Cancer. 2019; 5:724-41.
Poels T T, Vuijk F A, de Geus-Oei L-F, Vahrmeijer A L, Oprea-Lager D E, Swijnenburg R J. Molecular Targeted Positron Emission Tomography Imaging and Radionuclide Therapy of Pancreatic Ductal Adenocarcinoma. Cancers. 2021; 13:6164.
Poplawski S E, Lai J H, Li Y, Jin Z, Liu Y, Wu W, Wu Y, Zhou Y, Sudmeier J L, Sanford D G, Bachovchin W W. Identification of selective and potent inhibitors of fibroblast activation protein and prolyl oligopeptidase. J Med Chem. 2013 May 9; 56 (9): 3467-77.
Puré E, Blomberg R. Pro-tumorigenic roles of fibroblast activation protein in cancer: back to the basics. Oncogene. 2018; 37:4343-57.
Reilly R M, Lam K, Chan C, Levine M. Advancing Novel Molecular Imaging Agents from Preclinical Studies to First-in-Humans Phase I Clinical Trials in Academia-A Roadmap for Overcoming Perceived Barriers. Bioconjugate chemistry. 2015; 26:625-632.
Rettig W J, Garin-Chesa P, Healey J H, Su S L, Ozer H L, Schwab M, Albino A P, Old L J. Regulation and heteromeric structure of the fibroblast activation protein in normal and transformed cells of mesenchymal and neuroectodermal origin Cancer Res. 1993 Jul. 15; 53 (14): 3327-35.
Rini B I, Plimack E R, Stus V, Gafanov R, Hawkins R, Nosov D, et al. Pembrolizumab plus Axitinib versus Sunitinib for Advanced Renal-Cell Carcinoma. New England Journal of Medicine. 2019; 380:1116-27.
Röhrich M, Loktev A, Wefers A K, Altmann A, Paech D, Adeberg S, et al. IDH-wildtype glioblastomas and grade III/IV IDH-mutant gliomas show elevated tracer uptake in fibroblast activation protein-specific PET/CT. European Journal of Nuclear Medicine and Molecular Imaging. 2019; 46:2569-80.
Roy et al., Design and validation of fibroblast activation protein alpha targeted imaging and therapeutic agents, Theranostics 2020, 10 (13), 5778-5789.
Ryabtsova O, Jansen K, Van Goethem S, Joossens J, Cheng J D, Lambeir A M, De Meester I, Augustyns K, Van der Veken P. Acylated Gly-(2-cyano) pyrrolidines as inhibitors of fibroblast activation protein (FAP) and the issue of FAP/prolyl oligopeptidase (PREP)-selectivity. Bioorg Med Chem Lett. 2012 May 15; 22 (10): 3412-7.
Sanford D G, Bachovchin W W. Identification of selective and potent inhibitors of fibroblast activation protein and prolyl oligopeptidase. J Med Chem. 2013 May 9; 56 (9): 3467-77.
Scanlan M J, Raj B K, Calvo B, Garin-Chesa P, Sanz-Moncasi M P, Healey J H, Old L J, Rettig W J. Molecular cloning of fibroblast activation protein alpha, a member of the serine protease family selectively expressed in stromal fibroblasts of epithelial cancers. Proc Natl Acad Sci USA. 1994 Jun. 7; 91 (12): 5657-61.
Schuelke, N.; Varlamova, O. A.; Donovan, G. P.; Ma, D.; Gardner, J. P.; Morrissey, D. M.; Arrigale, R. R; Zhan, C; Chodera, A. J.; Surowitz, K. G; Maddon, P. J.; Heston, W. D. W.; Olson, W. C. Proc. Natl. Acad. Sci. 2003, 100, 12590.
Scott A M, Wiseman G, Welt S, Adjei A, Lee F T, Hopkins W, Divgi C R, Hanson L H, Mitchell P, Gansen D N, Larson S M, Ingle J N, Hoffman E W, Tanswell P, Ritter G, Cohen L S, Bette P, Arvay L, Amelsberg A, Vlock D, Rettig W J, Old L J. A Phase I dose-escalation study of sibrotuzumab in patients with advanced or metastatic fibroblast activation protein-positive cancer. Clin Cancer Res. 2003 May; 9 (5): 1639-47.
Shallal H M, Minn I, Banerjee S R, Lisok A, Mease R C, Pomper M G. Heterobivalent agents targeting PSMA and integrin-αvβ3. Bioconjugate Chemistry. 2014; 25:393-405.
Sheridan C. Amgen's bispecific antibody puffs across finish line. Nature Biotechnology. 2015; 33:219-21.
Silver D A, Pellicer I, Fair W R, Heston W D, Cordon-Cardo C. Prostate-specific membrane antigen expression in normal and malignant human tissues. Clinical Cancer Research. 1997; 3:81-5.
Siva S, Udovicich C, Tran B, Zargar H, Murphy D G, Hofman M S. Expanding the role of small-molecule PSMA ligands beyond PET staging of prostate cancer. Nature Reviews Urology. 2020; 17:107-18.
Slania S L, Das D, Lisok A, Du Y, Jiang Z, Mease R C, et al. Imaging of Fibroblast Activation Protein in Cancer Xenografts Using Novel (4-Quinolinoyl)-glycyl-2-cyanopyrrolidine-Based Small Molecules. Journal of Medicinal Chemistry. 2021; 64:4059-70.
Solano-Iturri J D, Beitia M, Errarte P, Calvete-Candenas J, Etxezarraga M C, Loizate A, et al. Altered expression of fibroblast activation protein-alpha; (FAP) in colorectal adenomacarcinoma sequence and in lymph node and liver metastases. Aging. 2020a; 12:10337-58.
Solano-Iturri J D, Errarte P, Etxezarraga M C, Echevarria E, Angulo J, López J I, et al. Altered tissue and plasma levels of fibroblast activation protein-α (FAP) in renal tumours. Cancers. 2020b; 12:3393.
Spatz S, Tolkach Y, Jung K, Stephan C, Busch J, Ralla B, et al. Comprehensive evaluation of prostate specific membrane antigen expression in the vasculature of renal tumors: Implications for imaging studies and prognostic role. Journal of Urology. 2018; 199:370-7.
Stock K, Steinestel K, Wiesch R, Mikesch J-H, Hansmeier A, Trautmann M, et al. Neovascular Prostate-Specific Membrane Antigen Expression Is Associated with Improved Overall Survival under Palliative Chemotherapy in Patients with Pancreatic Ductal Adenocarcinoma. BioMed Research International. 2017; 2017:2847303.
Tsai T Y, Yeh T K, Chen X, Hsu T, Jao Y C, Huang C H, Song J S, Huang Y C, Chien C H, Chiu J H, Yen S C, Tang H K, Chao Y S, Jiaang W T. Substituted 4-carboxymethylpyroglutamic acid diamides as potent and selective inhibitors of fibroblast activation protein. J Med Chem. 2010 Sep. 23; 53 (18): 6572-83.
Tuxhorn J A, Ayala G E, Smith M J, Smith V C, Dang T D, Rowley D R. Reactive stroma in human prostate cancer: induction of myofibroblast phenotype and extracellular matrix remodeling. Clin Cancer Res. 2002 September; 8 (9): 2912-23.
U.S. Patent Application Publication No. US2014/0357650 for Novel FAP Inhibitors to Jansen et al., published Dec. 4, 2014.
U.S. Pat. No. 9,346,814 for Novel FAP Inhibitors to Jansen et al., issued May 24, 2016.
Uijen M J M, Derks Y H W, Merkx R I J, Schilham M G M, Roosen J, Privé B M, et al. PSMA radioligand therapy for solid tumors other than prostate cancer: background, opportunities, challenges, and first clinical reports. European Journal of Nuclear Medicine and Molecular Imaging. 2021; 48:4350-68.
Watabe T, Liu Y, Kaneda-Nakashima K, Shirakami Y, Lindner T, Ooe K, et al. Theranostics targeting fibroblast activation protein in the tumor stroma: 64Cu- and 225Ac-labeled FAPI-04 in pancreatic cancer xenograft mouse models. Journal of Nuclear Medicine. 2020; 61:563-9.
Welt S, Divgi C R, Scott A M, Garin-Chesa P, Finn R D, Graham M, Carswell E A, Cohen A, Larson S M, Old L J, et al, Antibody targeting in metastatic colon cancer: a phase I study of monoclonal antibody F19 against a cell-surface protein of reactive tumor stromal fibroblasts. J Clin Oncol. 1994 June; 12 (6): 1193-203.
Wernicke A G, Kim S, Liu H, Bander N H, Pirog E C. Prostate-specific membrane antigen (PSMA) expression in the neovasculature of gynecologic malignancies: implications for PSMA-targeted therapy. Applied Immunohistochemistry & Molecular Morphology. 2017; 25:271-6.
Youn H., Hong K. In vivo noninvasive small animal molecular imaging. Osong Public Health Res Perspect. 2012; 3:48-59. PMCID: PMC3738683
Yu D M, Yao T W, Chowdhury S, Nadvi N A, Osborne B, Church W B, McCaughan G W, Gorrell M D. The dipeptidyl peptidase I V family in cancer and cell biology. FEBS J. 2010 March; 277 (5): 1126-44.
Zhang et al., Prostate-specific membrane antigen-targeted photoacoustic imaging of prostate cancer in vivo, J. of Biophotonics, 2018; 11: e201800021.
Zhou L, Chang Y, Xu L, Liu Z, Fu Q, Yang Y, et al. The Presence of Vascular Mimicry Predicts High Risk of Clear Cell Renal Cell Carcinoma after Radical Nephrectomy. Journal of Urology. 2016; 196:335-42.

Although the foregoing subject matter has been described in some detail by way of illustration and example for purposes of clarity of understanding, it will be understood by those skilled in the art that certain changes and modifications can be practiced within the scope of the appended claims.

1-41. (canceled)

42. A pharmaceutical composition comprising the compound of claim 53.

43. The composition of claim 42, further comprising one or more of pharmaceutically acceptable carriers, diluents, excipients, or adjuvants.

44. A method for imaging a disease or disorder associated with fibroblast-activation protein-α (FAP-α) and/or prostate-specific membrane antigen (PSMA), the method comprising administering a compound according to claim 53, or a pharmaceutical composition thereof, wherein the compound of formula (III) comprises an optical or radiolabeled functional group suitable for optical imaging, photoacoustic imaging, PET imaging, or SPECT imaging; and obtaining an image.

45. A method for inhibiting fibroblast-activation protein-α (FAP-α) and/or prostate-specific membrane antigen (PSMA), the method comprising administering to a subject in need thereof an effective amount of a compound according to any of claim 53, or a pharmaceutical composition thereof.

46. A method for treating a fibroblast-activation protein-α (FAP-α)- and/or a prostate-specific membrane antigen (PSMA)-related disease or disorder, the method comprising administering to a subject in need of treatment thereof an effective amount of a compound according to of claim 53, or a pharmaceutical composition thereof, wherein the compound of formula (III) comprises a radiolabeled functional group suitable for radiotherapy.

47. The method of claim 46, wherein the (FAP-α)-related disease or disorder is selected from the group consisting of a proliferative disease; diseases characterized by tissue remodeling and/or chronic inflammation; disorders involving endocrinological dysfunction; and blood clotting disorders.

48. The method of claim 47, wherein the proliferative disease is selected from the group consisting of breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma.

49. The method of claim 46, wherein the prostate-specific membrane antigen (PSMA)-related disease or disorder is selected from the group consisting of prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature.

50. The method of claim 49, wherein the prostate-specific membrane antigen (PSMA)-related disease or disorder comprises prostate cancer.

51-52. (canceled)

53. A compound of formula (III): 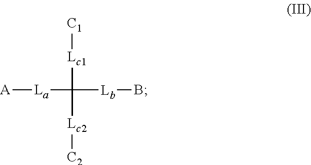
wherein:
A is a targeting moiety for fibroblast activation protein alpha (FAP-α);
B is absent or a targeting moiety for FAP-α, wherein A and B can be the same or different;
C₁ can be absent or present and when present is a chelating group;
C₂ is a prosthetic group;
Lₐ and L_{b} are each a bi-functionalized linker capable of forming a chemical bond with each other and A, B, C₁ and C₂;
L_{c1} and L_{c2} are each independently a bi-functionalized linker capable of forming a chemical bond with each other and A, B, Lₐ, and L_{b};
wherein if C₁ is absent, L_{c1} also is absent and wherein if B is absent, L_{b} also is absent;
and stereoisomers and pharmaceutically acceptable salts thereof.

54. The compound of claim 53, wherein A is or, if B is present, A and B are each an FAP-α targeting moiety having the structure of: 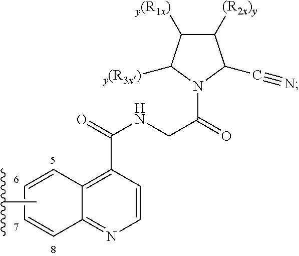
wherein [symbol] indicates a point of attachment of the FAP-α binding ligand to the linker Lₐ and/or L_{b} wherein the point of attachment can be through any of carbon atoms 5, 6, 7, or 8 of the quinolinyl ring thereof; and stereoisomers and pharmaceutically acceptable salts thereof.

55. The compound of claim 53, wherein C₂ is a prosthetic group comprising a radioisotope selected from the group consisting of ¹⁸F, ¹²⁴I, ¹²⁵I, ¹³¹I, and ²¹¹At.

56. The compound of claim 53, wherein the prosthetic group is selected from the group consisting of: 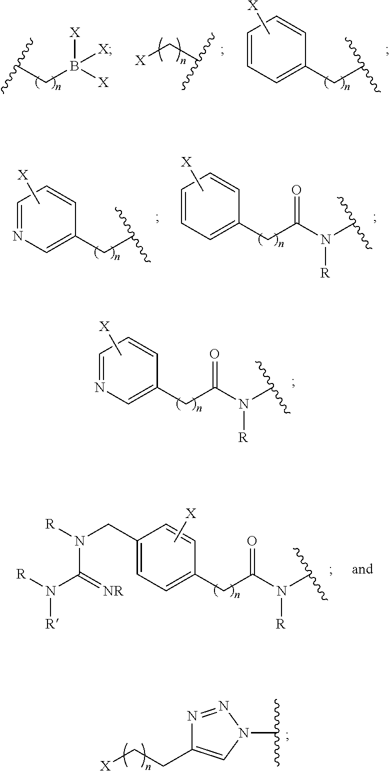
wherein each X is independently selected from a straightchain or branched C₁-C₈ alkyl, —SO₂, —C(═O)—, —C(═O)OR₂₀, wherein R₂₀ is H or C₁-C₄ alkyl, and a radioisotope selected from the group consisting of ¹⁸F, ¹²⁴I, ¹²⁵I, ¹³¹I, and ²¹¹At; each R and R′ is defined hereinabove; and each n is independently an integer selected from the group consisting of 0, 1, 2, 3, 4, 5, 6, 7, 8, 9, 10, 11, 12, 13, 14, 15, 16, 17, 18, 19, and 20, wherein each carbon of the 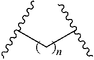
alkylene chain can be substituted with C₁-C₄ alkyl.

57. The compound of claim 56, wherein the prosthetic group is selected from the group consisting of:

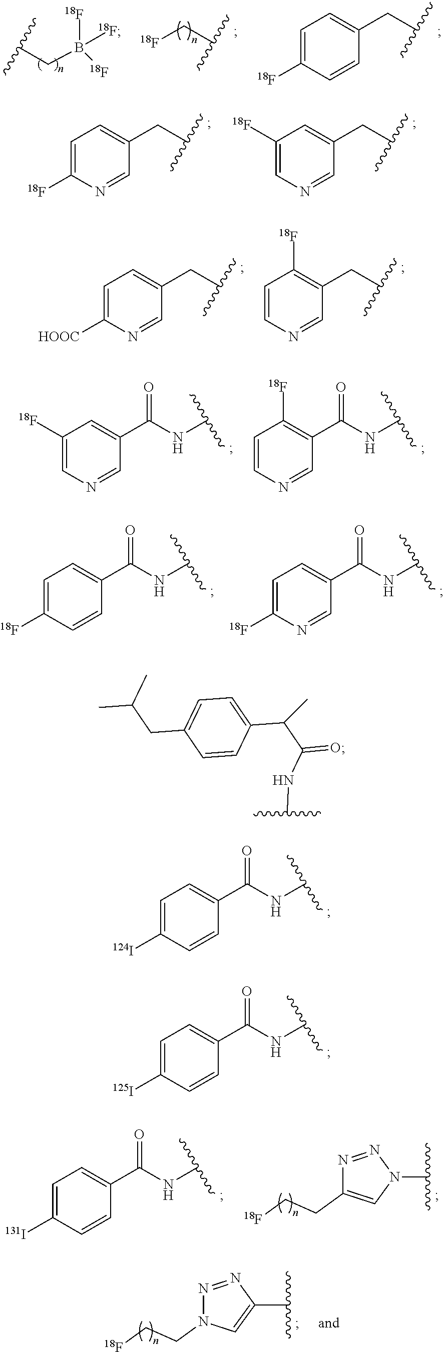

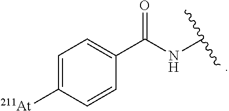

58. The compound of claim 53, wherein C₁ comprises a chelating agent selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), (NOTAGA) 1,4,7-triazonane-1,4-diyl)diacetic acid DFO (Desferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino) methyl) benzoic acid), and BaBaSar.

59. The compound of claim 53, wherein C₁ is a chelating agent is selected from the group consisting of:

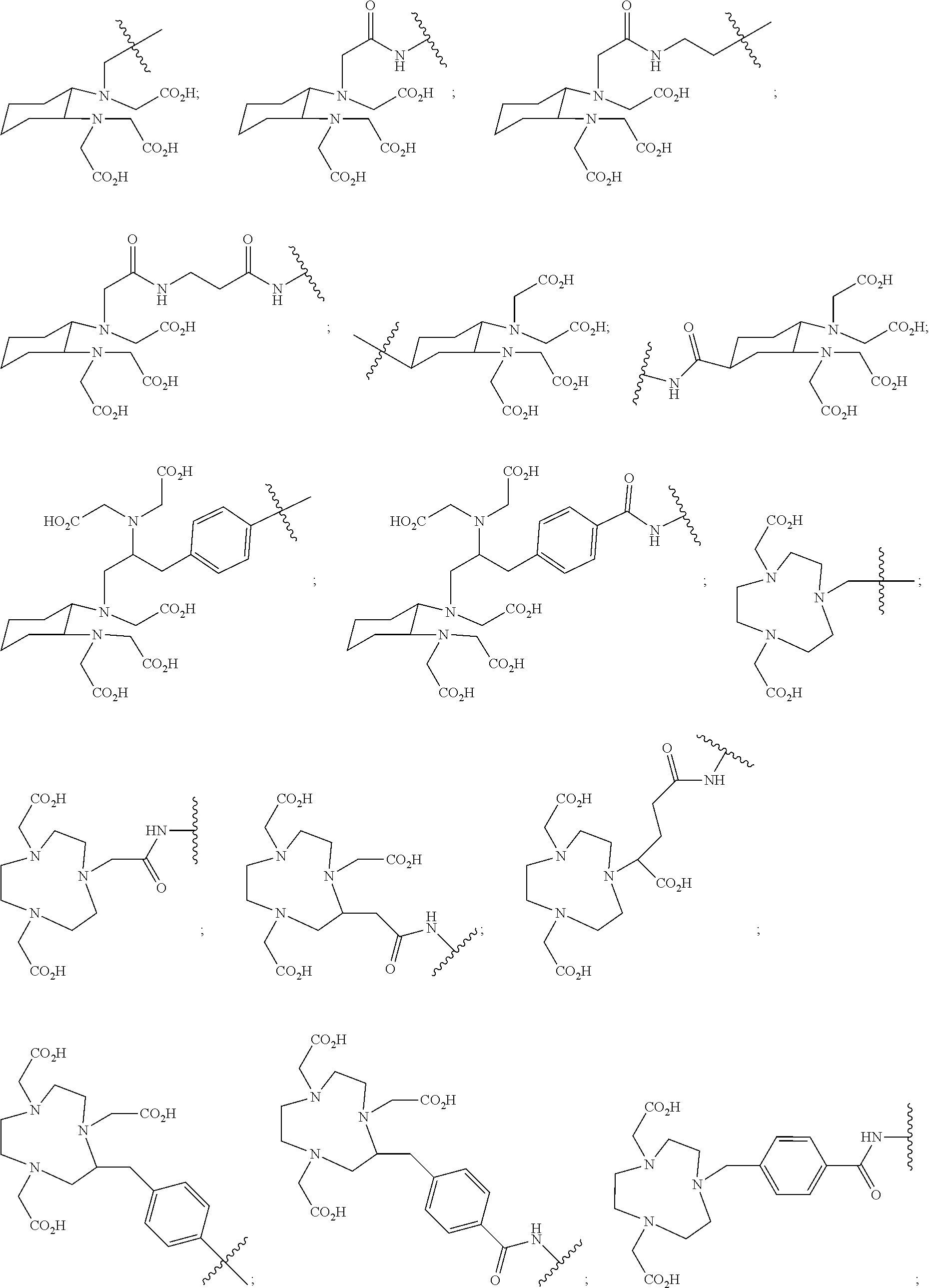

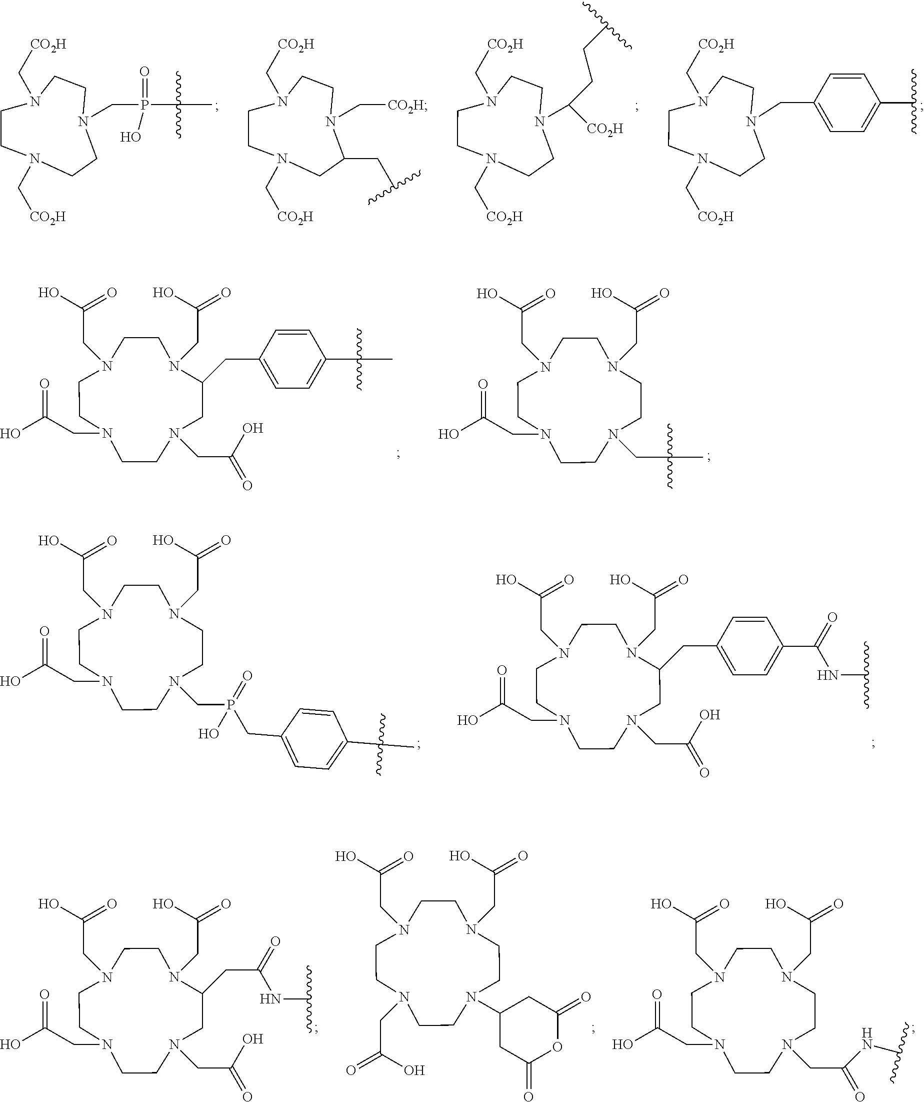

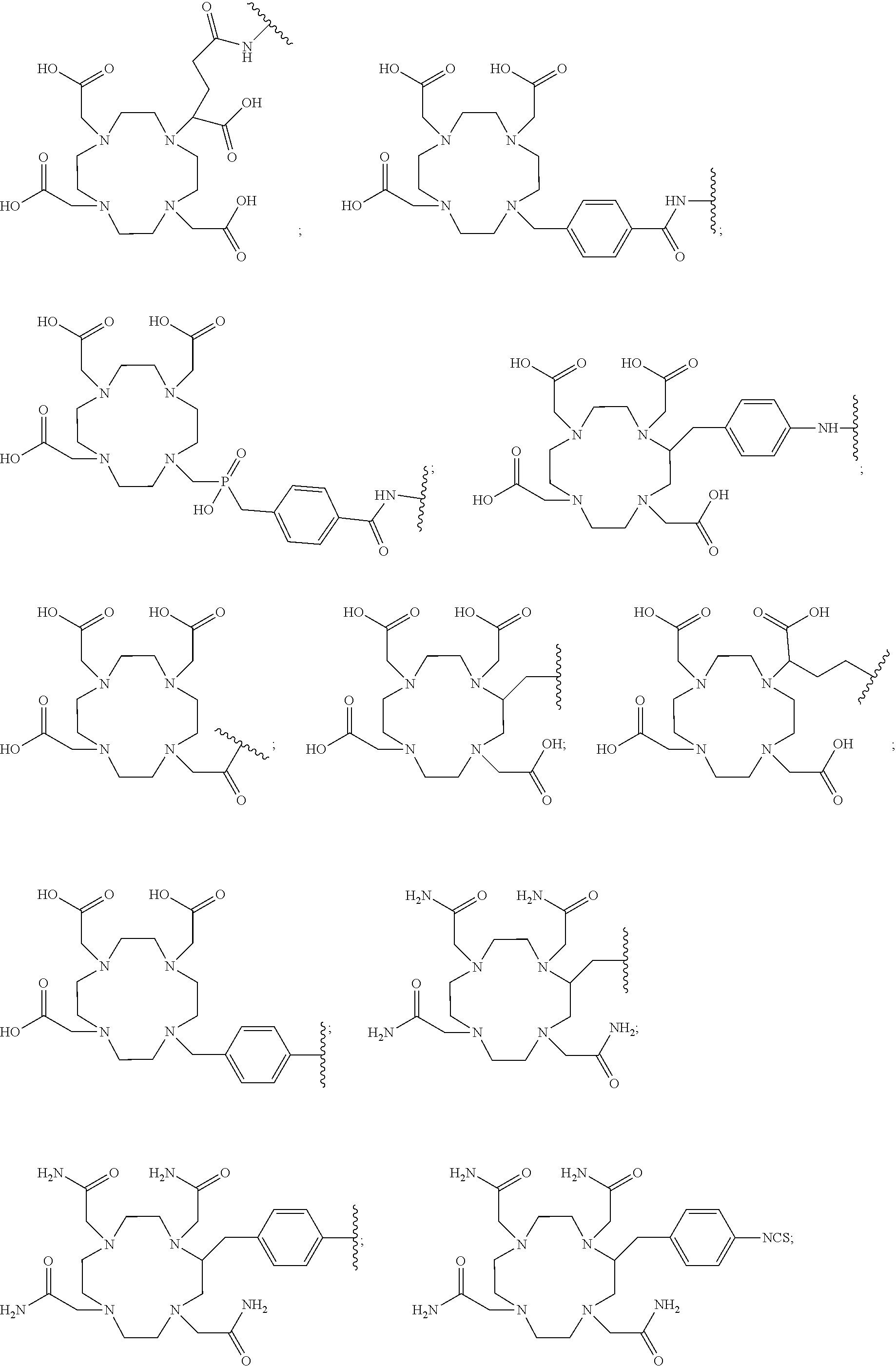

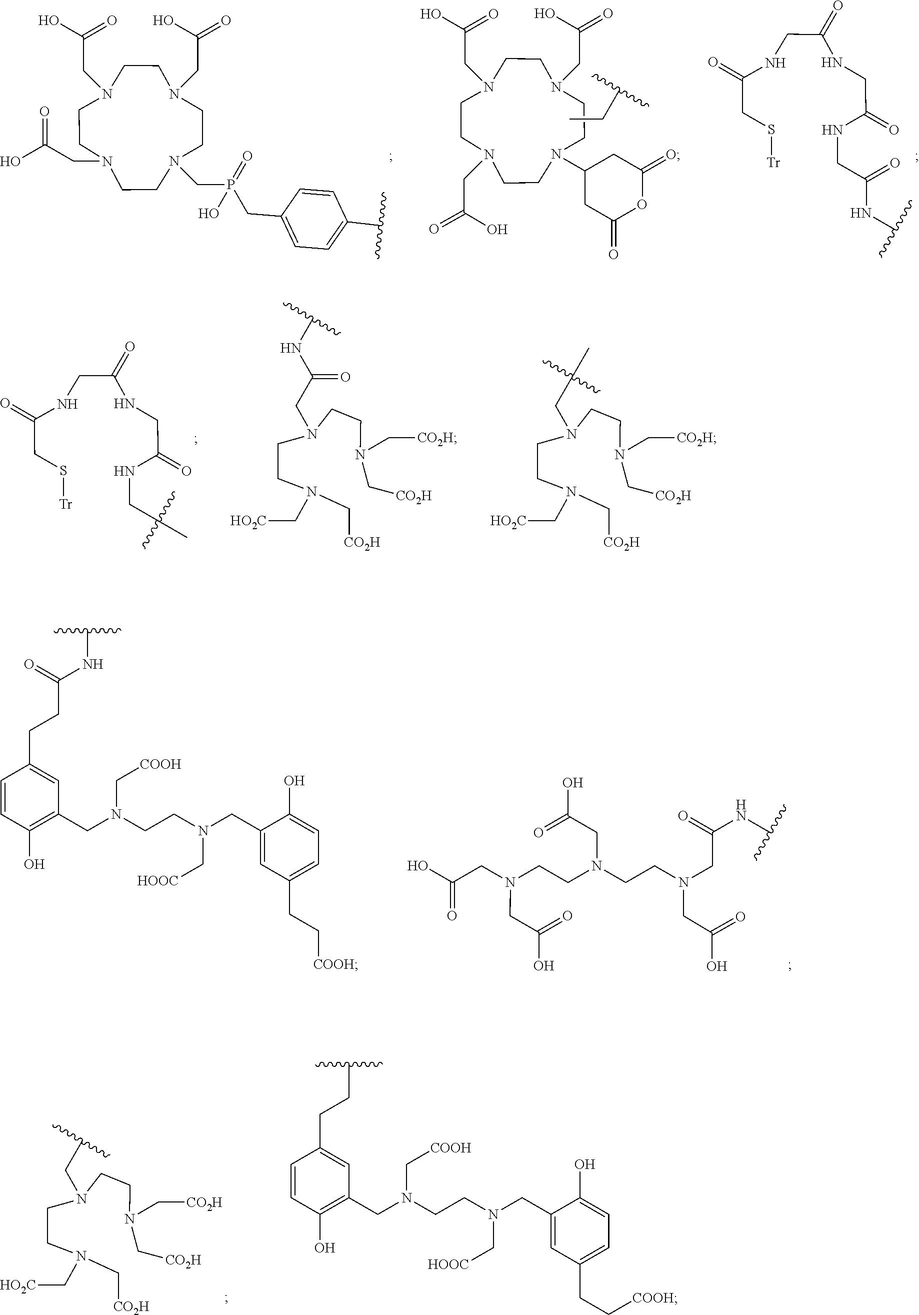

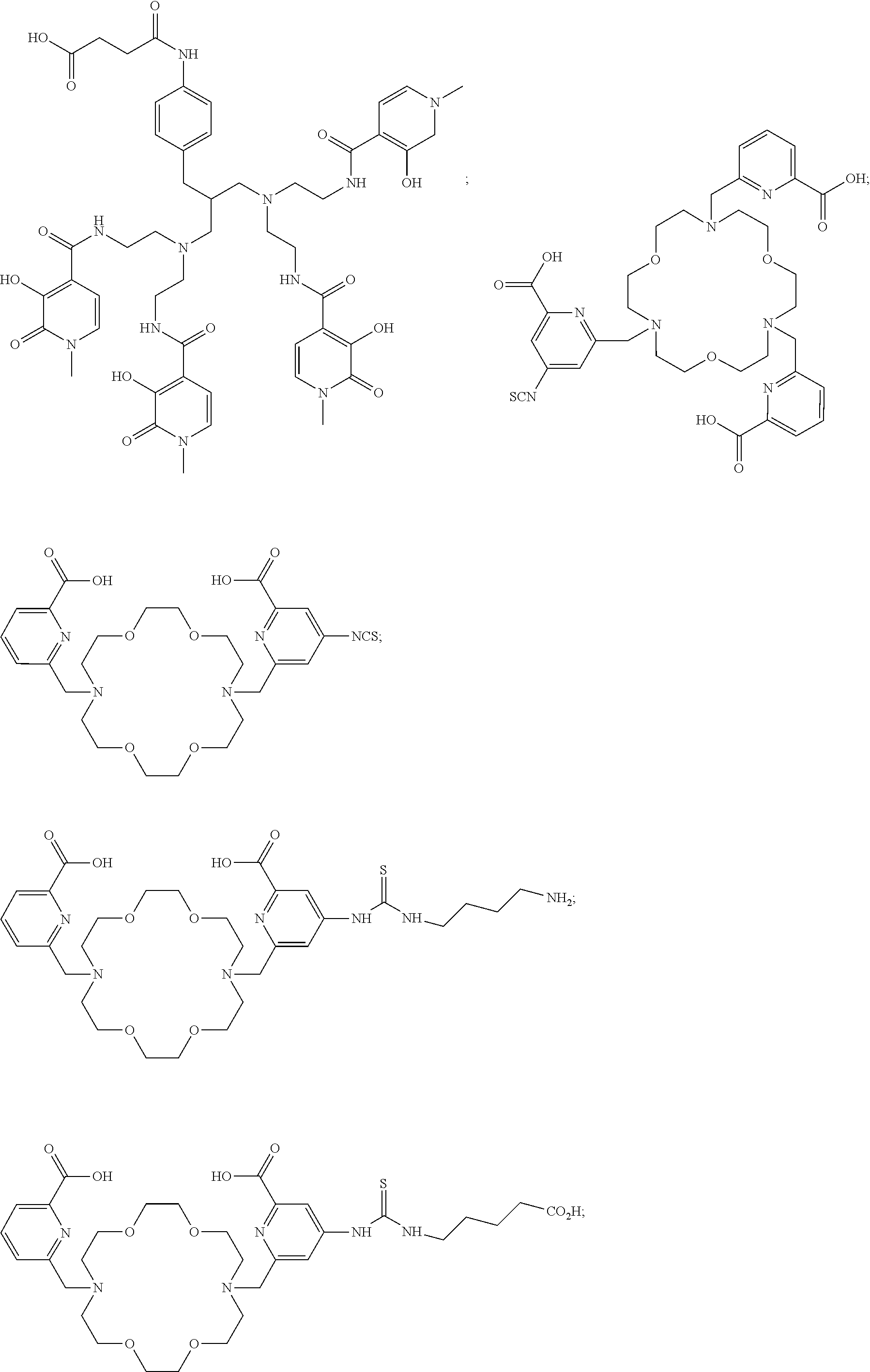

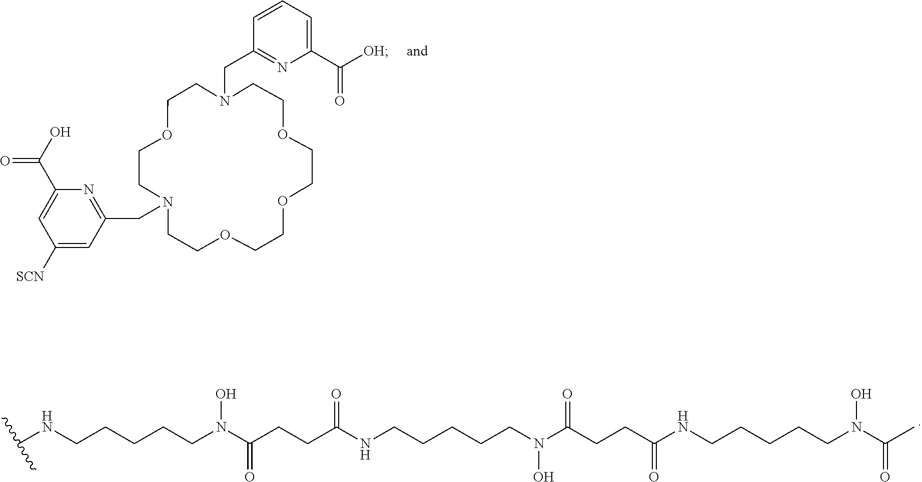

60. The compound of 53, wherein the chelating agent further comprises a radiometal.

61. The compound of claim 60, wherein the radiometal is selected from the group consisting of ⁶⁰Cu, ⁶²Cu, ⁶⁴Cu, ⁶⁷Cu, ²⁰³Ph, ²¹²Pb, ²²⁵Ac, ¹⁷⁷Lu, ^{99m}Tc, ⁶⁸Ga, ¹⁴⁹Th, ⁸⁶Y, ⁹⁰Y, ¹¹¹In, ¹¹⁵In, ¹⁸⁶Re, ¹⁸⁸Re, ¹⁵³Sm, ⁸⁹Zr, ²¹³Bi, ²¹²Bi, ²¹²Pb, ⁶⁷Ga, ⁴⁷Sc, ¹⁶⁶Ho, ⁴³Sc, ²²³Ra, ^{226/227}Th, Al-¹⁸F, and Sc-¹⁸F.

62. The compound of claim 53, wherein Lₐ, L_{b}, L_{c1}, and L_{c2} are each individually selected from the group consisting of (a), (b), (c), or (d):
(a) 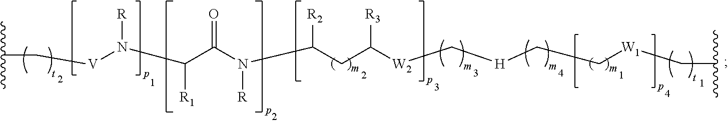
wherein:
p₁, p₂, p₃ and p₄ may be in any order;
t₁ and t₂ are each an integer selected from the group consisting of 0, 1, 2, 3, 4, 5, 6, 7, and 8;
p₁, p₃, and p₄ are each independently 0 or 1;
p₂ is an integer selected from the group consisting of 0, 1, 2, and 3, and when p₂ is 2 or 3, each R₁ is the same or different;
m₁, m₂, m₃, and m₄ are each an integer independently selected from the group consisting of 0, 1, 2, 3, 4, 5, 6, 7 and 8;
W₁ is selected from the group consisting of a bond, —S—, —C(═O)—, —C(═O)—NR—, and —NR—C(═O)—;
W₂ is selected from the group consisting of a bond, —S—, —CH₂—C(═O)—NR—, —C(═O)—, —NRC(═O)—, —NR′C(═O)NR—, —NRC(═S)NR′₂—, —NRC(═O)O—, —OC(═O)NR—, —OC(═O)—, —C(═O)NR—, —NR—C(═O)—, —C(═O)O, —(O—CH₂—CH₂)_{q}— and —(CH₂—CH₂—O)_{q}, wherein q is selected from the group consisting of 0, 1, 2, 3, 4, 5, 6, 7, and 8;
each R or R′ is independently H, alkyl, substituted alkyl, cycloalkyl, substituted cycloalkyl, heterocycloalkyl, substituted heterocycloalkyl, aryl, substituted aryl, heteroaryl, substituted heteroaryl, and —OR₄, wherein R₄ is selected from the group consisting of H, alkyl, substituted alkyl, cycloalkyl, substituted cycloalkyl, heterocycloalkyl, and substituted heterocycloalkyl, wherein q is defined as immediately hereinabove;
H can be present or absent and when present is selected from a nitrogen-containing heteroalkylene chain, a cyclic or bicylic heteroalkyl radical, and a triazole radical having the structure 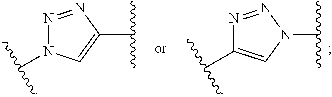 [SMILES: *c1cn(*)nn1 ; *c1cn(*)nn1]
each R₁ is independently H, C₁-C₆ alkyl, C₃-C₁₂ aryl, —(CH₂)_{q}—C₃-C₁₂ aryl, —C₄-C₁₆ alkylaryl, or —(CH₂)_{q}—C₄-C₁₆ alkylaryl; R₂ and R₃ are each independently H, —(CH₂)_{q}—C₃-C₁₂ aryl, and —CO₂R₅, wherein R₅ is selected from the group consisting of H, C₁-C₆ alkyl, C₃-C₁₂ aryl, and C₄-C₁₆ alkylaryl, wherein R₂ and R₃ can be the same or different, wherein q is selected from the group consisting of 0, 1, 2, 3, 4, 5, 6, 7, and 8;
V is selected from the group consisting of —C(O)—, —C(S)—, —NRC(O)—, —NRC(S)—, and —OC(O)—;
(b) 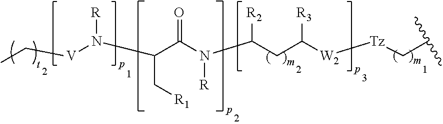
wherein p₁, p₂, p₃, m₁, m₂, Tz, W₂, R, R₁, R₂, R₃, and V are defined as hereinabove;
(c) -L₁-, -L₂-L₃-, or -L₁-L₂-L₃-, wherein:
L₁ is —NR—(CH₂)_{q}—[O—CH₂—CH₂—O]_{q}—(CH₂)_{q}—C(═O)—;
L₂ is —NR—(CH₂)_{q}—C(COOR₅)—NR—; and
L₃ is —(O═)C—(CH₂)_{q}—C(═O)—;
wherein each q is independently an integer selected from the group consisting of 1, 2, 3, 4, 5, 6, 7, and 8; and R and R₅ are as defined hereinabove; and
(d) —(CR⁶H)_{q}—(CH₂)_{q}—C(═O)—NR—(CH₂)_{q}—O— or —NR—(CH₂)_{q}—O—; wherein each q and R is defined hereinabove; and R⁶ is H or —COOR₅.

63. The compound of claim 62, wherein H is selected from: 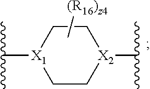
wherein X₁ and X₂ are each independently —CH— or N; each R₁₆ is independently H or —C(═O)—OR₁₇, wherein R₁₇ is C₁-C₄ alkyl;
—N(R ₁₈)—(CH₂)_{z5}—N(R₁₉)—; wherein R₁₈ and R₁₉ are each independently H or C₁-C₄ alkyl and Z5 is an integer selected from 1, 2, 3, 4, 5, 6, 7, and 8;

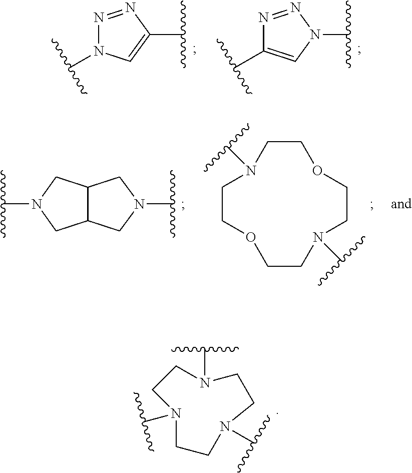 [SMILES: *c1cn(*)nn1 ; *c1cn(*)nn1 ; *N1CC2CN(*)CC2C1 ; *N1CCOCCN(*)CCOCC1 ; *N1CCN(*)CCN(*)CC1]

64. The compound of 62, wherein one or more of Lₐ, L_{b}, L_{c1}, and L_{c2} include one or more units selected from: 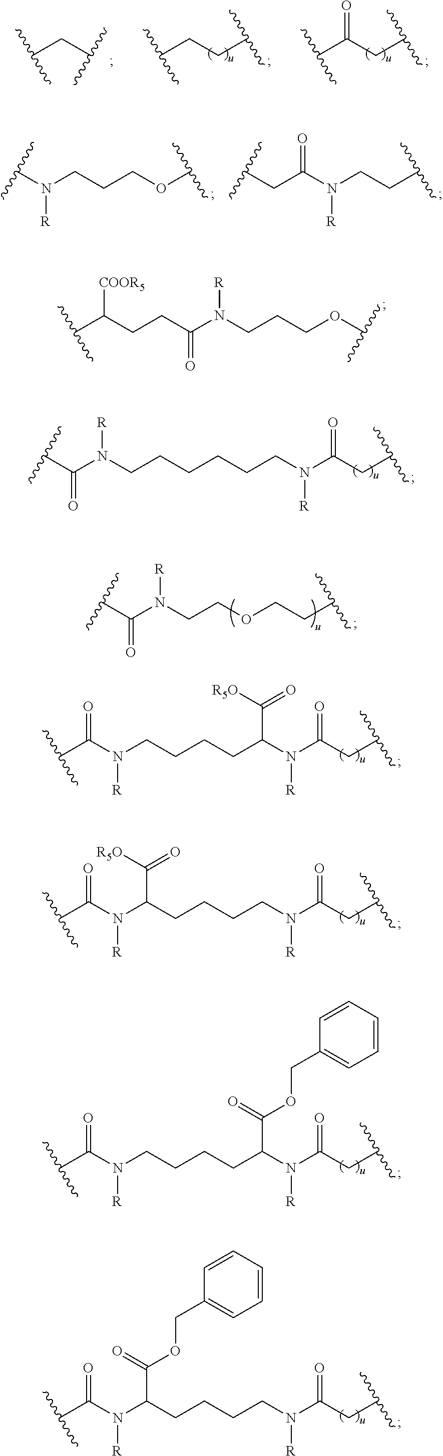 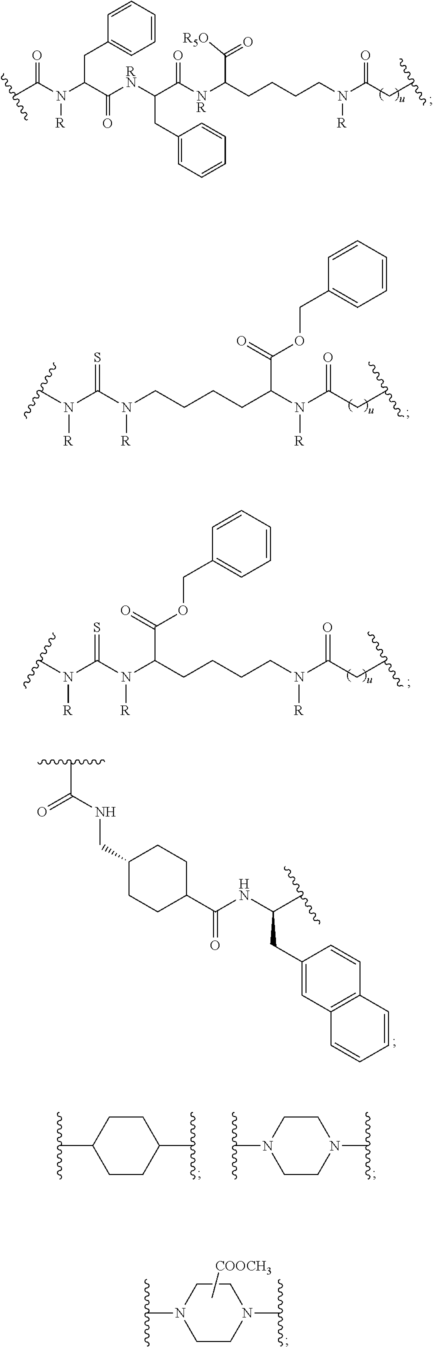 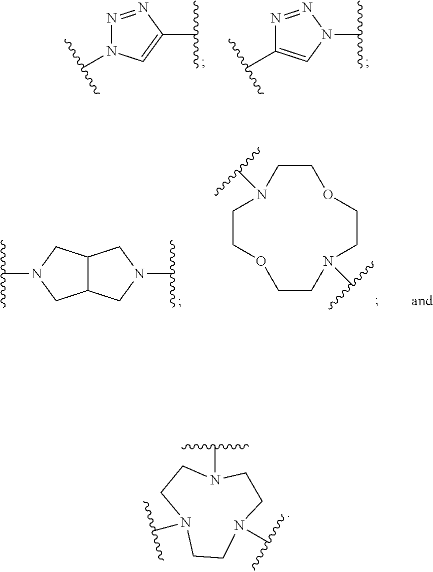
wherein u is an integer selected from 1, 2, 3, 4, 5, 6, 7, and 8; and R and R₅ are as defined hereinabove.

65. The compound of claim 53, wherein the compound of formula (III) is selected from:

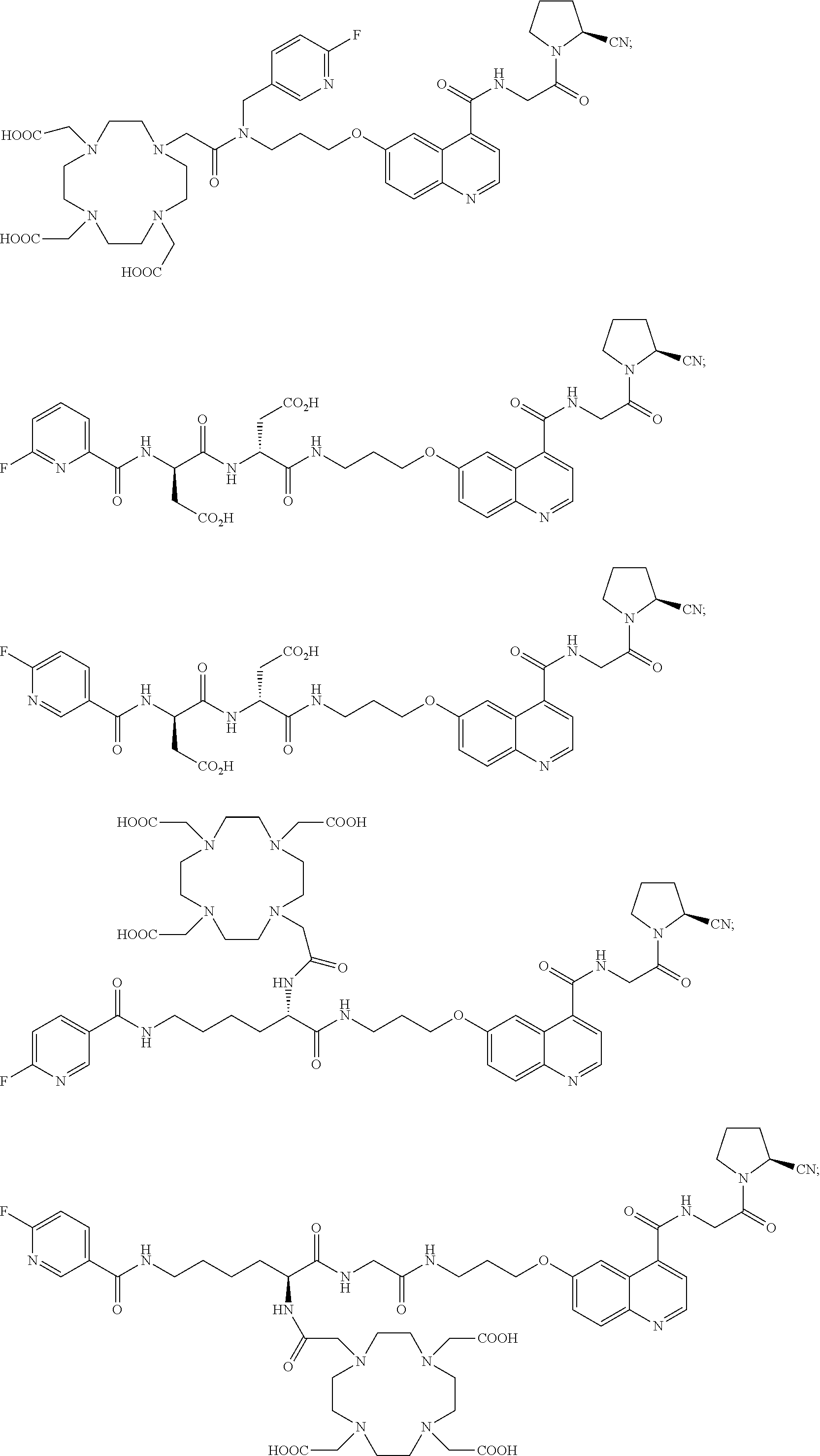

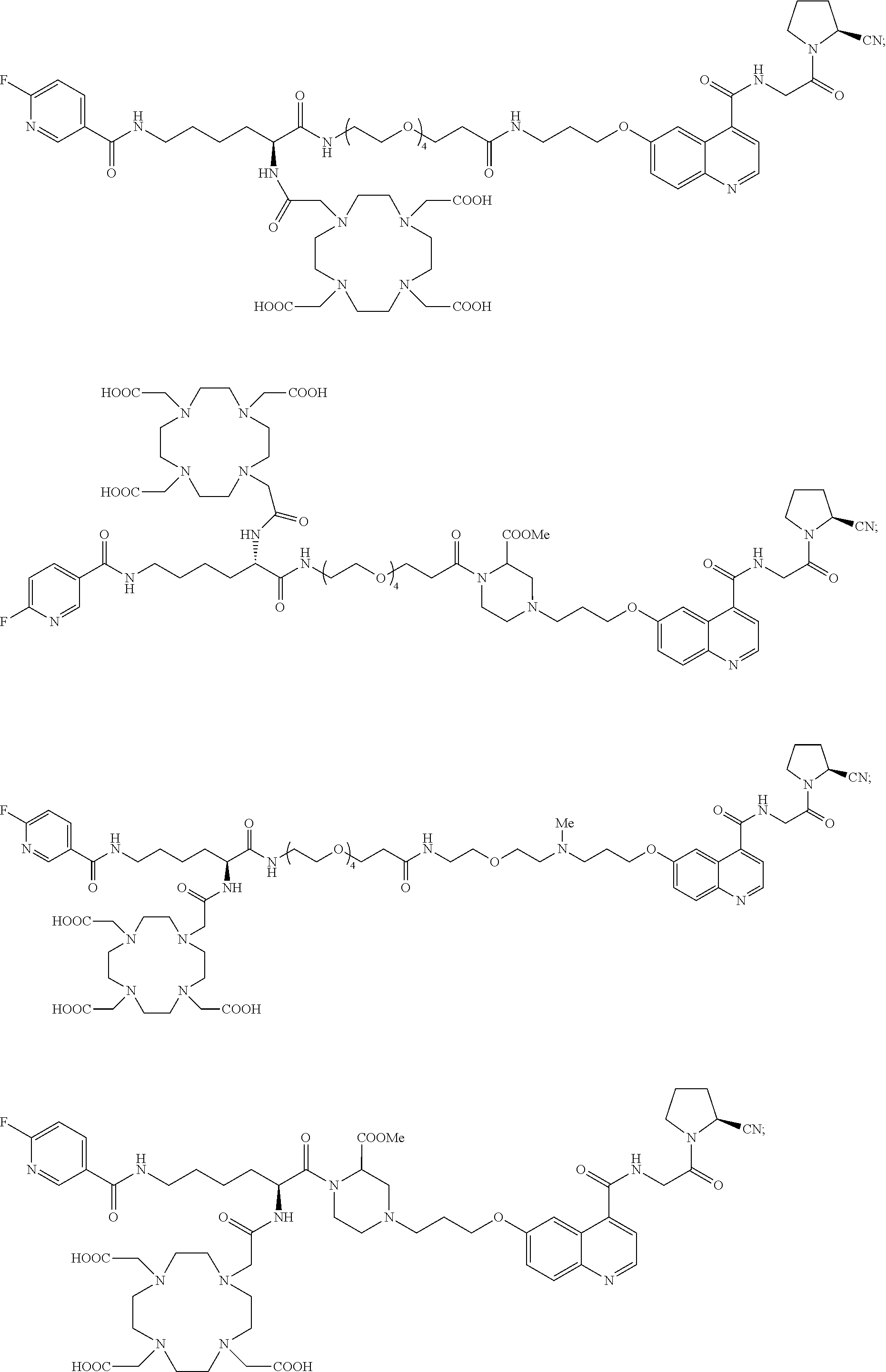

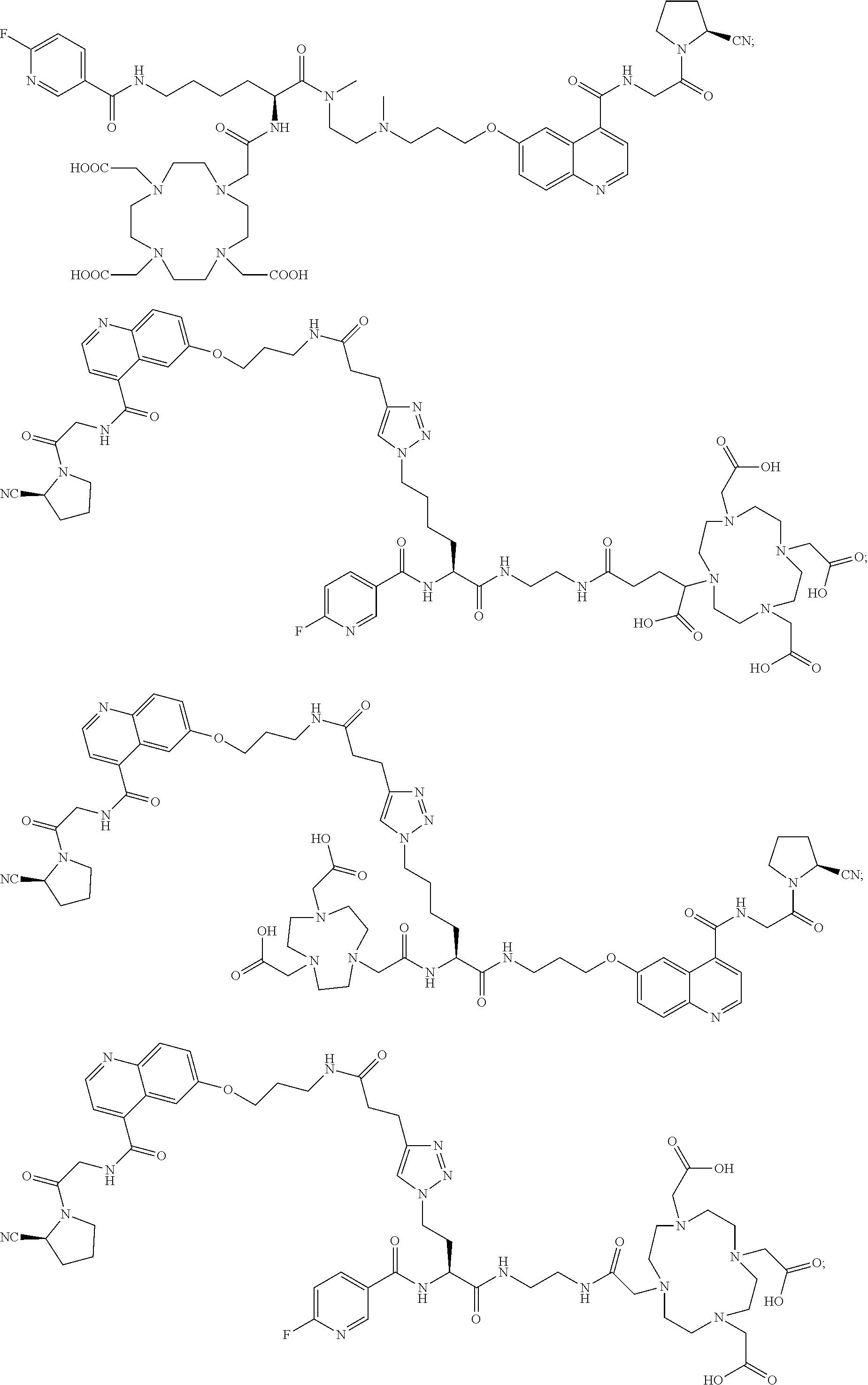

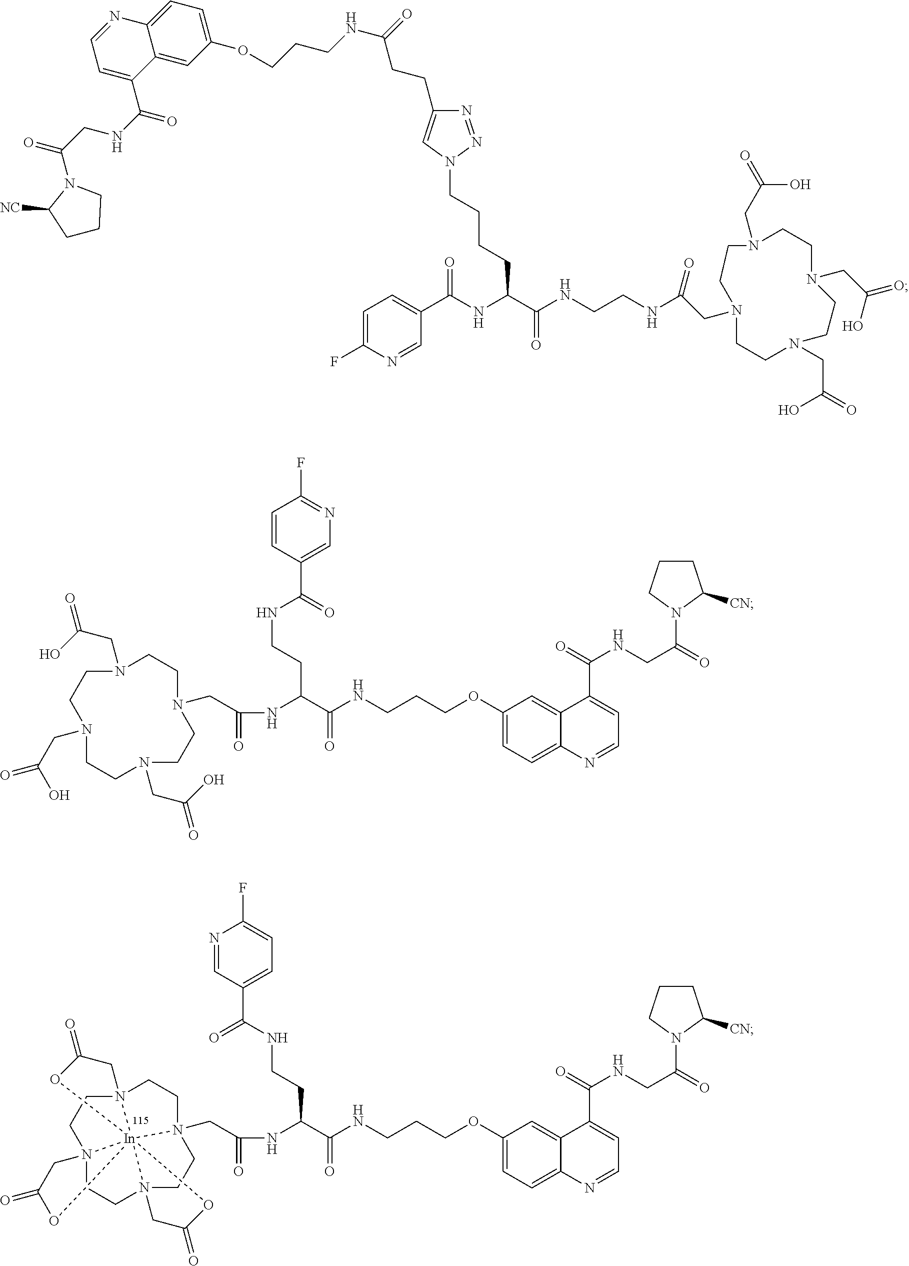

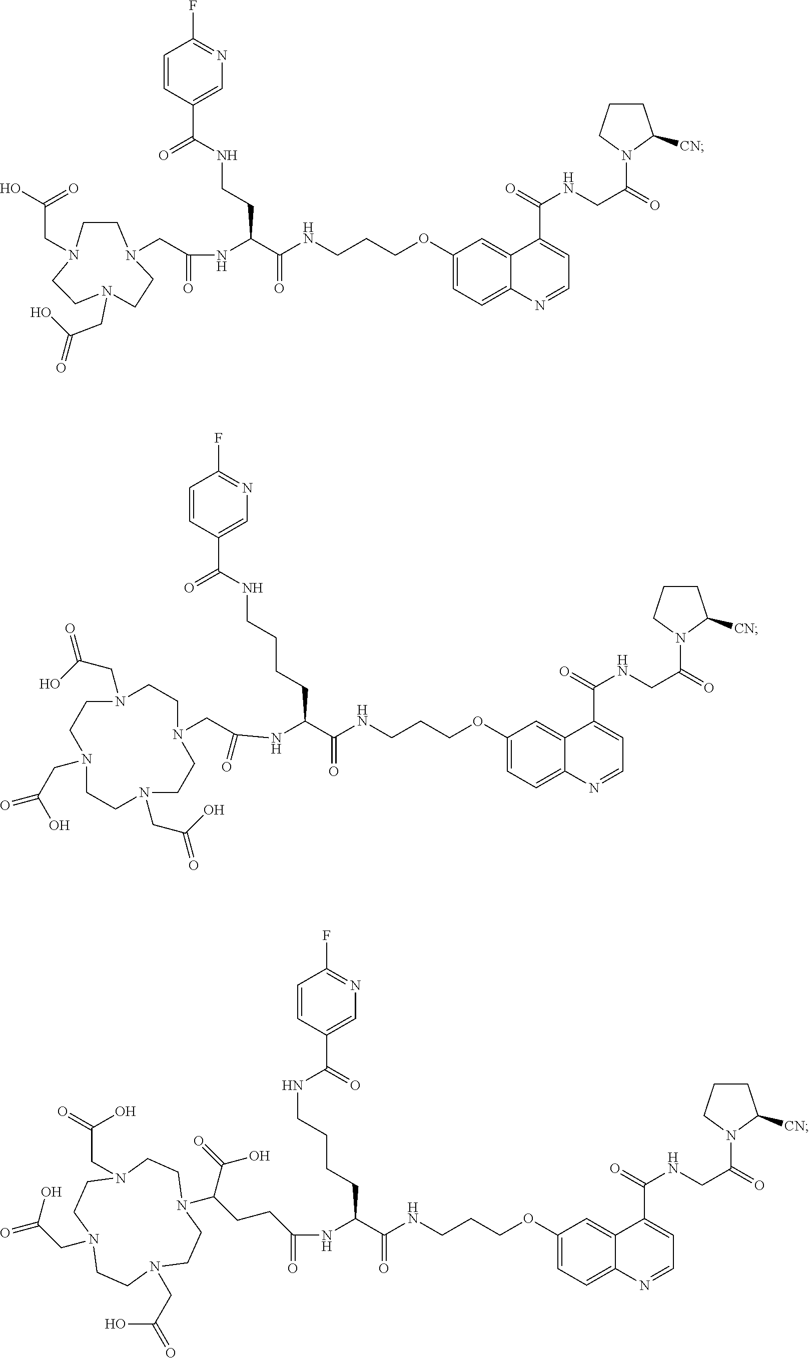 [SMILES: N#C[C@@H]1CCCN1C(=O)CNC(=O)c1ccnc2ccc(OCCCNC(=O)[C@H](CCNC(=O)c3ccc(F)nc3)NC(=O)CN3CCN(CC(=O)O)CCN(CC(=O)O)CC3)cc12 ; N#C[C@@H]1CCCN1C(=O)CNC(=O)c1ccnc2ccc(OCCCNC(=O)[C@H](CCCCNC(=O)c3ccc(F)nc3)NC(=O)CN3CCN(CC(=O)O)CCN(CC(=O)O)CCN(CC(=O)O)CC3)cc12 ; N#C[C@@H]1CCCN1C(=O)CNC(=O)c1ccnc2ccc(OCCCNC(=O)[C@H](CCCCNC(=O)c3ccc(F)nc3)NC(=O)CCC(C(=O)O)N3CCN(CC(=O)O)CCN(CC(=O)O)CCN(CC(=O)O)CC3)cc12]

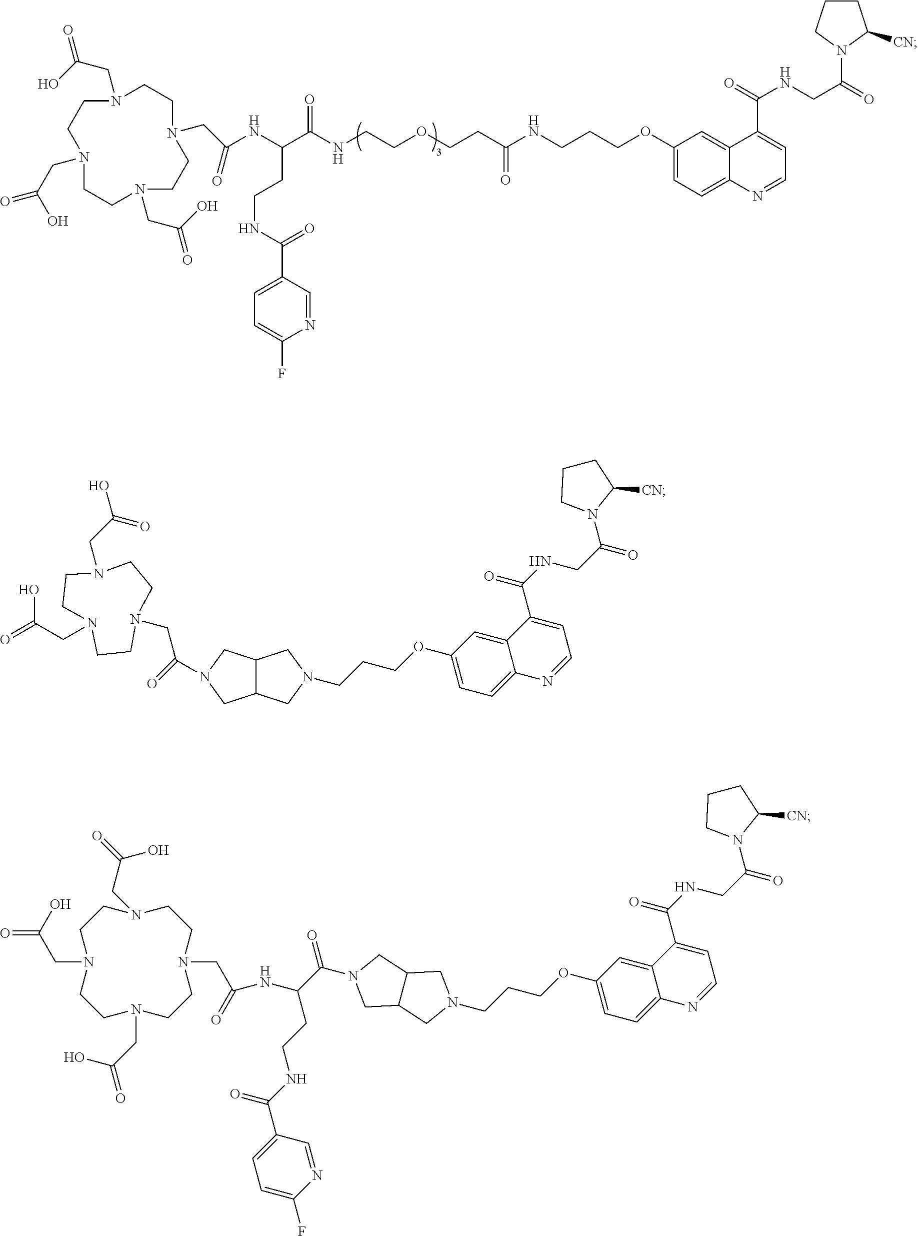

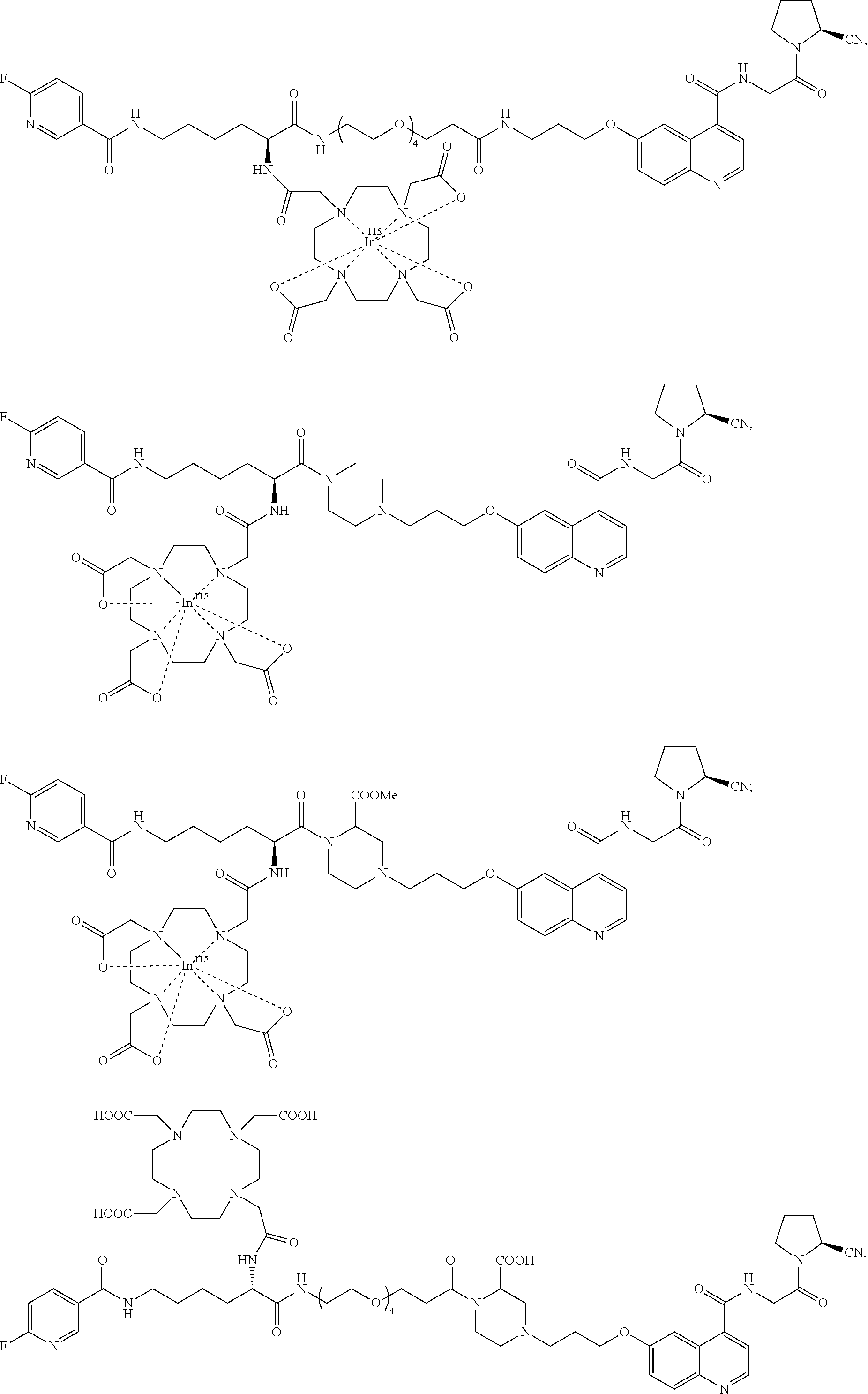

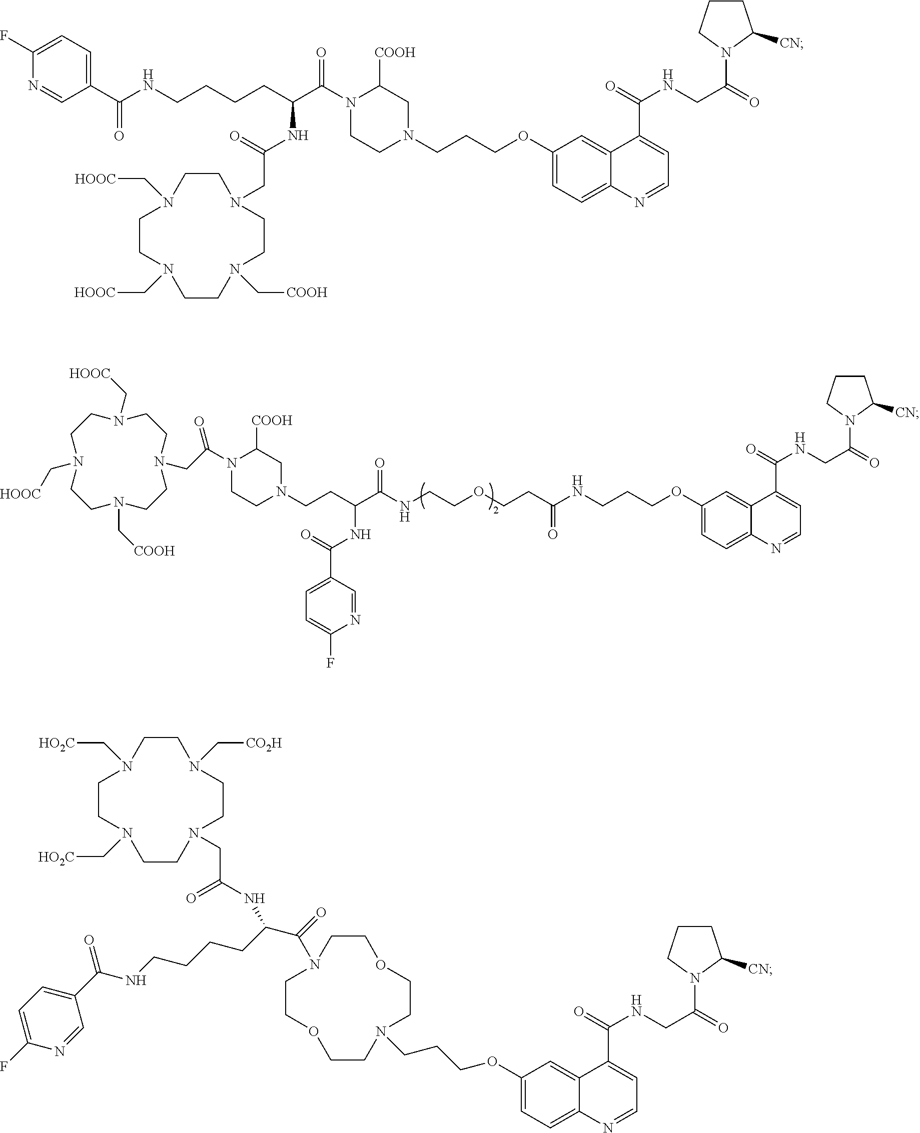

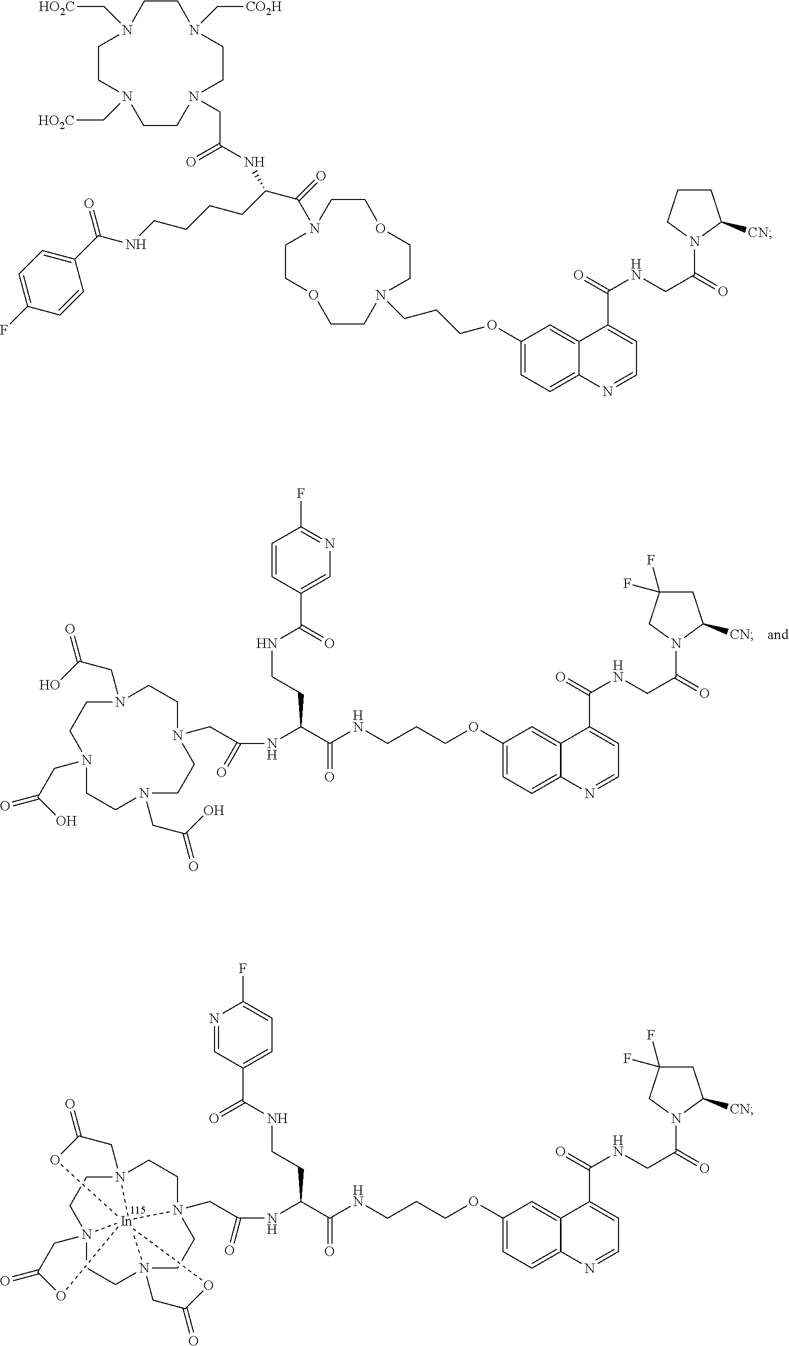

66. The compound of claim 53, wherein the prosthetic group C₂ is covalently bound to the chelating group C₁.

67. The compound of claim 66, wherein the compound is selected from:

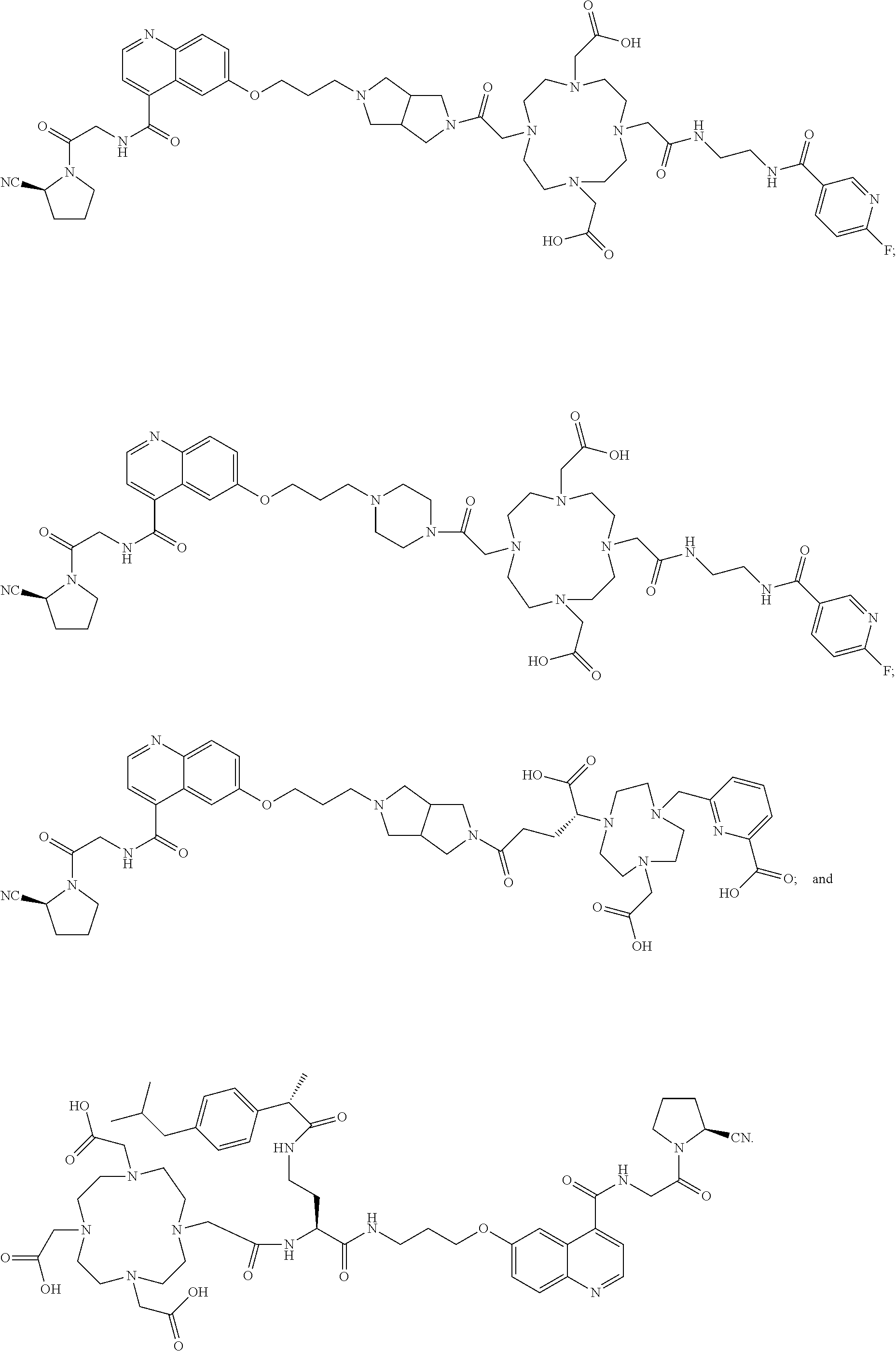

68-91. (canceled)
